SEQUENCE LISTING (3 sequences)

SEQ ID NO: 1 - DNA, 57 bp, synthetic construct
  source 1..57: mol_type = other DNA
aagcagtggtatcaacgcagagtacttttttttttttttttttttttttttttttvn

SEQ ID NO: 2 - DNA, 30 bp, synthetic construct
  source 1..30: mol_type = other DNA
  misc_feature 29..30: note = G29-G30 are ribonucleotides
aagcagtggtatcaacgcagactacatggg

SEQ ID NO: 3 - DNA, 23 bp, synthetic construct
  source 1..23: mol_type = other DNA
aagcagtggtatcaacgcagagt

(19) United States
(12) Patent Application Publication
(10) Pub. No.: US 2023/0112964 A1
(43) Pub. Date: Apr. 13, 2023

(54) ASSESSMENT OF MELANOMA THERAPY RESPONSE

(71) Applicant: UNIVERSITY OF UTAH RESEARCH FOUNDATION, Salt Lake City, UT (US)

(72) Inventors: Robert L. Judson-Torres, Salt Lake City, UT (US); Rachel Belote, Salt Lake City, UT (US)

(21) Appl. No.: 17/929,062

(22) Filed: Sep. 1, 2022

Related U.S. Application Data
(60) Provisional application No. 63/240,308, filed on Sep. 2, 2021.

(51) Int. Cl. C12Q 1/6886

(52) U.S. Cl. CPC C12Q 1/6886; C12Q 1/6869

(57) ABSTRACT

Described herein are methods for stratifying and evaluating melanoma treatment response in a subject using single cell RNA sequencing (scRNA-seq) and a two-step deconvolution analysis and optionally administering a treatment depending on the results. Embodiment described herein are methods for stratifying and evaluating melanoma treatment response in a subject based on single cell or bulk RNA sequencing, bulk transcriptome profiling and/or transcript counting and a two-step deconvolution analysis and optionally administering a treatment depending on the results.

### CROSS-REFERENCE TO RELATED APPLICATIONS

This application claims priority to U.S. Provisional Patent Application No. 63/240,308 filed on Sep. 2, 2021, which is incorporated by reference herein in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “026389-9325-WO01_sequence_listing_xml_24-AUG-2022.xml,” was created on Aug. 24, 2022, contains 3 sequences, has a file size of 3.92 Kbytes, and is incorporated by reference in its entirety into the specification.

### TECHNICAL FIELD

Described herein are methods for stratifying and evaluating melanoma treatment response in a subject using single cell RNA sequencing (scRNA-seq) and a two-step deconvolution analysis and optionally administering a treatment depending on the results. Embodiment described herein are methods for stratifying and evaluating melanoma treatment response in a subject based on single cell or bulk RNA sequencing, bulk transcriptome profiling and/or transcript counting and a two-step deconvolution analysis and optionally administering a treatment depending on the results.

### BACKGROUND

Epidermal melanocytes, the pigment producing cells of human skin, are responsible for skin tone and orchestrate the primary defense against ultraviolet (UV) radiation. Some anatomic site-specific differences in pigmentation are due to environmental factors, such as the tanning response to UV exposure. Others, like the hypopigmentation at volar sites (such as palms and soles), are present at birth. In adult skin, mesenchymal—melanocyte interactions are known to influence anatomic site-specific melanocyte survival and pigment production but melanocyte intrinsic factors that contribute to site-specific specialization remain unclear.

Model organisms are powerful tools for investigating melanocyte development. In chick and mouse, a transient, multipotent neural crest cell population gives rise to committed immature melanocyte precursors, called melanoblasts, via two spatially and temporally distinct pathways. Such studies focus primarily on melanocytes in skin appendages (hair follicle, feather, and sweat gland). However, despite constituting the predominate subtype in human skin, resident epidermal melanocytes have not been the subject of analogous investigations into developmental trajectories and anatomic-specializations.

Melanocytes can give rise to melanomas which present distinct phenotypic and genomic characteristics correlated with primary tumor location. Like many cancers, melanoma progression is coupled to dedifferentiation of the cell of origin. The aggressive nature of melanoma is proposed to be rooted in unique attributes of the melanocytic lineage. Decoding the transcriptome of epidermal melanocytes across the human body during development and in aged skin would provide insight into the precise origins of melanoma and the developmental programs reacquired during progression.

Single cell RNA sequencing (scRNA-seq) characterizes cell heterogeneity with unprecedented resolution. Pioneering studies of human skin with scRNA-seq focused on predominant cell types (keratinocytes, fibroblasts) from few and/or uniform samples and lacked substantial representation of rare cell types, including melanocytes. Consequently, the melanocytes captured were not characterized beyond inter-cell type comparisons. Additionally, single cell sequencing efforts for human fetal tissue have not included the melanocytic lineage.

What is needed is a cell atlas of human epidermal melanocytes during development and aging that captured diversity within and across anatomic locations, sex, and multiple skin tones. This will permit assessment of the response of melanoma cancers to various therapeutic agents.

### SUMMARY

One embodiment described herein is a method of stratifying and evaluating melanoma treatment response in a subject using single cell RNA sequencing (scRNA-seq) and a two-step deconvolution analysis, the method comprising: (a) obtaining a melanoma tumor sample from a subject; (b) performing scRNA-seq of the melanoma tumor sample and obtaining scRNA-seq sequence data; (c) on a processor, deconvoluting the scRNA-seq sequence data using a first gene signature to stratify the melanoma tumor sample into a specific melanoma cell subtype; and (d) deconvoluting the scRNA-seq sequence data using a second gene signature to calculate an estimate of the total number of cells in the melanoma tumor sample that express the second gene signature; wherein when the calculated estimate of total melanoma tumor expression of the second gene signature reaches a critical threshold value, the melanoma tumor will not respond to immune checkpoint inhibition (ICI) treatment. In one aspect, the melanoma is acral melanoma (AM). In another aspect, the method further comprises: when the calculated total melanoma tumor expression of the second gene signature is below the critical threshold value, an effective amount of an ICI treatment is administered to the subject; or when the calculated total melanoma tumor expression of the second gene signature is above the critical threshold value, an effective amount of an alternative non-ICI therapy is administered to the subject. In another aspect, the method further comprises: calculating a transcriptomic deconvolution-based predictor of ICI resistance (TD-IR) score value; wherein when the calculated TD-IR score value is positive, the melanoma tumor will not respond to ICI treatment; or wherein when the calculated TD-IR score value is negative, the melanoma tumor will respond to ICI treatment. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof. In another aspect, the specific melanoma cell subtype comprises volar-like (v-mel) or non-volar cutaneous-like (c-mel) melanocyte-derived melanoma. In another aspect, the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18. In another aspect, when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma. In another aspect, when the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma. In another aspect, the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.

Another embodiment described herein is a method of stratifying and evaluating melanoma treatment response in a subject using single cell RNA sequencing (scRNA-seq) and a two-step deconvolution analysis, the method comprising: (a) obtaining a melanoma tumor sample from a subject; (b) performing scRNA-seq of the melanoma tumor sample and obtaining scRNA-seq sequence data; (c) on a processor, deconvoluting the scRNA-seq sequence data using a first gene signature to stratify the melanoma tumor into a specific melanoma cell subtype; (d) deconvoluting the scRNA-seq sequence data using a second gene signature to calculate an estimate of the total number of cells in the melanoma tumor sample that express the second gene signature; and (e) calculating a transcriptomic deconvolution-based predictor of ICI resistance (TD-IR) score value; wherein when the calculated estimate of total melanoma tumor expression of the second gene signature reaches a critical threshold value, the melanoma tumor will not respond to immune checkpoint inhibition (ICI) treatment; when the calculated TD-IR score value is positive, the melanoma tumor will not respond to ICI treatment; or when the calculated TD-IR score value is negative, the melanoma tumor will respond to ICI treatment. In one aspect, the melanoma is acral melanoma (AM). In another aspect, the method further comprises: when it is determined that the melanoma tumor will respond to ICI treatment, an effective amount of an ICI treatment is administered to the subject; or when it is determined that the melanoma tumor will not respond to ICI treatment, an effective amount of an alternative non-ICI therapy is administered to the subject. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof. In another aspect, the specific melanoma cell subtype comprises volar-like (v-mel) or non-volar cutaneous-like (c-mel) melanocyte-derived melanoma.

Another embodiment described herein is a method of stratifying and evaluating melanoma treatment response in a subject using RNA hybridization, and a two-step deconvolution analysis, the method comprising: (a) obtaining a melanoma tumor sample from the subject; (b) performing RNA hybridization of the melanoma tumor sample using a targeted RNA probe panel to obtain targeted transcript expression data; (c) on a processor, deconvoluting the targeted transcript expression data using a first gene signature from the targeted RNA probe panel to stratify the melanoma into a specific melanoma cell subtype; and (d) deconvoluting the targeted transcript expression data using a second gene signature from the targeted RNA probe panel to calculate an estimate of the total number of cells in the tumor sample that express the second gene signature; wherein when the calculated estimate of total tumor expression of the second gene signature reaches a critical threshold value, the tumor will not respond to immune checkpoint inhibition (ICI) treatment. In one aspect, the melanoma is acral melanoma (AM). In another aspect, the melanoma tumor sample comprises one or more biopsy samples or one or more formalin fixed paraffin embedded (FFPE) tumor tissue samples from the subject. In another aspect, the targeted RNA probe panel comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, and CYC1. In another aspect, the method further comprises: calculating a transcriptomic deconvolution-based predictor of ICI resistance (TD-IR) score value; wherein when the calculated TD-IR score value is positive, the melanoma tumor will not respond to ICI treatment; or wherein when the calculated TD-IR score value is negative, the melanoma tumor will respond to ICI treatment. In another aspect, the method further comprises: when it is determined that the melanoma tumor will respond to ICI treatment, an effective amount of an ICI treatment is administered to the subject; or when it is determined that the melanoma tumor will not respond to ICI treatment, an effective amount of an alternative non-ICI therapy is administered to the subject. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof.

Another embodiment described herein is a method of stratifying and evaluating melanoma treatment response in a subject using bulk transcriptomic data and a two-step deconvolution analysis, the method comprising: (a) obtaining one or more melanoma tumor samples from a subject; (b) performing RNA sequencing of the one or more melanoma tumor samples and obtaining bulk transcriptomic data; (b) performing transcript counting on the bulk transcriptomic data to obtain transcript expression data; (c) on a processor, deconvoluting the transcript expression data using a first gene signature to stratify the melanoma into a specific melanoma cell subtype or origin; and (d) deconvoluting the transcript expression data using a second gene signature to calculate an estimate of the total number of cells in the melanoma tumor sample that express the second gene signature or determine the cell differentation state; wherein when the calculated estimate of total expression of the second gene signature reaches a critical threshold value, the melanoma will not respond to immune checkpoint inhibition (ICI) treatment. In one aspect, the melanoma is acral melanoma (AM). In another aspect, the method further comprises: when the calculated total tumor expression of the second gene signature is below the critical threshold value, an effective amount of an ICI treatment is administered to the subject; or when the calculated total tumor expression of the second gene signature is above the critical threshold value, an effective amount of an alternative non-ICI therapy is administered to the subject. In another aspect, the method further comprises: calculating a transcriptomic deconvolution-based predictor of ICI resistance (TD-IR) score value; wherein when the calculated TD-IR score value is positive, the melanoma tumor will not respond to ICI treatment; or wherein when the calculated TD-IR score value is negative, the melanoma tumor will respond to ICI treatment. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof. In another aspect, the specific melanoma cell subtype comprises volar-like (v-mel) or non-volar cutaneous-like (c-mel) melanocyte-derived melanoma. In another aspect, the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18. In another aspect, when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma. In another aspect, the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma. In another aspect, the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-L show melanocyte transcriptomic profiles differ based on development and anatomic location. FIG. 1A shows fresh from healthy human skin single cell isolation, enrichment, and sequencing pipeline. FIG. 1B shows UMAP visualization of the 9,719 cells (7088 melanocytes, 1865 keratinocytes, 636 eccrine, 76 dendritic, 25 mast, and 29 T-cells) that passed quality control. Colored by cell types identified from Louvain clustering and candidate genes. FIG. 1C shows a heat map showing the relative expression of top differentially expressed genes for 100 random selected cells from each cell-type cluster in FIG. 1B. FIG. 1D-H show UMAPs of all non-cycling melanocytes with Louvain clustering and demographic information overlays. Three low-resolution Louvain clusters correspond to developmental age: adult (cluster A), fetal (cluster B), and neonatal (cluster C). FIG. 1E shows the 11 high-resolution Louvain clusters (0-10) do not correspond to sex (FIG. 1F), skin tone (FIG. 1G), or donor (FIG. 1H). FIG. 1I shows a dot plot of the mean expression and fraction of cells expressing the top 5 ranked genes (two-sided Wilcoxon Rank-sum) for each high-resolution Louvain cluster in FIG. 1E with a hierarchical clustering dendrogram. Using the average expression of the top 15 melanocyte specific principal components, high-resolution Louvain clusters were binned into four groups: m1 (fetal cluster 10), m2 (fetal clusters 3,2,6), m3 (adult clusters 4,1,0,7,5), and m4 (neonatal (neo) clusters 9,8). FIG. 1J shows a dot plot showing group m1, from fetal hair-baring non-volar cutaneous skin, expresses known melanocyte stem cell (MSC) markers. FIG. 1K-L show UMAP of all non-cycling melanocytes with developmental age (FIG. 1K) and fetal MSC annotation based on hierarchical clustering of high-resolution Louvain clusters in FIG. 1I and anatomic location overlay (FIG. 1L).

FIG. 2A-H show the characterization of divergent pigment developmental trajectories in volar and non-volar melanocytes. FIG. 2A shows a schematic illustrating cohort of donor-matched non-volar and volar skin. n=6 donors and 17 total skin specimens. FIG. 2B shows raw and average normalized BSC values (FIG. 2C) of volar and non-volar cutaneous melanocytes prior to 18 weeks (pre-bifurcation) and at/after 18 weeks (post-bifurcation). Two-sided Wilcoxon Rank Sum Test, Bonferroni multiple testing adjusted p-value: ns, p-value=0.25; *p-value=3.6×10⁻¹⁶⁵. Box: interquartile range with median, standard deviation, and outliers (grey circles). FIG. 2D shows Fontana Masson staining for melanin/melanosomes in fetal and adult non-volar and volar skin. Representative images from n=3 for each age. Scale bars 50 μm. FIG. 2E shows increased pigment content coincides with upregulation of the pigment transcriptional program in cutaneous melanocytes at 18 wks. Normalized mean expression of 170 pigment associated genes (thin lines) in volar (blue) and non-volar cutaneous (red) melanocytes. Thick lines: average expression of all pigment associated genes. FIG. 2F shows mean expression of the 14 pigment genes with significant differential expression between non-volar and volar melanocytes from both adult donors with color and size corresponding to fold change between sites. FIG. 2G shows the fold change in expression of the differentially expressed genes in FIG. 2F for each donor matched age. Lineage genes: melanocyte lineage specific genes. Bifurcation-associated: genes with significant differential expression coinciding with pigment bifurcation (between 12 f.w. and 18 f.w.). Post-bifurcation: genes with significant differential expression only in donor matched adult. Unpaired two-tailed t-test * p-value=0.0278; ** p-value=0.0013; *** p-value=3.3×10⁻⁵. Box: full range of data values (min to max) and mean. FIG. 2H shows a schematic summarizing the identification of pigment genes associated with intra-individual pigmentation divergence between non-volar cutaneous and volar melanocytes.

FIG. 3A-K show anatomic site-specific melanocyte sub-population enrichment arises during development and persists in adulthood. FIG. 3A shows a volcano plot of genes enriched (two-sided Wilcoxon Rank Sum Test, Benjamini-Hochberg multiple testing) in donor matched non-volar cutaneous vs volar melanocytes. See also Table 1. FIG. 3B shows top site-specific DEGs. FIG. 3C shows fraction of melanocytes with v-mel or c-mel signature in each skin specimen (n=34) from all 22 donors. Box: 25%, 75%, and median. Two-sided Mann-Whitney U test with Bonferroni multiple testing correction, * p-value=0.12, ** p-value palm: 0.0061, sole: 0.0058 ***p-value=0.00021, ****p-value=8.2×10⁻⁶. FIG. 3D shows expression level of v-mel gene, NTRK2, and c-mel gene, HPGD, in all volar melanocytes (n=1,634 cells) compared to all non-volar cutaneous melanocytes (n=5,192 cells). Two-sided Mann-Whitney U test, ***p-value=1.9×10⁻¹⁰⁰, ****p-value=0. Interquartile range with median, standard deviation, and outliers (grey circles). FIG. 3E shows representative pseudo-colored fluorescent microscopy images from NTRK2, HPGD, and the melanocyte marker DCT (outlined in yellow) mRNA staining in adult volar and non-volar epidermis. Dashed line: epidermal-dermal junction. FIG. 3F shows quantification of NTRK2 and HPGD foci in DCT+ melanocytes in volar (n=44 cells) and non-volar cutaneous skin (n=22) in FIG. 3E. Two-tailed unpaired t-test ***p-value=5.5×10⁻⁶, ****p-value=4.1×10⁻⁷; box: 25%, 75%, median; whiskers: 10-90%, and outliers (grey circles). FIG. 3G shows the percent v-mel (NTRK2>HPGD) and c-mel (HPGD>NTRK2) at each site in FIG. 3E-F; j two-tailed, two-sample Z-test for proportions, v-mel: z=6.062, p-value=1.8×10⁻¹¹, and c-mel: z=7.885, p-value=1.6×10⁻¹⁵. FIG. 3H shows immunofluorescence co-staining of adult volar and non-volar skin cryo-sections with the c-mel marker HPGD (green) and melanocyte marker KIT (magenta). Dashed line: epidermal-dermal junction. FIG. 3I shows percent HPGD positive melanocytes per donor volar and non-volar skin. Adult skin: A1046, n=78 cells; A1038, n=39 cells; A1018, n=48 cells; A1026, n=15 cells. Fetal skin: 9WK07, n=41 cells; 16WK04, n=10 cells. Two-tailed unpaired t-test, ** p-value=0.001. FIG. 3J shows an illustration depicting the hypothesis that healthy melanocyte anatomic site-specific transcriptional programs are conserved in melanoma. FIG. 3K shows the ratio of the average expression of the top v-mel and c-mel genes in primary melanomas (acral: n=15, non-acral cutaneous: n=103), unpaired two-tailed t-test, ****p-value=1.5×10⁻⁶; box: 25%, 75%, median; whiskers: 10-90%, and outliers (grey circles).

FIG. 4A-F show defining human specific melanocyte developmental transcriptomic programs. FIG. 4A shows a heatmap of the median Normalized Enrichment Scores (NES)s of GO-bp terms enriched at each developmental stage. FIG. 4B shows a schematic of the Developmental stage Melanocyte logistical regression model (DevMel LOGIT) used to generate and validate unique transcription profiles for each developmental stage of normal human melanocytes. The bottom of FIG. 4B shows a heatmap of the relative expression (column z score) of genes in each DevMel program (prg). FIG. 4C-F show DevMel program expression is highly expressed by cells from all skin donors within each corresponding developmental stage. Program expression for each donor (black line, average) is the ratio of the mean expression of positively correlated genes and negatively correlated genes. Significant by one-sided Mann Whitney U test, MSC n=3; FET n=5; NEO n=2; ADT n=14 donors. FIG. 4C: prg[MSC]: MSC vs rest *** p-value=0.0005; FIG. 4D: prg[FET]: FET vs rest **** p-value=0.0001; FIG. 4E: prg[NEO]: FET vs rest ** p-value=0.0072; FIG. 4F: prg[ADT]: ADT vs rest **** p-value=5.1×10⁻⁷.

FIG. 5A-G shown the evaluation of model mammalian melanocyte developmental program expression in human non-volar cutaneous melanocyte developmental groups. FIG. 5A shows a schematic summarizing human and corresponding mouse melanocyte development. In hair-baring skin, both humans and mice develop follicular melanocytes (purple). Mice retain a dermal melanocyte population (blue) in fully developed skin, whereas humans develop resident epidermal melanocytes (red) within the skin at all anatomic locations. Pink bar indicates human fetal ages captured in this study's dataset. FIG. 5B-C show violin plots showing distribution of indicated transcriptional program expression scores for individual cells within each developmental group: ADT n=3281, NEO n=735, FET n=1176, and MSC n=63. Dashed line: mean expression. Program scores were generated from published signatures of: FIG. 5B shows mouse melanoblasts (cells committed to the melanocyte fate)**** p-value=1.5×10⁻¹⁴; ** p-value=1.0×10⁻⁸,*** p-value=2.3×10⁻¹⁹,**** p-value=5.9×10⁻²⁸; mouse melanocytes,**** p-value=2.8×10⁻¹³; melanocyte stem cells from mature hair follicles in adult mice, **** p-value=4.5×10⁻³⁸. FIG. 5C shows in vitro stages of differentiation of human pluripotent stem cells to melanocytes,**** p-value=2.8×10⁻²⁵. Significance determined by one-sided Man-Whitney U test, **** p-value <1×10⁻⁷. The variance (reported below the corresponding group for each violin plot) of the average program expression among donors within the MSC, FET, NEO, and ADT groups was low showing concordance across ages within each group. FIG. 5D-E show Venn diagrams showing the number of unique and overlapping genes of melanoblast-related gene signatures with the positive correlated component of the DevMel profiles prg[MSC] (FIG. 5D) and prg[FET] (FIG. 5E). FIG. 5F-G show Venn diagrams showing the number of unique and overlapping genes of differentiated melanocyte related gene signatures with the positively correlated component of the DevMel profiles prg[NEO] (FIG. 5GF) and prg[ADT] (FIG. 5G).

FIG. 6A-G show the identification of distinct patterns of developmental programs reacquired in metastasized melanomas. FIG. 6A shows DevMel LOGIT was used to classify individual melanoma cells by normal melanocyte developmental stages. Every melanoma cell (MAL) was categorized by the predominantly expressed developmental stage program. FIG. 6B shows individual tumors are a heterogeneous mix of malignant cells in different dedifferentiation states. Fraction of MAL^{ADT}, MAL^{NEO}, MAL^{FET} and MAL^{MSC} cells in each of the 14 tumors analyzed from Tirosh et al., Science 352(6282):189-196 (2016) and Jerby-Arnon et al., Cell 175(4):984-997 (2018) in FIG. 6A. FIG. 6C shows Top: Workflow to generate gene set (511 unique genes) used to identify patterns associated with melanoma dedifferentiation. Bottom: Percent of genes across MAL groups that exhibit patterns consistent with dedifferentiation categories in (FIGS. 6 D, E, and G. FIG. 6D-G show dedifferentiation can occur through several categories of cancer-associated transcriptional reprogramming: FIG. 6D shows sequential dedifferentiation, a reverse stepwise unfolding of development; FIG. 6E shows direct dedifferentiation, direct reacquisition of programs from early developmental stages; FIG. 6F shows melanoma specific, acquisition of programs not associated with the stages of melanocyte development identified here. FIG. 6G shows normal adult developmental stage programs that are lost and earlier developmental stage programs that are not readopted in metastatic melanoma. Examples of each category are visualized as heatmaps of the relative expression (row z score). See Table 3 for complete gene lists.

FIG. 7A-F show the reacquisition of specific developmental programs in heterogeneous melanoma is prognostic. FIG. 7A shows the hierarchical clustering of TOGA SKCM tumors based on fractional composition of normal melanocyte developmental stages assigned using CIBERSORT (top) with clinicopathological features (bottom panels). FIG. 7B shows Kaplan Meier curves (two-side, log-rank test) for each SKCM group from FIG. 7A. Enrichment for cells similar to ADT is associated with increased survival, whereas enrichment for NEO is associated with worse survival. FIG. 7C shows Kaplan Meier curve (two-side, log-rank test) showing enrichment of NEO fraction is associated with worse survival in second cohort (Lund University). FIG. 7D shows the fraction of MAL^{NEO} cells from the single cell tumors (untreated n=7, resistant n=7) from FIG. 5B correlates with post-ICI resistance. Unpaired one-sided t-test, ** p-value=0.0099. Black bar: mean. FIG. 7E shows expression of the MAL^{NEO} signature (Top 100 DEGs, see Table 4) is significantly higher in tumors from patients exhibiting only partial (PR, n=25) or no response (PD, n=49) to anti-PD1 treatment compared to those that responded (CR, n=14). Unpaired one-side t-test, * p-value=0.018; Box: median, 25%, 75%; whiskers: min-max. FIG. 7F shows a schematic summarizing the decoding of melanoma dedifferentiation using human developmental programs. The left panel of FIG. 7F show that individual melanoma tumors are comprised of a heterogeneous mix of malignant cells expressing defined melanocyte developmental programs. The fraction of cells expressing each program within the tumor is predictive of overall survival and correlates to signatures of immune infiltration, evasion, and potential therapeutic options. The right panel of FIG. 7F shows that each melanoma cell can occupy a different degree of dedifferentiation defined by sequential dedifferentiation transcriptional programs. See FIG. 6A-G and Table 3. The MSC- and adult-like programs are associated with previously described melanoma signatures whereas the fetal- and neonatal-like programs do not segregate with known melanoma signatures offering unique insight into previously uncharacterized melanoma transcriptional states (see FIG. 8). Melanoma specific genes: genes common to melanoma cells but not melanocytes, such as PRAME. Direct dedifferentiation genes: MSC or FET genes that can be expressed in melanoma cells regardless of the over-all differentiation state of the cell, such as AXL, EGR1 and HMGA2.

FIG. 8A-H show the characterization of melanoma cells and tumors classified by in situ human melanocyte developmental programs. FIG. 8A-B show density plots showing the expression of the Widmer et al. invasive and proliferative programs (FIG. 8A) and the Tirosh et al (FIG. 8B). AXL and MITF programs for individual cells in MALADT, MALNEO, MALFET and MALMSC groups. FIG. 8C shows pairwise Fisher (one-sided) exact test showing negative log 10 adjusted (Bonferroni multiple testing) p-values for the gene set enrichment analysis conducted using gene signatures from Akbani et al., Cell 161: 1681-1696 (2015); Cirenajwis et al., Oncotarget 6: 12297-12309 (2015); and Tsoi et al., Cancer Cell 33: 890-904.e5 (2018). Significant enrichment determined as adjusted p-value <0.05. FIG. 8D shows a heatmap illustrating the relative expression levels (row z score) of WNT5A high, TP53 high slow cycling cell associated genes in each normal melanocyte and MAL developmental group. FIG. 8E shows a heatmap illustrating the relative expression levels (row z score) of the four minimal residual disease states identified by Rambow et al., Cell 174: 843-855.e19 (2018) in each normal melanocyte and MAL developmental group. FIG. 8F shows pairwise Fisher (one-sided) exact test showing negative log 10 adjusted (Bonferroni multiple testing) p-values for clinicopathological feature and transcriptional categorization within each SKCM group (SKCMADT, SKCMNEO, SKCMFET, SKCMMSC). There is little to no difference in the enrichment of pigment level, mutation category, or tissue origin between SKCM groups in FIG. 7. FIG. 8G shows a heatmap illustrating the relative expression levels (row z-score) of immune infiltration program, immune evasion program and FDA-approved therapeutic targets in SKCM groups. FIG. 8H shows the MALNEO signature is enriched for genes down regulated in tumors that respond to Nivolumab treatment (green text). Pairwise Fisher (one-sided) exact test showing negative log 10 adjusted (Bonferroni multiple testing) p-values for the gene set enrichment analysis conducted using previously identified prognostic signatures (Table 4).

FIG. 9 shows a scheme for the discovery of two types of melanocytes in adult human skin affected by AM was used to develop classifier that determines the melanocyte cell of origin for individual AM tumors.

FIG. 10 shows a scheme for deconvolving melanoma tumors using healthy melanocyte developmental states identified a dedifferentiated state (neonatal) that was resistant to ICI (anti-PD1) and stratified tumors based on response to anti-PD1 therapy.

FIG. 11A shows one-step deconvolution to estimate percent of ICI-R melanoma cells does not predict response in AM. FIG. 11B shows that the TD-IR method uses a two-step deconvolution to (1) classify tumors as cAM or vAM prior to (2) estimating the percent ICI-R. When the TD-IR method is applied to pretreated AM tumor transcriptomic data the resulting single value score is predictive of anti-PD1 response.

FIG. 12A shows pooling of melanocyte specific RNAscope probes into a melanocyte cocktail provides robust cell-type (state) specific staining in FFPE sections. FIG. 12B shows exemplary RNAscope cocktails for classifying AM by cell of origin and ICI-R cell state.

FIG. 13 shows the sensitivity of NanoString probes are determined using sections from FFPE embedded cell pellets comprising different ratios of cAM/vAM/ICI-R cells mixed with non-cAM/vAM/ICI-R melanocytes.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “bulk transcriptomic data” refers to mRNA transcript data obtained using any technology for assessing average mRNA transcript levels of a mass, group, or population of cells averaged together. For example, “bulk transcriptomic data” could refer to mRNA transcript levels for the entire mixture of cells isolated from a tumor. Typically, such data would include RNA sequencing (RNAseq) and transcript counting (e.g., using NanoString as described herein).

As used herein, “transcript counting” refers to assessing the number of sequences or hybridization probes mapped to each gene as a measure of gene expression. For example, NanoString as described herein provides a hybridization-based technology that permits targeted transcript counting, without amplification and produces highly reproducible gene expression patterns.

As used herein, “immune checkpoint inhibitors” (las) are compounds activate the antitumor immune response by interrupting co-inhibitory signaling pathways and promote immune-mediated elimination of tumor cells. Immune checkpoint inhibitors (las) are approved to treat a variety of cancers, including: breast cancer, bladder cancer, cervical cancer, colon cancer, head and neck cancer, Hodgkin lymphoma, liver cancer, lung cancer, renal cell cancer, skin cancer, including melanoma, stomach cancer, rectal cancer, or any solid tumor that is not able to repair DNA errors occurring during DNA replication. Typical immune checkpoint inhibitors categorized by their targets. PD-1 inhibitors are monoclonal antibodies that target PD-1. Exemplary approved drugs include: Pembrolizumab (Keytruda®), Nivolumab (Opdivo®), and Cemiplimab (Libtayo®). D-L1 inhibitors are monoclonal antibodies that target PD-L1. Exemplary approved drugs include Atezolizumab (Tecentriq®), Avelumab (Bavencio®), and Durvalumab (Imfinzi®). LAG-3 inhibitors are monoclonal antibodies that target LAG-3. Relatlimab is often coadministered with given along with the PD-1 inhibitor Nivolumab (Opidivo®) in a combination therapeutic known as Opdualag® (nivolumab and relatlimab-rmbw). Opdualag is effective against melanoma and other cancers.

As used herein, “non-immune checkpoint inhibitor therapies” (non-ICI therapy) are cancer or melanoma treatments or therapies not involving immune checkpoint inhibitors. Typical therapies include one or more of surgical excision of the tumor; PARP inhibitors including olaparib (Lynparza®), niraparib (Zejula®), rucaparib (Rubraca®), talazoparib (Talzenna®); BRAF inhibitors including dabrafenib (Tafinlar®), encorafenib (Braftovi®), Vemurafenib (Zelboraf®, combinations of vemurafenib and atezolizumab (Tecentriq®); MEK inhibitors including: trametinib (Mekinist), cobimetinib (Cotellic), and binimetinib (Mektovi); KIT inhibitors including: dasatinib (Sprycel®), imatinib (Gleevec®), and nilotinib (Tasigna®); tumor-agnostic treatments: larotrectinib (Vitrakvi®) and entrectinib (Rozlytrek®); CTLA-4 inhibitors including ipilimumab (Yervoy®); interleukin-2 (IL-2, Proleukin); interferon alfa-2b (Intron A®) or pegylated interferon alfa-2b (Sylatron®); combinations of BRAF inhibitors and MEK inhibitors; chemotherapies including dacarbazine (DTIC®), temozolomide (Temodar®), cisplatin, carboplatin, fotemustine (Muphoran®), iomustine (Gleostine®), docetaxel (Taxotere®), paclitaxel (Taxol®), vinblastine, or combinations such as paclitaxel plus carboplatin or cisplatin plus vinblastine and dacarbazine; or combinations thereof.

In humans, epidermal melanocytes are responsible for skin pigmentation, defense against ultraviolet radiation, and the deadliest common skin cancer, melanoma. While there is substantial overlap in melanocyte, development pathways between different model organisms, species dependent differences are frequent and the conservation of these processes in human skin remains unresolved. Here, a single-cell enrichment and RNA-sequencing pipeline was used to study human epidermal melanocytes directly from skin, capturing transcriptomes across different anatomic sites, developmental age, sexes, and multiple skin tones. The study uncovered subpopulations of melanocytes exhibiting anatomic site-specific enrichment that occurs during gestation and persists through adulthood. The transcriptional signature of the volar-enriched subpopulation is retained in acral melanomas. In addition, human melanocyte differentiation transcriptional programs were identified that are distinct from gene signatures generated from model systems. Finally, these programs were used to define patterns of dedifferentiation that are predictive of melanoma prognosis and response to immune checkpoint inhibitor therapy.

One embodiment described herein is a method of stratifying and evaluating melanoma treatment response in a subject using single cell RNA sequencing (scRNA-seq) and a two-step deconvolution analysis, the method comprising: (a) obtaining a melanoma tumor sample from a subject; (b) performing scRNA-seq of the melanoma tumor sample and obtaining scRNA-seq sequence data; (c) on a processor, deconvoluting the scRNA-seq sequence data using a first gene signature to stratify the melanoma tumor sample into a specific melanoma cell subtype; and (d) deconvoluting the scRNA-seq sequence data using a second gene signature to calculate an estimate of the total number of cells in the melanoma tumor sample that express the second gene signature; wherein when the calculated estimate of total melanoma tumor expression of the second gene signature reaches a critical threshold value, the melanoma tumor will not respond to immune checkpoint inhibition (ICI) treatment. In one aspect, the melanoma is acral melanoma (AM). In another aspect, the method further comprises: when the calculated total melanoma tumor expression of the second gene signature is below the critical threshold value, an effective amount of an ICI treatment is administered to the subject; or when the calculated total melanoma tumor expression of the second gene signature is above the critical threshold value, an effective amount of an alternative non-ICI therapy is administered to the subject. In another aspect, the method further comprises: calculating a transcriptomic deconvolution-based predictor of ICI resistance (TD-IR) score value; wherein when the calculated TD-IR score value is positive, the melanoma tumor will not respond to ICI treatment; or wherein when the calculated TD-IR score value is negative, the melanoma tumor will respond to ICI treatment. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof. In another aspect, the specific melanoma cell subtype comprises volar-like (v-mel) or non-volar cutaneous-like (c-mel) melanocyte-derived melanoma. In another aspect, the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18. In another aspect, when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma. In another aspect, when the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma. In another aspect, the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.

Another embodiment described herein is a method of stratifying and evaluating melanoma treatment response in a subject using single cell RNA sequencing (scRNA-seq) and a two-step deconvolution analysis, the method comprising: (a) obtaining a melanoma tumor sample from a subject; (b) performing scRNA-seq of the melanoma tumor sample and obtaining scRNA-seq sequence data; (c) on a processor, deconvoluting the scRNA-seq sequence data using a first gene signature to stratify the melanoma tumor into a specific melanoma cell subtype; (d) deconvoluting the scRNA-seq sequence data using a second gene signature to calculate an estimate of the total number of cells in the melanoma tumor sample that express the second gene signature; and (e) calculating a transcriptomic deconvolution-based predictor of ICI resistance (TD-IR) score value; wherein when the calculated estimate of total melanoma tumor expression of the second gene signature reaches a critical threshold value, the melanoma tumor will not respond to immune checkpoint inhibition (ICI) treatment; when the calculated TD-IR score value is positive, the melanoma tumor will not respond to ICI treatment; or when the calculated TD-IR score value is negative, the melanoma tumor will respond to ICI treatment. In one aspect, the melanoma is acral melanoma (AM). In another aspect, the method further comprises: when it is determined that the melanoma tumor will respond to ICI treatment, an effective amount of an ICI treatment is administered to the subject; or when it is determined that the melanoma tumor will not respond to ICI treatment, an effective amount of an alternative non-ICI therapy is administered to the subject. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof. In another aspect, the specific melanoma cell subtype comprises volar-like (v-mel) or non-volar cutaneous-like (c-mel) melanocyte-derived melanoma.

Another embodiment described herein is a method of stratifying and evaluating melanoma treatment response in a subject using RNA hybridization, and a two-step deconvolution analysis, the method comprising: (a) obtaining a melanoma tumor sample from the subject; (b) performing RNA hybridization of the melanoma tumor sample using a targeted RNA probe panel to obtain targeted transcript expression data; (c) on a processor, deconvoluting the targeted transcript expression data using a first gene signature from the targeted RNA probe panel to stratify the melanoma into a specific melanoma cell subtype; and (d) deconvoluting the targeted transcript expression data using a second gene signature from the targeted RNA probe panel to calculate an estimate of the total number of cells in the tumor sample that express the second gene signature; wherein when the calculated estimate of total tumor expression of the second gene signature reaches a critical threshold value, the tumor will not respond to immune checkpoint inhibition (ICI) treatment. In one aspect, the melanoma is acral melanoma (AM). In another aspect, the melanoma tumor sample comprises one or more biopsy samples or one or more formalin fixed paraffin embedded (FFPE) tumor tissue samples from the subject. In another aspect, the targeted RNA probe panel comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, and CYC1. In another aspect, the method further comprises: calculating a transcriptomic deconvolution-based predictor of ICI resistance (TD-IR) score value; wherein when the calculated TD-IR score value is positive, the melanoma tumor will not respond to ICI treatment; or wherein when the calculated TD-IR score value is negative, the melanoma tumor will respond to ICI treatment. In another aspect, the method further comprises: when it is determined that the melanoma tumor will respond to ICI treatment, an effective amount of an ICI treatment is administered to the subject; or when it is determined that the melanoma tumor will not respond to ICI treatment, an effective amount of an alternative non-ICI therapy is administered to the subject. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof.

Another embodiment described herein is a method of stratifying and evaluating melanoma treatment response in a subject using bulk transcriptomic data and a two-step deconvolution analysis, the method comprising: (a) obtaining one or more melanoma tumor samples from a subject; (b) performing RNA sequencing of the one or more melanoma tumor samples and obtaining bulk transcriptomic data; (b) performing transcript counting on the bulk transcriptomic data to obtain transcript expression data; (c) on a processor, deconvoluting the transcript expression data using a first gene signature to stratify the melanoma into a specific melanoma cell subtype or origin; and (d) deconvoluting the transcript expression data using a second gene signature to calculate an estimate of the total number of cells in the melanoma tumor sample that express the second gene signature or determine the cell differentation state; wherein when the calculated estimate of total expression of the second gene signature reaches a critical threshold value, the melanoma will not respond to immune checkpoint inhibition (ICI) treatment. In one aspect, the melanoma is acral melanoma (AM). In another aspect, the method further comprises: when the calculated total tumor expression of the second gene signature is below the critical threshold value, an effective amount of an ICI treatment is administered to the subject; or when the calculated total tumor expression of the second gene signature is above the critical threshold value, an effective amount of an alternative non-ICI therapy is administered to the subject. In another aspect, the method further comprises: calculating a transcriptomic deconvolution-based predictor of ICI resistance (TD-IR) score value; wherein when the calculated TD-IR score value is positive, the melanoma tumor will not respond to ICI treatment; or wherein when the calculated TD-IR score value is negative, the melanoma tumor will respond to ICI treatment. In another aspect, the ICI treatment comprises: a PD-1 inhibitor selected from pembrolizumab, nivolumab, cemiplimab, or combinations thereof; a PD-L1 inhibitor selected from atezolizumab, avelumab, durvalumab, or combinations thereof; a LAG-3 inhibitor selected from relatlimab, relatlimab-RMBW, or combinations thereof; or combinations thereof. In another aspect, the alternative non-ICI therapy comprises: a PARP inhibitor selected from olaparib, niraparib, rucaparib, talazoparib, or combinations thereof; a BRAF inhibitor selected from dabrafenib, encorafenib, vemurafenib, or combinations thereof; a MEK inhibitor selected from trametinib, cobimetinib, binimetinib, or combinations thereof; a KIT inhibitor selected from dasatinib, imatinib, nilotinib, or combinations thereof; a tumor-agnostic therapy selected from larotrectinib, entrectinib, or combinations thereof; a CTLA-4 inhibitor selected from ipilimumab; aldesleukin (Interleukin-2; IL-2), Interferon alfa-2b, pegylated Interferon alfa-2b, or combinations thereof; a chemotherapeutic agent selected from dacarbazine, temozolomide, cisplatin, carboplatin, fotemustine, lomustine, docetaxel, paclitaxel, vinblastine, or combinations thereof; surgical excision; or combinations thereof. In another aspect, the specific melanoma cell subtype comprises volar-like (v-mel) or non-volar cutaneous-like (c-mel) melanocyte-derived melanoma. In another aspect, the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18. In another aspect, when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma. In another aspect, the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma. In another aspect, the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method of stratifying and evaluating melanoma treatment response in a subject using single cell RNA sequencing (scRNA-seq) and a two-step deconvolution analysis, the method comprising:
   (a) obtaining a melanoma tumor sample from a subject;
   (b) performing scRNA-seq of the melanoma tumor sample and obtaining scRNA-seq sequence data;
   (c) on a processor, deconvoluting the scRNA-seq sequence data using a first gene signature to stratify the melanoma tumor sample into a specific melanoma cell subtype; and
   (d) deconvoluting the scRNA-seq sequence data using a second gene signature to calculate an estimate of the total number of cells in the melanoma tumor sample that express the second gene signature;
   wherein when the calculated estimate of total melanoma tumor expression of the second gene signature reaches a critical threshold value, the melanoma tumor will not respond to immune checkpoint inhibition (ICI) treatment.
Clause 2. The method of clause 1, wherein the melanoma is acral melanoma (AM).
Clause 3. The method of clause 1 or 2, further comprising:
   when the calculated total melanoma tumor expression of the second gene signature is below the critical threshold value, an effective amount of an ICI treatment is administered to the subject; or
   when the calculated total melanoma tumor expression of the second gene signature is above the critical threshold value, an effective amount of an alternative non-ICI therapy is administered to the subject.
Clause 4. The method of any one of clauses 1-3, further comprising:
   calculating a transcriptomic deconvolution-based predictor of ICI resistance (TD-IR) score value;
   wherein when the calculated TD-IR score value is positive, the melanoma tumor will not respond to ICI treatment; or
   wherein when the calculated TD-IR score value is negative, the melanoma tumor will respond to ICI treatment.
Clause 5. The method of any one of clauses 1-4, wherein the ICI treatment comprises:
   a PD-1 inhibitor selected from pembrolizumab, nivolumab, cemiplimab, or combinations thereof;
   a PD-L1 inhibitor selected from atezolizumab, avelumab, durvalumab, or combinations thereof;
   a LAG-3 inhibitor selected from relatlimab, relatlimab-RMBW, or combinations thereof; or combinations thereof.
Clause 6. The method of any one of clauses 1-5, wherein the alternative non-ICI therapy comprises:
   a PARP inhibitor selected from olaparib, niraparib, rucaparib, talazoparib, or combinations thereof;
   a BRAF inhibitor selected from dabrafenib, encorafenib, vemurafenib, or combinations thereof;
   a MEK inhibitor selected from trametinib, cobimetinib, binimetinib, or combinations thereof;
   a KIT inhibitor selected from dasatinib, imatinib, nilotinib, or combinations thereof;
   a tumor-agnostic therapy selected from larotrectinib, entrectinib, or combinations thereof;
   a CTLA-4 inhibitor selected from ipilimumab;
   aldesleukin (Interleukin-2; IL-2), Interferon alfa-2b, pegylated Interferon alfa-2b, or combinations thereof;
   a chemotherapeutic agent selected from dacarbazine, temozolomide, cisplatin, carboplatin, fotemustine, lomustine, docetaxel, paclitaxel, vinblastine, or combinations thereof;
   surgical excision; or
   combinations thereof.
Clause 7. The method of any one of clauses 1-6, wherein the specific melanoma cell subtype comprises volar-like (v-mel) or non-volar cutaneous-like (c-mel) melanocyte-derived melanoma.
Clause 8. The method of any one of clauses 1-7, wherein the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18.
Clause 9. The method of any one of clauses 1-8, wherein when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma.
Clause 10. The method of any one of clauses 1-9, wherein when the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma.
Clause 11. The method of any one of clauses 1-10, wherein the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHCY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.
Clause 12. A method of stratifying and evaluating melanoma treatment response in a subject using single cell RNA sequencing (scRNA-seq) and a two-step deconvolution analysis, the method comprising:
   (a) obtaining a melanoma tumor sample from a subject;
   (b) performing scRNA-seq of the melanoma tumor sample and obtaining scRNA-seq sequence data;
   (c) on a processor, deconvoluting the scRNA-seq sequence data using a first gene signature to stratify the melanoma tumor into a specific melanoma cell subtype;
   (d) deconvoluting the scRNA-seq sequence data using a second gene signature to calculate an estimate of the total number of cells in the melanoma tumor sample that express the second gene signature; and
   (e) calculating a transcriptomic deconvolution-based predictor of ICI resistance (TD-IR) score value;
   wherein when the calculated estimate of total melanoma tumor expression of the second gene signature reaches a critical threshold value, the melanoma tumor will not respond to immune checkpoint inhibition (ICI) treatment;
   when the calculated TD-IR score value is positive, the melanoma tumor will not respond to ICI treatment; or
   when the calculated TD-IR score value is negative, the melanoma tumor will respond to ICI treatment.
Clause 13. The method of clause 12, wherein the melanoma is acral melanoma (AM).
Clause 14. The method of clause 12 or 13, further comprising:
   when it is determined that the melanoma tumor will respond to ICI treatment, an effective amount of an ICI treatment is administered to the subject; or
   when it is determined that the melanoma tumor will not respond to ICI treatment, an effective amount of an alternative non-ICI therapy is administered to the subject.
Clause 15. The method of any one of clauses 12-14, wherein the ICI treatment comprises:
   a PD-1 inhibitor selected from pembrolizumab, nivolumab, cemiplimab, or combinations thereof;
   a PD-L1 inhibitor selected from atezolizumab, avelumab, durvalumab, or combinations thereof;
   a LAG-3 inhibitor selected from relatlimab, relatlimab-RMBW, or combinations thereof; or combinations thereof.
Clause 16. The method of any one of clauses 12-15, wherein the alternative non-ICI therapy comprises:
   a PARP inhibitor selected from olaparib, niraparib, rucaparib, talazoparib, or combinations thereof;
   a BRAF inhibitor selected from dabrafenib, encorafenib, vemurafenib, or combinations thereof;
   a MEK inhibitor selected from trametinib, cobimetinib, binimetinib, or combinations thereof;
   a KIT inhibitor selected from dasatinib, imatinib, nilotinib, or combinations thereof;
   a tumor-agnostic therapy selected from larotrectinib, entrectinib, or combinations thereof;
   a CTLA-4 inhibitor selected from ipilimumab;
   aldesleukin (Interleukin-2; IL-2), Interferon alfa-2b, pegylated Interferon alfa-2b, or combinations thereof;
   a chemotherapeutic agent selected from dacarbazine, temozolomide, cisplatin, carboplatin, fotemustine, lomustine, docetaxel, paclitaxel, vinblastine, or combinations thereof;
   surgical excision; or
   combinations thereof.
Clause 17. The method of any one of clauses 12-16, wherein the specific melanoma cell subtype comprises volar-like (v-mel) or non-volar cutaneous-like (c-mel) melanocyte-derived melanoma.
Clause 18. A method of stratifying and evaluating melanoma treatment response in a subject using RNA hybridization, and a two-step deconvolution analysis, the method comprising:
   (a) obtaining a melanoma tumor sample from the subject;
   (b) performing RNA hybridization of the melanoma tumor sample using a targeted RNA probe panel to obtain targeted transcript expression data;
   (c) on a processor, deconvoluting the targeted transcript expression data using a first gene signature from the targeted RNA probe panel to stratify the melanoma into a specific melanoma cell subtype; and
   (d) deconvoluting the targeted transcript expression data using a second gene signature from the targeted RNA probe panel to calculate an estimate of the total number of cells in the tumor sample that express the second gene signature;
   wherein when the calculated estimate of total tumor expression of the second gene signature reaches a critical threshold value, the tumor will not respond to immune checkpoint inhibition (ICI) treatment.
Clause 19. The method of clause 18, wherein the melanoma is acral melanoma (AM).
Clause 20. The method of clause 18 or 19, wherein the melanoma tumor sample comprises one or more biopsy samples or one or more formalin fixed paraffin embedded (FFPE) tumor tissue samples from the subject.
Clause 21. The method of any one of clauses 18-20, wherein the targeted RNA probe panel comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ALP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, and CYC1.
Clause 22. The method of any one of clauses 18-21, further comprising:
   calculating a transcriptomic deconvolution-based predictor of ICI resistance (TD-IR) score value;
   wherein when the calculated TD-IR score value is positive, the melanoma tumor will not respond to ICI treatment; or
   wherein when the calculated TD-IR score value is negative, the melanoma tumor will respond to ICI treatment.
Clause 23. The method of any one of clauses 18-22, further comprising:
   when it is determined that the melanoma tumor will respond to ICI treatment, an effective amount of an ICI treatment is administered to the subject; or
   when it is determined that the melanoma tumor will not respond to ICI treatment, an effective amount of an alternative non-ICI therapy is administered to the subject.
Clause 24. The method of any one of clauses 18-23, wherein the ICI treatment comprises:
   a PD-1 inhibitor selected from pembrolizumab, nivolumab, cemiplimab, or combinations thereof;
   a PD-L1 inhibitor selected from atezolizumab, avelumab, durvalumab, or combinations thereof;
   a LAG-3 inhibitor selected from relatlimab, relatlimab-RMBW, or combinations thereof; or combinations thereof.
Clause 25. The method of any one of clauses 18-24, wherein the alternative non-ICI therapy comprises:
   a PARP inhibitor selected from olaparib, niraparib, rucaparib, talazoparib, or combinations thereof;
   a BRAF inhibitor selected from dabrafenib, encorafenib, vemurafenib, or combinations thereof;
   a MEK inhibitor selected from trametinib, cobimetinib, binimetinib, or combinations thereof;
   a KIT inhibitor selected from dasatinib, imatinib, nilotinib, or combinations thereof;
   a tumor-agnostic therapy selected from larotrectinib, entrectinib, or combinations thereof;
   a CTLA-4 inhibitor selected from ipilimumab;
   aldesleukin (Interleukin-2; IL-2), Interferon alfa-2b, pegylated Interferon alfa-2b, or combinations thereof;
   a chemotherapeutic agent selected from dacarbazine, temozolomide, cisplatin, carboplatin, fotemustine, lomustine, docetaxel, paclitaxel, vinblastine, or combinations thereof;
   surgical excision; or
   combinations thereof.
Clause 26. A method of stratifying and evaluating melanoma treatment response in a subject using bulk transcriptomic data and a two-step deconvolution analysis, the method comprising:
   (a) obtaining one or more melanoma tumor samples from a subject;
   (b) performing RNA sequencing of the one or more melanoma tumor samples and obtaining bulk transcriptomic data;
   (b) performing transcript counting on the bulk transcriptomic data to obtain transcript expression data;
   (c) on a processor, deconvoluting the transcript expression data using a first gene signature to stratify the melanoma into a specific melanoma cell subtype or origin; and
   (d) deconvoluting the transcript expression data using a second gene signature to calculate an estimate of the total number of cells in the melanoma tumor sample that express the second gene signature or determine the cell differentation state;
   wherein when the calculated estimate of total expression of the second gene signature reaches a critical threshold value, the melanoma will not respond to immune checkpoint inhibition (ICI) treatment.
Clause 27. The method of clause 26, wherein the melanoma is acral melanoma (AM).
Clause 28. The method of clause 26 or 27, further comprising:
   when the calculated total tumor expression of the second gene signature is below the critical threshold value, an effective amount of an ICI treatment is administered to the subject; or
   when the calculated total tumor expression of the second gene signature is above the critical threshold value, an effective amount of an alternative non-ICI therapy is administered to the subject.
Clause 29. The method of any one of clauses 26-28, further comprising:
   calculating a transcriptomic deconvolution-based predictor of ICI resistance (TD-IR) score value;
   wherein when the calculated TD-IR score value is positive, the melanoma tumor will not respond to ICI treatment; or
   wherein when the calculated TD-IR score value is negative, the melanoma tumor will respond to ICI treatment.
Clause 30. The method of any one of clauses 26-29, wherein the ICI treatment comprises:
   a PD-1 inhibitor selected from pembrolizumab, nivolumab, cemiplimab, or combinations thereof;
   a PD-L1 inhibitor selected from atezolizumab, avelumab, durvalumab, or combinations thereof;
   a LAG-3 inhibitor selected from relatlimab, relatlimab-RMBW, or combinations thereof; or combinations thereof.
Clause 31. The method of any one of clauses 26-30, wherein the alternative non-ICI therapy comprises:
   a PARP inhibitor selected from olaparib, niraparib, rucaparib, talazoparib, or combinations thereof;
   a BRAF inhibitor selected from dabrafenib, encorafenib, vemurafenib, or combinations thereof;
   a MEK inhibitor selected from trametinib, cobimetinib, binimetinib, or combinations thereof;
   a KIT inhibitor selected from dasatinib, imatinib, nilotinib, or combinations thereof;
   a tumor-agnostic therapy selected from larotrectinib, entrectinib, or combinations thereof;
   a CTLA-4 inhibitor selected from ipilimumab;
   aldesleukin (Interleukin-2; IL-2), Interferon alfa-2b, pegylated Interferon alfa-2b, or combinations thereof;
   a chemotherapeutic agent selected from dacarbazine, temozolomide, cisplatin, carboplatin, fotemustine, lomustine, docetaxel, paclitaxel, vinblastine, or combinations thereof;
   surgical excision; or
   combinations thereof.
Clause 32. The method of any one of clauses 26-31, wherein the specific melanoma cell subtype comprises volar-like (v-mel) or non-volar cutaneous-like (c-mel) melanocyte-derived melanoma.
Clause 33. The method of any one of clauses 26-32, wherein the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18.
Clause 34. The method of any one of clauses 26-33, wherein when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma.
Clause 35. The method any one of clauses 26-34, wherein when the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma.
Clause 36. The method of any one of clauses 26-35, wherein the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.

### EXAMPLES

### Example 1

### Human Subject Details

All skin was collected from surgical discards with informed consent and approval from the UCSF Institutional Review Board. The research conducted using human tissue is compliant with all relevant ethical regulations regarding human patients. All ages, races/ethnicities, and sexes were included in the eligibility criteria for this study. Participants were not compensated for their participation. Adult tissue was obtained from surgical remnants of heathy skin taken for reconstructive surgery or from amputations with heathy skin. Neonatal foreskins were obtained after routine circumcision. Anonymous fetal specimens were obtained from elective terminations and fetal age (stated as fetal weeks) was estimated by heel-toe length. When possible, fetal sex was determined by visual inspection using a dissecting microscope. All samples were collected in cold CO₂ Independent Media (Gibco-Thermo Fisher Scientific) or Medium 154 (Gibco) with 1× Antibiotic-Antimycotic (Gibco) at 4° C. until dissociation. Human melanoma data were obtained from previous studies: the TOGA Research Network; Lund University (GSE65904); Translational Genomics Research Institute (dbGAP phs001036.v1.p1); and Broad Institute (GSE72056, GSE115978.

### Skin Sample Preparation

Tissue dissociation was started the same day as sample acquisition. For adult and neonatal skin, the epidermis was enzymatically dissociated from the dermis with a dispase, neutral protease, grade II (Roche-Sigma-Aldrich), incubation for 14 hours at 4° C. Epidermal sheets were manually separated from the dermis, finely minced, and incubated with 0.5% trypsin (Gibco) for 3 minutes at 37° C. After manual trituration, trypsin was deactivated using ice cold soybean trypsin inhibitor (Gibco), then diluted 2:3 in ice cold Hanks' balanced salt solution, no Mg²⁺, no Ca²⁺ (Gibco). The dissociated cell suspension was centrifuged at 500×g, 4° C., for 4 minutes, resuspended in FACS buffer (0.1% bovine serum albumin (Sigma) and 25 mM Hepes (Gibco) in Dulbecco's phosphate-buffered saline (DPBS; Gibco) and strained with a 70 μM filter to achieve a single cell suspension. For fetal tissue, the developing epidermis was manually removed from the dermis following a 20-30-minute incubation with 10 mM EDTA (Invitrogen), DPBS at 37° C. The resulting epidermal layer was incubated with 0.5% trypsin (Gibco) for 1 min at 37° C. and manually triturated. Trypsin was deactivated using ice cold soybean trypsin inhibitor (Gibco), then diluted 2:3 in ice cold Hanks' balanced salt solution (Gibco). The dissociated cell suspension was centrifuged at 500×g, 4° C., for 4 minutes, resuspended in FACS buffer, and strained with a 70 μM filter to achieve a single cell suspension.

### FACS Analysis and Single Cell Sorting

Single cell suspensions were counted, diluted to 1×10⁶ cells/100 μL with ice cold FACS buffer containing dye conjugated antibodies (anti-KIT (104D2), 15 ng/100 μL (CD11705, Thermo Fisher Scientific), anti-ITGA6 (GoH3), 15 ng/100 μL (12-0495-82, Thermo Fisher Scientific) and CD11c, 1:20 dilution (46-0116-41, Thermo Fisher Scientific)) and incubated on ice for 25 minutes. Cells were washed one time with 10× volume of FACS buffer, centrifuged for 2 minutes at 500 g, resuspended in 30 ng/mL Dapi (D3571, Molecular Probes), FACS buffer. Resuspended cells were strained through a 35 μm nylon mesh filter and kept on ice until sorted.

Single cells were sorted into 384-well plates using the “Ultra purity” setting on a SH800S (Sony) sorter. For a typical sort, a tube containing 0.3-1 mL the pre-stained cell suspension was vortexed gently and loaded onto the FACS machine. A small number of cells were flowed at low pressure to check cell concentration and the amount of debris. Then the pressure was adjusted, flow was paused, the first destination plate was unsealed and loaded. Single cells were sorted into plates by gating to exclude dead/dying cells (DAPI+) and doublets. The majority of the plate contained melanocytes (CD11c-/KIT+) with 4-5 columns of basal keratinocytes (CD11c-/KIT-/ITGA6+) and other triple negative cells such as suprabasal keratinocytes (CD11c-/KIT-/ITGA6-). Immediately, after sorting, plates were sealed with a pre-labeled aluminum seal, centrifuged at 4° C. and flash frozen on dry ice, before storage at −80° C. for later use.

### Lysis Plate Preparation

Lysis plates were created by dispensing 0.4 μL lysis buffer (0.5U Recombinant RNase Inhibitor (Takara Bio, 2313B), 0.0625% Triton™ X-100 (Sigma, 93443-100ML), 3.125 mM dNTP mix (Thermo Fisher, R0193), 3.125 μM Oligo-dT30VN (commercially available from IDT, 5′-AAGCAGTGGTATCAACGCAGAGTACT₃₀VN-3′; SEQ ID NO: 1) and 1:600,000 ERCC RNA spike-in mix (Thermo Fisher, 4456740)) into 384-well hard-shell PCR plates (Biorad HSP3901) using a Tempest liquid handler (Formulatrix). All plates were then spun down for 1 minute at 3220×g and flash frozen on dry ice. Plates were stored at −80° C. until used for sorting.

cDNA Synthesis and Library Preparation

cDNA synthesis was performed using the Smart-seq2 protocol. Briefly, 384-well plates containing single-cell lysates were thawed on ice followed by first strand synthesis. 0.6 μL of reaction mix (16.7 U/μL SMARTScribe Reverse Transcriptase (Takara Bio, 639538), 1.67 U/μL Recombinant RNase Inhibitor (Takara Bio, 2313B), 1.67× First-Strand Buffer (Takara Bio, 639538), 1.67 μM TSO (commercially available from Exiqon, 5′-AAGCAGTGGTATCAACGCAGACTACATrGrG+G-3′; SEQ ID NO: 2), 8.33 mM DTT (Bioworld, 40420001-1), 1.67 M Betaine (Sigma, B0300-5VL), and 10 mM MgCl₂ (Sigma, M1028-10X1ML)) was added to each well using a Tempest liquid handler or Mosquito (TTP Labtech). Reverse transcription was carried out by incubating wells on a ProFlex 2×384 thermal-cycler (Thermo Fisher) at 42° C. for 90 min and stopped by heating at 70° C. for 5 min. Subsequently, 1.5 μL of PCR mix (1.67×KAPA HiFi HotStart ReadyMix (Kapa Biosystems, KK2602), 0.17 μM IS PCR primer (commercially available from IDT, 5′-AAGCAGTGGTATCAACGCAGAGT-3′; SEQ ID NO: 3), and 0.038 U/μL Lambda Exonuclease (NEB, M0262L)) was added to each well with a Mantis liquid handler (Formulatrix) or Mosquito, and second strand synthesis was performed on a ProFlex 2×384 thermal-cycler by using the following program: 37° C. for 30 minutes; 95° C. for 3 minutes; 23 cycles of: 98° C. for 20 seconds, 67° C. for 15 seconds, and 72° C. for 4 minutes; and 72° C. for 5 minutes. The amplified product was diluted with a ratio of 1 part cDNA to 10 parts 10 mM Tris-HCl (Thermo Fisher, 15568025). 0.6 μL of diluted product was transferred to a new 384-well plate using the Viaflow 384 channel pipette (Integra). Illumina sequencing libraries were prepared as follows. Briefly, tagmentation was carried out on double-stranded cDNA using the Nextera XT Library Sample Preparation kit (Illumina, FC-131-1096). Each well was mixed with 0.8 μL Nextera tagmentation DNA buffer (Illumina) and 0.4 μL Tn5 enzyme (Illumina), then incubated at 55° C. for 10 min. The reaction was stopped by adding 0.4 μL “Neutralize Tagment Buffer” (Illumina) and spinning at room temperature in a centrifuge at 3220×g for 5 min. Indexing PCR reactions were performed by adding 0.4 μL of 5 μM i5 indexing primer, 0.4 μL of 5 μM i7 indexing primer, and 1.2 μL of Nextera NPM mix (Illumina). All reagents were dispensed with the Mantis or Mosquito liquid handlers. PCR amplification was carried out on a ProFlex 2×384 thermal cycler using the following program: 72° C. for 3 minutes; 95° C. for 30 seconds; 12 cycles of: 95° C. for 10 seconds, 55° C. for 30 seconds, and 72° C. for 1 minute; and 72° C. for 5 minutes. Library pooling, quality control, and sequencing. Following library preparation, wells of each library plate were pooled using a Mosquito liquid handler. Pooling was followed by two purifications using 0.7× AMPure beads (Fisher, A63881). Library quality was assessed using capillary electrophoresis on a Fragment Analyzer (Agilent) or Tapestation (Agilent), and libraries were quantified by qPCR (Kapa Biosystems, KK4923) on a CFX96 Touch Real-Time PCR Detection System (Biorad). Plate pools were normalized to 2 nM and equal volumes from library plates were mixed together to make the sequencing sample pool.

Sequencing Libraries from 384-Well Plates

Libraries were sequenced on the NextSeq or NovaSeq 6000 Sequencing System (Illumina) using 2×100 bp paired-end reads and 2×8 bp or 2×12 bp index reads. NextSeq runs used high output kits, whereas NovaSeq runs used either a 200- or 300-cycle kit (Illumina, 20012860). PhiX control library was spiked in at 1%.

### Single-Cell Transcriptomic Processing and Analysis

Single cell RNAseq analysis was conducted in Jupyter (4.4.0)/Jupyter lab(2.1.0)/Python (3.7.3) using: Pandas(1.0.3), numpy (1.18.2), scanpy.api (1.4.4.post1), anndata (0.6.22rc1), plotnine (0.6.0), scipy (1.4.1), more_itertools (8.2.0), tqdm (4.45.0), sklearn (0.22.2.post1), lifelines (0.24.3), matplotlib (3.0.3). Single cell reads were mapped to the human reference hg38 containing ERCC sequences using STAR aligner. HTSeq was used to create gene count tables. These count tables were compiled and processed using Scanpy. Low-quality cells were filtered based on the following criteria: number of genes <500 or number of reads <50,000. Each gene in the transcriptome exhibited read counts in at least 3 cells. Cells exhibiting >2-fold higher number of genes than average were labeled as putative doublets and removed. Iterative Louvain clustering yielded cell type-specific clusters, which were annotated using published marker genes based on inter-cluster differential expression analysis (two-sided Mann Whitney U test, Benjamini-Hochberg FDR <5%). Briefly, Louvain clustering was performed on the k-nearest neighbor graph in principle component space of scaled highly variable genes. Cells were visualized using 2-dimensional UMAP embeddings. Cell cycle status was inferred by the mean ranked expression of marker genes, referred to as the cell cycle program score. Cells below the 95th-percentile of the cell cycle program score were labeled non-cycling; conversely, cells equal to or greater than 95th-percentile of the cell cycle program score were labeled cycling. In order to control for variance introduced by disproportionate populations of cycling cells across groups, non-cycling cells were considered for all downstream analyses. Thus, derivation of the four melanocyte developmental groups, anatomic site-specific analyses, and human melanocyte differentiation programs analyses were conducted on non-cycling cells.

Data and code availability: The resulting scRNA-seq data generated for this study available under accession number GSE151091 and code used to annotate and analyze the data can be found at: github.com/czbiohub/human_melanocytes.

### Melanocyte Specific Louvain Clustering

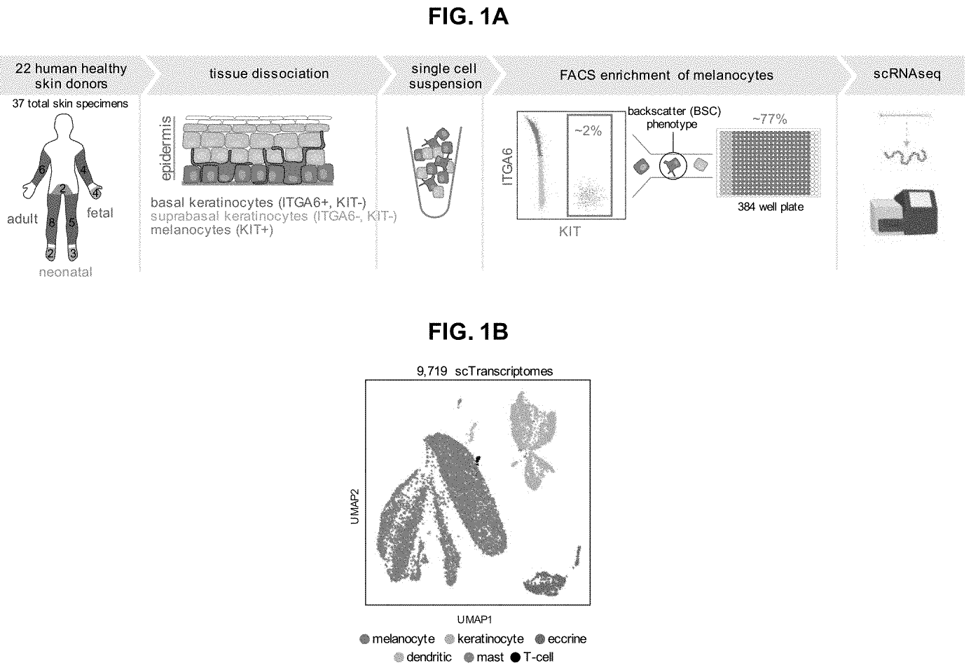

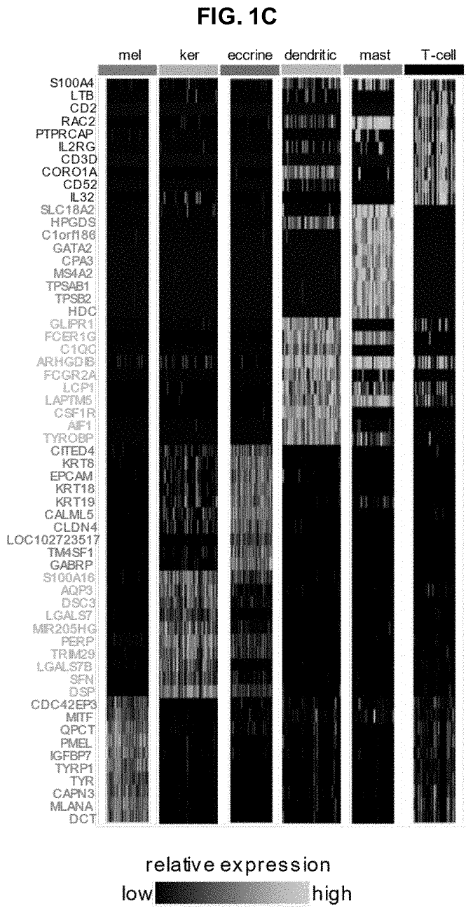

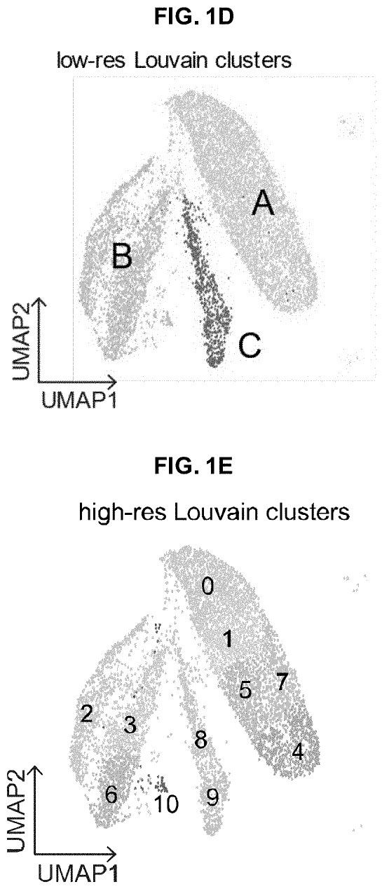

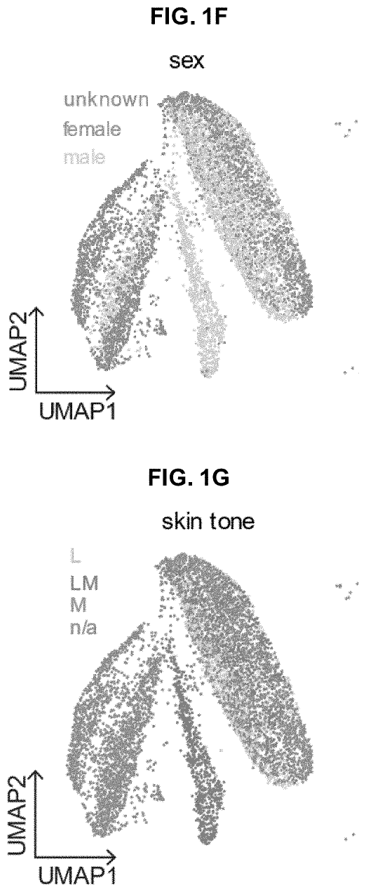

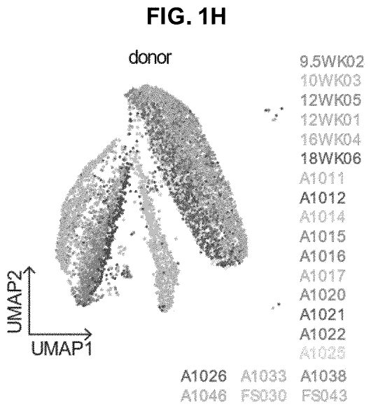

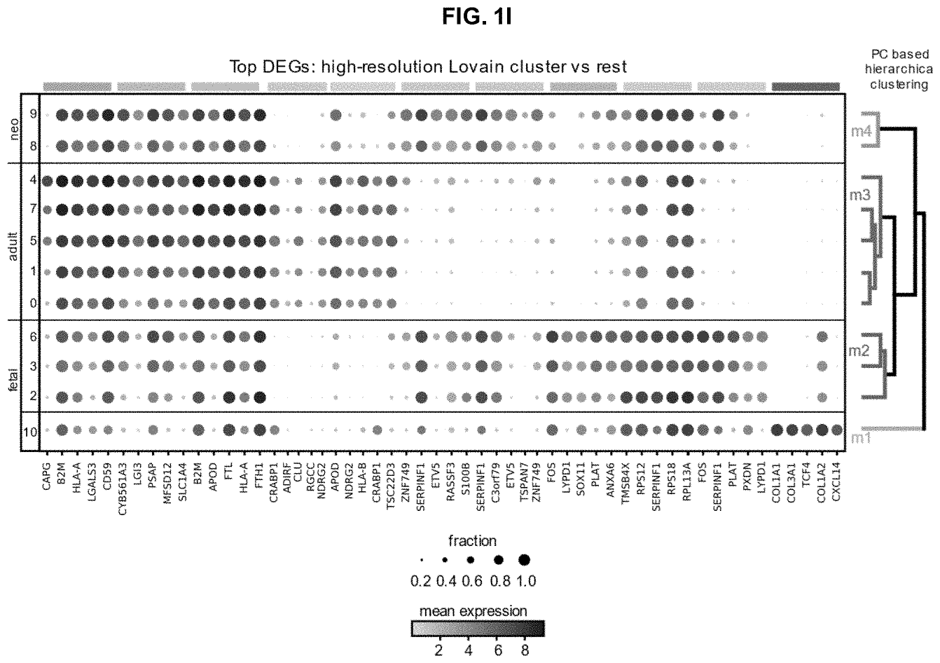

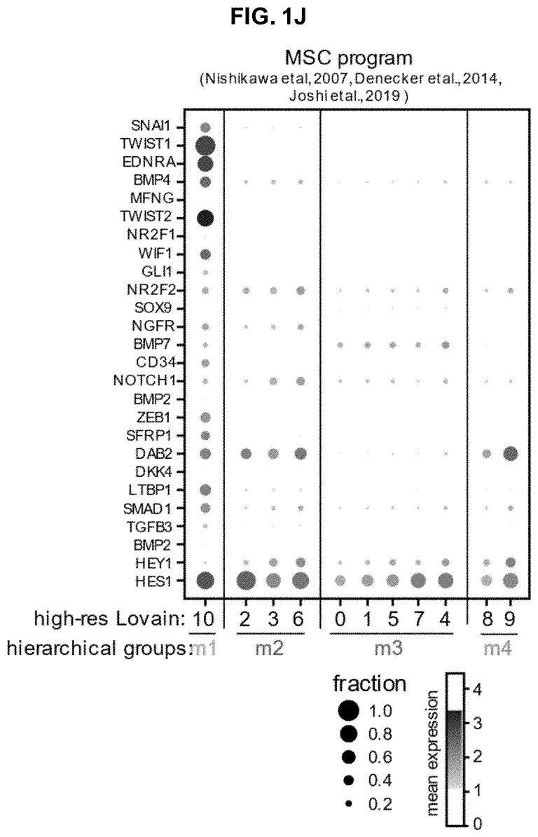

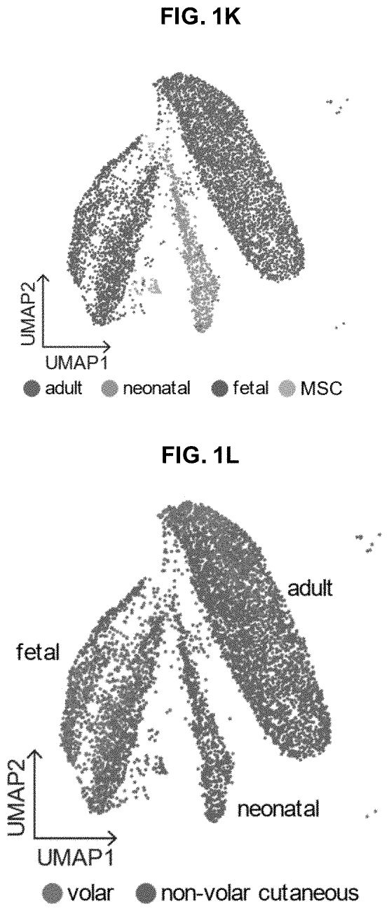

Louvain clustering on melanocytes was performed on the melanocyte only k-nearest neighbor graph in principle component space of scaled highly variable genes. Low-resolution clustering (FIG. 1D) was achieved using resolution=0.1. High-resolution Louvain clustering (FIG. 1E) was achieved independently of the low-resolution Louvain clustering using resolution=0.9.

### Identification of Four Melanocyte Developmental Stages

Differential gene expression analysis (Wilcoxon Rank-sum) of the high-resolution Louvain clusters indicated high similarity between clusters within each developmental age group (consistent with the low-resolution clustering), with the exception of fetal cluster 10. Unsupervised hierarchical clustering was employed to group the high-resolution clusters according to the median values of the first 15 PCs. PCs were chosen according to the elbow point in the variance explained PC plot. Cells were binned according to high-resolution Louvain clustering groups (0-10). For each group of cells, the median of individual PCs was computed, resulting in a matrix consisting of 11 high-resolution Louvain clustering groups by 15 median PCs. This matrix was mean-centered and scaled to unit variance before performing hierarchical clustering using Ward's criterion method. The four hierarchical clustering groups were established independent of the low-resolution Louvain clusters. However, as expected, they were consistent with the three low resolution Louvain clusters while revealing a small distinct group of fetal cells enriched for melanocyte stem cell markers. Thus, both independent methods revealed this forth cluster (“cluster 10” or “m4 cluster”) as a distinct group of cells ultimately defined as MSCs.

### Backscatter Analysis

Normalized FACS backscatter (BSC) was computed as the ratio of mean non-volar cutaneous cell BSC over mean volar cell BSC for each multi-site donor matched pair.

### Fontana-Masson Staining

Fontana-Masson staining was performed on fixed frozen sections, from patient matched volar and non-volar cutaneous skin, using the Fontana-Masson Stain Kit (ab150669, Abcam) following the manufacturer's protocol.

### Identification of Pigment Bifurcation and Post-Bifurcation Genes

Pigment associated genes identified by Baxter et al. were filtered for genes associated with a human phenotype and mean ranked expression greater than the 10th-percentile across each age of donor matched melanocytes. Baxter et al., Pigment Cell and Melanoma Research 32: 348-358 (2019). Next, the differentially expressed pigment genes between adult donor matched volar and non-volar melanocytes were identified (Mann-Whitney U test). Genes that were differentially expressed in both donors were further invested for divergent expression in the fetal donor matched volar/non-volar melanocytes. Genes coinciding with pigment bifurcation were identified as differentially expressed between volar and non-volar melanocytes at 18wks and/or 12wks with a greater than or equal to 1.3-fold higher expression in non-volar melanocytes. The remaining genes were categorized at post-bifurcation.

Percent v-Mel and c-Mel

Top-10 cutaneous and top-10 volar DEGs were identified from the site-enriched genes based on highest median per-patient log-fold-change between cutaneous and volar samples. Individual cells were classified as v-mel if 4 or more top-10 volar DEGs exhibited non-zero expression AND fewer than 4 top-10 cutaneous DEGs exhibited non-zero expression. Conversely, individual cells were classified as c-mel if 4 or more top-10 cutaneous DEGs exhibited non-zero expression AND fewer than 4 top-10 volar DEGs exhibited non-zero expression. Percent v-mel and c-mel were then calculated for each skin specimen of unique anatomic location from each individual patient.

To determine the percent of HPGD positive melanocytes in tissue sections, melanocytes (TYPR1+ cells) were manually counted. Fraction of cells was determined by the number of HPGD+ TYRP1+ cells divided by the total number of TYRP1+ cells from each fixed frozen section. To quantify the number of NTRK2 and HPGD foci per DCT+ cells from the RNAscope data, images were processed to correct for Opal 570 (HPGD) bleed-through into the Opal 620 (NTRK2) channel. After bleed-through correction, DCT and associated dapi signal was used to define the area of DCT+ cells. Then, NTRK2 and HPGD foci within DCT+ cells were counted manually. All Image analysis was performed in Fiji with statistical analysis performed in OriginPro and GraphPad Prism.

### Immunofluorescence

Skin samples were fixed in 4% paraformaldehyde (Electron Microscopy Sciences) at 4° C. overnight, washed with cold DPBS prior to paraffin or OCT embedding. Fixed frozen skin sections were incubated in blocking buffer: 2.5% donkey serum, 2.5% goat serum (Jackson ImmunoResearch Laboratories), 1% bovine serum albumin (Sigma-Aldrich), and 0.1% Triton X-100 (Sigma-Aldrich) for 1-2 hours at room temperature. The following primary antibodies were used at the indicated concentration in blocking buffer overnight at 4° C.: mouse monoclonal anti-TYRP1 1:200 (TA99, ab3312, Abcam), mouse monoclonal anti-KIT 1:100 (MA1-10072, Invitrogen-Thermo Fisher Scientific), rabbit polyclonal anti-HPGD 1:100 (HPA005679, Sigma-Aldrich). Secondary antibodies against mouse IgG Alexa Fluor 488(A21202,Thermo Fisher Scientific), or rabbit IgG conjugated to Dylight 594 (SA5-10040,Thermo Fisher Scientific) were used at a 1:1,000 dilution for 1-2 h at room temperature followed by Dapi, 1:1000 (Molecular Probes) for 1 minute. Sections were mounted in VECTASHIELD Vibrance (Vector Laboratories) prior to imaging.

Immunofluorescence images were acquired using Nikon NIS-Elements multi-platform acquisition software (5.30.01) on a fully automated Nikon Ti-E inverted microscope with an Apo TIRF, 60×, 1.49 NA, oil objective (Nikon) and a Clara CCD camera (Andor). All Image analysis was performed in Fiji with statistical analysis performed in OriginPro and GraphPad Prism.

### Multiplex RNA-FISH

Muliplex RNA-FISH was employed using the RNAscope Multiplex Fluorescent V2 assay (Bio-techne, cat. No. 323110) kit according to manufacturer's protocol on 10 μM FFPE tissue sections. Tissues were stained using probes purchased from ACD for HPGD (Channel 1, cat. no. 583651), NTRK2 (Channel 2, cat. no. 402621-C2), DCT (Channel 3, cat. no. 494361-C3) and TSA Opal 570 (Channel 1, Akoya Biosciences, cat. No. FP1488001KT), TSA Opal 620 (Channel 2, Akoya Biosciences, cat. No. FP1488001KT) and TSA Opal 690 (Channel 3, Akoya Biosciences, cat. No. FP1497001KT). TSA was used at a 1:1500 dilution. Cells were counterstained with DAPI and mounted with Prolong Gold Antifade Mountant (Thermo Fisher, P36930). Tissues were imaged using a Leica DMi8 microscope.

Melanoma v-Mel:c-Mel Score

Average log 2 normalized expression of the top 100 volar enriched and top 100 cutaneous enriched genes was calculated for each primary tumor from SKCM TCGA and dbGAP phs001036.v1.p1. a v-mel:c-mel ratio was then calculated for each tumor by dividing the v-mel score (average expression) by the c-mel score. Tumors were then grouped by reported anatomic subtype: acral (n=13 dbGAP phs001036.v1.p1, n=2 SKCM) and non-acral cutaneous (n=103 SKCM).

### Diffusion Pseudotime

Diffusion pseudotime analysis on all non-cycling melanocyte cells was performed using the “scanpy.tl.dpf” function. The pseudotime reference root cell was chosen from the youngest sample (9.5 f.w.). The diffusion map was computed from an n=30 neighborhood graph with a Gaussian kernel.

### Developmental Group GSEA Analyses

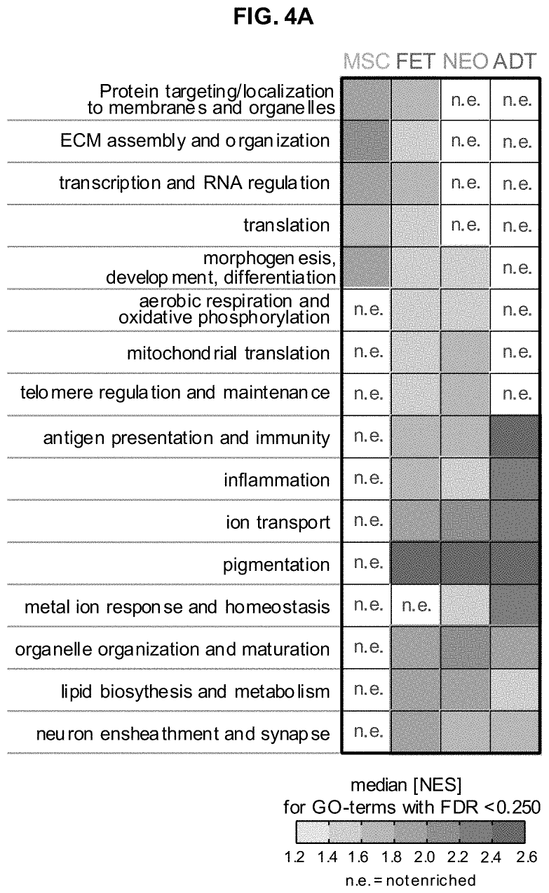

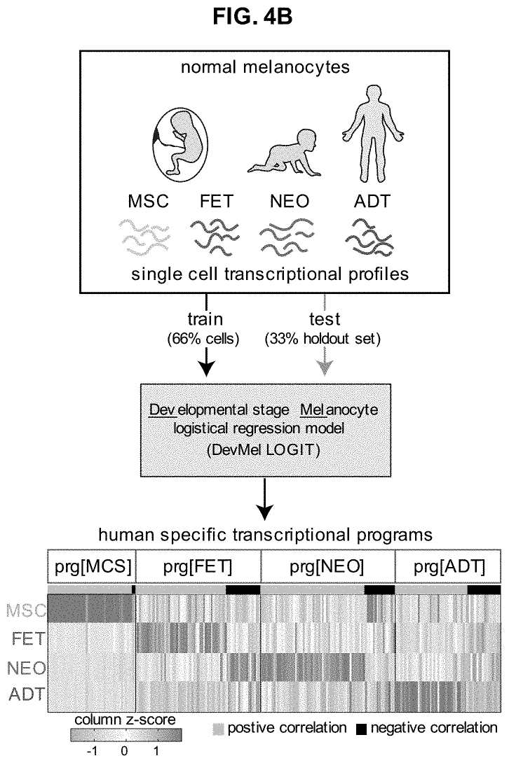

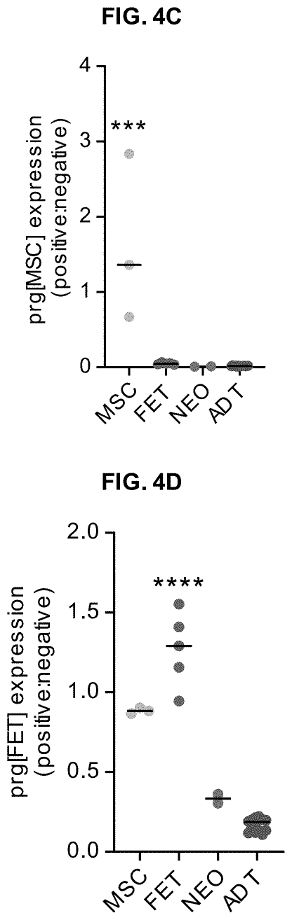

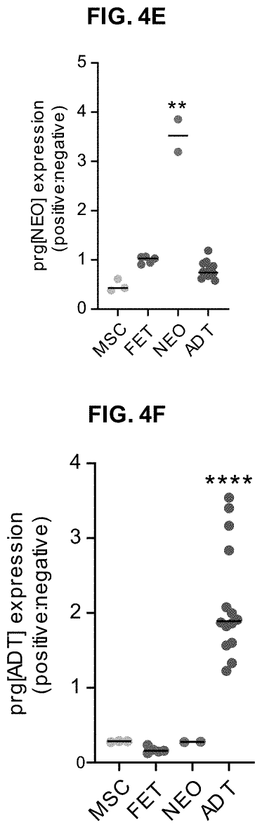

Gene set enrichment analyses for GO-biological processes (GO-bp) and previously identified neonatal foreskin and adult non-volar melanocyte cell-type DEG lists were conducted using the top differentially expressed genes (Mann-Whitney U test, Benjamini-Hochberg FDR <5%) between developmental group in GSEA4.1.0 using the GSEAPreranked tool with the weighted enrichment statistic, max size of 500 and min size of 10. Significantly enriched biological processes between temporally adjacent developmental groups (FET vs MSC, NEO vs FET and NEO vs ADT) were determined by grouping the top 50 GO-bp terms (top 50 terms with FDR q-value <0.250) for each developmental group in each pairwise comparison based on common biological themes. The above identified biological processes were then assessed for enrichment across all the human developmental stages using the differentially expressed genes for a) each developmental group compared to ADT and b) each developmental group compared to MSC and then calculating the median normalized enrichment score (for all GO-bp terms with an FDR q-value <0.250) for each common biological theme (FIG. 4A).

### DevMel Program Biological Pathway Analysis

PercayAl (v4.0, build 21) was used to identify relevant biological processes and pathways represented by the positive correlated genes within each DevMel program. The PercayAl software extracts all abstracts from PubMed that reference entities (genes) of interest (or their synonyms), using contextual language processing and a biological language dictionary that is not restricted to fixed pathway and ontology knowledge bases. Conditional probability analysis is utilized to compute the statistical enrichment of biological concepts (processes/pathways) over those that occur by random sampling. Related concepts built from the list of differentially expressed entities are further clustered into higher-level themes (e.g., biological pathways/processes, cell types and structures, etc.). Within the PercayAl software platform, scoring of gene, concept, and overall theme enrichment is accomplished using a multi-component function referred to as the Normalized Enrichment Score (NES). The first component utilizes an empirical p-value derived from several thousand random entity lists of comparable size to the users input entity list to define the rarity of a given entity-concept event. The second component, effectively representing the fold enrichment, is based on the ratio of the concept enrichment score to the mean of that concept's enrichment score across the set of randomized entity data. The top themes (chosen using the following settings: scale factor=5, visible theme threshold=7, connector threshold=70) were manually reviewed for quality control to ensure concepts within themes were based on key words linked to biological processes and pathways.

### Single Cell Developmental Stage Melanocyte (DevMel) Logistic Regression Model

Input data was composed of single cell transcriptomes from the following 4 non-volar cutaneous groups: MSC, FET, NEO, and ADT. The input examples were randomly sampled, and the number of examples was balanced among all labels. The combination of normal and melanoma transcriptomes was used to scale and center the data. The input data was split into testing and training partitions at a ratio 33:67. Elasticnet regularization was implemented with an 11 ratio=0.8. Single cell transcriptomes were evaluated by the model to yield a developmental stage label.

Data and code availability: The code for the logistical regression model can be found at: github.com/danledinh/human_melanocytes.

### Model Systems Melanocyte Program Scores

For each individual cell, the program score is the mean normalized expression for all genes in the indicated published gene signature.

### Classification of Genes in Melanoma Dedifferentiation Categories

Genes involved in melanoma dedifferentiation were identified from the normal melanocyte developmental programs (Logistic regression variables: [MSC]prg, [FET]prg, [NEO]prg, and [ADT]prg) and the top-100 differentially expressed genes for each DevMel melanoma cell population (MAL^{[MSC]} vs rest, etc. . . . ). To identify transcriptional programs associated with patterns of dedifferentiation, the mean ranked expression pattern of each gene was compared (1) across four normal melanocyte DevMel groups (MSC, FET, NEO, and ADT) and/or (2) across the four melanoma DevMel-based groups (MAL^{[MSC]} MAL^{[FET]} MAL^{[NEO]} MAL^{[ADT]}) and (3) between the normal melanocyte groups and the melanoma groups. Genes were then grouped into the following de-differentiation pathways based on the following expressing patterns:

### Direct Dedifferentiation

Met both of the following two criteria: (1) mean expression for MAL^{[MSC]}, MAL^{[FET]}, MAL^{[NEO]}, and MAL^{[ADT]} was greater than or equal to 4-fold the mean expression of MSC, FET, and NEO and (2) mean expression of ADT was less than the mean expression of MSC, FET, and NEO.

### Sequential Dedifferentiation

The DevMel stage with the highest mean expression in the normal melanocyte group was also the DevMel stage with the highest mean expression of the MAL^{[DevMel]} melanoma groups. Ex: MSC group has the highest expression of WNT5A compared all the normal melanocyte DevMel groups and MAL^{[MSC]} also has the highest expression of WNT5A compared to the other MAL^{[DevMel]} melanoma groups.

### Normal-Specific

Met all of the following criteria: (1) mean expression in each MAL^{[DevMel]} melanoma group was less than the corresponding normal melanocyte DevMel group, and (2) the mean expression was in the bottom 10th percentile for all MAL[D^{evMel]} melanoma groups, and (3) the mean expression was in the top 15^{th}-percentile for all normal melanocyte DevMel groups. Genes were then further classified into the following two groups:

### Down Regulated

Met all of the following criteria: (1) mean expression in each MAL^{[DevMel]} melanoma group was less than or equal to the mean expression of MSC, FET, and NEO normal melanocyte groups, and (2) mean expression of the ADT normal melanocyte group was greater than 1.5-old mean expression of MSC, FET, and NEO normal melanocyte groups.

### Not-Readopted

Met all of the following criteria: (1) mean expression in each MAL^{[DevMel]} was less than or equal to the mean expression of the ADT normal melanocyte group, and (2) mean expression in the MSC, FET, and NEO normal melanocyte groups were greater than 1.5-fold the mean expression of the ADT normal melanocyte group.

Melanoma-Specific (Identified from Full Transcriptomes of Normal and Malignant Cells)

Met all of the following criteria: (1) The mean expression for each MAL^{[DevMel]} melanoma group was greater than the corresponding normal melanocyte DevMel group, and (2) the mean expression was in the top 40th-percentile for each MAL^{[DevMel]} melanoma group, and (3) the mean expression was in the bottom 10th percentile for all normal melanocyte DevMel groups.

### Bulk Tumor Deconvolution

Bulk mRNAseq analysis was conducted in Python(3.7.3) using: Pandas (1.0.3), numpy (1.18.2), scanpy.api (1.4.4.post1), anndata (0.6.22rc1), plotnine (0.6.0), scipy (1.4.1), more_itertools (8.2.0), tqdm (4.45.0), sklearn (0.22.2. post1), lifelines (0.24.3), matplotlib (3.0.3), and CIBERSORT (1.06). CIBERSORT was used to deconvolve bulk RNA-seq from the SKCM-TCGA (https://www.cancer.gov/tcga) as well as the LUND dataset (GSE65904) cohorts. As input, CIBERSORT requires cell type-labeled transcriptomes to estimate the proportion of each cell type in a bulk RNA-seq sample. Here, trimmed both single cell and bulk RNA-seq transcriptomes to include only genes that are shared in both datasets. Adopting a k-fold cross-validation approach, 10 sets of single cell input transcriptomes from normal melanocytes were prepared across 4 developmental stages: MSC, FET, NEO, and ADT (balanced cell counts across all labels). Each input transcriptome set was used to devolve the SKCM-TCGA or LUND bulk RNA-seq samples, yielding 10 estimates of cell proportion. For each individual sample in the SKCM-TCGA or LUND dataset, the label means were used as the final estimate of label proportion. Hierarchical clustering was used to group SKCM-TCGA samples based on similar label proportions. One-sided Fisher Exact test was used to determine significant enrichment between two gene lists. The lifelines python package (10.5281/zenodo.3833188) was used to create Kaplan-Meier survival plots and perform logrank tests using curated SKCM-TCGA metadata.

Multi-Site scRNA-Seq of Normal Human Melanocytes

scRNA-seq was performed on 34 healthy skin specimens across multiple anatomic locations (leg, arm, foreskin, palm and sole) from 22 donors aged 9.5 fetal weeks (f.w.) to 81 years (FIG. 1A) representing multiple skin tones and sexes. Each epidermis was enzymatically removed from the dermis and dissociated into a single cell suspension. Since melanocytes comprise a small fraction of the total epidermal cell mass, FACS was used to increase the capture rate of KIT+ melanocytes within the basal layer (FIG. 1A). Sorted cells were processed using the Smartseq2 scRNA-seq protocol. After quality control and iterative Louvain clustering, differential expression was used to annotate 9,719 cells into the following cell-types: melanocytes, keratinocytes, eccrine sweat gland cells, and three immune cell populations (FIG. 1B-C. Gene expression within the melanocyte cluster presented agreement with differentially expressed genes (DEG)s from melanocyte clusters identified in previous fresh from human skin sequencing studies. Individual cells were designated as cycling or non-cycling based on expression of established marker genes. To investigate heterogeneity within melanocytes, Louvain clustering was performed on melanocytes alone. Low-resolution Louvain clustering yielded three major clusters (clusters A-C) that aligned with the three developmental ages collected in this study (cluster A: 97.5% adult, cluster B: 99.5% fetal, cluster C: 93.2% neonatal, FIG. 1D. High-resolution Louvain clustering was then performed. The resulting 11 high-resolution clusters (0-10, FIG. 1E), individually, did not correspond to skin tone, sex, or donor (FIG. 1F-H). Differential gene expression analysis of the high-resolution clusters indicated high similarity between clusters within each developmental age group, with the notable exception of fetal cluster 10 (FIG. 1I). Consistent with this observation, unsupervised hierarchical clustering using principal components binned the high-resolution clusters into four groups: m1, one fetal cluster (10); m2, the remaining fetal clusters (2, 3, 6); m3, encompassing all adult clusters (0, 1, 4, 5, 7); and m4, both neonatal clusters (8, 9) (FIG. 1I). Two of the top 5 ranked genes for the m1 group had known associations with stem- and progenitor-cell function (TC4F and CXCL14) (FIG. 1I). Established melanocyte stem cell (MSC) signatures were assessed and high expression in the m1 group was identified (FIG. 1J), indicating the melanocytes captured from this cohort encompass four developmental groups: adult, neonatal, fetal and MSC (FIG. 1K). Additionally, evaluation of anatomic location presented volar vs non-volar site as another possible source of heterogeneity within the adult and fetal groups (FIG. 1L).

### Site-Specific Pigment Associated Transcriptional Programs

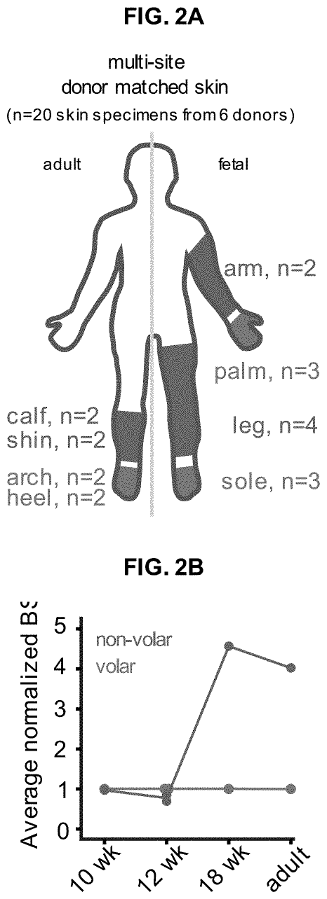

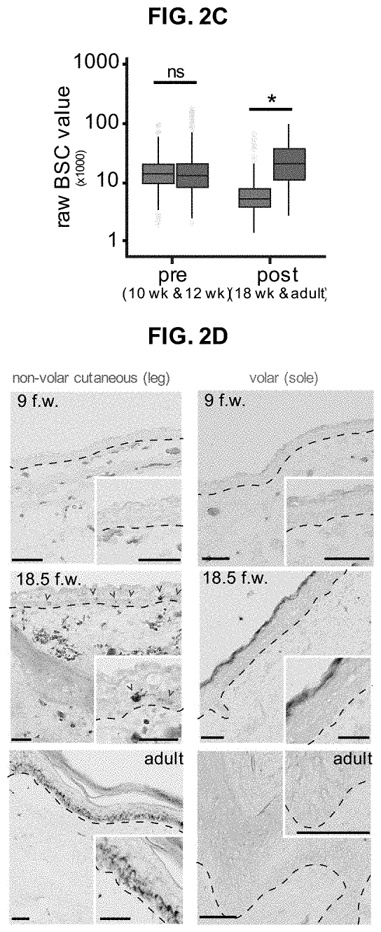

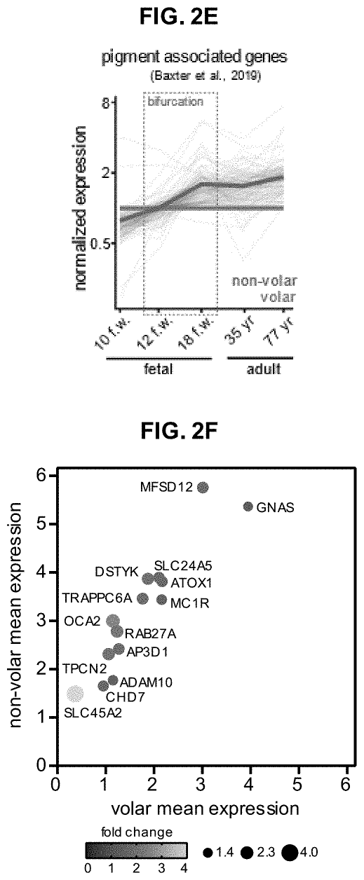

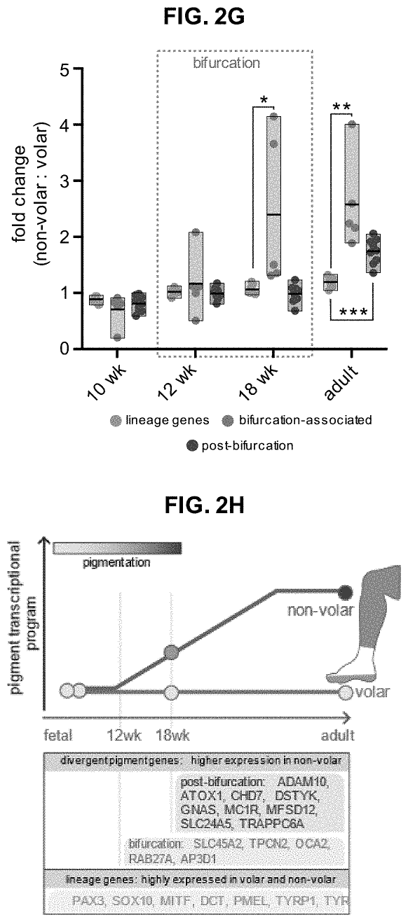

Hypopigmentation of palms and soles is present in neonates and continues through adulthood, indicating that site-specific pigmentation occurs during development, but the genes that regulate intra-individual pigmentation variation are poorly understood. Since anatomic location was a possible source of melanocyte heterogeneity (FIG. 1L), this study sought to identify the genes associated with differential pigmentation. As part of the single cell isolation pipeline, FACS back-scatter (BSC) measurements were indexed for individual cells (FIG. 1A). BSC values are an established correlate of relative pigmentation and pigment organelle (melanosome) content. Using BSC, relative pigmentation between donor-matched volar and non-volar melanocytes was queried (FIG. 2A). At 10 and 12 f.w., there were no detectable differences in BSC (FIG. 2B-C). In contrast, a striking increase in BSC was observed within the non-volar cutaneous-derived melanocytes at 18 f.w. and adulthood (FIG. 2B-C). Additionally, fetal skin presented an increase in Fontana-Masson staining at 18.5 f.w. in non-volar cutaneous skin with no evidence of staining in donor-matched volar skin (FIG. 2D). These observations are consistent with previous reports and indicate that the site-specific bifurcation of melanocyte pigmentation occurs between 12 and 18 f.w.

To identify genes correlated with intra-individual pigmentation, age-dependent expression of known pigment genes between donor-matched volar and non-volar cutaneous melanocytes were analyzed. Although an overall increase in the relative expression of pigment-associated genes in non-volar cutaneous melanocytes was observed compared to volar melanocytes after 12 f.w. (FIG. 2E, bold red line), the expression patterns of individual pigment-associated genes were varied (FIG. 2E, thin red lines). Pigment-associated genes were grouped based upon three expression patterns—lineage genes: melanocytic lineage specific genes highly expressed in volar and non-volar cutaneous melanocytes, bifurcation genes: upregulated in non-volar cutaneous melanocytes in concordance with pigment bifurcation at 12 f.w.-18 f.w., and post-bifurcation genes: upregulated in adult non-volar cutaneous melanocytes (FIG. 2F-G). Lineage genes included melanocyte differentiation genes and master regulators of melanin production (SOX10, PAX3, MITF, DCT, TYRP1, TYR, PMEL) whereas bifurcation genes and post-bifurcation genes were involved in melanosome biogenesis and function (SLC45A2, TPCN2, OCA2, RAB27A, AP3D1, ADAM10, TRAPPC6A, SLC24A5, ATOX1) and/or pigment signaling pathways/UV response (MC1R, GNAS, DSTYK) (FIG. 2H). Further supporting these finding, allelic variation and/or differential expression of several bifurcation and post-bifurcation genes, such as MFSD12, are known to regulate skin pigmentation variation between individuals. This approach pinpointed pigment genes with differential expression correlated to intra-individual pigment variation (FIG. 2H).

### Anatomic Site-Enriched Melanocyte Sub-Populations

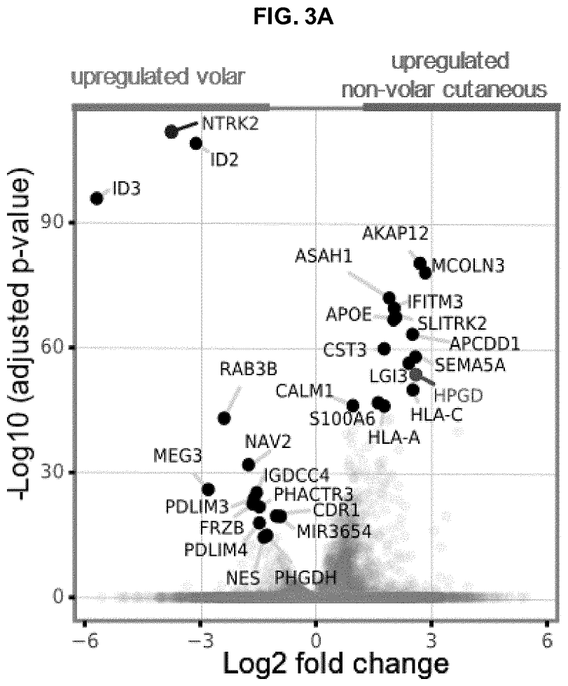

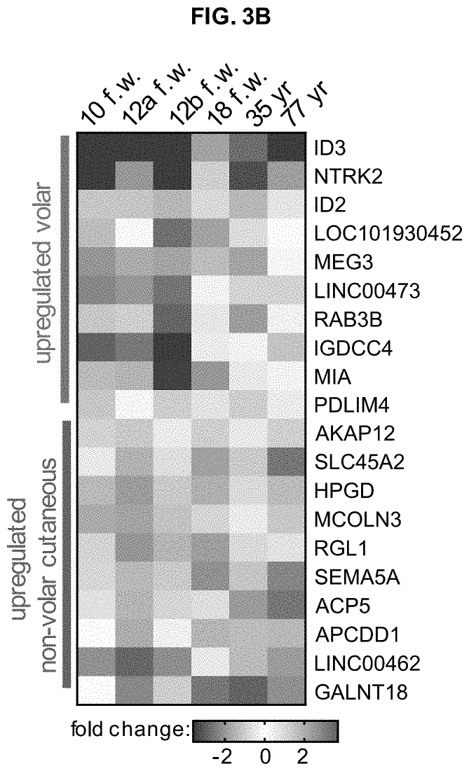

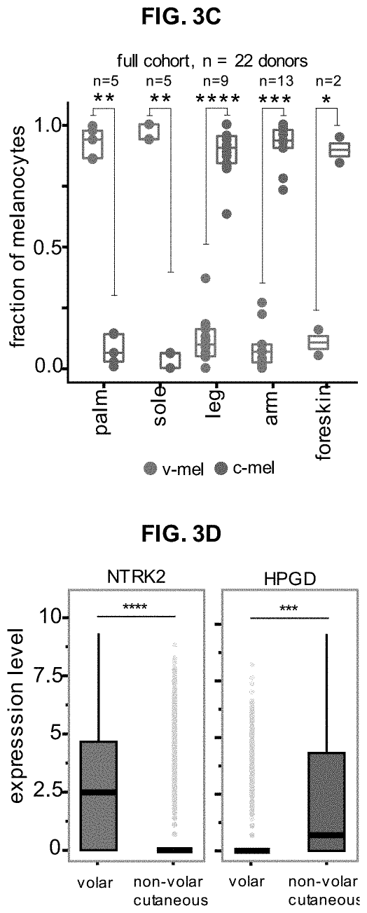

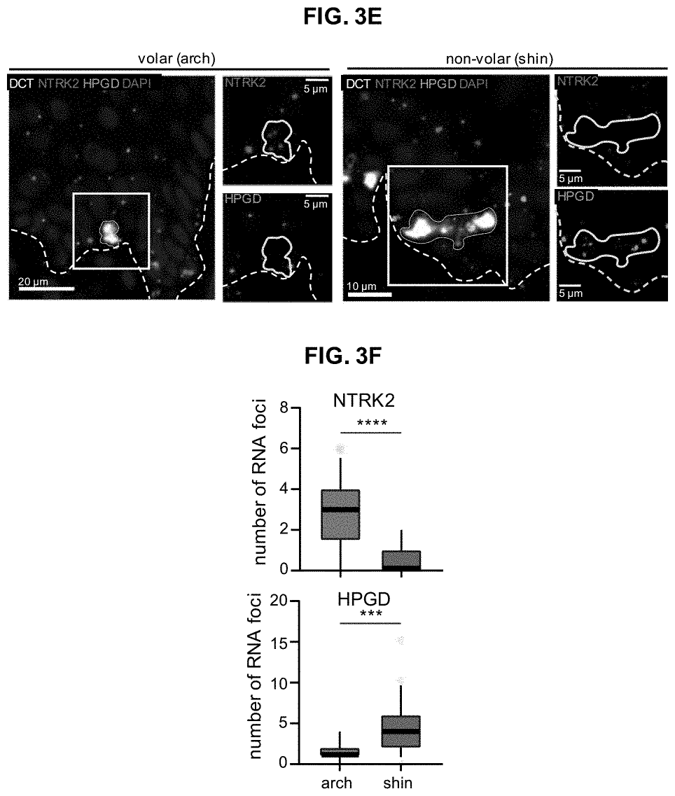

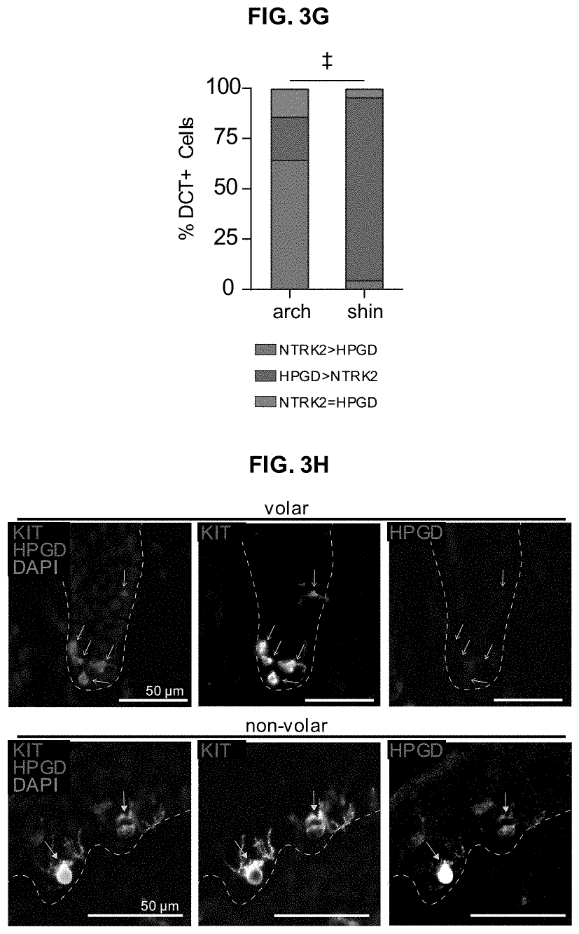

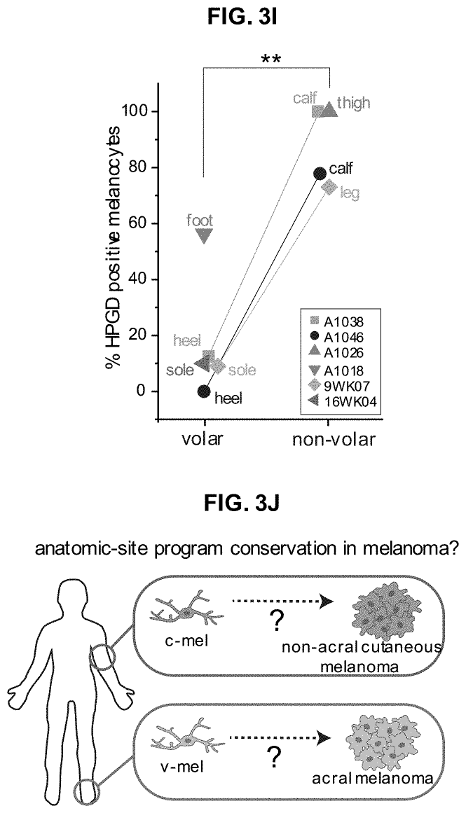

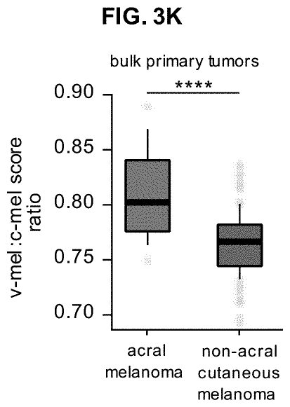

The anatomic location of skin influences melanocyte survival and function but it remains unclear how site-specific specialization arises during melanocyte maturation. To broaden the understanding of melanocyte intrinsic differences during development between anatomic sites, donor matched volar and non-volar cutaneous specimens that spanned 10 f.w. to 77 years, sexes, and skin tone were examined for transcriptional programs that distinguished volar vs non-volar cutaneous melanocytes across developmental ages (n=6 donors, n=17 skin specimens, FIG. 2A). Differential gene expression analysis (Mann-Whitney U test, Benjamini-Hochberg FDR <5%) revealed 2,042 transcripts with site-specific expression in both fetal and adult donors (FIG. 3A, Table 1). Volar melanocytes presented increased expression of NTRK2, ID2 and ID3- genes previously associated with a subset of melanomas and/or silenced in non-volar cutaneous melanocytes. As expected from the above analyses, non-volar melanocytes expressed genes involved in pigmentation. Using binary expression of the top 10 volar and non-volar cutaneous genes (FIG. 3B), individual cells were classified from the full cohort (n=22 donors, n=34 skin specimens) as volar-like (v-mel) and non-volar cutaneous-like (c-mel). While v-mels and c-mels were present in all anatomic locations for both adult and fetal skin (FIG. 3C), v-mels were enriched in volar skin (mean: 94%±5% s.d. volar sites, ˜7%±5% non-volar sites) and c-mels were enriched in non-volar cutaneous skin (mean: ˜89%±9% non-volar sites, 5%±5% volar sites). The presence of melanocytes with a c-mel signature in volar sites and melanocytes with the v-mel signature in cutaneous sites indicated: (1) two distinct sub-populations of epidermal melanocytes exist in human skin with anatomic site-specific enrichment, and (2) enrichment occurs during and persists after skin development. This discovery was validated via RNA FISH and immunofluorescence using the v-mel and c-mel signature genes that presented a striking level of inverse expression between volar and non-volar cutaneous melanocytes across all donor-matched skin: NTRK2 and HPGD, respectively (FIG. 3D-I). These observations further suggest the previously reported site specific mesenchymal-melanocyte interactions that drive the epidermal phenotype in fully-developed skin, provide more permissive, but non-exclusive, conditions for one melanocyte subpopulation over another.

Approximately 4% of primary cutaneous melanomas (CM), called acral melanomas (AM), arise from volar regions. The disease-specific death rate from AM is more than twice as high as that of CM in general. While AMs are, on average, diagnosed at more advanced stages and deeper Breslow depth, partially explaining the increased morbidity, when adjusted for Breslow depth and stage, AMs still have worse outcomes suggestive of a biologic etiology for this discrepancy. To determine whether AM may arise from v-mels (FIG. 3J), publicly available datasets were accessed to compare expression of v-mel to c-mel signatures in 103 primary non-acral CMs and 15 primary AMs. The v-mel signature was significantly elevated in the AM cohort (FIG. 3K, unpaired, two tailed t-test, p-value <0.0001), suggesting that AMs retain v-mel transcriptional programs and are therefore possibly derived from v-mels.

### Human-Specific Melanocyte Differentiation Programs

The transcriptional programs that changed with non-volar cutaneous melanocyte development were assessed. Diffusion pseudotime analysis, pairwise differential gene expression, and gene set enrichment analysis for Gene Ontology Biological Process (GO-bp) terms identified neonatal melanocytes (NEO) as an intermediate transcriptional state between fetal (FET) and adult (ADT) (FIG. 4A). Comparison to previously published transcriptomes of neonatal and adult human melanocytes demonstrated significant enrichment with the NEO (normalized enrichment score (NES)=−1.81 for Cheng et al., Cell Reports 25, 871-883 (2018) neonatal foreskin, FDR q-value=0.00) and ADT (normalized enrichment score (NES)=3.15 for Cheng et al. (2018) adult non-volar, FDR q-value=0.00) programs, respectively, further supporting this distinction. Melanocyte stem cells (MSC)s were enriched for terms associated with extracellular matrix (ECM) assembly and morphogenesis, development, and differentiation—the latter of which remained significant but trended downward in FET and NEO (FIG. 4A). In contrast, genes associated with immunity, inflammation, organelle maturation and pigmentation presented increasingly significant enrichment with each consecutive developmental group. Together, these data suggest the four main developmental groups represent distinct developmental stages along a differentiation trajectory. These analyses did not reveal significant further transcriptional changes associated with donor age in adult melanocytes.

To identify gene signatures that best distinguished each human melanocyte developmental group from each other, a regularized logistic regression model was trained using the single cell transcriptomes from 66% of the dataset representing the four developmental stages (FIG. 4B). The resultant Developmental stage Melanocyte (DevMel) model demonstrated excellent classification accuracy when tested on the holdout set, with f1-scores ranging from 0.93-1.00 (Table 2). Elastic net regularization yielded genes that collectively constituted developmental stage-specific expression programs: prg[MSC], prg[FET], prg[NEO] and prg[ADT] (FIG. 4B-F and Table 2). Each program is a relatively small (45-69 genes) expression program unique to the associated developmental group. Small gene sets are not amenable to reliable GO analyses, so an augmented artificial intelligence approach was employed to identify biological processes associated with each program (see methods). The prg[MSC] was again associated with ECM assembly, as well as neural crest cell fate specification, IGF signaling and a stem cell associated WNT-TCF-LEF-Beta-catenin program; prg[FET] with MAPK, PI3K and NFκβ signaling and chromatin remodeling; and prg[ADT] with inflammation, skin epidermis, and cell polarity. The prg[NEO], in particular, was least associated with unique known biological processes, potentially reflective of its intermediated status between FET and ADT.

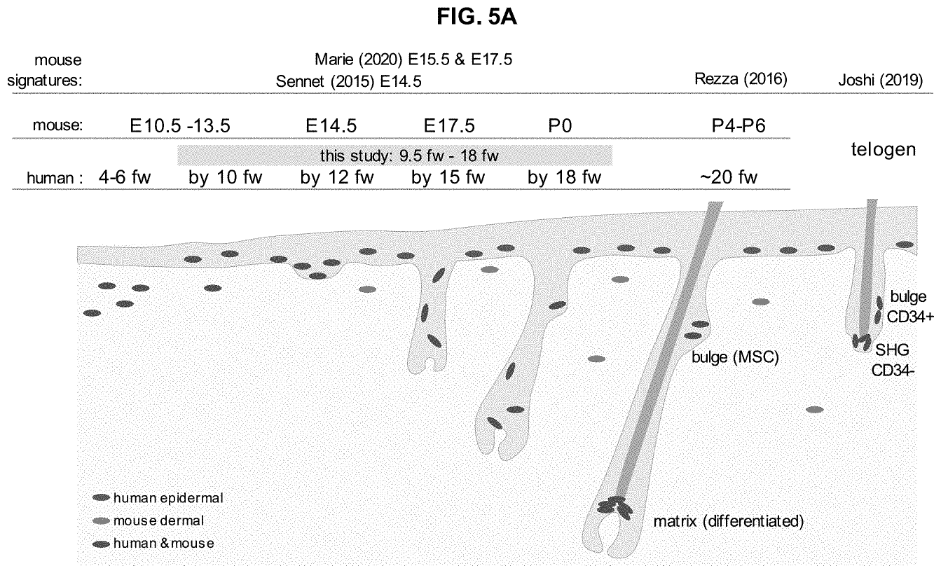

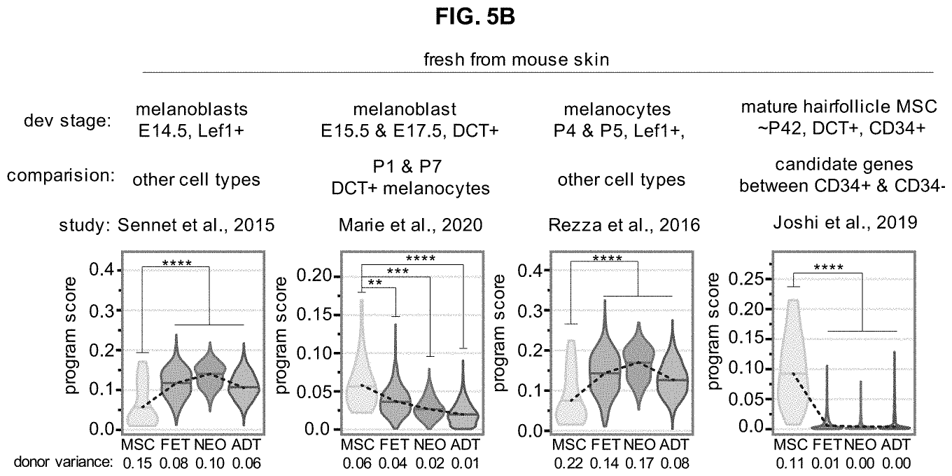

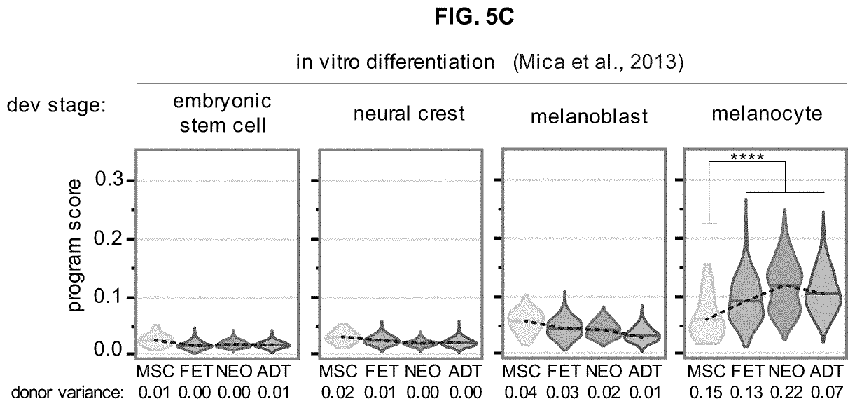

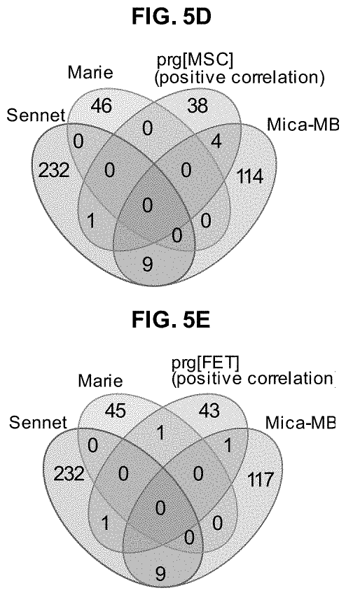

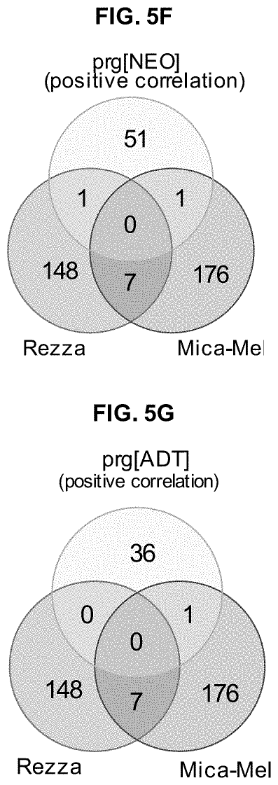

While there is substantial overlap in melanocyte development pathways between different model organisms, there are known species dependent differences and conservation of these processes in human skin remains unresolved. To benchmark human melanocyte development against known mammalian developmental systems, the expression of the gene signatures previously defined during mouse melanocyte development (FIG. 5A) and in vitro differentiation of human embryonic stem (ES) cells into mature melanocytes were assessed within ADT, NEO, FET and MSC non-volar cutaneous skin. Both the Sennet et al. E14.5 mouse melanoblast signature and the Rezza et al. P4 & P5 mouse melanocyte signature were more highly expressed in the FET, NEO and ADT melanocytes compared to MSCs (p-value <1×10⁻¹², FIG. 5B). In these studies, melanocytic cells were isolated using LEF1 and KIT expression, and the gene signatures were derived from the comparison of melanocytic cells to other skin cells. In mice, LEF1 is a marker of differentiated (and differentiating) melanocytes and is not expressed in MSC. Thus, the resulting gene signatures represent a general melanocytic cell-type specific program, exclusive of MSCs, at each mouse developmental time point. The observed low program expression in the human MSC group defined in the current study is therefore consistent with the experimental design for the Sennet and Rezza studies (FIG. 5B). Rezza et al., Cell Reports 14, 3001-3018 (2016); Sennett et al., Dev. Cell 34, 577-591 (2015). In contrast, the Marie et al. melanoblast signature was most highly expressed in MSC (>1.5-fold change when compared to each other group, p-value <1×10⁻⁷, FIG. 5B). Marie et al., Nature Comm. 11, 1-18 (2020). Unlike the Sennet et al. melanoblast signature, the Marie et al. melanoblast signature was derived from the comparison of DCT+ melanoblasts at E15.5 & E17.5 to P1 & P7 melanocytes and is therefore a melanoblast specific signature. DCT is expressed in differentiated (and differentiating) melanocytes as well as MSCs. Consistent with this finding, the CD34+ mouse hair follicle melanocyte stem cell gene set was most highly expressed in MSC (p-value=5.4×10⁻³⁸, FIG. 5B).

Mouse hair follicle morphogenesis occurs around E14 and is completed postnatally by P8 as a fully mature hair-bearing follicle in anagen phase. In humans, hair follicle formation is reported to start around 10 f.w. with mature hair follicles appearing around 20 f.w. depending on anatomic location and study. The fetal skin specimens in the dataset coincide with the onset and later stages of human hair follicle development, which would encompass morphological stages that resemble mouse hair follicle development at E15.5 and E17.5 from Marie et al. (FIG. 5A). Unlike mice, human hair-bearing skin contains both hair follicle-associated and epidermal-associated melanocytes. Therefore, it is reasonable that the mouse melanoblast-specific program from Marie et al. is most highly expressed in a subset of the human fetal melanocytes that express known follicular-associated gene signatures (FIG. 1J and FIG. 5B). Together, these data suggest the human MSCs are melanoblasts that give rise to follicular and perhaps also epidermal melanocytes.

Of the in vitro differentiation programs, the mature differentiated melanocyte program was expressed across all developmental groups with the highest expression in FET, NEO and ADT groups compared to MSC (p-value <1×10⁻¹⁴, FIG. 5C). These observations suggest that in vitro generation of melanocytes from pluripotent stem cells does not distinguish between differentiating, young and aged melanocytes. Differentiation protocols that better distinguish the in vivo profiles reported here, especially accounting for the effect of the aged adult developmental state, would be a valuable tool for the field.

Comparison of DevMel program genes to those identified in mouse or in human in vitro differentiation yielded sparse overlap (FIG. 5D-G), indicating that this approach revealed previously unidentified programs specific to human fetal, neonatal, and adult skin. The study therefore sought to determine whether profiles unique to human in vivo development could provide insight into melanoma dedifferentiation and aggression.

### Reacquisition of Developmental Programs During Tumorigenesis

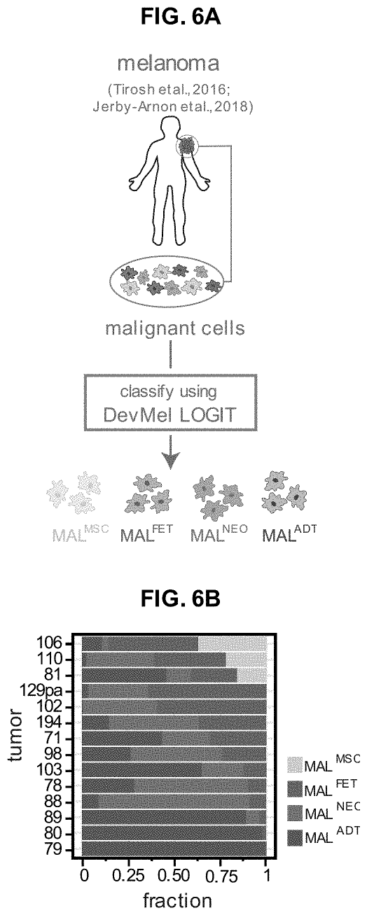

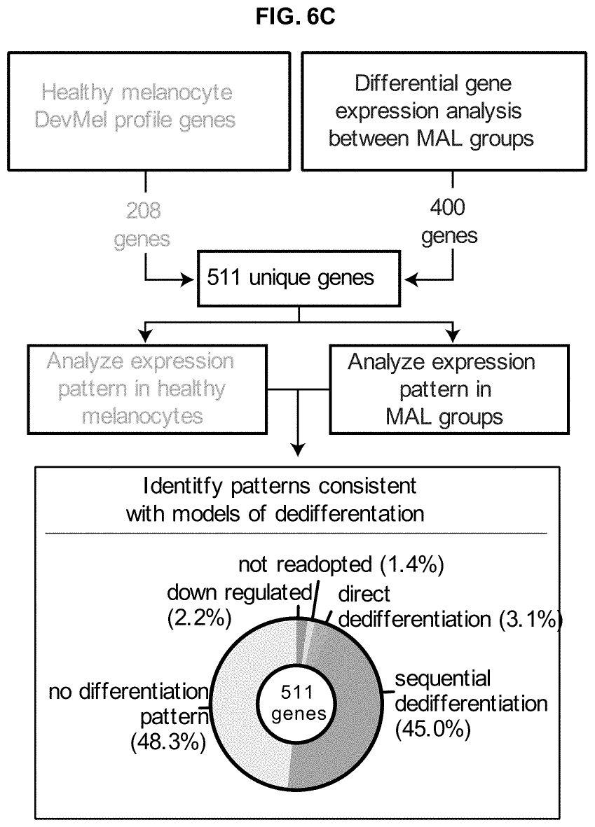

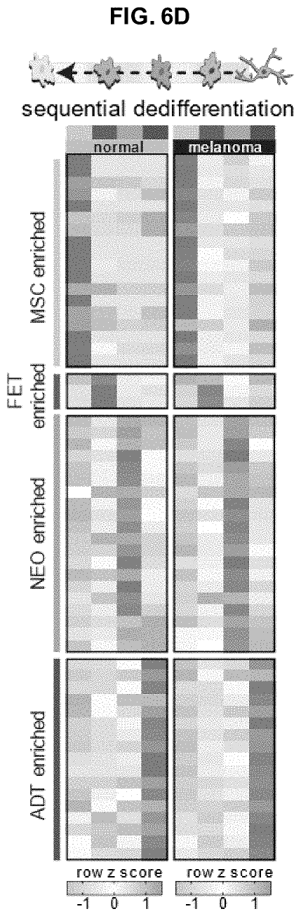

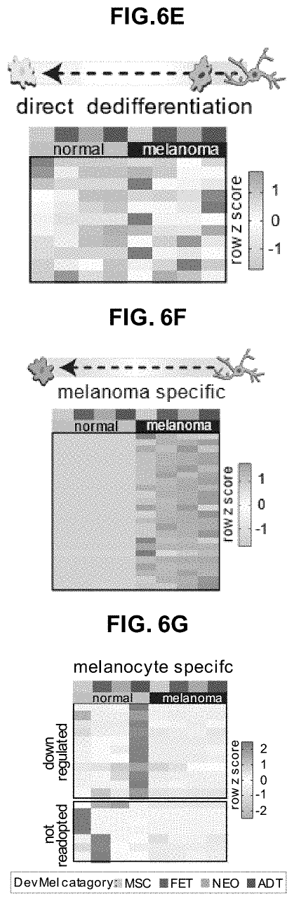

Melanoma progression often coincides with the loss of melanocyte differentiation markers and upregulation of genes associated with earlier stages of development. This process is broadly described as dedifferentiation. Given the substantial cell-to-cell intra-tumor heterogeneity of melanoma, it was reasoned that single cells within a tumor might occupy various stages of dedifferentiation and that the proportion of cells in each state potentially influences overall patient outcome. To assess tumor heterogeneity, published single-cell malignant melanoma samples were classified using the DevMel model. Each melanoma cell was classified by the similarity of its transcriptome to the human development-associated programs, resulting in four groups of melanoma cells—MAL^{MSC}, MAL^{FET}, MAL^{NEO}, and MAL^{ADT} (FIG. 6A). Inter- and intra-tumor heterogeneity was observed in the representation of each melanoma group (FIG. 6B), indicating tumors are composed of a mix of dedifferentiated states.

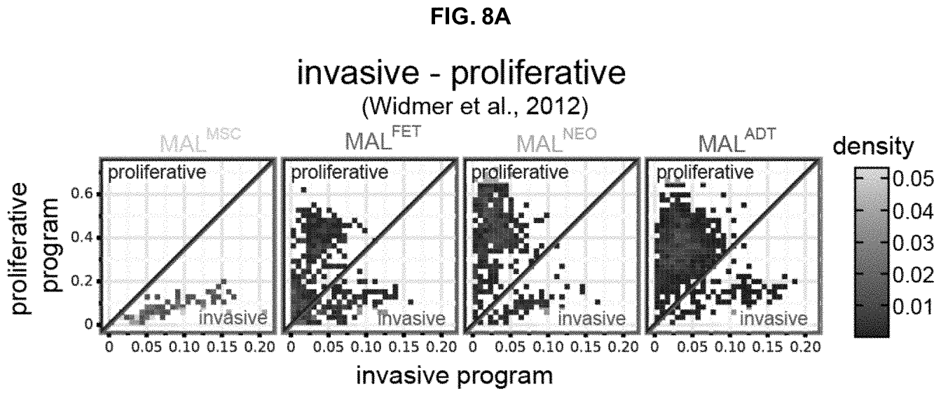

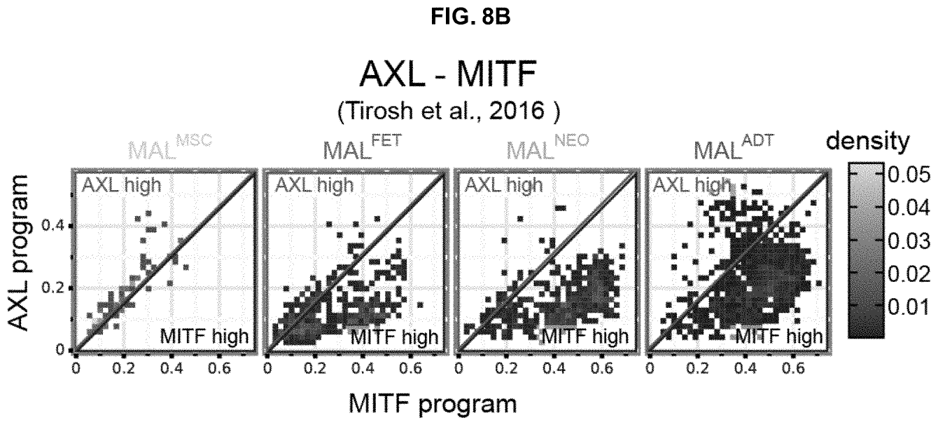

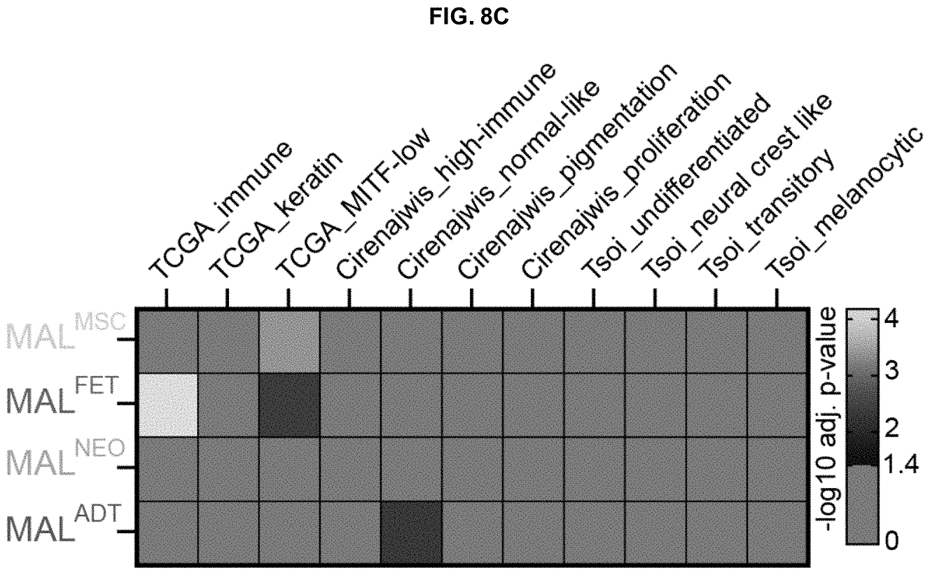

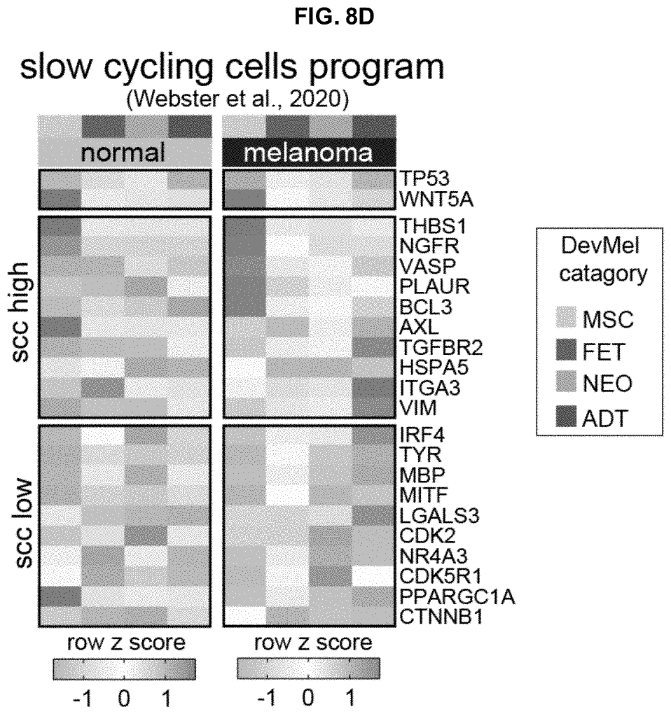

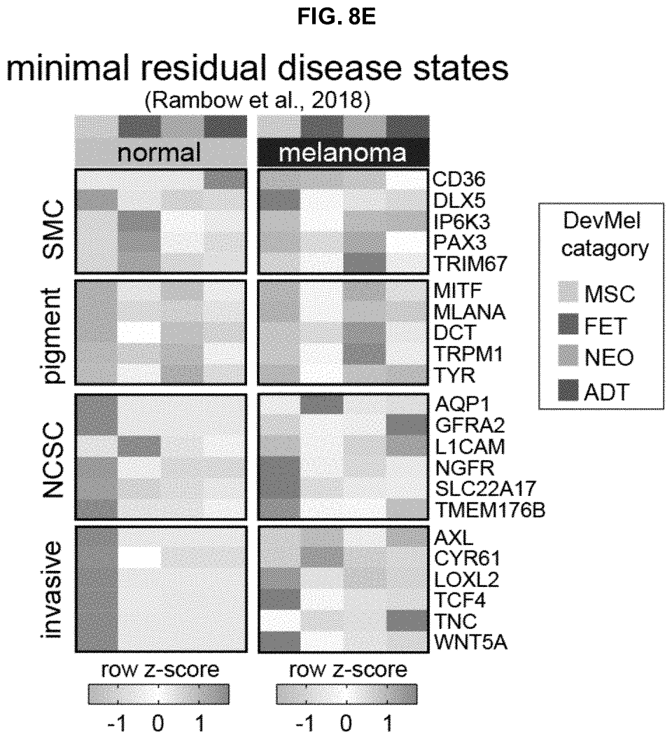

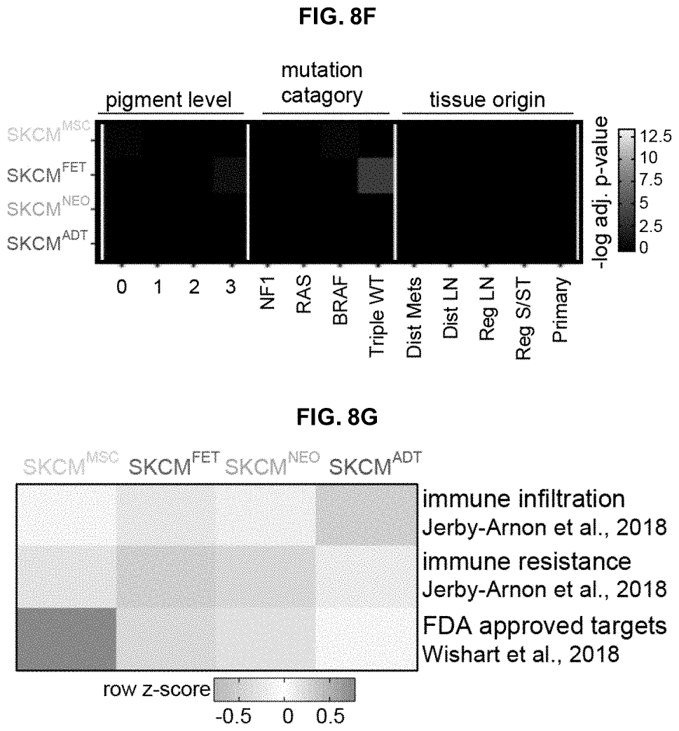

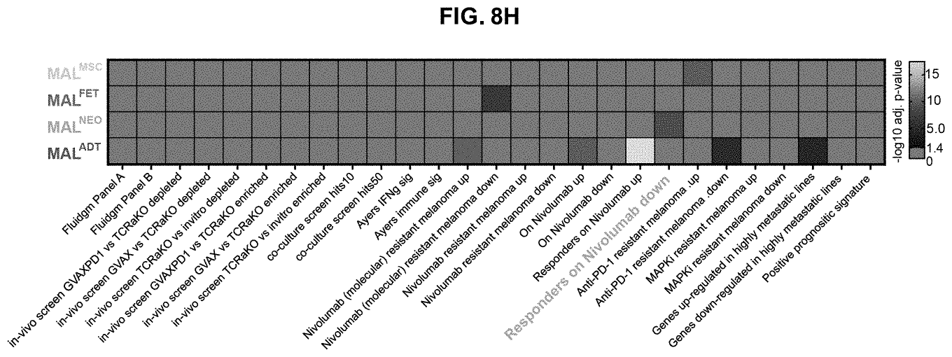

Previous reports have used bulk-tumor transcriptional signatures to classify cohorts of melanomas—most notably, the TOGA cohort can be classified as “immune,” “keratin,” or “MITF-low;” and the Cirenajwis et al. cohort as “Immune,” “Normal-like,” “Pigmented,” or “Proliferative,” Cirenajwis et al., Oncotarget 6: 12297-12309 (2015). Others have classified tumors based upon profiling of in vitro differentiation of human stem cells into melanocytes, resulting in signatures for “undifferentiated,” “neural crest like,” “transition,” and “melanocytic.” It was determined how the signatures derived from human melanocyte development corresponded with published melanoma states and signatures. Similar to the analysis of model system developmental programs, the MAL^{MSC} melanoma cells corresponded with previously identified stem cell-like transcriptional states (FIG. 8A-E). Significant enrichment was observed between the MALADT group and the Cirenajwis et al. “normal-like” signature, consistent with these cells retaining a substantial component of the differentiated melanocyte program. Surprisingly, neither the MAL^{FET} nor the MAL^{NEO} group exclusively segregated with previously defined signatures. For example, the MAL^{FET} group was significantly enriched for both the TOGA immune and MITF-low signatures, whereas the MAL^{NEO} group was not enriched for any previously defined signatures. These observations suggest that categorization of malignant melanoma cells by the human developmental stage categories defined here represents a different classification system, with the MAL^{MSC} and MAL^{ADT} groups reasonably aligned with previously reported MITF-low/stem cell and normal melanocytes, respectively, and the MAL^{FET} and MAL^{NEO} groups representing previously unreported signatures. Thus, it was reasoned that classification of melanoma cells by human developmental programs might offer further insight toward understanding dedifferentiation in melanoma.

To better define the course of dedifferentiation during melanoma progression, differential gene expression patterns were identified across each of the four MAL groups that were consistent with different forms of cellular reprogramming: (1) a retrograde unfolding of the differentiation cascade (sequential dedifferentiation). (2) direct reprogramming to a more pluripotent stage (direct dedifferentiation), or (3) the acquisition of a melanoma-specific program (FIG. 6C-G). Of 511 total unique genes, inclusive of DevMel model variables and MAL group top differentially expressed genes (Table 3), 45% exhibited expression patterns consistent with sequential dedifferentiation, in which the relative expression across healthy melanocyte developmental groups was conserved among MAL groups (FIG. 5D). 3.1% of genes exhibited a direct dedifferentiation pattern, indicating that expression of these genes may be a prerequisite for disease progression and metastasis (FIG. 5E). Supporting this interpretation, this small set of genes includes known markers of aggressive melanoma such as AXL and HMGA2. Similarly, recently identified therapeutic resistance programs were evident in both the MSC healthy and MAL^{MSC} populations (FIG. 8D-E). Genes expressed in healthy melanocyte groups were identified that were down-regulated in all melanoma groups (FIG. 5G), thus characterizing aspects of normal melanocyte expression that are either non-essential or potentially inhibitory to melanoma progression and/or metastasis. Although no significant enrichment was observed for the in vitro differentiation based gene signatures from Tsoi et al. in any of the MAL groups (FIG. 8C), the analyses suggest that sequential dedifferentiation, which recapitulates the ordered cascade of differentiation in reverse (FIG. 5B), is predominant in melanoma progression. Tsoi et al., Cancer Cell 33(5): 890-904 (2018). This discovery mirrors the findings of Tsoi et al. which show that development of therapeutic resistance in melanoma traverses a sequential dedifferentiation trajectory.

Finally, 52 highly expressed genes in melanoma were absent from each of the healthy melanocyte developmental groups (FIG. 5F and Table 3). Among the top differentially expressed genes was the melanoma-associated antigen PRAME, further supporting its use as a melanoma molecular diagnostic. Other of these “melanoma specific” genes might be important for melanoma progression such as the MTRNR2L family of transcripts, which encode for short peptides with anti-apoptotic activity, and were highly and exclusively expressed in all melanoma groups.

Developmental Stage Programs Correlate with Patient Survival

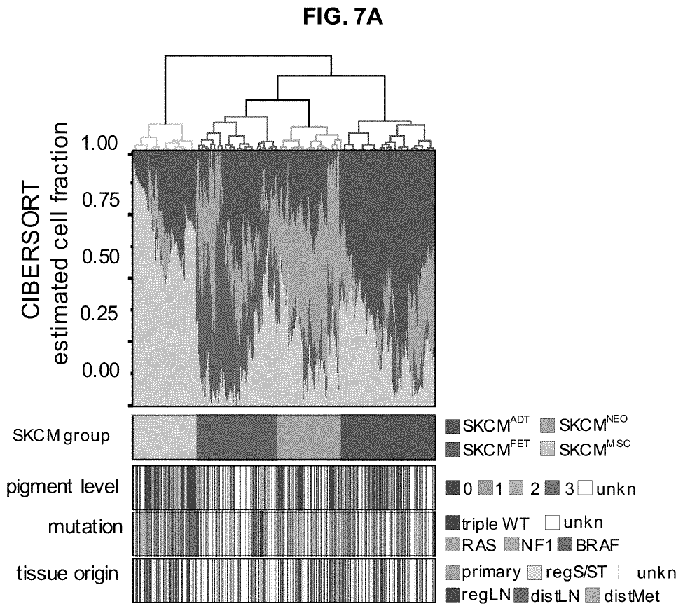

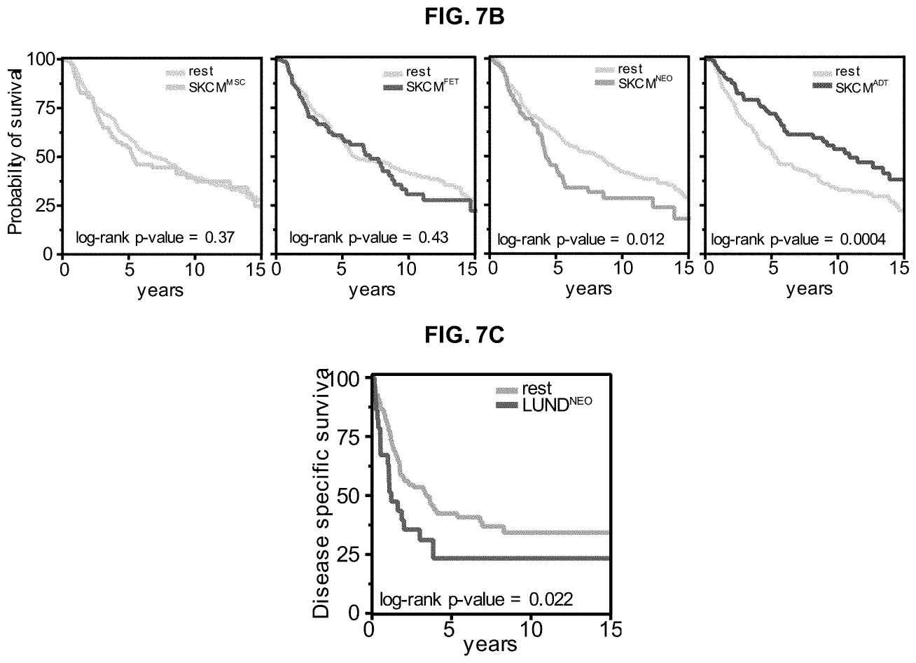

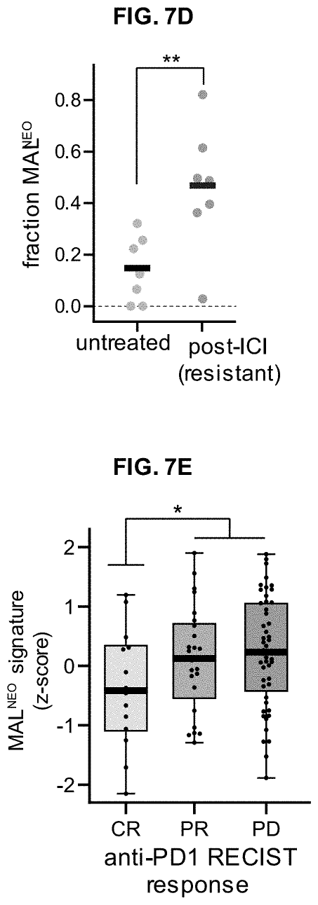

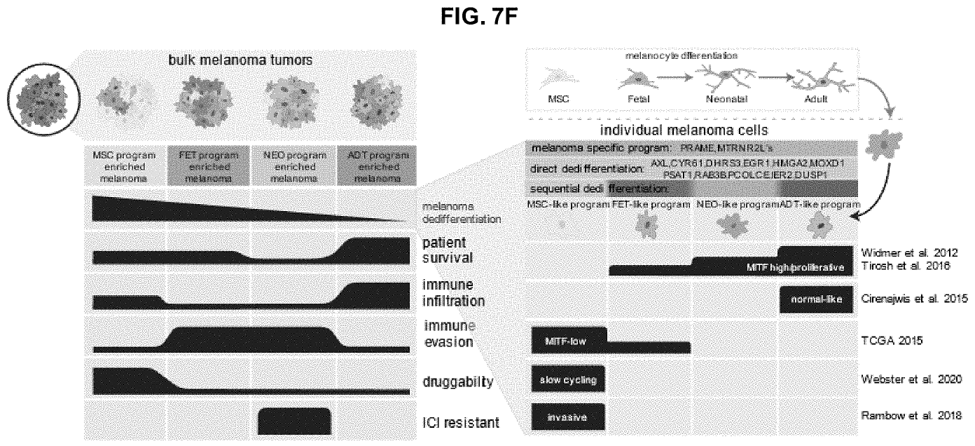

To determine whether gene expression programs characteristic of different human developmental ages offer prognostic value, CIBERSORT was applied to estimate the fraction of melanoma cells similar to ADT, NEO, FET, MSC for all skin cutaneous melanoma (SKCM) tumor samples from The Cancer Genome Atlas (TOGA). Similar to the single cell melanoma dataset (FIG. 6B), inter-tumor heterogeneity was observed in the fractional representation of the four developmental groups (FIG. 7A). Hierarchical clustering of SKCM label distributions classified tumor samples according to the observed predominant developmental group: SKCMADT, SKCM^{NEO}, SKCM^{FET}, SKCM^{MSC}. Neither genetic driver nor tumor site correlated with the developmental group classification of the tumor (FIG. 6A; FIG. 8F).

Using developmentally-defined subclasses of melanoma tumors, the correlation of bulk tumor differentiation status was evaluated with patient outcome. As expected, the most differentiated group (SKCM^{ADT}) exhibited best median overall survival (FIG. 7B, SKCM^{ADT}=11.0 yr vs rest=5.3 yr). Surprisingly, the most dedifferentiated groups (SKCM^{FET}, SKCM^{MSC}) were not associated with worse outcomes; rather, the intermediately differentiated group (SKCM^{NEO}) exhibited the shortest median overall survival (SKCM^{NEO}=4.2 yr vs rest=8.2 yr). To validate this observation, specimens from the Lund University dataset were developmentally-classified and again found that tumors comprised of predominantly NEO-like cells (LUND^{NEO}) were associated with worse outcomes (FIG. 7C, LUND^{NEO}=1.21 yr vs rest=3.43 yr). To better understand this unexpected finding, the expression of transcriptional programs associated with clinical response to therapeutics were evaluated. Indeed, SKCM^{NEO} tumors expressed higher levels of transcripts associated with immune resistance (p-value=1.6×10⁻², SKCM^{NEO} vs rest) and a dearth in immune infiltration signatures (p-value=5.5×10⁻⁴, SKCM^{NEO} vs rest) as well as FDA-approved therapeutic targets (p-value=1.6×10⁻⁵, SKCM^{NEO} vs rest) (FIG. 8G). The SKCM^{MSC} tumor group was unique in its increased expression of FDA-approved therapeutic targets (p-value=4.6×10−21, SKCM^{MSC} vs rest) in agreement with previous studies of stem-like melanoma cells. The MAL^{NEO} signature presented virtually no enrichment for previously published prognostic signatures, with the striking exception of genes that are poorly expressed in tumors that respond to the immune checkpoint inhibitor (ICI), nivolumab (FIG. 8H, Table 4). Consistent with this finding, the fraction of MAL^{NEO} cells within a melanoma was a strong predictor of ICI-resistant versus treatment naïve tumors (FIG. 7D, unpaired one-sided t-test, p-value=0.0099) and, importantly, the MAL^{NEO} signature was significantly more expressed in tumors exhibiting only partial or no response to ICI therapy as compared to full response (FIG. 7E, unpaired, one-sided t-test p-value=0.018).

Taken together, classification using human epidermal melanocyte developmental stage signatures revealed that at least four states of dedifferentiation constitute individual tumors (FIG. 7F). The proportion of melanocytes that have readopted a neonatal-like signature is associated with worse prognosis and higher likelihood of resistance to ICI therapy, demonstrating that while some amount of dedifferentiation is associated with worse prognosis, overall survival, immune evasion, and immune resistance are not linearly correlated with dedifferentiation (FIG. 7F).

This study has provided a fresh-from-skin human epidermal melanocyte dataset that encompasses human development, sex and diverse race/ethnicities and includes multiple donor-matched anatomic locations. These findings deliver a unique perspective on human melanocyte biology through the characterization of distinct transcriptional programs specific to development and function. Thus, the transcriptional programs identified here are valuable for understanding the diversity and malignant transformation of human melanocytes.

An additional population of epidermal melanocytes was identified that appear early during human development. It is possible epidermal v-mels are hypopigmented descendants of previously defined sweat gland stem cells. It is also possible that v-mels derive from Schwann-cell derived melanoblasts which undergo a distinct lineage specification pathway. Having identified genes in each subtype that are conserved through fetal development to adulthood, markers that permit exploration of these hypotheses in future studies have been identified. As the predominant class of melanocytes in volar regions, it was speculated these v-mels could represent a distinct cell of origin of acral melanomas and demonstrated that the v-mel signature was significantly elevated in primary AMs compared to other primary cutaneous melanomas (FIG. 3J-K). Since AM is associated with poor therapeutic response and overall survival, assessing whether the v-mel origin confers therapeutic vulnerabilities unique to AMs could be clinically valuable.

By characterizing melanoma dedifferentiation using human-specific developmental programs, this work sheds light on the relationships among developmental stages, tumor characteristics, and melanoma cell transcriptional states (FIG. 6 and FIG. 7). For example, with 63 years as the average age of melanoma diagnosis, the in situ adult melanocyte transcriptome provides a relevant basis for interrogating disease etiology and progression. Moreover, these analyses identified the transcriptional state associated with neonatal melanocytes correlated to worst overall survival and predicted response to immune checkpoint inhibitors. One limitation of the cohort is the neonatal samples are limited to a single anatomic location (foreskin). The pseudotime analysis and the frequency of observed sequential dedifferentiation in melanoma both support the hypothesis that neonatal melanocytes represent an intermediate developmental stage, but the contribution of the foreskin tissue environment on the transcriptional programs cannot ruled out. Indeed, foreskin is established as immunologically hyperactive, it is therefore possible that foreskin melanocytes evolved to express more immune-evasive programs. Regardless, the discovery of the prognostic value of this expression program could prove clinically valuable in the a priori prediction of therapeutic response to immune checkpoint inhibition. Due to tissue availability and ease of culture, the neonatal melanocyte transcriptome is often considered the baseline “normal differentiated program” for comparison to melanoma transcriptomes. This technical artifact can explain why this program has been previously underappreciated. Melanoma-specific genes directly acquired in all stages of dedifferentiation were identified (FIG. 6G, FIG. 7G, Table 3), suggesting that these genes may undergo positive selection during early metastatic dissemination. Along with the widely-accepted diagnostic melanoma biomarker PRAME and an established marker of invasion AXL, additional melanoma-associated genes were identified. Further investigation into the mechanistic roles of these gene sets could reveal previously uncharacterized drivers of melanoma metastasis.

TABLE 1
Differentially expressed genes between donor-matched volar and non-volar cutaneous melanocytes in FIG. 3. Differentially expressed genes
were determined by Two-sided Wilcoxon Rank Sum Test. Adjusted p-value computed using Benjamini-Hochberg multiple testing procedure.
2,042 site-enriched Non-volar Cutaneous vs Volar DE genes from FIG. 3A

| | | | | | | | | | |
|---|---|---|---|---|---|---|---|---|---|
| gene | log2 fold chage | p-value | adj p-value | neg log10(adj p-value) | gene | log2 fold chage | p-value | adj p-value | neg log10(adj p-value) |

Volar melanocyte enriched genes (ordered by adj p-value)

| | | | | | | | | | |
|---|---|---|---|---|---|---|---|---|---|
| NTRK2 | −3.754142284 | 6.0618E−117 | 1.4848E−112 | 111.8283411 | MILR1 | −1.915088654 | 0.000230505 | 0.003987284 | 2.399322812 |
| ID2 | −3.118757248 | 7.3139E−114 | 8.9573E−110 | 109.0478211 | REC8 | −0.483075172 | 0.000250983 | 0.004320157 | 2.364500478 |
| ID3 | −5.690807819 | 1.8069E−100 | 1.4753E−96 | 95.8311202 | POLH | −0.671879053 | 0.00025536 | 0.004386244 | 2.357907191 |
| RAB3B | −2.387235165 | 7.54019E−47 | 8.79473E−44 | 43.05577743 | RASSF1 | −0.513198197 | 0.000284417 | 0.004847961 | 2.314440919 |
| NAV2 | −1.751379967 | 2.37292E−35 | 1.38387E−32 | 31.85890558 | GABBR1 | −0.532790005 | 0.000296878 | 0.005039315 | 2.297628512 |
| MEG3 | −2.796798229 | 3.07647E−29 | 1.17742E−26 | 25.92906722 | RCC1 | −0.853792727 | 0.000298043 | 0.00505558 | 2.296228999 |
| IGDCC4 | −1.539427519 | 1.78188E−28 | 6.41843E−26 | 25.19257112 | TPD52 | −0.492767602 | 0.000299608 | 0.005075111 | 2.294554451 |
| PDLIM3 | −1.612020731 | 6.19953E−27 | 1.92217E−24 | 23.71620878 | BTG1 | −0.407893956 | 0.000301619 | 0.005105643 | 2.291949539 |
| FRZB | −1.646557808 | 1.34414E−25 | 3.50248E−23 | 22.45562373 | BAZ2B | −0.533544838 | 0.00031108 | 0.005247661 | 2.280034191 |
| PHACTR3 | −1.470532894 | 7.049E−25 | 1.76182E−22 | 21.75403896 | CFAP61 | −1.924341798 | 0.000315875 | 0.005317551 | 2.274288376 |
| CDR1 | −1.032910705 | 1.3114E−22 | 2.67679E−20 | 19.57238491 | BRD2 | −0.437898695 | 0.000329578 | 0.005525457 | 2.257631822 |
| MIR3654 | −0.924516261 | 1.63998E−22 | 3.29259E−20 | 19.48246274 | CDK12 | −0.473014653 | 0.000329827 | 0.005525836 | 2.257602028 |
| PDLIM4 | −1.470458388 | 7.73993E−21 | 1.29851E−18 | 17.88655592 | FBXO9 | −0.50252986 | 0.000334136 | 0.005586569 | 2.252854857 |
| PHGDH | −1.276817083 | 1.00025E−17 | 1.2069E−15 | 14.91832984 | CTGF | −0.960378349 | 0.000350917 | 0.005831321 | 2.234233071 |
| NES | −1.352597833 | 2.85687E−17 | 3.28527E−15 | 14.48342937 | TULP2 | −0.907613456 | 0.000354464 | 0.005878297 | 2.2307485 |
| UACA | −1.034484386 | 3.68537E−17 | 4.16104E−15 | 14.38079777 | CCND2 | −1.739438057 | 0.000365307 | 0.006025474 | 2.220008765 |
| EIF5AL1 | −1.093874812 | 6.40036E−17 | 7.03006E−15 | 14.15304085 | MRPS25 | −0.48961851 | 0.000374311 | 0.006140904 | 2.211767676 |
| SHC4 | −1.831962585 | 3.49658E−16 | 3.56855E−14 | 13.44750809 | MGRN1 | −0.600480974 | 0.000374847 | 0.006145582 | 2.211436962 |
| PTN | −1.60205555 | 3.01005E−15 | 2.85768E−13 | 12.54398693 | DLGAP1 | −0.827429175 | 0.000425877 | 0.006903656 | 2.160920852 |
| TOX3 | −1.691528678 | 3.84395E−15 | 3.57999E−13 | 12.44611824 | GSTM1 | −2.121361017 | 0.00044287 | 0.007146025 | 2.14593549 |
| DAAM1 | −1.109912515 | 5.98286E−15 | 5.42756E−13 | 12.26539513 | SPDYE6 | −0.695437849 | 0.000450701 | 0.007234258 | 2.14060598 |
| LOC284454 | −1.21387291 | 8.93756E−15 | 7.81845E−13 | 12.10687917 | LDB1 | −0.582141876 | 0.000470401 | 0.00749642 | 2.125146108 |
| CECR2 | −1.307452559 | 1.11969E−13 | 8.847E−12 | 11.05320419 | PDLIM7 | −0.55500263 | 0.000480093 | 0.007631023 | 2.11741722 |
| ARID5A | −1.309638143 | 4.36493E−13 | 3.11705E−11 | 10.50625683 | CDK2 | −0.477404356 | 0.000506735 | 0.008028438 | 2.095368922 |
| RUNX3 | −0.877994716 | 6.86589E−13 | 4.76411E−11 | 10.32201809 | C5orf45 | −0.813335001 | 0.000516337 | 0.008143689 | 2.08917882 |
| SLC12A5 | −1.70690608 | 1.0757E−12 | 7.29867E−11 | 10.13675644 | UST | −0.571886778 | 0.000523076 | 0.008228782 | 2.084664417 |
| CREB5 | −0.968270242 | 1.15405E−12 | 7.78711E−11 | 10.10862374 | CREBBP | −0.440704614 | 0.000528288 | 0.008294799 | 2.081194157 |
| IGFBP3 | −1.045001268 | 3.24344E−12 | 2.06662E−10 | 9.684739789 | ZKSCAN1 | −0.465340495 | 0.000530477 | 0.008323829 | 2.07967685 |
| HIST1H1E | −0.909290969 | 6.55059E−12 | 4.01125E−10 | 9.396719976 | ARHGAP24 | −0.506841421 | 0.000540726 | 0.008452173 | 2.073031637 |
| ERBB3 | −0.917368829 | 6.82942E−12 | 4.16119E−10 | 9.38078258 | PXMP4 | −0.583741486 | 0.00059183 | 0.00917486 | 2.037400571 |
| FAM210B | −0.871610045 | 7.57578E−12 | 4.54807E−10 | 9.342172899 | HSPB2 | −0.531414628 | 0.000605254 | 0.009353364 | 2.02903215 |
| TXNIP | −0.860300303 | 4.33145E−11 | 2.37348E−09 | 8.624614925 | BID | −0.678387821 | 0.000674465 | 0.010323091 | 1.98619024 |
| PLAT | −1.019935846 | 5.26297E−11 | 2.85834E−09 | 8.543886335 | LOC101927481 | −1.164224505 | 0.000676727 | 0.010340458 | 1.985460216 |
| KIAA1683 | −1.209673762 | 1.1385E−10 | 5.97138E−09 | 8.223925331 | ZSWIM4 | −1.092056274 | 0.000680255 | 0.010374948 | 1.984014051 |
| LINC00473 | −1.922710896 | 1.58793E−10 | 8.18839E−09 | 8.086801734 | PHF23 | −0.494915485 | 0.000686337 | 0.010448188 | 1.98095901 |
| CXXC5 | −0.940584302 | 3.5077E−10 | 1.71835E−08 | 7.764887888 | TBC1D7 | −0.520888388 | 0.000703376 | 0.010661193 | 1.972194213 |
| KCNQ1OT1 | −1.014119387 | 5.78022E−10 | 2.71748E−08 | 7.565833652 | GABPB1-AS1 | −0.512583017 | 0.000717554 | 0.010849238 | 1.964600767 |
| TUBB2B | −0.821122766 | 5.85632E−10 | 2.74273E−08 | 7.561817072 | MYCBP2 | −0.444684952 | 0.000719222 | 0.010867511 | 1.963869903 |
| TTYH1 | −1.230690241 | 9.3237E−10 | 4.23701E−08 | 7.372940889 | MED 15 | −0.45761013 | 0.000748485 | 0.011261298 | 1.948411557 |
| ANK2 | −0.817035079 | 1.10343E−09 | 4.98661E−08 | 7.302194728 | CCT5 | −0.443800718 | 0.000749152 | 0.011264408 | 1.94829161 |
| ATP10A | −0.775570571 | 1.30535E−09 | 5.82391E−08 | 7.23478535 | SMOX | −0.58307451 | 0.000781159 | 0.01168114 | 1.932514787 |
| DCLK1 | −1.574236512 | 1.76998E−09 | 7.81152E−08 | 7.107264536 | PPIP5K2 | −0.444087595 | 0.000823705 | 0.012279875 | 1.91080606 |
| LOC100190986 | −0.646294057 | 2.84269E−09 | 1.21942E−07 | 6.913847124 | RAB22A | −0.432175994 | 0.000828574 | 0.012344943 | 1.908510897 |
| PHACTR1 | −0.800598741 | 3.49783E−09 | 1.47972E−07 | 6.829820437 | HEXIM1 | −0.397823662 | 0.000838342 | 0.012460162 | 1.904476304 |
| GLCCI1 | −0.903584361 | 8.73357E−09 | 3.51265E−07 | 6.454365739 | IFFO2 | −1.111767173 | 0.000840566 | 0.012485635 | 1.903589349 |
| FJX1 | −1.34338522 | 1.35162E−08 | 5.26338E−07 | 6.278735367 | ADARB1 | −0.424865156 | 0.000860522 | 0.012751141 | 1.894450959 |
| CDH19 | −0.714088798 | 2.62708E−08 | 9.85416E−07 | 6.006380188 | CECR1 | −0.886685908 | 0.000874327 | 0.012924416 | 1.888589084 |
| BMPR1B | −0.762638211 | 2.65854E−08 | 9.95693E−07 | 6.001874589 | SPINT2 | −1.828564048 | 0.000884231 | 0.013031496 | 1.885005709 |
| IGF2BP3 | −1.123615265 | 3.09079E−08 | 1.14359E−06 | 5.941729146 | IRX5 | −0.494660467 | 0.000897651 | 0.013207758 | 1.879170903 |
| NR4A3 | −1.113756299 | 3.42846E−08 | 1.25902E−06 | 5.89996715 | FHL1 | −0.461762488 | 0.00092923 | 0.013629082 | 1.865533386 |
| SEMA6A | −0.677349985 | 3.5274E−08 | 1.29148E−06 | 5.88891177 | DBNDD1 | −0.566572726 | 0.000935822 | 0.013717542 | 1.862723702 |
| TRIO | −0.768395305 | 4.76421E−08 | 1.71862E−06 | 5.764819115 | TRIM27 | −0.496750176 | 0.000941247 | 0.01377877 | 1.860789539 |
| NRP2 | −1.247081518 | 5.56544E−08 | 1.98717E−06 | 5.701764962 | CGN | −1.103968859 | 0.000944714 | 0.013806577 | 1.859913967 |
| NEDD4L | −0.779282808 | 7.39636E−08 | 2.57339E−06 | 5.589494749 | ADGRG6 | −0.728225589 | 0.000971014 | 0.014165591 | 1.848765292 |
| WNT4 | −1.060254097 | 7.61719E−08 | 2.63897E−06 | 5.578565065 | PCF11 | −0.427823395 | 0.000983301 | 0.014327769 | 1.843821437 |
| POTEM | −2.180957079 | 8.02417E−08 | 2.76433E−06 | 5.558409504 | CDK15 | −0.541725755 | 0.001006085 | 0.014624948 | 1.834905678 |
| LOC101930452 | −0.76753062 | 1.39367E−07 | 4.65709E−06 | 5.331885434 | MCHR1 | −0.527775466 | 0.001060832 | 0.015329807 | 1.814463324 |
| RAB6B | −0.802143812 | 1.48199E−07 | 4.93875E−06 | 5.30638307 | HNRNPAB | −0.353627503 | 0.001087782 | 0.0156546 | 1.805358018 |
| CTSH | −0.838142276 | 1.522E−07 | 5.06505E−06 | 5.295416075 | RASSF8 | −0.4402273 | 0.001164624 | 0.016682048 | 1.777750629 |
| SYT17 | −1.624316216 | 2.3863E−07 | 7.6807E−06 | 5.114599267 | HRK | −0.479664654 | 0.001180785 | 0.016893785 | 1.77227304 |
| MIA | −0.658910453 | 2.61287E−07 | 8.38789E−06 | 5.076347217 | STOX2 | −0.482516289 | 0.00118282 | 0.016913016 | 1.771778928 |
| AMOTL1 | −0.81366688 | 2.78676E−07 | 8.86479E−06 | 5.052331593 | LDLRAD3 | −0.426682949 | 0.001200177 | 0.017121226 | 1.766465133 |
| LINC00632 | −0.811102152 | 3.4611E−07 | 1.09107E−05 | 4.962146696 | EIF4G3 | −0.40801996 | 0.001256252 | 0.01779678 | 1.749658577 |
| KLHL24 | −0.672709584 | 5.91357E−07 | 1.79488E−05 | 4.745963892 | ANKRD44-IT1 | −0.461289287 | 0.001360508 | 0.019118927 | 1.71853649 |
| MYC | −0.730143487 | 9.47784E−07 | 2.78692E−05 | 4.554875696 | PLK2 | −0.486807287 | 0.001408623 | 0.019715891 | 1.70518359 |
| ENC1 | −1.312815905 | 1.34786E−06 | 3.84337E−05 | 4.415287309 | DDIT4 | −0.524279058 | 0.001412616 | 0.019760487 | 1.704202365 |
| ZNF608 | −0.917593718 | 1.52493E−06 | 4.3032E−05 | 4.366208724 | NAB2 | −0.551342607 | 0.001418625 | 0.019833217 | 1.702606845 |
| SOBP | −1.174356341 | 2.33231E−06 | 6.29852E−05 | 4.200761577 | GNL3 | −0.457763195 | 0.001430309 | 0.019955532 | 1.699936696 |
| SNTB2 | −0.718637288 | 2.36878E−06 | 6.38996E−05 | 4.194502047 | DLGAP4 | −0.451495916 | 0.001482614 | 0.020561134 | 1.686952932 |
| BACH1 | −0.74126488 | 2.41373E−06 | 6.48977E−05 | 4.187770667 | FRMD4B | −0.577405989 | 0.001491248 | 0.020656332 | 1.684946804 |
| IRX3 | −0.877258003 | 2.51052E−06 | 6.71317E−05 | 4.173072251 | KIF21A | −0.433043897 | 0.001533913 | 0.021167131 | 1.674337996 |
| CSNK2B | −0.365997672 | 2.74397E−06 | 7.32943E−05 | 4.134929521 | LRRN4CL | −0.478344798 | 0.001553873 | 0.021370338 | 1.67018861 |
| DENND2A | −0.970220387 | 2.88662E−06 | 7.69368E−05 | 4.113866129 | CDIP1 | −0.586156309 | 0.001558594 | 0.021411221 | 1.669358574 |
| FMNL2 | −0.848814726 | 3.09759E−06 | 8.19356E−05 | 4.086527566 | CAMSAP2 | −0.556104779 | 0.001559557 | 0.021412445 | 1.669333748 |
| BANP | −0.80249989 | 3.35108E−06 | 8.78814E−05 | 4.056102989 | WDR46 | −0.579849482 | 0.001579736 | 0.021665208 | 1.664237133 |
| CHP1 | −0.162610397 | 3.44472E−06 | 9.01442E−05 | 4.045062211 | CHST2 | −0.933671594 | 0.001582486 | 0.021690778 | 1.663724861 |
| ZNF429 | −0.7869609 | 3.52913E−06 | 9.22546E−05 | 4.035012015 | HN1 | −0.479145765 | 0.00159899 | 0.021880253 | 1.659947654 |
| LOC389906 | −1.091573715 | 3.7587E−06 | 9.7838E−05 | 4.009492593 | FOXK1 | −0.459331453 | 0.001672289 | 0.02281191 | 1.641838345 |
| CSGALNACT1 | −1.370385528 | 3.9718E−06 | 0.000102947 | 3.987385093 | NOP58 | −0.448678046 | 0.001672662 | 0.02281191 | 1.641838345 |
| CTBP2 | −0.52206099 | 5.48286E−06 | 0.000137741 | 3.86093778 | ZNF814 | −0.349755406 | 0.001712996 | 0.023297131 | 1.632697552 |
| MYLIP | −0.661476552 | 5.55912E−06 | 0.000139228 | 3.856272714 | RNF41 | −0.495777637 | 0.00172046 | 0.023373987 | 1.631267199 |
| LINC00518 | −0.736257374 | 5.5946E−06 | 0.000139974 | 3.853954017 | SNHG3 | −0.423139095 | 0.001728146 | 0.023464088 | 1.629596315 |
| CHD1 | −0.586650491 | 6.77846E−06 | 0.000165866 | 3.780243189 | ZFC3H1 | −0.502495468 | 0.001757936 | 0.023789439 | 1.623615803 |
| PPP1R10 | −0.649186611 | 7.35214E−06 | 0.000178654 | 3.747986974 | KLHL6 | −1.532083273 | 0.001764991 | 0.023832237 | 1.622835195 |
| OPN3 | −0.598926663 | 9.41352E−06 | 0.000223859 | 3.650025454 | PCCB | −0.523212433 | 0.001785914 | 0.024084305 | 1.618265873 |
| POLR2A | −0.476708978 | 9.71862E−06 | 0.00023089 | 3.636594131 | SESN3 | −0.343697339 | 0.001786608 | 0.024084305 | 1.618265873 |
| CTSC | −0.605605066 | 1.06201E−05 | 0.000250607 | 3.601007376 | IMPDH2 | −0.450109422 | 0.001843815 | 0.024760096 | 1.606247677 |
| PSAT1 | −0.578512132 | 1.07734E−05 | 0.000253246 | 3.596457229 | NOP14 | −0.538847208 | 0.001868903 | 0.025006583 | 1.601945652 |
| PEAK1 | −0.736703396 | 1.13281E−05 | 0.000265776 | 3.575484584 | LINC01004 | −0.750608683 | 0.001871077 | 0.025016463 | 1.601774093 |
| RASIP1 | −1.189228892 | 1.1853E−05 | 0.000277559 | 3.556645278 | LMCD1 | −0.497713119 | 0.001937594 | 0.025807199 | 1.588259134 |
| WIPI1 | −0.633573592 | 1.75472E−05 | 0.000395038 | 3.403360719 | LMO4 | −0.34315443 | 0.001965042 | 0.026115979 | 1.583093685 |
| BCL2 | −0.54893446 | 1.79577E−05 | 0.000403908 | 3.39371714 | SETD2 | −0.421881348 | 0.002049542 | 0.027112592 | 1.566828954 |
| FADS1 | −0.561894119 | 1.96181E−05 | 0.000438435 | 3.358094301 | RC3H1 | −0.406871885 | 0.002049993 | 0.027112592 | 1.566828954 |
| TSPAN14 | −0.582472086 | 2.06375E−05 | 0.000459541 | 3.337675782 | IGF2BP1 | −0.773612142 | 0.00205823 | 0.02720684 | 1.565321895 |
| NRIP1 | −0.614210427 | 2.22747E−05 | 0.00049286 | 3.307276454 | FNBP1L | −0.466912508 | 0.002083008 | 0.027489874 | 1.560827249 |
| DAB2 | −0.66539824 | 2.40752E−05 | 0.00053078 | 3.27508526 | VHL | −0.44554311 | 0.002091023 | 0.02756896 | 1.55957961 |
| ZNF276 | −0.567812324 | 2.42435E−05 | 0.00053401 | 3.272450648 | CDK6 | −0.385241359 | 0.002096075 | 0.027604339 | 1.559022651 |
| ORAI2 | −0.686376274 | 2.61078E−05 | 0.00056843 | 3.245323181 | TFRC | −0.676267922 | 0.002172023 | 0.028479253 | 1.54547141 |
| TTN | −0.929804564 | 2.76918E−05 | 0.000598661 | 3.222819238 | E4F1 | −0.576384068 | 0.002192059 | 0.028697112 | 1.542161814 |
| TOB2 | −0.563909054 | 2.82439E−05 | 0.000607913 | 3.216158365 | JMY | −0.549129188 | 0.002218553 | 0.029012937 | 1.537408311 |
| CKS2 | −0.530575454 | 2.95787E−05 | 0.000635527 | 3.196865975 | LINC01000 | −0.448901206 | 0.002270319 | 0.029610858 | 1.52854901 |
| PART1 | −0.585063338 | 3.08515E−05 | 0.000659403 | 3.180848917 | SOX12 | −0.835556686 | 0.002309082 | 0.030025417 | 1.522510957 |
| CIRH1A | −0.70634228 | 3.21854E−05 | 0.000686715 | 3.163223325 | BOK | −0.59395057 | 0.002364575 | 0.030646049 | 1.51362551 |
| LOC728392 | −1.1147089 | 3.29916E−05 | 0.000702081 | 3.153612535 | FAM210A | −0.518214941 | 0.002447388 | 0.031633947 | 1.499846619 |
| LOC100505938 | −1.422665238 | 3.92821E−05 | 0.000820968 | 3.085673527 | LOC100126784 | −1.240818024 | 0.002601911 | 0.033419617 | 1.475998534 |
| BOP1 | −0.766389728 | 4.31102E−05 | 0.000891843 | 3.04971172 | KDM5B | −0.462473303 | 0.002649807 | 0.033967216 | 1.468940052 |
| ANKRD44 | −0.582318068 | 4.43391E−05 | 0.000914177 | 3.038969731 | ACVR2B | −0.504360676 | 0.002774207 | 0.035408428 | 1.450893359 |
| SERTAD1 | −0.698935449 | 5.17339E−05 | 0.001046822 | 2.98012705 | MRAS | −0.453637511 | 0.002839221 | 0.036107937 | 1.44239733 |
| NID1 | −0.597748458 | 5.4009E−05 | 0.001089701 | 2.962692632 | NCS1 | −0.661911011 | 0.002884309 | 0.036605315 | 1.436455856 |
| LRP6 | −0.522486329 | 5.54763E−05 | 0.00111563 | 2.952479703 | LOC105747689 | −0.924577296 | 0.002895875 | 0.03669507 | 1.435392278 |
| PRICKLE1 | −1.495618582 | 5.63513E−05 | 0.001132295 | 2.946040228 | PAX8-AS1 | −0.598881483 | 0.00290826 | 0.036794894 | 1.434212448 |
| HIST1H1D | −1.286005497 | 5.67838E−05 | 0.001139117 | 2.943431791 | LYPD1 | −0.386095554 | 0.002907174 | 0.036794894 | 1.434212448 |
| SENP6 | −0.601767957 | 6.53928E−05 | 0.001294851 | 2.887780312 | DHX33 | −0.354392052 | 0.003009089 | 0.038011664 | 1.420083113 |
| ID1 | −2.44384861 | 7.56247E−05 | 0.001470121 | 2.832646987 | BMPR2 | −0.318912804 | 0.003109468 | 0.038938295 | 1.409623072 |
| NR4A2 | −0.50006777 | 7.63317E−05 | 0.001481512 | 2.829294963 | WDR74 | −0.594340622 | 0.003151602 | 0.039385378 | 1.404664985 |
| LDB3 | −0.802973986 | 8.21827E−05 | 0.001585025 | 2.799963789 | THSD7A | −0.777302563 | 0.003153772 | 0.039392403 | 1.404587525 |
| NR2F2 | −0.570629954 | 8.28458E−05 | 0.001595303 | 2.797156935 | ZNF195 | −0.489423782 | 0.003180597 | 0.039686981 | 1.401351939 |
| NLN | −0.594447315 | 0.00010111 | 0.001906528 | 2.719756921 | CARHSP1 | −0.395971119 | 0.003234016 | 0.040230573 | 1.395443777 |
| FBXO17 | −0.595686913 | 0.000105884 | 0.001990422 | 2.701054894 | PPARGC1B | −0.579691708 | 0.003355842 | 0.041535111 | 1.381584621 |
| RSRP1 | −0.606843889 | 0.00010692 | 0.002008355 | 2.697159434 | GOLIM4 | −0.373602271 | 0.003375156 | 0.041710928 | 1.379750145 |
| ID4 | −0.54593128 | 0.00011378 | 0.00211773 | 2.674129431 | PYCR1 | −0.647311985 | 0.003412113 | 0.042103928 | 1.375677383 |
| MYO9B | −0.505093813 | 0.000117639 | 0.002176316 | 2.662278043 | DMXL2 | −0.57409811 | 0.003482015 | 0.042836999 | 1.368180959 |
| SOX2 | −3.345513344 | 0.000124455 | 0.002281744 | 2.641733038 | CHD8 | −0.441651225 | 0.003510265 | 0.043162862 | 1.364889765 |
| SLC10A1 | −1.008467793 | 0.000130547 | 0.002389852 | 2.621629055 | XIAP | −0.422707021 | 0.003609656 | 0.044296055 | 1.353634954 |
| PAMR1 | −0.511576355 | 0.000132563 | 0.002423136 | 2.615622124 | RGS10 | −0.306042314 | 0.003616083 | 0.044352693 | 1.353080002 |
| W1PI2 | −0.576881826 | 0.000135342 | 0.0024684 | 2.607584511 | ENOSF1 | −0.404787689 | 0.003625173 | 0.044441932 | 1.352207073 |
| PRPF8 | −0.484960914 | 0.000139442 | 0.002541289 | 2.594945996 | OSBP | −0.458655208 | 0.003643986 | 0.044650228 | 1.350176323 |
| PDE8A | −0.517045498 | 0.000142695 | 0.002592855 | 2.586221728 | SELO | −0.566946089 | 0.003664612 | 0.044876826 | 1.347977868 |
| GFOD2 | −0.735636413 | 0.000151785 | 0.002741765 | 2.561969826 | TOB1 | −0.382177025 | 0.00372771 | 0.045516713 | 1.341829108 |
| BCHE | −0.575244129 | 0.000152958 | 0.002760911 | 2.558947646 | GOLGA3 | −0.451195419 | 0.003776024 | 0.045992011 | 1.337317603 |
| KLF13 | −0.399398565 | 0.000166465 | 0.002982738 | 2.525384893 | TCEAL7 | −0.802196622 | 0.003837866 | 0.046560021 | 1.33198683 |
| CRLF3 | −0.682515323 | 0.000171122 | 0.003057236 | 2.514671073 | NECAB2 | −1.256868005 | 0.003849062 | 0.046629 | 1.331343895 |
| GIT2 | −0.501495779 | 0.00017194 | 0.003069609 | 2.512916872 | SFSWAP | −0.359725833 | 0.003861895 | 0.046738228 | 1.330327753 |
| SYT11 | −0.471145779 | 0.000176338 | 0.003129873 | 2.504473271 | DUSP16 | −0.444329172 | 0.003966786 | 0.04789432 | 1.319715991 |
| ATM | −0.448352098 | 0.000204908 | 0.003592709 | 2.444578004 | LIMS1 | −0.407905877 | 0.003967403 | 0.04789432 | 1.319715991 |
| SQLE | −0.507528722 | 0.000211126 | 0.003691164 | 2.432836666 | FAM212A | −0.432342947 | 0.003973581 | 0.047945271 | 1.319254226 |
| PRR14L | −0.529761672 | 0.000215666 | 0.003754452 | 2.425453433 | TMEM164 | −0.48448503 | 0.004062677 | 0.048923905 | 1.310478886 |
| NT5DC2 | −0.599480212 | 0.000217843 | 0.003784295 | 2.422014999 | TRABD | −0.388524652 | 0.004123433 | 0.049582415 | 1.304672321 |
| TIMP3 | −0.803760767 | 0.000223896 | 0.003876193 | 2.411594558 | DARS2 | −0.654618859 | 0.004148437 | 0.049785313 | 1.30289876 |

Non-volar cutaneous melanocyte enriched genes (ordered by adj p-value)

| | | | | | | | | | |
|---|---|---|---|---|---|---|---|---|---|
| AKAP12 | 2.699370384 | 9.46219E−85 | 5.79417E−81 | 80.23700882 | RUNX2 | 1.118347764 | 4.65509E−06 | 0.000118526 | 3.926187623 |
| MCOLN3 | 2.833744526 | 2.33043E−82 | 1.14163E−78 | 77.94247443 | TRAPPC6A | 0.616699755 | 4.69528E−06 | 0.000119425 | 3.922905173 |
| ASAH1 | 1.898617983 | 3.00869E−76 | 1.22825E−72 | 71.91071352 | RPS6 | 0.163113698 | 4.72693E−06 | 0.000120105 | 3.920437814 |
| SLITRK2 | 2.027208567 | 1.05148E−73 | 3.67929E−70 | 69.43423586 | PRKACB | 0.715507805 | 4.8469E−06 | 0.000123026 | 3.910003391 |
| IFITM3 | 2.069860458 | 1.67316E−71 | 5.12281E−68 | 67.29049201 | NGFRAP1 | 0.551466346 | 4.87915E−06 | 0.000123716 | 3.90757289 |
| APOE | 2.012315035 | 7.66686E−71 | 2.08658E−67 | 66.68056517 | TMEM248 | 0.52988708 | 5.0294E−06 | 0.000127394 | 3.89485086 |
| APCDD1 | 2.50354743 | 2.56261E−67 | 6.27685E−64 | 63.20225852 | RAB6A | 0.500867665 | 5.10319E−06 | 0.00012913 | 3.888974017 |
| CST3 | 1.76763618 | 9.00599E−64 | 2.00539E−60 | 59.69780134 | CLPTM1 | 0.615574181 | 5.21846E−06 | 0.00013191 | 3.879721565 |
| SEMA5A | 2.578830957 | 8.59542E−62 | 1.75447E−58 | 57.75585437 | GNAS | 0.335209876 | 5.22943E−06 | 0.000132051 | 3.87925737 |
| LGI3 | 2.40937376 | 3.1473E−60 | 5.92999E−57 | 56.22694595 | ABLIM1 | 1.204136848 | 5.29079E−06 | 0.000133463 | 3.874638836 |
| HPGD | 2.589627504 | 1.77195E−57 | 3.10016E−54 | 53.50861605 | DSTYK | 0.516884863 | 5.33042E−06 | 0.000134324 | 3.871844931 |
| HLA-C | 2.514072895 | 8.54598E−54 | 1.3955E−50 | 49.85526956 | RHOBTB3 | 0.449962467 | 5.37117E−06 | 0.000135212 | 3.868984108 |
| S100A6 | 1.612758279 | 1.19085E−50 | 1.82305E−47 | 46.73920224 | ATP2C1 | 0.80351615 | 5.47903E−06 | 0.000137741 | 3.86093778 |
| CALM1 | 0.955999255 | 6.16728E−50 | 8.88596E−47 | 46.05129562 | RPL32 | 0.181783408 | 5.49607E−06 | 0.000137931 | 3.860337905 |
| HLA-A | 1.768987179 | 8.28804E−50 | 1.12782E−46 | 45.94776105 | ROBO1 | 0.570848763 | 5.55051E−06 | 0.000139155 | 3.856502326 |
| CD99 | 1.023284674 | 5.00759E−49 | 6.45557E−46 | 45.19006532 | RNF13 | 0.602058768 | 5.67191E−06 | 0.000141763 | 3.848436928 |
| CD59 | 0.904966891 | 5.66713E−49 | 6.94053E−46 | 45.15860738 | MRPL53 | 0.537935197 | 5.67983E−06 | 0.000141816 | 3.848273906 |
| GMPR | 1.911767364 | 1.15917E−46 | 1.29058E−43 | 42.88921655 | ALG14 | 0.917375922 | 5.81114E−06 | 0.000144947 | 3.838790631 |
| VAMP8 | 1.84511435 | 3.5539E−46 | 3.78475E−43 | 42.42196289 | SLC25A3 | 0.440015107 | 5.85721E−06 | 0.000145948 | 3.835802966 |
| PSAP | 1.161190629 | 4.02854E−46 | 4.11146E−43 | 42.38600372 | TRIM47 | 1.069660306 | 5.87083E−06 | 0.00014599 | 3.835677166 |
| GSTP1 | 1.082921386 | 5.66959E−46 | 5.55484E−43 | 42.25532847 | ATPIF1 | 0.454298139 | 5.87083E−06 | 0.00014599 | 3.835677166 |
| EMP1 | 1.647157192 | 9.92185E−44 | 9.34715E−41 | 40.029321 | GBA | 0.67298305 | 5.87947E−06 | 0.000146056 | 3.835479177 |
| S100A10 | 0.941818833 | 1.30788E−43 | 1.18649E−40 | 39.92573614 | TFAP2B | 0.526278913 | 5.89815E−06 | 0.000146372 | 3.834541364 |
| KIF1A | 3.677368402 | 1.63038E−43 | 1.42623E−40 | 39.84581077 | IDI2-AS1 | 0.742687285 | 5.94489E−06 | 0.000147383 | 3.831553646 |
| ANXA2 | 0.793747187 | 7.9711E−43 | 6.73256E−40 | 39.17182003 | RPL17 | 0.268229842 | 5.99383E−06 | 0.000148446 | 3.828432303 |
| MLANA | 0.887632787 | 3.6701E−42 | 2.99652E−39 | 38.52338307 | EEF1A1 | 0.21566771 | 6.05906E−06 | 0.00014991 | 3.824170329 |
| CISD1 | 1.459638119 | 5.81089E−41 | 4.59135E−38 | 37.33805947 | CLTB | 0.612687886 | 6.10418E−06 | 0.000150874 | 3.821386595 |
| PPIA | 0.935300708 | 1.62419E−40 | 1.24321E−37 | 36.90545418 | OLMALINC | 1.010815859 | 6.12923E−06 | 0.00015134 | 3.820046134 |
| RAB2A | 1.314177632 | 8.00067E−40 | 5.93844E−37 | 36.22632762 | PELI1 | 0.793637514 | 6.23522E−06 | 0.000153802 | 3.813037641 |
| ABCA1 | 2.152475834 | 3.52145E−39 | 2.5369E−36 | 35.59569741 | ATP1A1 | 0.586060345 | 6.25982E−06 | 0.000154254 | 3.81176497 |
| DBI | 1.116612196 | 3.06471E−38 | 2.14477E−35 | 34.66861859 | ZDHHC2 | 1.071432352 | 6.3459E−06 | 0.000156218 | 3.806269885 |
| STXBP6 | 1.739797592 | 2.0595E−36 | 1.40126E−33 | 32.85348111 | C12orf57 | 0.452799588 | 6.42172E−06 | 0.000157925 | 3.801548001 |
| ARL6IP5 | 1.220052719 | 2.29528E−36 | 1.51948E−33 | 32.81830566 | SNHG18 | 1.417284846 | 6.45996E−06 | 0.000158706 | 3.799405743 |
| CDC42 | 0.849657595 | 2.46582E−36 | 1.58942E−33 | 32.79876178 | S100A11 | 0.569003701 | 6.50392E−06 | 0.000159467 | 3.797330509 |
| SCD | 1.657971382 | 9.85905E−36 | 6.19199E−33 | 32.20816997 | ANXA4 | 0.551753938 | 6.50342E−06 | 0.000159467 | 3.797330509 |
| HLA-H | 1.625596285 | 1.60064E−35 | 9.80154E−33 | 32.00870559 | H2AFV | 0.5828439 | 6.52098E−06 | 0.000159725 | 3.796627193 |
| NUCKS1 | 0.873907804 | 1.87603E−35 | 1.12077E−32 | 31.95048509 | OSBPL1A | 0.534100354 | 6.88563E−06 | 0.00016832 | 3.773864028 |
| HMGB1 | 0.840564966 | 6.54562E−35 | 3.72857E−32 | 31.42845816 | CCDC53 | 0.696893215 | 6.92917E−06 | 0.000169215 | 3.771560067 |
| ENPP2 | 1.690974116 | 1.75084E−34 | 9.7466E−32 | 31.01114665 | GUSB | 0.645039558 | 6.95798E−06 | 0.00016975 | 3.770190729 |
| SAT1 | 1.017593622 | 2.85559E−34 | 1.55433E−31 | 30.80845737 | RABIF | 0.726716399 | 6.9853E−06 | 0.000170247 | 3.768921376 |
| PHPT1 | 1.295602441 | 6.82435E−34 | 3.63382E−31 | 30.43963692 | ESRP1 | 0.649079442 | 7.05919E−06 | 0.000171877 | 3.764783494 |
| SLC1A4 | 1.714347124 | 7.11524E−34 | 3.7081E−31 | 30.43084875 | PFKM | 0.705948234 | 7.15796E−06 | 0.000174108 | 3.759180721 |
| B2M | 1.079897523 | 9.06545E−34 | 4.62602E−31 | 30.33479209 | EMC2 | 0.651475906 | 7.37981E−06 | 0.000179149 | 3.74678656 |
| CDC42EP3 | 0.870583653 | 1.31501E−33 | 6.57346E−31 | 30.18220606 | COL4A1 | 0.9590047 | 7.43714E−06 | 0.000180362 | 3.743855655 |
| IFITM2 | 1.55622685 | 5.51423E−33 | 2.70131E−30 | 29.56842523 | RPN1 | 0.521993697 | 7.46625E−06 | 0.00018071 | 3.743018031 |
| CALD1 | 1.063532829 | 6.93923E−33 | 3.33274E−30 | 29.47719921 | C8orf59 | 0.489566684 | 7.46225E−06 | 0.00018071 | 3.743018031 |
| ATP1B1 | 1.973482847 | 8.53945E−33 | 4.02241E−30 | 29.39551398 | FAH | 1.289369226 | 7.61871E−06 | 0.000184218 | 3.73466836 |
| MLEC | 1.288105965 | 8.90238E−33 | 4.11424E−30 | 29.38571005 | HNRNPH2 | 0.778444171 | 7.73382E−06 | 0.000186817 | 3.72858445 |
| TIMP1 | 1.904671788 | 4.53171E−32 | 2.05555E−29 | 28.68707198 | ANP32B | 0.469415933 | 7.80272E−06 | 0.000188295 | 3.725160267 |
| ATP5E | 1.008920789 | 4.78847E−32 | 2.07856E−29 | 28.6822372 | RALBP1 | 0.552542269 | 7.87221E−06 | 0.000189785 | 3.721737162 |
| ANXA5 | 0.978220284 | 4.70375E−32 | 2.07856E−29 | 28.6822372 | TOMM5 | 0.512061536 | 7.98793E−06 | 0.000192386 | 3.715827186 |
| CAV1 | 0.933490217 | 4.83702E−32 | 2.07856E−29 | 28.6822372 | C19orf71 | 0.73940742 | 8.01971E−06 | 0.000192961 | 3.714529399 |
| TJP1 | 1.374222875 | 1.53824E−31 | 6.49615E−29 | 28.18734401 | EFNA5 | 0.789746046 | 8.54564E−06 | 0.000205414 | 3.687369953 |
| ADI1 | 1.555323601 | 2.32053E−31 | 9.63373E−29 | 28.01620565 | UGCG | 0.571954191 | 8.55736E−06 | 0.000205494 | 3.687200702 |
| GAS7 | 1.757321477 | 4.34674E−31 | 1.77448E−28 | 27.7509281 | RAP2B | 0.433253556 | 8.60047E−06 | 0.000206327 | 3.685444039 |
| APOD | 1.807666302 | 1.46984E−30 | 5.90201E−28 | 27.22899983 | TREX1 | 0.826975048 | 8.62932E−06 | 0.000206817 | 3.684414641 |
| PTGDS | 2.127863884 | 3.87051E−30 | 1.5291E−27 | 26.81556348 | VDAC2 | 0.448020518 | 8.64181E−06 | 0.000206913 | 3.684211343 |
| CD82 | 1.738316894 | 1.25782E−29 | 4.89031E−27 | 26.31066403 | GPX1 | 0.439471215 | 8.76697E−06 | 0.000209705 | 3.67839056 |
| VAMP5 | 1.459658027 | 6.10148E−29 | 2.29922E−26 | 25.63841856 | GSTO1 | 0.477269739 | 8.78366E−06 | 0.000209899 | 3.677988694 |
| SH3BGRL3 | 1.098842263 | 1.0872E−28 | 4.03483E−26 | 25.39417472 | NBPF1 | 0.673107922 | 8.99651E−06 | 0.000214776 | 3.668013407 |
| REXO2 | 1.374169111 | 1.5788E−28 | 5.77179E−26 | 25.23868914 | UQCRH | 0.4225564 | 9.14045E−06 | 0.000218 | 3.661543086 |
| TPPP3 | 1.690592289 | 2.27022E−28 | 8.05895E−26 | 25.09372173 | POP4 | 0.784631968 | 9.22689E−06 | 0.000219848 | 3.657878192 |
| PRKCDBP | 1.687404037 | 3.01605E−28 | 1.05536E−25 | 24.97659938 | TMEM261 | 0.587527514 | 9.38359E−06 | 0.000223364 | 3.650986673 |
| TYRP1 | 1.289903283 | 4.10722E−28 | 1.41693E−25 | 24.84865072 | RCBTB2 | 0.991737485 | 9.85119E−06 | 0.000233813 | 3.631131256 |
| CSTB | 1.047525644 | 4.22038E−28 | 1.43575E−25 | 24.84292163 | KDELR2 | 0.599416673 | 1.01821E−05 | 0.000241434 | 3.617202261 |
| LGALS3BP | 1.202749133 | 2.68357E−27 | 9.00428E−25 | 24.04555092 | WBP5 | 0.506096721 | 1.03184E−05 | 0.000244428 | 3.611849238 |
| SOD1 | 0.936190724 | 3.01127E−27 | 9.96731E−25 | 24.00142216 | RAD23B | 0.615690112 | 1.03519E−05 | 0.000244986 | 3.610859067 |
| ACP5 | 1.904941559 | 3.2691E−27 | 1.06765E−24 | 23.97157312 | NCSTN | 0.713413417 | 1.04272E−05 | 0.000246478 | 3.608222582 |
| NDUFA1 | 0.965797782 | 4.03743E−27 | 1.30122E−24 | 23.88564878 | RPS15A | 0.24368903 | 1.04351E−05 | 0.000246478 | 3.608222582 |
| NPC2 | 0.84890914 | 5.03338E−27 | 1.60114E−24 | 23.79557119 | KIT | 0.072548807 | 1.06643E−05 | 0.000251406 | 3.599624482 |
| COX8A | 0.991601944 | 5.99213E−27 | 1.88168E−24 | 23.72545331 | SNF8 | 0.588225126 | 1.06771E−05 | 0.000251467 | 3.599518384 |
| H3F3AP4 | 0.858687878 | 6.8109E−27 | 2.08533E−24 | 23.68082609 | TIMP2 | 0.341778815 | 1.07644E−05 | 0.000253246 | 3.596457229 |
| LAMTOR5 | 1.101730585 | 8.63705E−27 | 2.57995E−24 | 23.58838857 | PYGL | 1.933088303 | 1.10486E−05 | 0.000259468 | 3.585915873 |
| SRP14 | 0.762441456 | 8.61267E−27 | 2.57995E−24 | 23.58838857 | ARL6IP1 | 0.443365693 | 1.16737E−05 | 0.000273623 | 3.562847434 |
| LGALS1 | 0.677863419 | 1.52678E−26 | 4.50567E−24 | 23.34624072 | PKM | 0.450106412 | 1.1877E−05 | 0.000277856 | 3.556180455 |
| HLA-B | 2.144397736 | 1.62796E−26 | 4.6912E−24 | 23.3287161 | OSTM1 | 0.514372826 | 1.19943E−05 | 0.000280332 | 3.552327448 |
| LY6E | 1.65532887 | 1.6123E−26 | 4.6912E−24 | 23.3287161 | PRDX5 | 0.415558398 | 1.20105E−05 | 0.000280443 | 3.552155414 |
| EID1 | 0.754281223 | 1.69778E−26 | 4.83551E−24 | 23.31555778 | CPEB4 | 0.643920839 | 1.21974E−05 | 0.000284536 | 3.54586261 |
| PARK7 | 1.082201719 | 2.3516E−26 | 6.62069E−24 | 23.17909691 | MRPL57 | 0.479111999 | 1.22285E−05 | 0.000284991 | 3.545169564 |
| PFN1 | 0.796626091 | 4.29993E−26 | 1.19685E−23 | 22.9219618 | TOMM22 | 0.532216668 | 1.24307E−05 | 0.000289428 | 3.538458753 |
| IGFBP7 | 1.089352965 | 6.63297E−26 | 1.82548E−23 | 22.73862248 | RPS18 | 0.192804843 | 1.27682E−05 | 0.000297004 | 3.527237531 |
| PPP1CB | 1.036500096 | 7.2493E−26 | 1.95126E−23 | 22.70968571 | CRTAP | 0.43452239 | 1.27969E−05 | 0.000297389 | 3.526675515 |
| AHNAK | 0.999482095 | 7.23297E−26 | 1.95126E−23 | 22.70968571 | EIF2S2 | 0.490307748 | 1.28737E−05 | 0.000298889 | 3.524489838 |
| MAL | 1.371539593 | 7.60211E−26 | 2.02398E−23 | 22.69379384 | PSMB7 | 0.515392184 | 1.30042E−05 | 0.000301634 | 3.520519589 |
| VEPH1 | 1.46886754 | 1.29922E−25 | 3.42183E−23 | 22.46574173 | PRDX4 | 0.460111946 | 1.31429E−05 | 0.000304562 | 3.51632394 |
| TSPO | 0.825918555 | 1.62087E−25 | 4.17911E−23 | 22.37891633 | TALDO1 | 0.37966764 | 1.32929E−05 | 0.000307747 | 3.511805975 |
| MFSD12 | 1.178993106 | 1.75386E−25 | 4.4749E−23 | 22.34921658 | KBTBD2 | 0.662996471 | 1.34205E−05 | 0.000310407 | 3.508067824 |
| DMKN | 2.301013947 | 4.93609E−25 | 1.24644E−22 | 21.90432873 | ERGIC3 | 0.46488443 | 1.35553E−05 | 0.00031323 | 3.504136781 |
| NDUFA4 | 0.854060471 | 1.57879E−24 | 3.90614E−22 | 21.40825174 | VPS37B | 0.87341094 | 1.36232E−05 | 0.000314206 | 3.502785709 |
| PSMB4 | 0.787421882 | 2.4586E−24 | 6.0221E−22 | 21.22025231 | SNRPF | 0.483279228 | 1.3612E−05 | 0.000314206 | 3.502785709 |
| SKP1 | 0.689387739 | 2.51922E−24 | 6.10949E−22 | 21.21399523 | ADAM10 | 0.496815711 | 1.38608E−05 | 0.000319385 | 3.495685245 |
| COX6A1 | 0.857328117 | 3.28436E−24 | 7.88698E−22 | 21.10308941 | TRAM1 | 0.542603076 | 1.39447E−05 | 0.000321016 | 3.493473269 |
| PRSS23 | 1.795303226 | 4.1983E−24 | 9.98379E−22 | 21.00070454 | PCBP1 | 0.366541445 | 1.40113E−05 | 0.000322247 | 3.491811373 |
| COMT | 1.001740932 | 5.492E−24 | 1.29347E−21 | 20.88824279 | SZRD1 | 0.59373641 | 1.4156E−05 | 0.000325269 | 3.487757354 |
| MYEOV2 | 0.990652919 | 1.20808E−23 | 2.81817E−21 | 20.55003242 | ATP5D | 0.484262079 | 1.46682E−05 | 0.000336723 | 3.472726953 |
| COL4A2 | 1.40993917 | 1.27545E−23 | 2.94725E−21 | 20.53058346 | TMTC2 | 0.914530396 | 1.47283E−05 | 0.000337785 | 3.471359123 |
| GNG2 | 1.278860092 | 1.77865E−23 | 4.0716E−21 | 20.39023456 | TOMM7 | 0.324899942 | 1.4782E−05 | 0.0003387 | 3.470184796 |
| DAD1 | 0.865105867 | 1.97418E−23 | 4.47736E−21 | 20.34897755 | ASB11 | 0.954565048 | 1.52897E−05 | 0.000349353 | 3.456735968 |
| NDUFA13 | 0.932585597 | 2.78002E−23 | 6.24714E−21 | 20.20431898 | ORMDL2 | 0.637868404 | 1.52829E−05 | 0.000349353 | 3.456735968 |
| RPL23 | 0.668311417 | 2.8917E−23 | 6.43902E−21 | 20.19118045 | SRPK2 | 0.51665324 | 1.52784E−05 | 0.000349353 | 3.456735968 |
| ITM2B | 0.6643942 | 3.56028E−23 | 7.85634E−21 | 20.10477969 | MYO1D | 0.762114227 | 1.53636E−05 | 0.000350713 | 3.455047656 |
| PRELP | 1.005093336 | 3.84874E−23 | 8.41706E−21 | 20.07483944 | FAM96B | 0.483148724 | 1.54951E−05 | 0.000353387 | 3.451749927 |
| NENF | 1.195331573 | 5.17129E−23 | 1.12093E−20 | 19.95042001 | GAS5 | 0.414231628 | 1.56277E−05 | 0.000356079 | 3.448453293 |
| SSR4 | 0.831053674 | 6.17063E−23 | 1.32582E−20 | 19.87751549 | EZR | 0.591603279 | 1.58082E−05 | 0.000359856 | 3.443871372 |
| SLC45A2 | 1.557040691 | 9.10268E−23 | 1.93879E−20 | 19.71246874 | ADD3 | 0.606239736 | 1.58691E−05 | 0.000360909 | 3.442602772 |
| TMBIM6 | 0.891980529 | 9.24403E−23 | 1.95192E−20 | 19.70953688 | HSP90AB1 | 0.36540854 | 1.62578E−05 | 0.000369404 | 3.432498213 |
| VMP1 | 1.161426187 | 1.00148E−22 | 2.09661E−20 | 19.67848302 | POMC | 1.059912443 | 1.62939E−05 | 0.000369539 | 3.432339802 |
| CRYL1 | 1.256764531 | 1.1037E−22 | 2.29102E−20 | 19.63997188 | NFIC | 0.649380207 | 1.62806E−05 | 0.000369539 | 3.432339802 |
| GPNMB | 1.022765875 | 1.30043E−22 | 2.6767E−20 | 19.57240058 | YIF1A | 0.573261142 | 1.6382E−05 | 0.000371195 | 3.430397872 |
| LINC00462 | 3.379219532 | 1.34119E−22 | 2.71498E−20 | 19.56623395 | IAH1 | 0.524902463 | 1.64208E−05 | 0.00037173 | 3.429772176 |
| TMSB10 | 0.701115787 | 2.0881E−22 | 4.15821E−20 | 19.38109347 | XRN2 | 0.581026256 | 1.68124E−05 | 0.000380243 | 3.419939079 |
| COX4I1 | 0.718603432 | 2.54851E−22 | 5.03413E−20 | 19.29807595 | OS9 | 0.490287066 | 1.69445E−05 | 0.000382876 | 3.416941674 |
| CFL1 | 0.56077683 | 2.62447E−22 | 5.1427E−20 | 19.28880848 | L1CAM | 0.504581928 | 1.71343E−05 | 0.000386809 | 3.412503414 |
| FTL | 0.578958273 | 3.65412E−22 | 7.10349E−20 | 19.14852802 | MAPRE1 | 0.578903854 | 1.71571E−05 | 0.000386966 | 3.412326869 |
| BSG | 0.731443644 | 4.19986E−22 | 8.10012E−20 | 19.09150877 | MICAL1 | 0.760659993 | 1.73748E−05 | 0.000391516 | 3.407250387 |
| SKAP2 | 2.200052023 | 5.5686E−22 | 1.0656E−19 | 18.97240459 | PSMD7 | 0.525504231 | 1.82346E−05 | 0.000409761 | 3.387469398 |
| TMEM147 | 0.959870815 | 5.74601E−22 | 1.09103E−19 | 18.96216353 | FCGRT | 0.390538871 | 1.83665E−05 | 0.000412346 | 3.384737861 |
| GAPDH | 0.858291924 | 6.63563E−22 | 1.25025E−19 | 18.90300179 | EIF2AK4 | 0.577619433 | 1.86508E−05 | 0.000418346 | 3.37846456 |
| TMBIM4 | 0.980383813 | 7.83714E−22 | 1.46537E−19 | 18.83405383 | RAB1B | 0.467691749 | 1.90649E−05 | 0.000427241 | 3.36932658 |
| MGST3 | 0.920697987 | 1.02223E−21 | 1.88588E−19 | 18.72448566 | TMEM120A | 0.576262057 | 1.92363E−05 | 0.000430689 | 3.365835856 |
| FTH1 | 0.482964933 | 1.02401E−21 | 1.88588E−19 | 18.72448566 | COA4 | 0.615577757 | 1.95449E−05 | 0.000437198 | 3.359321408 |
| PCSK2 | 0.973265409 | 1.13648E−21 | 2.07739E−19 | 18.68248297 | CAP1 | 0.480936587 | 1.98814E−05 | 0.000443916 | 3.352698916 |
| PLP1 | 0.899620533 | 1.39406E−21 | 2.52934E−19 | 18.59699359 | LINC00681 | 1.557628751 | 2.04258E−05 | 0.000455655 | 3.341363909 |
| HIGD2A | 1.080669522 | 1.47593E−21 | 2.65819E−19 | 18.57541398 | RPL5 | 0.184221923 | 2.05787E−05 | 0.000458649 | 3.338519295 |
| HINT1 | 0.790259242 | 2.08429E−21 | 3.72646E−19 | 18.42870299 | PPIE | 0.68467319 | 2.08711E−05 | 0.000464321 | 3.33318156 |
| TXN | 0.875577569 | 2.2822E−21 | 4.05075E−19 | 18.39246497 | FRG1 | 0.633555055 | 2.15746E−05 | 0.000479101 | 3.319572496 |
| RNASEK | 0.785449445 | 2.73761E−21 | 4.82411E−19 | 18.31658277 | DNAJC3 | 0.374100536 | 2.15715E−05 | 0.000479101 | 3.319572496 |
| TMEM59 | 0.616931379 | 3.38428E−21 | 5.92103E−19 | 18.2276024 | RPL22 | 0.388623536 | 2.16235E−05 | 0.000479751 | 3.31898382 |
| CBR1 | 1.154143691 | 4.0704E−21 | 7.07096E−19 | 18.1505218 | EEF1B2 | 0.306186676 | 2.1731E−05 | 0.000481701 | 3.317222814 |
| SERF2 | 0.631174088 | 4.24799E−21 | 7.32749E−19 | 18.13504504 | EEA1 | 0.393598408 | 2.2039E−05 | 0.000488085 | 3.311504307 |
| RPS11 | 0.37531969 | 5.1069E−21 | 8.74744E−19 | 18.05811905 | SUPT4H1 | 0.499374896 | 2.24195E−05 | 0.000495618 | 3.304853242 |
| POLR2L | 0.952073395 | 5.27147E−21 | 8.96663E−19 | 18.04737086 | SNRPD3 | 0.482543677 | 2.36026E−05 | 0.000521301 | 3.282911659 |
| ZFYVE21 | 1.144884348 | 6.9781E−21 | 1.17877E−18 | 17.92857141 | EDNRB | 0.389129281 | 2.40402E−05 | 0.000530488 | 3.275324589 |
| TYR | 0.841504514 | 8.2968E−21 | 1.38246E−18 | 17.8593472 | VTI1B | 0.549896359 | 2.45211E−05 | 0.00053964 | 3.267895538 |
| RAC1 | 0.733914554 | 1.03543E−20 | 1.71364E−18 | 17.76608057 | ANKS1B | 1.294572353 | 2.45977E−05 | 0.00054084 | 3.266931546 |
| RPS25 | 0.526731908 | 1.09388E−20 | 1.79822E−18 | 17.7451567 | EIF4EBP1 | 0.563168824 | 2.46959E−05 | 0.000542513 | 3.265589637 |
| ATP6V0E1 | 0.958368123 | 1.28912E−20 | 2.10505E−18 | 17.67673743 | SF3B6 | 0.45095858 | 2.49297E−05 | 0.000547158 | 3.261887464 |
| SEPT7 | 0.8664487 | 1.30108E−20 | 2.11051E−18 | 17.6756125 | UBE2B | 0.353586882 | 2.51219E−05 | 0.000550882 | 3.258941685 |
| UQCRQ | 0.814134598 | 1.61722E−20 | 2.60606E−18 | 17.58401521 | PSMB9 | 2.537453413 | 2.51747E−05 | 0.000551439 | 3.258502521 |
| UBB | 0.574596882 | 2.07603E−20 | 3.32355E−18 | 17.47839742 | CHD1L | 0.693206549 | 2.52148E−05 | 0.000551439 | 3.258502521 |
| ATOX1 | 0.916679502 | 2.17625E−20 | 3.46136E−18 | 17.46075304 | NAMPT | 0.649378121 | 2.51947E−05 | 0.000551439 | 3.258502521 |
| GNG5 | 0.934737027 | 2.2017E−20 | 3.47925E−18 | 17.45851374 | RAB9A | 0.511231661 | 2.5479E−05 | 0.000556721 | 3.25436269 |
| RABAC1 | 1.019441485 | 2.23462E−20 | 3.50863E−18 | 17.45486187 | RER1 | 0.530742884 | 2.55492E−05 | 0.000557756 | 3.253555582 |
| SYNGR1 | 0.956699789 | 3.09303E−20 | 4.82552E−18 | 17.31645613 | CHCHD10 | 0.5819965 | 2.56641E−05 | 0.000559765 | 3.251994147 |
| RPS27L | 1.04148531 | 3.41287E−20 | 5.29081E−18 | 17.27647816 | GTF2H5 | 0.491772532 | 2.57813E−05 | 0.000561822 | 3.250401379 |
| CD63 | 0.64383924 | 3.82015E−20 | 5.88495E−18 | 17.23025717 | CHPF | 0.555068672 | 2.63847E−05 | 0.000573949 | 3.241126565 |
| AHNAK2 | 2.164326191 | 3.95968E−20 | 6.06178E−18 | 17.21739974 | GLG1 | 0.368060172 | 2.64878E−05 | 0.000575681 | 3.239817799 |
| H3F3A | 0.681281149 | 5.74761E−20 | 8.74422E−18 | 17.05827881 | EPHX1 | 0.934535921 | 2.68325E−05 | 0.000582138 | 3.234973878 |
| NSMF | 1.230613947 | 7.34299E−20 | 1.11024E−17 | 16.95458251 | CYB5R1 | 0.532831252 | 2.68266E−05 | 0.000582138 | 3.234973878 |
| MYL12B | 0.918736696 | 7.6379E−20 | 1.14775E−17 | 16.94015399 | TGFBR3 | 0.943657696 | 2.7105E−05 | 0.00058753 | 3.230970063 |
| NDUFB11 | 1.020690322 | 8.30823E−20 | 1.24086E−17 | 16.90627548 | MAP3K3 | 0.723381579 | 2.715E−05 | 0.000587985 | 3.230633495 |
| GPM6B | 0.772643805 | 9.84885E−20 | 1.46205E−17 | 16.83503889 | ZDHHC7 | 0.80971384 | 2.75325E−05 | 0.000595744 | 3.224940458 |
| ATP6V0E2 | 1.319425344 | 1.33178E−19 | 1.9651E−17 | 16.70661628 | NPM1 | 0.449071586 | 2.77597E−05 | 0.0005996 | 3.222138098 |
| RAB27A | 1.194572926 | 1.39092E−19 | 2.04007E−17 | 16.69035569 | TOLLIP | 0.594495773 | 2.78519E−05 | 0.000601061 | 3.221081196 |
| OAZ1 | 0.686629772 | 1.45308E−19 | 2.11856E−17 | 16.67395916 | COMMD3 | 0.638926268 | 2.79222E−05 | 0.000602049 | 3.220368392 |
| SH3BGRL | 1.158374429 | 1.93746E−19 | 2.80805E−17 | 16.55159489 | YWHAH | 0.496766061 | 2.80513E−05 | 0.000604299 | 3.218748092 |
| HLA-DPA1 | 1.970546126 | 1.96863E−19 | 2.83645E−17 | 16.54722477 | GCSH | 0.463813752 | 2.83376E−05 | 0.000609395 | 3.215101344 |
| RPS20 | 0.534705162 | 2.40686E−19 | 3.44758E−17 | 16.46248542 | PDZRN3 | 0.661841869 | 2.96894E−05 | 0.000637347 | 3.195624381 |
| TPP1 | 0.896618187 | 2.73804E−19 | 3.89916E−17 | 16.40902909 | ZNHIT1 | 0.474387139 | 2.98262E−05 | 0.000639722 | 3.194008468 |
| C4orf3 | 0.91715771 | 2.95874E−19 | 4.1891E−17 | 16.3778789 | KRT10 | 0.401700109 | 3.0482E−05 | 0.000653216 | 3.184943193 |
| TMA7 | 0.699146748 | 3.05151E−19 | 4.29561E−17 | 16.36697522 | ABCG1 | 2.525199175 | 3.07345E−05 | 0.000658051 | 3.181740766 |
| NDUFB1 | 0.802649319 | 3.31381E−19 | 4.6382E−17 | 16.33365091 | CCSER2 | 0.655194581 | 3.08117E−05 | 0.000659128 | 3.181030279 |
| H2AFJ | 0.965035617 | 3.47835E−19 | 4.84084E−17 | 16.31507936 | NDUFV2 | 0.438857526 | 3.18874E−05 | 0.000680949 | 3.166885137 |
| LAMTOR4 | 0.830777824 | 3.52556E−19 | 4.87882E−17 | 16.31168525 | ADAM17 | 0.465740561 | 3.24049E−05 | 0.000690796 | 3.160650139 |
| HSPB1 | 0.464148283 | 3.83373E−19 | 5.27548E−17 | 16.27773834 | EEF1G | 0.145939738 | 3.28974E−05 | 0.000700686 | 3.15447648 |
| CHCHD2 | 0.785480678 | 4.31646E−19 | 5.90655E−17 | 16.22866593 | ACSS1 | 1.053247809 | 3.30364E−05 | 0.00070211 | 3.153594858 |
| NDUFA6 | 0.921058893 | 4.41123E−19 | 6.00271E−17 | 16.22165289 | LSM3 | 0.511060476 | 3.306E−05 | 0.00070211 | 3.153594858 |
| HMGN3 | 0.992473245 | 4.70043E−19 | 6.3609E−17 | 16.19648147 | GSTK1 | 0.486371726 | 3.30789E−05 | 0.00070211 | 3.153594858 |
| SNHG6 | 0.825036049 | 5.00391E−19 | 6.73438E−17 | 16.17170237 | EPB41L3 | 0.756706178 | 3.31641E−05 | 0.000703309 | 3.152854048 |
| SCARB1 | 1.053153515 | 6.9483E−19 | 9.30009E−17 | 16.0315127 | S100A4 | 1.496086359 | 3.34161E−05 | 0.00070753 | 3.15025533 |
| OCA2 | 1.731920958 | 7.11668E−19 | 9.47369E−17 | 16.02348081 | COPE | 0.472071648 | 3.34209E−05 | 0.00070753 | 3.15025533 |
| OST4 | 0.760849535 | 7.20925E−19 | 9.54505E−17 | 16.02022201 | PDE4B | 1.155525923 | 3.39693E−05 | 0.000718518 | 3.143562362 |
| WDR63 | 3.06368804 | 9.02394E−19 | 1.18835E−16 | 15.92505702 | TXNDC17 | 0.464057326 | 3.4396E−05 | 0.000726916 | 3.138516023 |
| GABARAP | 0.595390856 | 1.14127E−18 | 1.49489E−16 | 15.82539185 | NEAT1 | 0.021474367 | 3.45927E−05 | 0.000730443 | 3.136413438 |
| RPL15 | 0.482807785 | 1.18941E−18 | 1.54965E−16 | 15.80976599 | MTDH | 0.340117037 | 3.48054E−05 | 0.000734302 | 3.134125537 |
| HSP90AA1 | 0.579884887 | 1.48913E−18 | 1.92988E−16 | 15.71446909 | PDAP1 | 0.387150526 | 3.48725E−05 | 0.000735083 | 3.133663829 |
| XRCC5 | 0.881445646 | 2.2679E−18 | 2.92368E−16 | 15.53406994 | TMEM179B | 0.487425238 | 3.55091E−05 | 0.000747859 | 3.126180392 |
| CHCHD6 | 0.938161612 | 2.52892E−18 | 3.24311E−16 | 15.48903797 | FAM200B | 0.556892812 | 3.55496E−05 | 0.000748068 | 3.126058967 |
| FBLN7 | 1.612652779 | 2.74445E−18 | 3.50117E−16 | 15.4557863 | KIFAP3 | 0.709772766 | 3.68287E−05 | 0.00077432 | 3.111079718 |
| COX6C | 0.782659054 | 3.60306E−18 | 4.57271E−16 | 15.33982592 | MT1G | 1.61437428 | 3.71071E−05 | 0.000779504 | 3.108181577 |
| MT1F | 1.46675992 | 3.74418E−18 | 4.72732E−16 | 15.32538522 | DCK | 1.269013405 | 3.8065E−05 | 0.00079894 | 3.097485737 |
| POMP | 0.891595662 | 5.43292E−18 | 6.8243E−16 | 15.16594169 | ACTR1A | 0.536726654 | 3.84203E−05 | 0.000805708 | 3.093822489 |
| NDUFB4 | 0.81092602 | 5.4696E−18 | 6.83532E−16 | 15.1652409 | RPL26 | 0.182495385 | 3.86936E−05 | 0.000810746 | 3.091115107 |
| PIR | 1.287799001 | 5.49786E−18 | 6.83576E−16 | 15.1652129 | KTN1 | 0.39933759 | 3.90435E−05 | 0.000817377 | 3.087577662 |
| SOX4 | 0.581649363 | 6.55784E−18 | 8.11251E−16 | 15.09084484 | CTSS | 2.371190548 | 3.91848E−05 | 0.000819635 | 3.086379525 |
| MBP | 1.045734286 | 7.621E−18 | 9.38034E−16 | 15.02778136 | PEA15 | 0.608195961 | 3.93712E−05 | 0.00082213 | 3.085059622 |
| CTNNAL1 | 1.428975105 | 7.78346E−18 | 9.53241E−16 | 15.02079733 | ACTG1 | 0.399410754 | 4.0009E−05 | 0.000834737 | 3.078450546 |
| CYSTM1 | 0.934885621 | 7.85254E−18 | 9.56915E−16 | 15.01912645 | PMF1 | 0.52551806 | 4.01963E−05 | 0.00083793 | 3.076792204 |
| LAMTOR2 | 0.990105271 | 8.36159E−18 | 1.01391E−15 | 14.99400265 | LIMA1 | 0.471411824 | 4.02874E−05 | 0.000839115 | 3.076178507 |
| DSTN | 0.814635932 | 1.43257E−17 | 1.72007E−15 | 14.76445402 | MSX1 | 2.116905928 | 4.06135E−05 | 0.000845189 | 3.07304609 |
| SNRPD2 | 0.687391639 | 1.51246E−17 | 1.80713E−15 | 14.74300967 | MAP6 | 1.254459143 | 4.17159E−05 | 0.000867393 | 3.06178413 |
| CEP72 | 1.660027623 | 1.66204E−17 | 1.97622E−15 | 14.70416474 | CMC2 | 0.463078529 | 4.18221E−05 | 0.000868863 | 3.061048701 |
| FAM124A | 1.633755803 | 1.75356E−17 | 2.07496E−15 | 14.68299106 | TCTN2 | 0.593632102 | 4.19107E−05 | 0.000869967 | 3.060497061 |
| UQCR11 | 0.732141495 | 1.84587E−17 | 2.17369E−15 | 14.66280284 | PHLDA1 | 0.42486617 | 4.25035E−05 | 0.000881524 | 3.054765853 |
| EMX2 | 2.300971508 | 1.87364E−17 | 2.19584E−15 | 14.65840016 | SLC19A2 | 0.665811002 | 4.28315E−05 | 0.000887576 | 3.051794582 |
| MAP1B | 1.140796542 | 2.34884E−17 | 2.73964E−15 | 14.56230633 | A1BG | 0.467788815 | 4.29525E−05 | 0.00088933 | 3.050936914 |
| PTTG1IP | 0.743923068 | 2.51574E−17 | 2.92041E−15 | 14.53455666 | TMSB4X | 0.534632862 | 4.31497E−05 | 0.000891907 | 3.049680377 |
| CALM2 | 0.603281319 | 2.80452E−17 | 3.24028E−15 | 14.48941684 | MIR4458HG | 0.600424945 | 4.34396E−05 | 0.000897142 | 3.047138888 |
| PSMB1 | 0.830644488 | 2.91589E−17 | 3.33747E−15 | 14.47658285 | PPP1CA | 0.554171622 | 4.35714E−05 | 0.000899106 | 3.046189072 |
| ATP5O | 0.974675357 | 3.28738E−17 | 3.74517E−15 | 14.4265284 | PPT1 | 0.594768822 | 4.45268E−05 | 0.000917274 | 3.037501073 |
| GPR143 | 0.777115405 | 3.6864E−17 | 4.16104E−15 | 14.38079777 | FLNA | 0.551986039 | 4.47907E−05 | 0.000921936 | 3.03529915 |
| EDF1 | 0.748168051 | 4.34027E−17 | 4.87663E−15 | 14.31187997 | CAPN1 | 0.612809837 | 4.48569E−05 | 0.000922524 | 3.035022315 |
| GTF2F2 | 1.190070033 | 4.88261E−17 | 5.46094E−15 | 14.26273245 | COTL1 | 0.619974732 | 4.54124E−05 | 0.000933165 | 3.030041749 |
| SNCA | 0.759882092 | 5.57232E−17 | 6.20401E−15 | 14.20732722 | SEZ6L2 | 1.567870975 | 4.57874E−05 | 0.000940082 | 3.026834276 |
| TPI1 | 0.920636535 | 6.1329E−17 | 6.79725E−15 | 14.16766653 | NAPA | 0.475968003 | 4.62953E−05 | 0.000949713 | 3.022407801 |
| USMG5 | 0.770136178 | 6.35617E−17 | 7.01298E−15 | 14.15409768 | POLR2F | 0.375527263 | 4.72872E−05 | 0.000969249 | 3.013564487 |
| ATP6V0B | 0.948582113 | 7.1184E−17 | 7.78384E−15 | 14.10880603 | TMED2 | 0.509700954 | 4.8046E−05 | 0.000983979 | 3.007014003 |
| BACE2 | 0.717782736 | 9.31425E−17 | 1.01397E−14 | 13.99397493 | C7orf50 | 0.565492451 | 4.81776E−05 | 0.000985849 | 3.006189722 |
| HEXA | 0.961841762 | 1.01057E−16 | 1.09526E−14 | 13.96048348 | SDHD | 0.455037892 | 4.84586E−05 | 0.000990772 | 3.004026235 |
| SON | 0.676162422 | 1.0308E−16 | 1.11226E−14 | 13.95379193 | SLC16A10 | 0.977722347 | 4.85776E−05 | 0.000992376 | 3.003323583 |
| KRTCAP2 | 0.801041007 | 1.06409E−16 | 1.14315E−14 | 13.94189567 | GAS6 | 0.371127576 | 4.88574E−05 | 0.000997261 | 3.001190996 |
| DPP6 | 2.027810812 | 1.2029E−16 | 1.28663E−14 | 13.89054507 | RPS4X | 0.103819363 | 4.92042E−05 | 0.001003504 | 2.998481101 |
| RPL27 | 0.51071012 | 1.23504E−16 | 1.31526E−14 | 13.88098719 | HOXB7 | 0.755761087 | 4.94182E−05 | 0.00100703 | 2.996957551 |
| CTSA | 1.054712892 | 1.26907E−16 | 1.34565E−14 | 13.87106771 | GRAMD3 | 0.707031786 | 4.96853E−05 | 0.00101163 | 2.994978241 |
| MYL6 | 0.598981023 | 1.55635E−16 | 1.64315E−14 | 13.78432189 | COPRS | 0.540202379 | 5.01533E−05 | 0.001020311 | 2.991267643 |
| COX5B | 0.731863081 | 1.57434E−16 | 1.65502E−14 | 13.78119791 | PARP14 | 0.749527812 | 5.08737E−05 | 0.001033252 | 2.985793945 |
| NFATC2 | 1.491753101 | 1.91354E−16 | 2.003E−14 | 13.69831887 | DGUOK | 0.507428944 | 5.08453E−05 | 0.001033252 | 2.985793945 |
| KLF6 | 0.704577506 | 2.07104E−16 | 2.15864E−14 | 13.66581977 | TIMM13 | 0.510233462 | 5.10982E−05 | 0.001036951 | 2.984241825 |
| PSME1 | 1.013999462 | 2.40695E−16 | 2.49813E−14 | 13.6023848 | SNU13 | 0.416409343 | 5.13523E−05 | 0.001041245 | 2.982447131 |
| ARPC3 | 0.842056513 | 2.55914E−16 | 2.64488E−14 | 13.57759383 | TTC1 | 0.531439245 | 5.15104E−05 | 0.001043586 | 2.981471682 |
| RNF145 | 0.770240664 | 2.89372E−16 | 2.9781E−14 | 13.52606096 | GJA1 | 2.001495838 | 5.17556E−05 | 0.001046822 | 2.98012705 |
| ROPN1B | 1.104176641 | 3.18261E−16 | 3.26171E−14 | 13.48655449 | VPS28 | 0.418588728 | 5.27549E−05 | 0.001066154 | 2.972180159 |
| TSPAN4 | 1.030030727 | 5.94933E−16 | 6.04659E−14 | 13.21848954 | SLC18B1 | 0.830502629 | 5.28359E−05 | 0.001066911 | 2.971871846 |
| COX7B | 0.811161518 | 6.95333E−16 | 7.03781E−14 | 13.15256272 | SEC61A1 | 0.602198005 | 5.46485E−05 | 0.001101697 | 2.957938024 |
| COX6B1 | 0.7094028 | 7.09632E−16 | 7.15297E−14 | 13.14551363 | C19orf53 | 0.392784774 | 5.4862E−05 | 0.001105091 | 2.956602035 |
| RNF7 | 0.811091602 | 8.67333E−16 | 8.70674E−14 | 13.0601442 | ASNA1 | 0.500286937 | 5.49729E−05 | 0.001106414 | 2.956082295 |
| C11orf31 | 0.785324395 | 1.10524E−15 | 1.10497E−13 | 12.95664892 | MCUR1 | 0.592690825 | 5.66143E−05 | 0.001136649 | 2.944373771 |
| TCEB2 | 0.65186739 | 1.32251E−15 | 1.31681E−13 | 12.88047617 | SRSF5 | 0.312133849 | 5.77945E−05 | 0.001158443 | 2.936125355 |
| ATP5L | 0.728517592 | 1.37838E−15 | 1.36689E−13 | 12.86426783 | COX7A2L | 0.37859267 | 5.78948E−05 | 0.001159506 | 2.935726861 |
| LRRC16A | 1.378694415 | 1.4877E−15 | 1.46935E−13 | 12.83287608 | LOC654342 | 0.544074357 | 5.80478E−05 | 0.001161619 | 2.934936261 |
| SET | 0.564666927 | 1.58299E−15 | 1.55717E−13 | 12.80766274 | BCL6 | 0.580954194 | 5.82981E−05 | 0.001165676 | 2.933422203 |
| COX 17 | 0.870893657 | 1.71732E−15 | 1.68256E−13 | 12.77403015 | RPL7 | 0.106590442 | 5.88875E−05 | 0.001176501 | 2.92940783 |
| IFI27 | 1.841121316 | 1.72687E−15 | 1.68518E−13 | 12.77335363 | SRPR | 0.57796669 | 5.90838E−05 | 0.001179462 | 2.928316228 |
| NINJ1 | 1.014419556 | 1.80524E−15 | 1.75466E−13 | 12.75580587 | SLC39A11 | 1.043327332 | 5.92479E−05 | 0.001181774 | 2.92746548 |
| MYDGF | 0.950088084 | 1.8555E−15 | 1.79639E−13 | 12.74560044 | FGD5-AS1 | 0.451381445 | 6.08749E−05 | 0.001213238 | 2.916053836 |
| ATRAID | 0.807048261 | 1.97784E−15 | 1.90729E−13 | 12.71958328 | FRG2DP | 2.40166235 | 6.10818E−05 | 0.001215383 | 2.915286793 |
| ANXA2P2 | 0.512970865 | 2.22623E−15 | 2.1384E−13 | 12.66991062 | KIAA1715 | 0.562832534 | 6.10437E−05 | 0.001215383 | 2.915286793 |
| IFITM1 | 1.860036135 | 2.2754E−15 | 2.17709E−13 | 12.6621228 | CRISPLD2 | 2.072827339 | 6.18302E−05 | 0.001228814 | 2.910513789 |
| RPL21 | 0.452848881 | 2.88907E−15 | 2.75349E−13 | 12.56011594 | TDRD7 | 0.912105143 | 6.18901E−05 | 0.001228814 | 2.910513789 |
| NDUFC1 | 0.785579741 | 3.6071E−15 | 3.41128E−13 | 12.4670824 | PDCD6 | 0.447008938 | 6.19073E−05 | 0.001228814 | 2.910513789 |
| AHR | 0.983914316 | 3.64119E−15 | 3.43028E−13 | 12.46467081 | AMFR | 0.634195089 | 6.23283E−05 | 0.001236169 | 2.907922063 |
| NSG1 | 0.518368304 | 3.68424E−15 | 3.45754E−13 | 12.46123233 | CIB1 | 0.6225757 | 6.48581E−05 | 0.001285303 | 2.890994347 |
| LINC00869 | 1.136823773 | 3.76793E−15 | 3.52259E−13 | 12.45313824 | HTATIP2 | 1.06083405 | 6.72808E−05 | 0.00133116 | 2.87576985 |
| TMEM14C | 0.76008898 | 4.19446E−15 | 3.89163E−13 | 12.4098684 | FGFBP2 | 1.900160432 | 6.78904E−05 | 0.001341053 | 2.872553902 |
| BRI3 | 0.613031089 | 4.66965E−15 | 4.31616E−13 | 12.36490211 | CKAP4 | 0.672939301 | 6.78904E−05 | 0.001341053 | 2.872553902 |
| STOM | 1.231079817 | 4.87385E−15 | 4.48797E−13 | 12.34795021 | CORO2B | 0.846568406 | 6.84251E−05 | 0.001349439 | 2.869846682 |
| SEPW1 | 0.55191797 | 5.02963E−15 | 4.61407E−13 | 12.33591535 | NDUFB5 | 0.444406629 | 6.83968E−05 | 0.001349439 | 2.869846682 |
| HP1BP3 | 0.784286559 | 5.21262E−15 | 4.7641E−13 | 12.32201919 | C18orf32 | 0.472491652 | 6.85759E−05 | 0.001351325 | 2.869240085 |
| ZNF706 | 0.811532259 | 5.34147E−15 | 4.86372E−13 | 12.31303183 | ARGLU1 | 0.332099319 | 6.96499E−05 | 0.001371387 | 2.862839899 |
| YWHAE | 0.531483412 | 6.01864E−15 | 5.43988E−13 | 12.26441106 | BOC | 1.126646042 | 7.01978E−05 | 0.001381063 | 2.859786431 |
| RRAS | 1.247763634 | 6.1352E−15 | 5.5046E−13 | 12.25927434 | GTF2A2 | 0.508305788 | 7.08663E−05 | 0.001393033 | 2.856038652 |
| RPS12 | 0.417209536 | 6.1352E−15 | 5.5046E−13 | 12.25927434 | SDF4 | 0.443287641 | 7.09199E−05 | 0.001393033 | 2.856038652 |
| APOA1BP | 0.984113395 | 7.27144E−15 | 6.50024E−13 | 12.18707059 | MEA1 | 0.489862293 | 7.15066E−05 | 0.001403431 | 2.8528088 |
| MALAT1 | 0.296129793 | 7.30063E−15 | 6.5026E−13 | 12.18691288 | CALM3 | 0.447917104 | 7.23655E−05 | 0.001419151 | 2.847971405 |
| PTRF | 1.372162938 | 7.57835E−15 | 6.72551E−13 | 12.17227473 | CYFIP1 | 0.619206846 | 7.28485E−05 | 0.001427481 | 2.845429807 |
| HSBP1 | 0.869912803 | 7.78967E−15 | 6.88809E−13 | 12.16190102 | LINC01138 | 0.776723683 | 7.34905E−05 | 0.001438911 | 2.84196618 |
| NDUFB8 | 0.712562859 | 7.84517E−15 | 6.91222E−13 | 12.16038265 | RPL35A | 0.152758226 | 7.4006E−05 | 0.001447846 | 2.839277666 |
| PAG1 | 0.704228997 | 8.08047E−15 | 7.09402E−13 | 12.14910766 | BAX | 0.52432549 | 7.41278E−05 | 0.001449071 | 2.838910332 |
| RPL27A | 0.372294545 | 9.08145E−15 | 7.91605E−13 | 12.10149142 | HES6 | 0.687318623 | 7.42396E−05 | 0.001450099 | 2.838602285 |
| GLUL | 1.130079508 | 9.22643E−15 | 8.0139E−13 | 12.09615584 | TRIM9 | 2.857247829 | 7.45606E−05 | 0.001454051 | 2.837420395 |
| MSANTD3 | 1.044047952 | 9.50121E−15 | 8.22342E−13 | 12.08494759 | NT5C3A | 0.538684011 | 7.45504E−05 | 0.001454051 | 2.837420395 |
| TGFBI | 3.234729528 | 9.7376E−15 | 8.39833E−13 | 12.07580686 | EBP | 0.606148064 | 7.46833E−05 | 0.001455284 | 2.837052247 |
| LRRFIP2 | 1.13446784 | 1.04055E−14 | 8.94291E−13 | 12.04852135 | SH3GLB1 | 0.431151301 | 7.515E−05 | 0.001463215 | 2.834691749 |
| NDUFA11 | 0.665408432 | 1.07493E−14 | 9.20605E−13 | 12.03592681 | RPS2 | 0.128720686 | 7.5449E−05 | 0.001467869 | 2.833312701 |
| SEC61B | 0.790967107 | 1.08924E−14 | 9.29612E−13 | 12.03169806 | AP3M2 | 0.486658394 | 7.61856E−05 | 0.001479849 | 2.829782464 |
| COX5A | 0.842416465 | 1.11171E−14 | 9.4549E−13 | 12.02434313 | HIPK1 | 0.694231331 | 7.65303E−05 | 0.001484191 | 2.828510268 |
| NDUFB7 | 0.725215435 | 1.42774E−14 | 1.21007E−12 | 11.91718891 | AP2B1 | 0.472168326 | 7.85223E−05 | 0.001521618 | 2.817694497 |
| KIAA0930 | 0.99893409 | 1.45278E−14 | 1.22705E−12 | 11.91113863 | AURKAIP1 | 0.387147963 | 7.892E−05 | 0.001528117 | 2.815843507 |
| OLFM1 | 1.327364445 | 1.56031E−14 | 1.31334E−12 | 11.88162134 | CHURC1 | 0.408841282 | 7.94497E−05 | 0.001537158 | 2.813281465 |
| CRIP2 | 1.101250768 | 1.65993E−14 | 1.39241E−12 | 11.85623325 | KIF5B | 0.389677703 | 7.96832E−05 | 0.001540459 | 2.81234997 |
| FBXO32 | 0.990670204 | 1.82038E−14 | 1.52179E−12 | 11.81764506 | PTRHD1 | 0.535453439 | 8.04534E−05 | 0.001554121 | 2.80851518 |
| TSPAN10 | 1.066144109 | 1.8633E−14 | 1.55237E−12 | 11.80900532 | FDFT1 | 0.282441378 | 8.1519E−05 | 0.001573464 | 2.803143072 |
| ITGB1BP1 | 0.936465323 | 2.15806E−14 | 1.79184E−12 | 11.74669965 | STX8 | 0.676751375 | 8.23452E−05 | 0.00158691 | 2.799447704 |
| ASPA | 1.114335418 | 2.38168E−14 | 1.97084E−12 | 11.70534835 | CANX | 0.361062676 | 8.40613E−05 | 0.001617438 | 2.791172389 |
| RPS7 | 0.471603215 | 4.14187E−14 | 3.41585E−12 | 11.4665006 | EHD4 | 0.677356243 | 8.45028E−05 | 0.001624656 | 2.789238532 |
| GPX4 | 0.744344115 | 4.50027E−14 | 3.69898E−12 | 11.43191768 | ARL4C | 1.223694801 | 8.54795E−05 | 0.001642144 | 2.784588666 |
| PPIB | 0.736930966 | 4.52121E−14 | 3.70377E−12 | 11.43135656 | CES1 | 1.697817922 | 8.60156E−05 | 0.001651149 | 2.782213694 |
| EIF3K | 0.857389808 | 4.91924E−14 | 4.00305E−12 | 11.39760893 | FAM127A | 0.527288973 | 8.61267E−05 | 0.001651988 | 2.781993231 |
| COX7C | 0.656610847 | 4.91369E−14 | 4.00305E−12 | 11.39760893 | CAPZA1 | 0.409783304 | 8.64081E−05 | 0.001656087 | 2.780916897 |
| TRPM4 | 1.357009768 | 5.16416E−14 | 4.18844E−12 | 11.37794739 | MT1E | 0.758553326 | 8.87865E−05 | 0.001700341 | 2.769464069 |
| UQCR10 | 0.772781432 | 5.50235E−14 | 4.448E−12 | 11.35183502 | PLA2G4C | 1.262204528 | 8.93119E−05 | 0.001709067 | 2.767240962 |
| H3F3B | 0.291507214 | 6.04592E−14 | 4.87134E−12 | 11.31235136 | EFNB2 | 0.966145575 | 9.10353E−05 | 0.001740152 | 2.759412703 |
| S100A13 | 0.900659204 | 6.10432E−14 | 4.90227E−12 | 11.30960299 | TRAPPC5 | 0.434509516 | 9.10784E−05 | 0.001740152 | 2.759412703 |
| LIMD2 | 0.897961259 | 7.08409E−14 | 5.67052E−12 | 11.2463774 | ZC3H15 | 0.528942585 | 9.15981E−05 | 0.001748716 | 2.757280641 |
| PEBP1 | 0.564732194 | 8.16818E−14 | 6.51699E−12 | 11.18595315 | PITHD1 | 0.462939709 | 9.26332E−05 | 0.001767101 | 2.752738742 |
| BCAN | 0.749105275 | 8.55325E−14 | 6.80205E−12 | 11.16735999 | SEC31A | 0.537380338 | 9.32179E−05 | 0.001776871 | 2.750344051 |
| RDX | 0.97198689 | 8.67188E−14 | 6.87408E−12 | 11.16278533 | DDX5 | 0.295726627 | 9.3578E−05 | 0.001782348 | 2.749007426 |
| MITF | 0.583578289 | 1.14133E−13 | 8.98901E−12 | 11.04628794 | BZW1 | 0.616345644 | 9.3749E−05 | 0.001784219 | 2.748551961 |
| DYNLL1 | 0.757417798 | 1.17291E−13 | 9.20807E−12 | 11.03583139 | SEMA3C | 0.378002495 | 9.45831E−05 | 0.001798694 | 2.745042694 |
| ARSB | 1.009860516 | 1.21925E−13 | 9.52264E−12 | 11.02124257 | CYTH3 | 0.397750586 | 9.50766E−05 | 0.001806676 | 2.743119664 |
| NFIB | 0.932283878 | 1.22075E−13 | 9.52264E−12 | 11.02124257 | SNRPE | 0.415916383 | 9.56823E−05 | 0.001816777 | 2.74069828 |
| NME2 | 0.66022259 | 1.25944E−13 | 9.79325E−12 | 11.00907298 | SEC11A | 0.423288703 | 9.63893E−05 | 0.001828783 | 2.737837724 |
| LINC01314 | 1.852920532 | 1.27538E−13 | 9.88579E−12 | 11.00498853 | SIRPA | 0.633098423 | 9.78313E−05 | 0.001852838 | 2.732162514 |
| TMEM50A | 0.76158452 | 1.33981E−13 | 1.03525E−11 | 10.98495657 | SLC35B2 | 0.404316574 | 9.77785E−05 | 0.001852838 | 2.732162514 |
| YWHAB | 0.733088195 | 1.35474E−13 | 1.04349E−11 | 10.98151102 | CLCN7 | 0.393751472 | 9.78841E−05 | 0.001852838 | 2.732162514 |
| POLR2G | 0.849233389 | 1.40415E−13 | 1.07816E−11 | 10.96731754 | TMEM219 | 0.366783053 | 9.83407E−05 | 0.001860045 | 2.730476577 |
| BRK1 | 0.711579204 | 1.44694E−13 | 1.10754E−11 | 10.9556399 | CAPN2 | 0.505916655 | 9.90462E−05 | 0.001871943 | 2.727707451 |
| NDUFB2 | 0.788868546 | 1.66915E−13 | 1.27365E−11 | 10.89494919 | TRIM8 | 0.634671211 | 0.000100377 | 0.001895627 | 2.72224704 |
| PRNP | 1.370777845 | 1.82609E−13 | 1.38908E−11 | 10.857274 | TBC1D16 | 0.409138411 | 0.000100804 | 0.001902223 | 2.720738679 |
| NDUFB6 | 0.754124045 | 1.84678E−13 | 1.40047E−11 | 10.85372676 | LRRC2 | 1.900245667 | 0.000101937 | 0.001920644 | 2.716553073 |
| CTSK | 1.379814625 | 1.95663E−13 | 1.47919E−11 | 10.82997667 | PAPSS1 | 0.717161953 | 0.000104176 | 0.001961325 | 2.707450391 |
| TAPBP | 0.888468385 | 1.96527E−13 | 1.48115E−11 | 10.82940223 | RBM39 | 0.304089099 | 0.000105515 | 0.001985005 | 2.702238504 |
| UBL5 | 0.618393719 | 1.9737E−13 | 1.48294E−11 | 10.82887694 | SBDS | 0.372636616 | 0.000107337 | 0.002014645 | 2.69580155 |
| WSB2 | 0.873756766 | 2.02084E−13 | 1.51372E−11 | 10.81995567 | MXD3 | 0.542868197 | 0.000107669 | 0.002019326 | 2.694793497 |
| ATP5G3 | 0.822960734 | 2.19566E−13 | 1.63965E−11 | 10.78524967 | HDDC2 | 0.445139557 | 0.000108328 | 0.002030139 | 2.692474305 |
| PARVA | 0.870565951 | 2.26342E−13 | 1.68511E−11 | 10.77337138 | TGFBR2 | 0.735537589 | 0.000108627 | 0.002034173 | 2.691612082 |
| C6orf1 | 0.859267652 | 2.27783E−13 | 1.6907E−11 | 10.77193362 | SRGAP2B | 0.390785456 | 0.000110789 | 0.002073076 | 2.683384847 |
| SLIRP | 0.658799648 | 2.43852E−13 | 1.8045E−11 | 10.74364225 | EXOC2 | 0.712153256 | 0.000111758 | 0.002088016 | 2.680266282 |
| SLC24A5 | 0.826314688 | 2.45971E−13 | 1.8147E−11 | 10.74119464 | JMJD8 | 0.507090688 | 0.000111713 | 0.002088016 | 2.680266282 |
| C3orf70 | 1.031989455 | 2.57373E−13 | 1.89312E−11 | 10.72282193 | AGTRAP | 0.633523226 | 0.000113279 | 0.002113217 | 2.675055976 |
| YWHAZ | 0.746937096 | 2.58315E−13 | 1.89436E−11 | 10.72253785 | RPSA | 0.156427011 | 0.000113211 | 0.002113217 | 2.675055976 |
| LINC01420 | 0.884212136 | 2.68019E−13 | 1.95966E−11 | 10.70782015 | NANS | 0.595627069 | 0.000113514 | 0.00211594 | 2.674496587 |
| B4GALT1 | 0.941307008 | 2.75455E−13 | 2.00804E−11 | 10.6972283 | GNAI1 | 0.471438557 | 0.000113598 | 0.00211594 | 2.674496587 |
| WDR83OS | 0.77330786 | 2.98024E−13 | 2.16611E−11 | 10.66431861 | NARS | 0.496550769 | 0.000114735 | 0.002133887 | 2.670828609 |
| PSMB6 | 0.798742354 | 3.28922E−13 | 2.38361E−11 | 10.62276447 | CYR61 | 1.206548095 | 0.000115204 | 0.002139644 | 2.669658407 |
| UBE2L3 | 0.810989201 | 3.7113E−13 | 2.68155E−11 | 10.57161363 | APOBEC3C | 0.558024108 | 0.00011522 | 0.002139644 | 2.669658407 |
| DKK3 | 2.12528038 | 3.89192E−13 | 2.80378E−11 | 10.55225552 | TMTC1 | 3.938338041 | 0.000115605 | 0.002145181 | 2.668536091 |
| SLC25A4 | 0.822105527 | 3.99242E−13 | 2.86775E−11 | 10.54245815 | ASB9 | 0.979350567 | 0.000116552 | 0.002160432 | 2.665459405 |
| RPL12 | 0.388610542 | 4.05022E−13 | 2.90077E−11 | 10.53748726 | MREG | 0.669823825 | 0.000116676 | 0.002160432 | 2.665459405 |
| TTYH3 | 0.894651413 | 4.46933E−13 | 3.18232E−11 | 10.49725657 | C7orf73 | 0.378016919 | 0.000116692 | 0.002160432 | 2.665459405 |
| PSMB2 | 0.869120717 | 5.11584E−13 | 3.6321E−11 | 10.43984212 | UPP1 | 0.57408309 | 0.00011815 | 0.002184131 | 2.660721333 |
| NEDD8 | 0.709221005 | 5.40799E−13 | 3.81739E−11 | 10.41823391 | NBPF8 | 0.362063408 | 0.000118379 | 0.002186713 | 2.660208306 |
| ST13 | 0.674700022 | 5.40604E−13 | 3.81739E−11 | 10.41823391 | RAB32 | 0.537716568 | 0.000120426 | 0.002222851 | 2.653089662 |
| SERPINB6 | 0.914572954 | 5.77373E−13 | 4.06384E−11 | 10.3910634 | ATP6V1E1 | 0.472015917 | 0.000120869 | 0.002229337 | 2.65182425 |
| COPS8 | 0.824326813 | 5.87787E−13 | 4.1135E−11 | 10.38578815 | FLII | 0.654322386 | 0.000121151 | 0.002232863 | 2.651137879 |
| FKBP9 | 0.587365448 | 5.86871E−13 | 4.1135E−11 | 10.38578815 | MAF | 0.463726044 | 0.000121596 | 0.002239378 | 2.649872521 |
| TTC3 | 0.693936706 | 6.61824E−13 | 4.61844E−11 | 10.33550491 | PGGT1B | 0.676456988 | 0.000121864 | 0.002242622 | 2.649243885 |
| FAM114A1 | 1.117810965 | 6.64205E−13 | 4.62188E−11 | 10.33518092 | TBL1XR1 | 0.534770548 | 0.000122384 | 0.002250515 | 2.647718177 |
| TSC22D3 | 1.021483898 | 7.3835E−13 | 5.1088E−11 | 10.29168136 | IL6ST | 0.666593015 | 0.000122695 | 0.002254526 | 2.646944764 |
| PTMA | 0.365496367 | 7.83023E−13 | 5.40264E−11 | 10.26739436 | PARVB | 0.530256689 | 0.000123137 | 0.002260953 | 2.6457085 |
| RPS13 | 0.454698056 | 9.30794E−13 | 6.40417E−11 | 10.19353687 | FUNDC2 | 0.486482292 | 0.000123572 | 0.002267247 | 2.644501231 |
| CRYM | 1.117062211 | 9.42715E−13 | 6.46803E−11 | 10.18922809 | MRPL54 | 0.595739186 | 0.000128134 | 0.002347424 | 2.629408509 |
| RBX1 | 0.723724723 | 9.50821E−13 | 6.50542E−11 | 10.18672453 | IFIT5 | 1.08909452 | 0.000132142 | 0.002417239 | 2.616680403 |
| TXNDC5 | 1.332899928 | 9.8335E−13 | 6.70924E−11 | 10.17332644 | TXNDC12 | 0.473647892 | 0.00013333 | 0.002435337 | 2.613440968 |
| TBCA | 0.757002234 | 9.94041E−13 | 6.76334E−11 | 10.16983867 | EPAS1 | 1.156866431 | 0.000133578 | 0.002438043 | 2.612958633 |
| GNPTAB | 0.808806896 | 1.13251E−12 | 7.66287E−11 | 10.11560827 | CISD3 | 0.478044301 | 0.000139608 | 0.002542423 | 2.594752156 |
| DNAJC13 | 0.991593122 | 1.29138E−12 | 8.68983E−11 | 10.06098888 | PSMB5 | 0.424723327 | 0.000140962 | 0.002565169 | 2.590883977 |
| ARL2 | 0.664188683 | 1.30905E−12 | 8.76063E−11 | 10.05746462 | TIMMDC1 | 0.54003036 | 0.000141512 | 0.002573276 | 2.589513574 |
| RPS28 | 0.386180103 | 1.30843E−12 | 8.76063E−11 | 10.05746462 | WDR13 | 0.521604836 | 0.000145702 | 0.002645529 | 2.577487445 |
| ECM1 | 1.056805611 | 1.33199E−12 | 8.88986E−11 | 10.05110498 | RTF1 | 0.433546543 | 0.00014625 | 0.002653525 | 2.576176855 |
| GALNT18 | 2.45523262 | 1.47379E−12 | 9.80954E−11 | 10.00835117 | ADGRA3 | 0.76331228 | 0.000147218 | 0.002669098 | 2.573635426 |
| NDUFS7 | 0.739266515 | 1.53932E−12 | 1.02179E−10 | 9.990638103 | CADPS | 0.846083283 | 0.000147529 | 0.002672755 | 2.573040838 |
| RPS8 | 0.423106045 | 1.56087E−12 | 1.0333E−10 | 9.985775514 | NAPG | 0.701556385 | 0.000148582 | 0.002689853 | 2.570271512 |
| GRN | 0.651506126 | 1.57491E−12 | 1.03978E−10 | 9.983059693 | DNAJC15 | 0.36029771 | 0.000148739 | 0.0026907 | 2.570134648 |
| SUB1 | 0.764939725 | 1.75352E−12 | 1.15459E−10 | 9.937572962 | CABLES1 | 0.463192582 | 0.000149958 | 0.002710761 | 2.56690881 |
| LRPAP1 | 0.742829621 | 1.97383E−12 | 1.29617E−10 | 9.887338816 | TNIP1 | 0.742573142 | 0.000153079 | 0.002761056 | 2.558924849 |
| NDUFA7 | 0.776345253 | 2.00392E−12 | 1.31241E−10 | 9.881931966 | PSMA2 | 0.407117903 | 0.000153684 | 0.00276993 | 2.557531181 |
| SAR1A | 0.677712381 | 2.25317E−12 | 1.47171E−10 | 9.83217788 | SOAT1 | 0.495386153 | 0.000154942 | 0.002790545 | 2.554310956 |
| ATP5J | 0.735955834 | 2.30542E−12 | 1.50183E−10 | 9.823378703 | DTX3L | 1.031688333 | 0.000156702 | 0.00282018 | 2.549723104 |
| NDUFB10 | 0.756456673 | 2.35692E−12 | 1.53131E−10 | 9.814936163 | RPL8 | 0.113135353 | 0.000157032 | 0.002824038 | 2.549129504 |
| CTNNA1 | 0.780731738 | 2.4135E−12 | 1.56392E−10 | 9.805784285 | RTTN | 0.580073178 | 0.000159619 | 0.002868461 | 2.542351068 |
| MRPL33 | 0.695313692 | 2.47315E−12 | 1.59834E−10 | 9.796329772 | LPP | 0.4245224 | 0.000159755 | 0.002868803 | 2.542299234 |
| SNW1 | 0.859416187 | 2.65097E−12 | 1.70876E−10 | 9.767318913 | TCN2 | 0.833893299 | 0.000164839 | 0.002957924 | 2.529013031 |
| ABCA5 | 1.237149239 | 2.93069E−12 | 1.8841E−10 | 9.724895316 | MRPS24 | 0.423728913 | 0.000165195 | 0.002962151 | 2.528392786 |
| PFDN5 | 0.536692381 | 3.07458E−12 | 1.97144E−10 | 9.705217167 | NAP1L4 | 0.440535128 | 0.000166913 | 0.002988564 | 2.524537414 |
| UQCC2 | 0.928892553 | 3.09107E−12 | 1.97683E−10 | 9.704029602 | TFAP2A | 0.245466962 | 0.000170152 | 0.003044347 | 2.516505841 |
| RPL13A | 0.324521899 | 3.24834E−12 | 2.06662E−10 | 9.684739789 | HNMT | 0.408183634 | 0.000170899 | 0.003055472 | 2.514921646 |
| SPECC1 | 2.320668459 | 3.34092E−12 | 2.12001E−10 | 9.673661238 | TMEM141 | 0.561117411 | 0.000172356 | 0.003074794 | 2.51218391 |
| UBA52 | 0.500758588 | 3.50209E−12 | 2.21654E−10 | 9.654323519 | HABP4 | 0.452160954 | 0.000172886 | 0.003082 | 2.511167316 |
| RPL10 | 0.373298109 | 3.58635E−12 | 2.25898E−10 | 9.646086755 | RPL37 | 0.195185751 | 0.00017318 | 0.003084991 | 2.510746098 |
| RPL23A | 0.296635747 | 3.58759E−12 | 2.25898E−10 | 9.646086755 | ATP6V0A1 | 0.663006723 | 0.000173383 | 0.003086373 | 2.51055152 |
| C4orf48 | 0.552130342 | 3.60427E−12 | 2.26367E−10 | 9.645187826 | BCAP29 | 0.561958194 | 0.000173768 | 0.003090984 | 2.509903197 |
| PLS3 | 0.955374181 | 3.75911E−12 | 2.35488E−10 | 9.628031852 | LNP1 | 0.594593108 | 0.000175833 | 0.003125434 | 2.505089644 |
| PSMB8 | 2.112588882 | 4.09004E−12 | 2.55565E−10 | 9.59249929 | HIGD1A | 0.391730964 | 0.000176189 | 0.003129489 | 2.504526636 |
| MINOS1 | 0.666468024 | 4.28308E−12 | 2.66946E−10 | 9.573576398 | MEF2C | 0.449040443 | 0.000177041 | 0.003138822 | 2.503233264 |
| HMGN2 | 0.736269355 | 4.6559E−12 | 2.89446E−10 | 9.538432451 | ZC3H4 | 0.357115 | 0.000177099 | 0.003138822 | 2.503233264 |
| EMP3 | 0.49256292 | 4.93272E−12 | 3.05879E−10 | 9.514450613 | FAM3C | 0.59075737 | 0.000179025 | 0.003170671 | 2.498848794 |
| PON2 | 0.75796783 | 5.08326E−12 | 3.14418E−10 | 9.50249299 | ST5 | 0.54321003 | 0.00018258 | 0.003231302 | 2.490622419 |
| ABCB5 | 2.197893143 | 5.57073E−12 | 3.43701E−10 | 9.463818813 | TCEAL8 | 0.422591865 | 0.000184575 | 0.003264241 | 2.486217806 |
| BANF1 | 0.652234137 | 5.93129E−12 | 3.65028E−10 | 9.437674035 | MSRA | 0.551275849 | 0.000189521 | 0.003349307 | 2.475045042 |
| LRRC75A-AS1 | 0.503461957 | 6.32267E−12 | 3.88139E−10 | 9.411012769 | PSMD10 | 0.438784987 | 0.000190929 | 0.003371753 | 2.472144271 |
| MORF4L1 | 0.70589906 | 6.77491E−12 | 4.13827E−10 | 9.383181161 | ZNF428 | 0.47308284 | 0.000191463 | 0.003378739 | 2.471245348 |
| C14orf2 | 0.710772514 | 6.95643E−12 | 4.22806E−10 | 9.373859088 | TUBB6 | 0.546634436 | 0.000192597 | 0.003396299 | 2.468994023 |
| NDUFC2 | 0.605518699 | 7.06713E−12 | 4.28471E−10 | 9.368078483 | SDC2 | 0.658910871 | 0.000194593 | 0.003429031 | 2.464828625 |
| RPL36AL | 0.516065359 | 7.13047E−12 | 4.31243E−10 | 9.365277449 | GRINA | 0.408472151 | 0.000195174 | 0.003436798 | 2.463845965 |
| DUT | 0.762745857 | 7.15825E−12 | 4.31857E−10 | 9.364659707 | CSPG4 | 0.648697734 | 0.000195579 | 0.003441459 | 2.463257346 |
| ABR | 0.941336215 | 7.21743E−12 | 4.34358E−10 | 9.362152337 | PPFIBP2 | 1.031066537 | 0.000195795 | 0.00344278 | 2.463090763 |
| GUK1 | 0.678512752 | 7.92438E−12 | 4.74572E−10 | 9.32369825 | GLRX5 | 0.441764265 | 0.000200491 | 0.003522824 | 2.453109082 |
| RPL18 | 0.32535845 | 8.15925E−12 | 4.87446E−10 | 9.312073668 | ADAP1 | 1.04056108 | 0.000203733 | 0.003574659 | 2.446765384 |
| CTSD | 0.413331032 | 8.36558E−12 | 4.98556E−10 | 9.302285823 | BBS5 | 0.519138515 | 0.000203588 | 0.003574659 | 2.446765384 |
| PRDX2 | 0.784907103 | 8.7637E−12 | 5.21015E−10 | 9.28314989 | SAP30 | 0.42720595 | 0.000205491 | 0.003600361 | 2.443653943 |
| TMEM205 | 0.905851543 | 9.10234E−12 | 5.39837E−10 | 9.26773709 | LRRC8D | 0.824615777 | 0.000210935 | 0.003690466 | 2.432918797 |
| RPLP1 | 0.295068562 | 9.3405E−12 | 5.52624E−10 | 9.257570295 | RAPH1 | 0.558872521 | 0.000210922 | 0.003690466 | 2.432918797 |
| GNPTG | 0.834547102 | 1.03041E−11 | 6.08166E−10 | 9.215977617 | TMEM64 | 1.266702652 | 0.000211699 | 0.003698537 | 2.431969981 |
| RPL41 | 0.340564042 | 1.09365E−11 | 6.4394E−10 | 9.191154382 | THUMPD1 | 0.54675746 | 0.000212314 | 0.003706643 | 2.431019251 |
| SSR2 | 0.720680118 | 1.09788E−11 | 6.44877E−10 | 9.190522897 | MICA | 1.430611014 | 0.000212602 | 0.003709022 | 2.430740583 |
| PRELID1 | 0.766710877 | 1.1256E−11 | 6.59579E−10 | 9.180733186 | BCAP31 | 0.369226992 | 0.000213839 | 0.00372795 | 2.4285299 |
| ALX1 | 0.989362061 | 1.14593E−11 | 6.6989E−10 | 9.173996463 | INSR | 0.419311285 | 0.000214737 | 0.003740938 | 2.427019524 |
| RPS26 | 0.442385733 | 1.38422E−11 | 8.07266E−10 | 9.092983365 | DTX4 | 1.169619322 | 0.000216264 | 0.003759522 | 2.424867316 |
| POLR2E | 0.79901433 | 1.45498E−11 | 8.46513E−10 | 9.072366533 | UQCRHL | 0.475858659 | 0.000216166 | 0.003759522 | 2.424867316 |
| ATP5I | 0.523076057 | 1.48999E−11 | 8.64829E−10 | 9.063069722 | SFXN3 | 0.539713323 | 0.000222047 | 0.00385458 | 2.414022963 |
| QPCT | 0.395058602 | 1.51402E−11 | 8.76697E−10 | 9.057150455 | C17orf49 | 0.454065084 | 0.000223005 | 0.00386848 | 2.412459611 |
| GRB2 | 0.729496121 | 1.53617E−11 | 8.87428E−10 | 9.051866761 | UBXN4 | 0.441596359 | 0.000223479 | 0.003873953 | 2.411845703 |
| ITGB1 | 0.754933655 | 1.5952E−11 | 9.1936E−10 | 9.036514368 | BMP1 | 0.850818038 | 0.000223925 | 0.003876193 | 2.411594558 |
| ATP5H | 0.675718904 | 1.6492E−11 | 9.48251E−10 | 9.023076598 | RPL13 | 0.110551044 | 0.000233691 | 0.004039534 | 2.393668693 |
| JUN | 0.941374898 | 1.65533E−11 | 9.49547E−10 | 9.022483571 | BNIP2 | 0.569132328 | 0.000235314 | 0.004064726 | 2.390968773 |
| RMND5A | 0.794564009 | 1.66935E−11 | 9.55349E−10 | 9.019837874 | ZSWIM7 | 0.585825086 | 0.000239204 | 0.00412901 | 2.384154016 |
| RPL24 | 0.384114712 | 1.89916E−11 | 1.08434E−09 | 8.964835866 | NDFIP1 | 0.46133101 | 0.000244183 | 0.004211983 | 2.375513411 |
| PSMA7 | 0.717040062 | 1.90599E−11 | 1.0857E−09 | 8.96428913 | PNRC1 | 0.284688652 | 0.000247324 | 0.004263163 | 2.370268017 |
| RAN | 0.752148092 | 2.05844E−11 | 1.16982E−09 | 8.931879909 | SNRPB2 | 0.470882118 | 0.000248861 | 0.004286642 | 2.367882822 |
| HNRNPH1 | 0.408778787 | 2.15987E−11 | 1.22462E−09 | 8.911997042 | VAMP3 | 0.398044288 | 0.000253656 | 0.004363092 | 2.360205667 |
| RPS16 | 0.33868593 | 2.22903E−11 | 1.26092E−09 | 8.899313093 | PDS5B | 0.533208907 | 0.000255278 | 0.004386244 | 2.357907191 |
| NDUFB9 | 0.694542706 | 2.25105E−11 | 1.27044E−09 | 8.896045316 | LSM1 | 0.507180274 | 0.000257337 | 0.004417108 | 2.354862003 |
| IFI27L2 | 0.640802205 | 2.31918E−11 | 1.30588E−09 | 8.884095849 | VIMP | 0.421383291 | 0.000259031 | 0.004439968 | 2.352620184 |
| ATP5G2 | 0.606312096 | 2.44813E−11 | 1.37533E−09 | 8.861592251 | LSM4 | 0.382922232 | 0.000258866 | 0.004439968 | 2.352620184 |
| MOB3B | 1.290115833 | 2.52069E−11 | 1.41285E−09 | 8.84990304 | ZFAS1 | 0.335448593 | 0.000266269 | 0.004560838 | 2.340955362 |
| ATP6V1G1 | 0.620355308 | 2.62353E−11 | 1.46714E−09 | 8.833528195 | SLC7A5 | 0.581118286 | 0.000266829 | 0.004567224 | 2.340347645 |
| LINC00623 | 1.536426425 | 2.82029E−11 | 1.57358E−09 | 8.803110549 | DDX17 | 0.319777161 | 0.000273054 | 0.004670523 | 2.33063447 |
| PSMB3 | 0.743638277 | 3.03383E−11 | 1.68888E−09 | 8.772401258 | EPS15 | 0.443231046 | 0.000278655 | 0.004762998 | 2.322119623 |
| CD44 | 0.300985187 | 3.1414E−11 | 1.7448E−09 | 8.758255354 | RPS27 | 0.146370471 | 0.000281674 | 0.004811244 | 2.317742586 |
| MYEF2 | 0.747854769 | 3.23401E−11 | 1.79217E−09 | 8.746621707 | ARPP19 | 0.366247147 | 0.00028221 | 0.004813685 | 2.317522322 |
| CCDC80 | 0.829238236 | 3.28494E−11 | 1.81628E−09 | 8.740816156 | TMED4 | 0.339955121 | 0.000282067 | 0.004813685 | 2.317522322 |
| PLOD3 | 0.764856339 | 3.79228E−11 | 2.09208E−09 | 8.679422654 | FBLN1 | 0.836228967 | 0.000286641 | 0.004882464 | 2.311360966 |
| GAPDHS | 1.393696666 | 3.88684E−11 | 2.13942E−09 | 8.669703175 | JTB | 0.358362585 | 0.000291598 | 0.00496344 | 2.304217184 |
| TM9SF2 | 0.930609167 | 4.05109E−11 | 2.22483E−09 | 8.652703356 | TMCO3 | 0.746726811 | 0.000291819 | 0.004963758 | 2.30418937 |
| BTF3 | 0.461730897 | 4.91776E−11 | 2.68874E−09 | 8.570451386 | LINC00987 | 0.642604709 | 0.000292428 | 0.004970673 | 2.303584782 |
| RAB7A | 0.543390036 | 5.05687E−11 | 2.75864E−09 | 8.55930483 | PSMD4 | 0.351861864 | 0.000294227 | 0.004997773 | 2.301223439 |
| RPL34 | 0.302993238 | 5.11999E−11 | 2.78687E−09 | 8.554883668 | SMC3 | 0.374514103 | 0.000298551 | 0.005060701 | 2.295789299 |
| OSBPL9 | 0.813868403 | 5.42766E−11 | 2.94127E−09 | 8.531465721 | POMGNT1 | 0.637447655 | 0.000304026 | 0.005142829 | 2.288797884 |
| TMEM140 | 1.57858932 | 5.46168E−11 | 2.95317E−09 | 8.529711983 | ANAPC11 | 0.454937786 | 0.000307728 | 0.00520186 | 2.283841347 |
| PRPF6 | 0.854774833 | 5.66029E−11 | 3.05381E−09 | 8.515157385 | MIR3074 | 0.397342443 | 0.000310083 | 0.005238049 | 2.28083044 |
| TRPM1 | 0.720565975 | 5.70135E−11 | 3.06921E−09 | 8.512973883 | WTAP | 0.398579121 | 0.000310493 | 0.005241367 | 2.28055546 |
| ATP5C1 | 0.641188979 | 5.96172E−11 | 3.20233E−09 | 8.494533315 | SP100 | 0.493695885 | 0.000313045 | 0.005277169 | 2.277598992 |
| ADCY2 | 1.407117605 | 6.02532E−11 | 3.22941E−09 | 8.490876625 | TSPAN5 | 1.822309971 | 0.000314605 | 0.005299824 | 2.275738545 |
| NDUFA12 | 0.701633513 | 6.31462E−11 | 3.37708E−09 | 8.471458813 | MRPL27 | 0.515141189 | 0.000317069 | 0.005333991 | 2.272947708 |
| RPL19 | 0.295767784 | 6.62327E−11 | 3.53443E−09 | 8.451680639 | NRXN3 | 0.58290118 | 0.000323084 | 0.005431455 | 2.265083844 |
| CLEC12B | 1.310696125 | 7.36926E−11 | 3.92397E−09 | 8.406274043 | QKI | 0.383665532 | 0.000325242 | 0.005463984 | 2.262490606 |
| SERINC1 | 0.643859208 | 7.63462E−11 | 4.05645E−09 | 8.391853775 | BSCL2 | 0.433433861 | 0.000327455 | 0.005497385 | 2.25984388 |
| RTFDC1 | 0.721319199 | 8.21327E−11 | 4.35445E−09 | 8.3610662 | POP5 | 0.648589551 | 0.000328938 | 0.005518497 | 2.25817916 |
| RPS24 | 0.430135608 | 8.33639E−11 | 4.41019E−09 | 8.355543031 | TMEM106C | 0.632210493 | 0.000331673 | 0.005552971 | 2.255474585 |
| COL6A2 | 0.871650398 | 9.17539E−11 | 4.84358E−09 | 8.314833715 | HLA-G | 2.26785326 | 0.000332798 | 0.005568 | 2.254300778 |
| SERPINE2 | 0.875655651 | 9.25627E−11 | 4.87577E−09 | 8.311957122 | ANKRD40 | 0.342999756 | 0.000335017 | 0.005597474 | 2.252007951 |
| HLA-E | 0.705374599 | 9.66497E−11 | 5.08013E−09 | 8.294125482 | APOLD1 | 1.567027569 | 0.000335921 | 0.005608751 | 2.251133823 |
| ETFB | 0.772463918 | 1.17795E−10 | 6.16509E−09 | 8.210060397 | POLR2I | 0.408921808 | 0.000336806 | 0.0056197 | 2.250286906 |
| SF3B5 | 0.705650926 | 1.20672E−10 | 6.30221E−09 | 8.200507439 | PGRMC2 | 0.503021598 | 0.000346276 | 0.005773777 | 2.238539984 |
| RPL30 | 0.314372987 | 1.22439E−10 | 6.38088E−09 | 8.195119168 | ETFA | 0.433319718 | 0.000346883 | 0.005779961 | 2.238075084 |
| RGL1 | 1.960083961 | 1.24579E−10 | 6.47865E−09 | 8.188515747 | ARRDC3 | 0.750768721 | 0.000349211 | 0.0058148 | 2.235465255 |
| CDH1 | 0.605099201 | 1.25373E−10 | 6.5061E−09 | 8.186679428 | SNURF | 0.409648359 | 0.00034956 | 0.005816663 | 2.235326082 |
| GTF3C6 | 0.781549275 | 1.35842E−10 | 7.03448E−09 | 8.152767809 | INPP4B | 0.347926348 | 0.000349888 | 0.005818166 | 2.235213879 |
| TAX1BP1 | 0.697759569 | 1.45876E−10 | 7.53814E−09 | 8.12273602 | CAPN3 | 0.171938032 | 0.000353138 | 0.005864252 | 2.231787363 |
| RPS15 | 0.280946553 | 1.60536E−10 | 8.26086E−09 | 8.082974482 | DPM3 | 0.402705103 | 0.000354022 | 0.005874937 | 2.230996764 |
| FAU | 0.446727753 | 1.64922E−10 | 8.46878E−09 | 8.072178915 | TMCO1 | 0.424786985 | 0.000357263 | 0.005920709 | 2.227626313 |
| LPPR4 | 1.349840879 | 1.72016E−10 | 8.81457E−09 | 8.054799098 | SNX3 | 0.262625009 | 0.000358313 | 0.005934085 | 2.226646229 |
| NDUFAB1 | 0.714878142 | 1.8316E−10 | 9.36602E−09 | 8.02844477 | MX2 | 1.536661983 | 0.000361095 | 0.005973581 | 2.223765207 |
| TMEM14B | 0.733836412 | 1.89276E−10 | 9.65859E−09 | 8.015086101 | SGK1 | 0.451893628 | 0.000361185 | 0.005973581 | 2.223765207 |
| PSMD9 | 0.801653385 | 1.92798E−10 | 9.81787E−09 | 8.007982523 | TSEN15 | 0.682245374 | 0.000362065 | 0.005981917 | 2.223159621 |
| MDH1 | 0.693605959 | 1.95009E−10 | 9.90983E−09 | 8.003933622 | WDR61 | 0.450386524 | 0.000362178 | 0.005981917 | 2.223159621 |
| TAX1BP3 | 0.621565998 | 1.98149E−10 | 1.00486E−08 | 7.997895443 | ICAM1 | 1.24238801 | 0.000362471 | 0.005982734 | 2.223100325 |
| SAP18 | 0.603723586 | 2.00483E−10 | 1.0146E−08 | 7.993707232 | CDC42BPA | 0.742003083 | 0.000367041 | 0.006049702 | 2.218266005 |
| SEC11C | 0.759819686 | 2.02349E−10 | 1.02192E−08 | 7.990581669 | TSN | 0.461002231 | 0.00036727 | 0.006049702 | 2.218266005 |
| TECR | 0.687431157 | 2.02953E−10 | 1.02286E−08 | 7.990181965 | SUPT16H | 0.387697518 | 0.000367934 | 0.006056568 | 2.217773372 |
| TRMT112 | 0.578607857 | 2.04296E−10 | 1.02752E−08 | 7.988209168 | MAD2L2 | 0.587536156 | 0.00036922 | 0.006073649 | 2.216550318 |
| YWHAQ | 0.579188168 | 2.25834E−10 | 1.13352E−08 | 7.945570696 | RPL6 | 0.211419657 | 0.000370717 | 0.006094189 | 2.215084046 |
| MIEN1 | 0.76034236 | 2.31145E−10 | 1.1578E−08 | 7.936365039 | RAB1A | 0.32327342 | 0.000371086 | 0.006096172 | 2.214942792 |
| SERPINB1 | 1.051168561 | 2.43473E−10 | 1.21707E−08 | 7.914685684 | CYCS | 0.402787775 | 0.000373404 | 0.006130125 | 2.212530679 |
| ATP1B3 | 0.629047871 | 2.62983E−10 | 1.31192E−08 | 7.882093959 | NOL7 | 0.373892754 | 0.000379303 | 0.006214474 | 2.206595612 |
| DTYMK | 0.967122197 | 2.75303E−10 | 1.37041E−08 | 7.863150305 | TXN2 | 0.455533236 | 0.000379822 | 0.00621882 | 2.206292043 |
| COMMD6 | 0.568412483 | 2.75827E−10 | 1.37041E−08 | 7.863150305 | UBE2H | 0.507721424 | 0.000387256 | 0.006336298 | 2.198164393 |
| MT2A | 0.898296416 | 2.86457E−10 | 1.42034E−08 | 7.847608084 | CNIH1 | 0.471934527 | 0.000390732 | 0.006388916 | 2.194572841 |
| NDUFS5 | 0.565566361 | 2.9802E−10 | 1.47469E−08 | 7.831299861 | NUTM2A-AS1 | 0.482429206 | 0.000391776 | 0.006400221 | 2.193805052 |
| SPRY1 | 1.623767018 | 3.23312E−10 | 1.59458E−08 | 7.797354328 | AP1G1 | 0.347509265 | 0.000391946 | 0.006400221 | 2.193805052 |
| DNAJC8 | 0.6686185 | 3.23551E−10 | 1.59458E−08 | 7.797354328 | NEO1 | 0.557470322 | 0.000395904 | 0.006459039 | 2.189832103 |
| WLS | 0.92164588 | 3.32526E−10 | 1.63552E−08 | 7.786343724 | CBX6 | 0.268816918 | 0.000396076 | 0.006459039 | 2.189832103 |
| HMCN1 | 1.902261615 | 3.34245E−10 | 1.64068E−08 | 7.784976193 | CCNDBP1 | 0.40290013 | 0.000397971 | 0.006485622 | 2.188048372 |
| AP2S1 | 0.750058472 | 3.55146E−10 | 1.73286E−08 | 7.76123763 | LMBRD1 | 0.570271134 | 0.000398365 | 0.006487739 | 2.187906602 |
| TLE1 | 0.747913659 | 3.55108E−10 | 1.73286E−08 | 7.76123763 | TAP1 | 0.931718528 | 0.000403132 | 0.006556644 | 2.183318418 |
| DPYSL2 | 0.804835379 | 3.70106E−10 | 1.80226E−08 | 7.744181938 | PTBP1 | 0.580061674 | 0.000402882 | 0.006556644 | 2.183318418 |
| ANXA2P1 | 0.825463116 | 3.71078E−10 | 1.80341E−08 | 7.743905136 | SYPL1 | 0.609084487 | 0.000403956 | 0.006565695 | 2.182719316 |
| MT1M | 1.448841214 | 3.89909E−10 | 1.89117E−08 | 7.723268299 | GSTM2 | 0.669403136 | 0.000408835 | 0.006640594 | 2.177793085 |
| CTSB | 0.510485291 | 4.10015E−10 | 1.98476E−08 | 7.702291326 | FKBP9P1 | 0.435956299 | 0.000413156 | 0.006706329 | 2.173515124 |
| MRPL51 | 0.678442478 | 4.10831E−10 | 1.98479E−08 | 7.702284961 | NRDE2 | 0.591873884 | 0.0004219 | 0.006843725 | 2.164707424 |
| RPL31 | 0.348705888 | 4.16328E−10 | 2.00739E−08 | 7.697367891 | PTK7 | 0.733278513 | 0.000426771 | 0.006913577 | 2.160297216 |
| STAT2 | 0.458592266 | 4.58258E−10 | 2.20522E−08 | 7.65654844 | ATG14 | 0.651173234 | 0.000427904 | 0.006927357 | 2.159432434 |
| NDUFS4 | 0.723379076 | 4.59791E−10 | 2.20826E−08 | 7.655949887 | WBSCR22 | 0.473588258 | 0.000429464 | 0.006948016 | 2.158139171 |
| CDS2 | 0.71995914 | 4.63502E−10 | 2.22172E−08 | 7.653309747 | PIN1 | 0.372850686 | 0.000430206 | 0.006955428 | 2.157676132 |
| TPT1 | 0.382009178 | 4.64422E−10 | 2.22179E−08 | 7.65329753 | BCAS3 | 1.110972881 | 0.000433507 | 0.007004172 | 2.154643204 |
| EIF1 | 0.31746161 | 4.7234E−10 | 2.25526E−08 | 7.646803065 | RAB4B | 0.651771367 | 0.00043522 | 0.007027212 | 2.153216954 |
| COL27A1 | 0.971150577 | 4.84212E−10 | 2.30745E−08 | 7.636868018 | LTBR | 0.47995165 | 0.000443416 | 0.007150114 | 2.145687007 |
| SRPX | 0.804776788 | 4.94052E−10 | 2.34977E−08 | 7.628974434 | CD81 | 0.358287305 | 0.000445603 | 0.007180665 | 2.14383532 |
| AP1S2 | 0.425751388 | 4.99176E−10 | 2.36954E−08 | 7.625336517 | PARP9 | 1.909301043 | 0.000446399 | 0.007184032 | 2.143631714 |
| PET100 | 0.571542084 | 5.05982E−10 | 2.3972E−08 | 7.620295708 | RNF213 | 0.449580759 | 0.000446152 | 0.007184032 | 2.143631714 |
| ETHE1 | 0.97571522 | 5.08147E−10 | 2.40281E−08 | 7.61928034 | MFAP3L | 0.789176643 | 0.000447856 | 0.007202754 | 2.142501425 |
| NUCB1 | 0.701004088 | 5.11759E−10 | 2.41522E−08 | 7.617042463 | SCAMP3 | 0.463671088 | 0.000449042 | 0.007217084 | 2.141638257 |
| RNF130 | 0.739968002 | 5.48934E−10 | 2.58569E−08 | 7.587423436 | TMEM255A | 1.810063362 | 0.000450646 | 0.007234258 | 2.14060598 |
| SFTPC | 1.846952677 | 5.82601E−10 | 2.73376E−08 | 7.563239855 | BAD | 0.422875464 | 0.000454875 | 0.007296463 | 2.136887597 |
| CD55 | 1.361979246 | 6.05025E−10 | 2.82815E−08 | 7.548498226 | SCIN | 3.090935946 | 0.000457312 | 0.007325959 | 2.1351355 |
| GLMP | 0.763043761 | 6.53916E−10 | 3.05086E−08 | 7.515577459 | ANXA7 | 0.531219542 | 0.000457227 | 0.007325959 | 2.1351355 |
| SELK | 0.704958737 | 6.63529E−10 | 3.08982E−08 | 7.510066255 | RPS19 | 0.078740746 | 0.00046041 | 0.007370779 | 2.132486609 |
| RPL9 | 0.305623293 | 6.7656E−10 | 3.14453E−08 | 7.502444442 | ATF6B | 0.621507585 | 0.000460976 | 0.007375012 | 2.132237268 |
| CTNNB1 | 0.608740628 | 6.93344E−10 | 3.21643E−08 | 7.492625248 | C5orf15 | 0.656372607 | 0.000462222 | 0.007390126 | 2.131348176 |
| AK2 | 0.763328016 | 7.18653E−10 | 3.32754E−08 | 7.477876434 | GALK1 | 0.52811408 | 0.000463358 | 0.007401351 | 2.130689027 |
| SPAG7 | 0.837044954 | 7.39884E−10 | 3.41938E−08 | 7.466052804 | AIMP1 | 0.478464186 | 0.000463529 | 0.007401351 | 2.130689027 |
| MYO10 | 0.478778422 | 7.73757E−10 | 3.56919E−08 | 7.44743014 | CNPY2 | 0.369750708 | 0.000465323 | 0.007425168 | 2.129293687 |
| ZEB2 | 0.589832902 | 7.99862E−10 | 3.68267E−08 | 7.433836856 | FAM198B | 1.248951077 | 0.00046681 | 0.007444036 | 2.128191537 |
| TRAPPC1 | 0.597944796 | 8.15415E−10 | 3.74724E−08 | 7.426289061 | STAT3 | 0.55542618 | 0.000472944 | 0.007532053 | 2.123086633 |
| TMEM258 | 0.474837601 | 8.51954E−10 | 3.90782E−08 | 7.408065531 | S100A1 | 0.792594075 | 0.000474163 | 0.007546551 | 2.122251471 |
| NDUFA2 | 0.662420392 | 8.77556E−10 | 4.01773E−08 | 7.396019241 | IRF1 | 1.130978703 | 0.000477222 | 0.007590305 | 2.119740801 |
| RPS14 | 0.255933702 | 9.11738E−10 | 4.16644E−08 | 7.38023487 | LMAN1 | 0.614187658 | 0.000482774 | 0.00766866 | 2.115280506 |
| RND3 | 0.747841895 | 9.24841E−10 | 4.21845E−08 | 7.374847432 | SMIM7 | 0.452709436 | 0.000489852 | 0.007776046 | 2.109241167 |
| RPS29 | 0.400920391 | 9.28074E−10 | 4.22532E−08 | 7.374139958 | POLR2K | 0.503406346 | 0.0004973 | 0.007889161 | 2.102969177 |
| GADD45GIP1 | 0.709824681 | 1.07812E−09 | 4.89028E−08 | 7.310666129 | ESYT2 | 0.556663334 | 0.000499582 | 0.007920237 | 2.101261799 |
| COLGALT1 | 0.810695231 | 1.09433E−09 | 4.95464E−08 | 7.304987768 | P4HA2 | 0.521531701 | 0.000507725 | 0.00803892 | 2.094802295 |
| RPL14 | 0.290335268 | 1.10761E−09 | 4.99628E−08 | 7.30135333 | KANK2 | 1.03199625 | 0.00050853 | 0.008046473 | 2.094394466 |
| KIAA1456 | 0.983334124 | 1.12001E−09 | 5.04294E−08 | 7.297316233 | UFSP2 | 0.438294291 | 0.000509772 | 0.008060911 | 2.093615885 |
| KCNE4 | 1.418795943 | 1.14452E−09 | 5.14385E−08 | 7.28871157 | RRAGD | 0.460464776 | 0.000512919 | 0.00810545 | 2.091222861 |
| ROMO1 | 0.550616324 | 1.22823E−09 | 5.50994E−08 | 7.258852759 | PCMTD2 | 0.497438252 | 0.000513451 | 0.008108619 | 2.091053109 |
| PLA2G16 | 1.156492949 | 1.24702E−09 | 5.58401E−08 | 7.253053986 | SELT | 0.392944276 | 0.000515771 | 0.008140007 | 2.089375215 |
| PFDN1 | 0.8115924 | 1.25902E−09 | 5.62744E−08 | 7.249688882 | RNF181 | 0.324664891 | 0.000519743 | 0.008192146 | 2.08660232 |
| NOP10 | 0.642263174 | 1.36564E−09 | 6.08183E−08 | 7.215965602 | STT3A | 0.42645365 | 0.000521423 | 0.008212552 | 2.085521885 |
| HEBP1 | 0.776765406 | 1.46789E−09 | 6.52532E−08 | 7.185398408 | MARCKS | 0.201730579 | 0.000521709 | 0.008212552 | 2.085521885 |
| LINC00998 | 0.683173299 | 1.47177E−09 | 6.5307E−08 | 7.185040336 | RPL36A | 0.244550452 | 0.000525628 | 0.008263625 | 2.082829399 |
| PRKDC | 0.686728537 | 1.51339E−09 | 6.70325E−08 | 7.17371468 | DCTN3 | 0.414965421 | 0.000527967 | 0.008294799 | 2.081194157 |
| CDH3 | 0.428084522 | 1.67041E−09 | 7.38538E−08 | 7.131626863 | MRPS15 | 0.46817103 | 0.000532059 | 0.008343314 | 2.078661394 |
| WFS1 | 1.021361828 | 1.77948E−09 | 7.83931E−08 | 7.105722372 | TAOK3 | 0.430815637 | 0.000533743 | 0.008364368 | 2.077566848 |
| SDHB | 0.684752226 | 1.79011E−09 | 7.87198E−08 | 7.103916085 | TMED7 | 0.648157597 | 0.000534327 | 0.008368168 | 2.077369593 |
| S100B | 0.875911832 | 1.80298E−09 | 7.91439E−08 | 7.101582815 | SEC14L1 | 0.724489212 | 0.000536083 | 0.008390304 | 2.076222323 |
| TCEAL4 | 0.662527978 | 1.84699E−09 | 8.09304E−08 | 7.091888096 | MEGF6 | 1.281494975 | 0.000540595 | 0.008452173 | 2.073031637 |
| MRPL41 | 0.672435582 | 1.88632E−09 | 8.25065E−08 | 7.08351181 | NCL | 0.434315771 | 0.000542798 | 0.008479149 | 2.07164774 |
| FBXL5 | 0.967761099 | 1.89547E−09 | 8.27589E−08 | 7.082185332 | DAG1 | 0.414707005 | 0.000543557 | 0.008485582 | 2.071318351 |
| APLP2 | 0.697575867 | 1.91294E−09 | 8.33728E−08 | 7.078975766 | CPEB2 | 0.490182102 | 0.000548792 | 0.008561862 | 2.067431787 |
| ST8SIA6 | 1.215541124 | 1.93913E−09 | 8.43644E−08 | 7.073840698 | B3GNT2 | 0.865657628 | 0.000555184 | 0.008656072 | 2.062679147 |
| FAM46A | 1.13553822 | 2.08361E−09 | 9.04891E−08 | 7.043403502 | ZBED5-AS1 | 0.958973527 | 0.000570381 | 0.008887354 | 2.051227526 |
| SUMO2 | 0.544288278 | 2.18657E−09 | 9.47927E−08 | 7.023225211 | IFI16 | 0.354881227 | 0.000574223 | 0.008941528 | 2.04858824 |
| CBX3 | 0.689852893 | 2.35792E−09 | 1.0204E−07 | 6.991227715 | WASH7P | 0.458838105 | 0.000578823 | 0.009007433 | 2.045398957 |
| CLU | 1.658089757 | 2.40372E−09 | 1.03839E−07 | 6.983638955 | TMEM185A | 0.568716109 | 0.000582929 | 0.00906557 | 2.042604875 |
| SNRPG | 0.591850579 | 2.54945E−09 | 1.09941E−07 | 6.958841836 | ETS1 | 0.376926422 | 0.000585859 | 0.009105344 | 2.040703636 |
| USP22 | 0.54169786 | 2.66356E−09 | 1.1466E−07 | 6.940589842 | SRI | 0.367246181 | 0.000587701 | 0.009128183 | 2.039615662 |
| MRPL20 | 0.704763889 | 2.82968E−09 | 1.21597E−07 | 6.915078559 | CAST | 0.392394125 | 0.000588802 | 0.009139485 | 2.03907828 |
| SH3BP4 | 1.003214359 | 2.87978E−09 | 1.23317E−07 | 6.908976831 | PIGT | 0.401897788 | 0.000591508 | 0.00917486 | 2.037400571 |
| GSN | 0.599892676 | 3.01185E−09 | 1.28748E−07 | 6.890261005 | FSTL4 | 2.570472956 | 0.000593224 | 0.009190657 | 2.036653452 |
| DAZAP2 | 0.433351904 | 3.16773E−09 | 1.35175E−07 | 6.869104571 | BTF3L4 | 0.48592034 | 0.000594191 | 0.00919982 | 2.036220688 |
| LAMP2 | 0.614622951 | 3.19112E−09 | 1.35936E−07 | 6.866664508 | BLVRA | 0.467644542 | 0.000598002 | 0.009252971 | 2.0337188 |
| HSPE1 | 0.543538034 | 3.23682E−09 | 1.37643E−07 | 6.86124482 | EFHD1 | 2.315544367 | 0.00060267 | 0.009319315 | 2.030615986 |
| GABARAPL2 | 0.576479912 | 3.25294E−09 | 1.38089E−07 | 6.85983984 | DEK | 0.43154338 | 0.000613916 | 0.00948125 | 2.023134387 |
| RPL35 | 0.329265505 | 3.41517E−09 | 1.44725E−07 | 6.839455418 | MYADM | 0.424624711 | 0.000614989 | 0.009491827 | 2.022650167 |
| HLA-F | 1.515982866 | 3.61274E−09 | 1.5257E−07 | 6.816531617 | COPS6 | 0.379299045 | 0.000618105 | 0.009533914 | 2.020728774 |
| CAP2 | 2.086347342 | 3.75356E−09 | 1.58244E−07 | 6.800673338 | THYN1 | 0.66306591 | 0.000620601 | 0.009566389 | 2.01925198 |
| KLF9 | 1.283636689 | 3.82974E−09 | 1.61178E−07 | 6.792693532 | KCNN3 | 0.900473356 | 0.000629921 | 0.009703957 | 2.01305115 |
| SEC61G | 0.567168593 | 4.35542E−09 | 1.82987E−07 | 6.737579048 | RAP1GAP | 0.847214282 | 0.000634364 | 0.009760114 | 2.010545128 |
| ADGRG1 | 0.465326995 | 4.58534E−09 | 1.92317E−07 | 6.715981728 | HSPA8 | 0.377990097 | 0.000634211 | 0.009760114 | 2.010545128 |
| LDHB | 0.780707717 | 4.59438E−09 | 1.92323E−07 | 6.715969599 | ANAPC16 | 0.347303212 | 0.000640909 | 0.00985317 | 2.006424038 |
| CPQ | 0.603915334 | 4.60117E−09 | 1.92323E−07 | 6.715969599 | HNRNPL | 0.220828697 | 0.000641216 | 0.00985317 | 2.006424038 |
| 44076 | 0.681917429 | 4.61796E−09 | 1.92696E−07 | 6.715127801 | OXCT1 | 0.6267519 | 0.000646275 | 0.009924678 | 2.003283593 |
| RPLP0 | 0.390155524 | 4.64441E−09 | 1.9347E−07 | 6.713387001 | RPS9 | 0.163317993 | 0.000657055 | 0.010083898 | 1.996371568 |
| IRS2 | 0.526293099 | 4.69312E−09 | 1.95167E−07 | 6.709593495 | VLDLR | 1.195731997 | 0.000663817 | 0.010181302 | 1.992196676 |
| SLC6A15 | 0.894570649 | 4.70746E−09 | 1.95432E−07 | 6.70900521 | GPC1 | 0.656397104 | 0.000669322 | 0.010259304 | 1.98888212 |
| MFGE8 | 0.724413455 | 4.80432E−09 | 1.99115E−07 | 6.700895871 | ASXL1 | 0.441753924 | 0.00067382 | 0.010321798 | 1.986244624 |
| COMMD1 | 0.827085316 | 4.87332E−09 | 2.01633E−07 | 6.695437347 | PRKD1 | 0.611034214 | 0.000674748 | 0.010323091 | 1.98619024 |
| ESYT1 | 0.923043668 | 4.93842E−09 | 2.03983E−07 | 6.690406809 | ATG4B | 0.507546902 | 0.000676565 | 0.010340458 | 1.985460216 |
| GNG11 | 0.753622115 | 5.39724E−09 | 2.22559E−07 | 6.652554864 | CBR3 | 0.681693137 | 0.000678509 | 0.010361225 | 1.984588885 |
| TMEM98 | 0.824052334 | 5.70277E−09 | 2.34762E−07 | 6.629371776 | FAM189A2 | 0.723243415 | 0.000678996 | 0.010362199 | 1.984548092 |
| ISCU | 0.686388314 | 5.87325E−09 | 2.41375E−07 | 6.617307946 | HSPA12A | 0.588326156 | 0.000682209 | 0.010398272 | 1.983038846 |
| RBM8A | 0.643823743 | 6.16024E−09 | 2.52745E−07 | 6.59731679 | NFU1 | 0.566298962 | 0.000682902 | 0.010402364 | 1.982867945 |
| NARF | 0.761276484 | 6.52101E−09 | 2.671E−07 | 6.573326285 | CCDC107 | 0.523123145 | 0.000688142 | 0.010469169 | 1.980087803 |
| ATP5J2 | 0.577804565 | 6.64815E−09 | 2.71853E−07 | 6.565666097 | SEMA6D | 0.738139153 | 0.000689088 | 0.010477047 | 1.979761099 |
| TCEA1 | 0.633507848 | 6.74936E−09 | 2.75531E−07 | 6.559828959 | CCL2 | 3.439841986 | 0.000696489 | 0.010576437 | 1.9756606 |
| SSNA1 | 0.711164892 | 6.76187E−09 | 2.75583E−07 | 6.559748046 | AKR1B1 | 0.397513777 | 0.000696406 | 0.010576437 | 1.9756606 |
| PLBD2 | 1.018955112 | 6.93667E−09 | 2.82237E−07 | 6.549385645 | CCDC25 | 0.585574627 | 0.000699361 | 0.010613476 | 1.974142353 |
| UFC1 | 0.712689161 | 6.9651E−09 | 2.82924E−07 | 6.548330297 | MAPKAP1 | 0.5003075 | 0.000702831 | 0.010659529 | 1.972261966 |
| AMOTL2 | 1.183326602 | 7.20504E−09 | 2.92186E−07 | 6.534340767 | SUMO1 | 0.30620876 | 0.000707707 | 0.010720209 | 1.969796761 |
| RNF144B | 0.825903416 | 7.2585E−09 | 2.93867E−07 | 6.531848682 | IQCK | 0.381798536 | 0.00071236 | 0.010784022 | 1.967219229 |
| ARNT2 | 0.769370019 | 7.47477E−09 | 3.02124E−07 | 6.519814789 | HIBCH | 0.43618536 | 0.000715549 | 0.010825604 | 1.965547844 |
| ILVBL | 0.664249361 | 7.72201E−09 | 3.11603E−07 | 6.506398447 | COA6 | 0.550274551 | 0.00071965 | 0.010867511 | 1.963869903 |
| DYNC1I2 | 0.609551847 | 8.32162E−09 | 3.35246E−07 | 6.474635931 | UBQLN2 | 0.697792053 | 0.000725195 | 0.010944501 | 1.960804048 |
| PSME2 | 0.870562136 | 8.78527E−09 | 3.52765E−07 | 6.452515046 | CDC25B | 0.540728152 | 0.000730997 | 0.011025271 | 1.957610745 |
| MESDC2 | 0.747751176 | 9.17507E−09 | 3.67814E−07 | 6.43437228 | LGALS3 | 0.201724812 | 0.000731997 | 0.011033562 | 1.957284263 |
| PSMA6 | 0.639119446 | 9.23198E−09 | 3.6949E−07 | 6.432396898 | PBXIP1 | 0.515148818 | 0.000741012 | 0.01116258 | 1.952235431 |
| GOLM1 | 0.850989461 | 9.2543E−09 | 3.6978E−07 | 6.432057016 | BCL10 | 0.472834498 | 0.0007464 | 0.011236827 | 1.94935631 |
| CORO1C | 0.738921523 | 9.33691E−09 | 3.72119E−07 | 6.429318573 | SMC6 | 0.441757202 | 0.00074973 | 0.011266186 | 1.94822307 |
| CDV3 | 0.632633626 | 9.34323E−09 | 3.72119E−07 | 6.429318573 | ATAD1 | 0.686810732 | 0.000757599 | 0.01137745 | 1.943955047 |
| ZFP36L1 | 0.513053119 | 9.53369E−09 | 3.79088E−07 | 6.421260185 | CSRP2 | 2.05749011 | 0.000761246 | 0.011425217 | 1.942135531 |
| PTEN | 0.614432216 | 9.79572E−09 | 3.88876E−07 | 6.410189267 | SCAND1 | 0.372254997 | 0.000765 | 0.01147453 | 1.940265095 |
| LEF1 | 0.859762728 | 9.84877E−09 | 3.90349E−07 | 6.408546952 | MRPS36 | 0.394344956 | 0.000770869 | 0.011555482 | 1.937211922 |
| CTSO | 1.242852926 | 9.86588E−09 | 3.90395E−07 | 6.40849525 | WWC3 | 0.55744493 | 0.000771508 | 0.011557994 | 1.93711753 |
| ALDOA | 0.422914386 | 1.06122E−08 | 4.19252E−07 | 6.377524793 | ACADS | 0.552390635 | 0.000773933 | 0.011587239 | 1.936020043 |
| ATP5F1 | 0.640120506 | 1.07365E−08 | 4.23479E−07 | 6.373168291 | PDIA6 | 0.476896465 | 0.000781113 | 0.01168114 | 1.932514787 |
| SPTBN1 | 0.404987723 | 1.09514E−08 | 4.31258E−07 | 6.365262445 | ARHGAP31 | 1.101248503 | 0.000796834 | 0.011908262 | 1.924151634 |
| RPL38 | 0.324875504 | 1.19813E−08 | 4.7106E−07 | 6.32692377 | DNM2 | 0.482607037 | 0.00080075 | 0.011959498 | 1.922287053 |
| ZCRB1 | 0.785244882 | 1.20829E−08 | 4.74291E−07 | 6.323955324 | MAFIP | 1.129866838 | 0.000818767 | 0.012221125 | 1.912888802 |
| ECH1 | 0.693313658 | 1.22861E−08 | 4.81497E−07 | 6.317406645 | PQLC2L | 2.463640213 | 0.000823171 | 0.012279389 | 1.910823251 |
| C9orf3 | 0.390638351 | 1.27135E−08 | 4.97452E−07 | 6.303248769 | CLNS1A | 0.413777888 | 0.000829453 | 0.01235053 | 1.908314408 |
| GDI2 | 0.620795131 | 1.30576E−08 | 5.101E−07 | 6.29234483 | PQLC3 | 0.423848987 | 0.000835435 | 0.012432041 | 1.905457569 |
| PHB | 0.64775151 | 1.31504E−08 | 5.12906E−07 | 6.289962464 | MMGT1 | 0.658735871 | 0.000838243 | 0.012460162 | 1.904476304 |
| NDUFS8 | 0.628571093 | 1.35524E−08 | 5.26911E−07 | 6.278263028 | CAPZB | 0.326253474 | 0.000849765 | 0.012614629 | 1.89912552 |
| CUTA | 0.481335044 | 1.3697E−08 | 5.31685E−07 | 6.274345247 | MRPS7 | 0.421972215 | 0.000851766 | 0.012636679 | 1.898367061 |
| PCBD1 | 0.795430779 | 1.39678E−08 | 5.41341E−07 | 6.266528959 | WASH2P | 0.47800988 | 0.000853972 | 0.012661744 | 1.897506484 |
| MME | 1.368176818 | 1.47246E−08 | 5.69772E−07 | 6.244299203 | DYNLT1 | 0.41208756 | 0.000864018 | 0.012795195 | 1.892953089 |
| HSBP1L1 | 1.032973409 | 1.49353E−08 | 5.77011E−07 | 6.238816084 | PPP1R15A | 0.408996314 | 0.000868444 | 0.012852969 | 1.890996533 |
| DECR1 | 0.681937337 | 1.54667E−08 | 5.96599E−07 | 6.224317287 | INPP5K | 0.508225799 | 0.000869873 | 0.012866343 | 1.890544866 |
| BIRC3 | 2.196219921 | 1.61525E−08 | 6.21099E−07 | 6.20683889 | PAQR8 | 0.680358291 | 0.000877205 | 0.012959138 | 1.887423886 |
| SPSB1 | 1.286089897 | 1.6151E−08 | 6.21099E−07 | 6.20683889 | SDC4 | 0.787303686 | 0.000881435 | 0.013005941 | 1.885858215 |
| PAFAH1B2 | 0.791041255 | 1.62852E−08 | 6.25218E−07 | 6.203968838 | PLA2G15 | 0.603113532 | 0.000881331 | 0.013005941 | 1.885858215 |
| TMED9 | 0.683480918 | 1.6974E−08 | 6.50642E−07 | 6.186657621 | ACSL1 | 0.851944625 | 0.000882935 | 0.013020239 | 1.885381059 |
| WASF2 | 0.55248946 | 1.75373E−08 | 6.71186E−07 | 6.17315685 | GMFG | 1.788087368 | 0.000884905 | 0.013033592 | 1.884935863 |
| PSMD1 | 0.72898525 | 1.76508E−08 | 6.74476E−07 | 6.171033487 | UQCRFS1 | 0.29504481 | 0.000897808 | 0.013207758 | 1.879170903 |
| HERC2 | 0.956747353 | 1.78427E−08 | 6.80746E−07 | 6.167015163 | STMN1 | 0.208255261 | 0.000903452 | 0.013282804 | 1.876710231 |
| ARSA | 0.853154182 | 1.95278E−08 | 7.4388E−07 | 6.1284973 | ACOT7 | 0.426820338 | 0.000909075 | 0.013357462 | 1.874276065 |
| IFNGR2 | 0.648304641 | 1.99401E−08 | 7.58407E−07 | 6.120097836 | MAGOH | 0.463456392 | 0.000918752 | 0.013491558 | 1.869937899 |
| CTSZ | 0.672708452 | 2.02784E−08 | 7.70075E−07 | 6.113466935 | ADK | 0.62916702 | 0.000924248 | 0.013564123 | 1.867608278 |
| H2AFZ | 0.480883539 | 2.08374E−08 | 7.90081E−07 | 6.102328401 | C21orf33 | 0.38718316 | 0.000936587 | 0.013720551 | 1.86262846 |
| NAP1L1 | 0.380236477 | 2.23814E−08 | 8.47309E−07 | 6.071958161 | APH1B | 0.611713409 | 0.000941686 | 0.01377877 | 1.860789539 |
| GNB1 | 0.464463174 | 2.25379E−08 | 8.51919E−07 | 6.069601878 | RAB18 | 0.451515526 | 0.000942676 | 0.01378502 | 1.860592599 |
| CNPY3 | 0.737265944 | 2.2779E−08 | 8.59705E−07 | 6.065650438 | MYO1E | 0.475840032 | 0.000955633 | 0.013957827 | 1.855182188 |
| TMEM230 | 0.595962405 | 2.32381E−08 | 8.75683E−07 | 6.057653258 | RASGEF1B | 0.36298725 | 0.000962678 | 0.014052345 | 1.852251195 |
| HPS4 | 0.851876438 | 2.37865E−08 | 8.94973E−07 | 6.048189934 | PPM1B | 0.649869323 | 0.000977937 | 0.014258083 | 1.845938872 |
| ECHS1 | 0.626672745 | 2.43293E−08 | 9.13989E−07 | 6.039059004 | ESD | 0.28625536 | 0.000985764 | 0.014355119 | 1.842993203 |
| BICC1 | 1.683460593 | 2.66503E−08 | 9.96598E−07 | 6.001479906 | BMP7 | 1.007565498 | 0.000992668 | 0.014447069 | 1.840220267 |
| SGCE | 0.698387444 | 2.77533E−08 | 1.03469E−06 | 5.98519152 | SLCO4A1 | 0.501950145 | 0.001001361 | 0.014564929 | 1.836691617 |
| CTTN | 0.445470333 | 2.77377E−08 | 1.03469E−06 | 5.98519152 | TMEM184B | 0.493982494 | 0.001013885 | 0.014729598 | 1.831809094 |
| NDUFA3 | 0.499722004 | 2.83623E−08 | 1.05579E−06 | 5.976424059 | ARHGEF40 | 0.492768347 | 0.001017304 | 0.014770512 | 1.830604454 |
| LIPA | 1.340698242 | 2.93908E−08 | 1.09241E−06 | 5.961613736 | NIPA2 | 0.524401963 | 0.001025661 | 0.014883025 | 1.827308799 |
| SLCO4A1-AS1 | 0.757601619 | 3.03052E−08 | 1.12299E−06 | 5.949625039 | KIAA0556 | 1.039207339 | 0.001039911 | 0.015071946 | 1.821830683 |
| TXNL4A | 0.589421868 | 3.02769E−08 | 1.12299E−06 | 5.949625039 | CD99L2 | 0.725026369 | 0.001039851 | 0.015071946 | 1.821830683 |
| TJP2 | 1.141427755 | 3.10582E−08 | 1.1474E−06 | 5.940284075 | ITGA3 | 0.786168933 | 0.001044198 | 0.015125121 | 1.820301138 |
| ZNF652 | 0.682116985 | 3.11046E−08 | 1.1474E−06 | 5.940284075 | ARHGEF9 | 0.73713392 | 0.001046438 | 0.015148613 | 1.81962714 |
| FIS1 | 0.729519188 | 3.38598E−08 | 1.24716E−06 | 5.904076939 | SNRPC | 0.413831383 | 0.001056911 | 0.015291181 | 1.815558973 |
| DDT | 0.677693963 | 3.41954E−08 | 1.25763E−06 | 5.900446657 | LPCAT1 | 0.769713998 | 0.001060525 | 0.015329807 | 1.814463324 |
| NHP2 | 0.63341254 | 3.52543E−08 | 1.29148E−06 | 5.88891177 | IFT81 | 0.610623419 | 0.001072429 | 0.015488259 | 1.809997391 |
| CHST9 | 1.477501154 | 3.72673E−08 | 1.36243E−06 | 5.86568669 | ANKRD28 | 0.52598691 | 0.001078272 | 0.015554296 | 1.808149632 |
| CAV2 | 0.617839813 | 3.7751E−08 | 1.37805E−06 | 5.860734646 | C11orf73 | 0.471376836 | 0.00107821 | 0.015554296 | 1.808149632 |
| NDUFB3 | 0.572153151 | 4.02135E−08 | 1.46576E−06 | 5.833937906 | TMEM54 | 1.530860186 | 0.001081954 | 0.015598226 | 1.806924792 |
| RETSAT | 0.599687934 | 4.29067E−08 | 1.5616E−06 | 5.806430353 | UBA6 | 0.43157059 | 0.001084395 | 0.015615026 | 1.806457278 |
| EMC4 | 0.604271829 | 4.30138E−08 | 1.56318E−06 | 5.805991929 | GTF3A | 0.39045909 | 0.001084081 | 0.015615026 | 1.806457278 |
| RPS17 | 0.412584215 | 4.41486E−08 | 1.60204E−06 | 5.795326708 | SDCBP | 0.455851257 | 0.001089165 | 0.015665297 | 1.805061367 |
| NOTCH2NL | 0.783808231 | 4.53877E−08 | 1.64457E−06 | 5.783948528 | GALE | 0.488427043 | 0.001102256 | 0.01584428 | 1.800127484 |
| FXYD3 | 1.437506795 | 4.5952E−08 | 1.66179E−06 | 5.779424966 | SLC35A5 | 0.761637151 | 0.001107993 | 0.015917405 | 1.79812772 |
| RAB13 | 0.697258949 | 4.59986E−08 | 1.66179E−06 | 5.779424966 | RTN4 | 0.400013238 | 0.001124139 | 0.016139894 | 1.792099311 |
| TMED10 | 0.578586817 | 4.78752E−08 | 1.72449E−06 | 5.763338725 | CTSF | 0.173648223 | 0.001128031 | 0.016186282 | 1.790852895 |
| COX7A2 | 0.511314034 | 4.88411E−08 | 1.7567E−06 | 5.755302164 | ALDH1B1 | 1.124852896 | 0.001163621 | 0.01667743 | 1.777870867 |
| LAMTOR1 | 0.571969867 | 5.28388E−08 | 1.8977E−06 | 5.721771903 | CBFB | 0.549068809 | 0.001163621 | 0.01667743 | 1.777870867 |
| MZT2B | 0.519026816 | 5.31013E−08 | 1.90434E−06 | 5.720255576 | FAM126A | 0.375172168 | 0.001167437 | 0.016712574 | 1.776956659 |
| RPL10A | 0.341183871 | 5.36401E−08 | 1.92085E−06 | 5.716506951 | EIF4B | 0.211700261 | 0.001186082 | 0.016949768 | 1.770836243 |
| HPS1 | 0.670031786 | 5.5466E−08 | 1.98333E−06 | 5.702604213 | NTPCR | 0.60508126 | 0.001193658 | 0.017048083 | 1.768324437 |
| MTUS1 | 0.932735205 | 5.75783E−08 | 2.05287E−06 | 5.687638355 | OR7A5 | 1.484264016 | 0.001195713 | 0.017067484 | 1.767830498 |
| MTHFD1L | 0.816458106 | 5.84598E−08 | 2.08127E−06 | 5.681671224 | SCARB2 | 0.387297899 | 0.0012059 | 0.01719285 | 1.764652123 |
| IFIT3 | 1.049758911 | 5.90188E−08 | 2.09812E−06 | 5.678169383 | ELOVL5 | 0.567289948 | 0.001213593 | 0.017282406 | 1.762395785 |
| ADAM9 | 0.928937078 | 5.93327E−08 | 2.10623E−06 | 5.676495049 | DIP2C | 0.406671137 | 0.001213106 | 0.017282406 | 1.762395785 |
| FKBP2 | 0.553104937 | 6.04776E−08 | 2.14376E−06 | 5.668824076 | RPS18P9 | 0.231293768 | 0.001217212 | 0.017323876 | 1.761354934 |
| PEPD | 0.671213925 | 6.13236E−08 | 2.17061E−06 | 5.663418904 | EIF3H | 0.255139947 | 0.001218607 | 0.017333654 | 1.761109876 |
| GANAB | 0.659878075 | 6.44523E−08 | 2.27806E−06 | 5.642435199 | TRAPPC4 | 0.477441072 | 0.0012228 | 0.0173832 | 1.759870263 |
| SMDT1 | 0.594753146 | 6.47647E−08 | 2.2858E−06 | 5.640961617 | PRDX1 | 0.263645262 | 0.001231719 | 0.017499836 | 1.756966014 |
| UBE2I | 0.581882119 | 6.56388E−08 | 2.31332E−06 | 5.635764585 | ANXA1 | 0.628599226 | 0.001234893 | 0.01753477 | 1.756099923 |
| COX14 | 0.644691825 | 6.68586E−08 | 2.35292E−06 | 5.628392192 | NAA20 | 0.341873854 | 0.001239209 | 0.017585853 | 1.75483655 |
| TOMM6 | 0.574760795 | 7.00305E−08 | 2.46102E−06 | 5.608885711 | SOD2 | 0.560766757 | 0.00124724 | 0.017689576 | 1.752282571 |
| PYCARD | 1.521375179 | 7.05036E−08 | 2.47409E−06 | 5.606584223 | RNASE4 | 0.697884977 | 0.001249737 | 0.017714737 | 1.751665285 |
| NSRP1 | 0.657296479 | 7.08315E−08 | 2.48204E−06 | 5.605191055 | CAPRIN1 | 0.525518239 | 0.001260132 | 0.017841435 | 1.748570223 |
| MBNL2 | 0.709334135 | 7.15762E−08 | 2.50455E−06 | 5.601269546 | TRAF3 | 0.877681792 | 0.001272784 | 0.018010157 | 1.744482509 |
| MYL12A | 0.653023601 | 7.17783E−08 | 2.50804E−06 | 5.600665313 | C11orf58 | 0.268485367 | 0.001274456 | 0.018023397 | 1.74416335 |
| BLOC1S1 | 0.557074308 | 7.23812E−08 | 2.5255E−06 | 5.597651904 | SRSF1 | 0.476376355 | 0.001298071 | 0.018346771 | 1.736440355 |
| LOC101243545 | 0.361014187 | 7.31616E−08 | 2.5491E−06 | 5.593612433 | MAFF | 0.733260214 | 0.001304075 | 0.018420997 | 1.73468686 |
| CHMP2A | 0.604333639 | 7.41722E−08 | 2.57698E−06 | 5.588888258 | MRPL14 | 0.418225288 | 0.001320661 | 0.018644538 | 1.729448385 |
| VKORC1 | 0.641132116 | 7.44895E−08 | 2.58434E−06 | 5.5876499 | PLA2G7 | 0.970813394 | 0.001322541 | 0.018660323 | 1.729080847 |
| SCUBE2 | 1.640160561 | 7.68941E−08 | 2.66023E−06 | 5.575080339 | SCCPDH | 0.551659226 | 0.00132812 | 0.018722802 | 1.727629161 |
| RPS10 | 0.27310735 | 7.71177E−08 | 2.6642E−06 | 5.574432463 | AHCYL1 | 0.359017611 | 0.001328498 | 0.018722802 | 1.727629161 |
| SMPD1 | 0.951208472 | 7.98284E−08 | 2.75397E−06 | 5.560041413 | FAM160B1 | 0.675868511 | 0.001332811 | 0.01877279 | 1.726471177 |
| ATP6V1B2 | 0.732837141 | 8.31564E−08 | 2.86072E−06 | 5.543524555 | RN7SK | 0.347018152 | 0.001341172 | 0.018879694 | 1.724005045 |
| RAB30 | 0.888944268 | 8.48666E−08 | 2.91546E−06 | 5.535293078 | SMUG1 | 0.580817759 | 0.001346135 | 0.018938676 | 1.722650395 |
| NDUFS6 | 0.588175714 | 8.59168E−08 | 2.9474E−06 | 5.530560626 | KXD1 | 0.392245531 | 0.001350808 | 0.018993511 | 1.721394738 |
| DNPH1 | 0.6196298 | 8.65013E−08 | 2.96023E−06 | 5.528673823 | DPY30 | 0.444095105 | 0.001371595 | 0.019263672 | 1.715260924 |
| MRPS21 | 0.550809801 | 8.65325E−08 | 2.96023E−06 | 5.528673823 | DNAJC1 | 0.361051887 | 0.001377677 | 0.019338008 | 1.713588269 |
| HADHA | 0.620715141 | 8.87898E−08 | 3.03322E−06 | 5.518096437 | MVP | 0.51050669 | 0.001379867 | 0.019357647 | 1.713147432 |
| GYPC | 0.532649696 | 8.89583E−08 | 3.03474E−06 | 5.517878039 | SARNP | 0.423430741 | 0.001383078 | 0.019391597 | 1.712386412 |
| UTRN | 1.141355395 | 9.37804E−08 | 3.1948E−06 | 5.495556905 | PRDX3 | 0.346877962 | 0.001388971 | 0.01946308 | 1.710788423 |
| EIF4G2 | 0.492413908 | 9.39497E−08 | 3.19612E−06 | 5.495377412 | PRKCH | 1.153917074 | 0.001406153 | 0.019692567 | 1.705697667 |
| EPS8 | 0.67844224 | 9.6559E−08 | 3.28033E−06 | 5.48408263 | FEM1B | 0.467718035 | 0.001422 | 0.019869065 | 1.70182258 |
| ATP6V1F | 0.591918707 | 9.97398E−08 | 3.38369E−06 | 5.470608827 | SMARCA2 | 0.422211617 | 0.001428773 | 0.019952317 | 1.700006668 |
| TRAK2 | 0.592544675 | 1.02791E−07 | 3.48038E−06 | 5.458373692 | BBX | 0.387924224 | 0.001430633 | 0.019955532 | 1.699936696 |
| RPLP2 | 0.243088096 | 1.02874E−07 | 3.48038E−06 | 5.458373692 | LOC100996447 | 0.411565363 | 0.001434521 | 0.019998378 | 1.699005218 |
| KIAA1033 | 0.969338059 | 1.03105E−07 | 3.4834E−06 | 5.457997162 | CD47 | 0.340240628 | 0.001451072 | 0.020206122 | 1.694517027 |
| CCDC124 | 0.617655396 | 1.03896E−07 | 3.50527E−06 | 5.455278004 | TOMM20 | 0.32188195 | 0.001450253 | 0.020206122 | 1.694517027 |
| MATN2 | 1.387170076 | 1.04158E−07 | 3.50927E−06 | 5.454783059 | PCNP | 0.415438294 | 0.001452959 | 0.020220894 | 1.694199644 |
| APRT | 0.57141602 | 1.05599E−07 | 3.55295E−06 | 5.449410571 | SNRNP27 | 0.473485529 | 0.001460527 | 0.020314678 | 1.692190061 |
| NAA38 | 0.588605762 | 1.06189E−07 | 3.56789E−06 | 5.447588476 | PKHD1 | 0.952828765 | 0.001463994 | 0.020351345 | 1.691406878 |
| IRF2BPL | 0.596535683 | 1.2731E−07 | 4.27169E−06 | 5.369400107 | TMOD1 | 0.560194552 | 0.001464903 | 0.020352435 | 1.691383626 |
| RAB14 | 0.689265966 | 1.28245E−07 | 4.29719E−06 | 5.36681568 | GSTM3 | 0.566012502 | 0.001466144 | 0.020358126 | 1.691262197 |
| CYB561A3 | 0.524803936 | 1.38033E−07 | 4.61883E−06 | 5.335468251 | TRIM5 | 0.761355221 | 0.001467717 | 0.020368422 | 1.691042612 |
| SPATS2L | 0.507076085 | 1.46354E−07 | 4.88391E−06 | 5.311232703 | SMPDL3A | 0.650407195 | 0.001483283 | 0.020561134 | 1.686952932 |
| BLVRB | 0.560459673 | 1.52402E−07 | 5.06505E−06 | 5.295416075 | HIPK2 | 0.400419444 | 0.001491837 | 0.020656332 | 1.684946804 |
| ARPC5 | 0.485333353 | 1.52904E−07 | 5.07483E−06 | 5.294578875 | ABCC2 | 0.446914554 | 0.001494361 | 0.020679597 | 1.684457919 |
| 44077 | 0.860058367 | 1.5618E−07 | 5.17656E−06 | 5.285958998 | MTHFS | 0.464651227 | 0.001503906 | 0.020799928 | 1.68193816 |
| SLC35F6 | 0.586712778 | 1.59171E−07 | 5.26857E−06 | 5.278306958 | ZSCAN16-AS1 | 0.440720767 | 0.001516833 | 0.020966883 | 1.678466137 |
| SHFM1 | 0.47755143 | 1.59878E−07 | 5.28483E−06 | 5.276969114 | GATM | 1.006914377 | 0.001527283 | 0.021099422 | 1.675729443 |
| TFF3 | 1.549136877 | 1.62261E−07 | 5.35637E−06 | 5.271129582 | F8A1 | 0.581438601 | 0.001532361 | 0.021157633 | 1.674532927 |
| BNIP3L | 0.704238832 | 1.68035E−07 | 5.5395E−06 | 5.256529588 | SRSF6 | 0.645352304 | 0.001540136 | 0.021241038 | 1.67282426 |
| SIGIRR | 0.613581419 | 1.724E−07 | 5.67575E−06 | 5.245976483 | METRN | 0.411207438 | 0.001542562 | 0.021262529 | 1.672385079 |
| AEBP1 | 0.617488146 | 1.72888E−07 | 5.68419E−06 | 5.245331464 | RALA | 0.449469358 | 0.00154395 | 0.021269692 | 1.672238791 |
| ATP6V0D1 | 0.645439506 | 1.78348E−07 | 5.85585E−06 | 5.232409717 | HMGN5 | 1.231239557 | 0.001547425 | 0.021297205 | 1.671677391 |
| WSCD1 | 1.416603804 | 1.843E−07 | 6.04317E−06 | 5.218735057 | C6orf48 | 0.274605632 | 0.001547686 | 0.021297205 | 1.671677391 |
| OPTN | 0.583661973 | 1.89774E−07 | 6.21433E−06 | 5.206605722 | PHYH | 0.817574024 | 0.001555445 | 0.021379956 | 1.669993202 |
| TBCB | 0.596639454 | 1.90897E−07 | 6.24277E−06 | 5.20462241 | UGT2B7 | 6.278800488 | 0.001567988 | 0.021516129 | 1.667235862 |
| LOC729732 | 0.539223373 | 1.95456E−07 | 6.38333E−06 | 5.194952779 | FXYD5 | 0.412808567 | 0.001590317 | 0.021785919 | 1.661824115 |
| HES4 | 0.542455733 | 1.96387E−07 | 6.4052E−06 | 5.193467319 | PIP4K2C | 0.633575261 | 0.001598542 | 0.021880253 | 1.659947654 |
| CYGB | 1.035890579 | 2.02243E−07 | 6.58741E−06 | 5.181285464 | MRPL43 | 0.409499317 | 0.001618096 | 0.022129342 | 1.655031501 |
| SEL1L | 0.668210626 | 2.06155E−07 | 6.70593E−06 | 5.173541145 | KLF2 | 0.627043843 | 0.001631103 | 0.022289821 | 1.651893425 |
| GHITM | 0.581679583 | 2.10141E−07 | 6.82651E−06 | 5.165801409 | TM9SF3 | 0.425929606 | 0.001631651 | 0.022289821 | 1.651893425 |
| NSMCE1 | 0.711767852 | 2.15388E−07 | 6.9877E−06 | 5.155665519 | NQO1 | 0.852332056 | 0.001648164 | 0.02250286 | 1.647762273 |
| C14orf166 | 0.549316585 | 2.19874E−07 | 7.12379E−06 | 5.147289173 | EVA1A | 1.363569736 | 0.001691067 | 0.023050075 | 1.637327655 |
| COA3 | 0.637836695 | 2.20956E−07 | 7.14939E−06 | 5.145731124 | SEC24C | 0.590666354 | 0.001699201 | 0.023148064 | 1.635485327 |
| ASAP2 | 0.655915022 | 2.27238E−07 | 7.34298E−06 | 5.134127603 | DUSP23 | 0.514520347 | 0.001710897 | 0.023294442 | 1.632747681 |
| CRABP1 | 1.115592837 | 2.32348E−07 | 7.4982E−06 | 5.125043004 | CREBL2 | 0.647610724 | 0.001711946 | 0.023295784 | 1.632722677 |
| ABHD2 | 0.711188972 | 2.33632E−07 | 7.52973E−06 | 5.123220661 | ANP32E | 0.417618632 | 0.001720556 | 0.023373987 | 1.631267199 |
| ARPC2 | 0.566159964 | 2.58545E−07 | 8.31077E−06 | 5.080358914 | YIPF2 | 0.567141593 | 0.001733063 | 0.023517805 | 1.628603218 |
| RPS23 | 0.197833583 | 2.62062E−07 | 8.40177E−06 | 5.075629037 | MRPL55 | 0.451234788 | 0.001735574 | 0.023538843 | 1.628214885 |
| CYB5A | 0.838325143 | 2.64172E−07 | 8.45833E−06 | 5.072715293 | DMD | 0.581283569 | 0.001739735 | 0.023582216 | 1.627415395 |
| SKA2 | 0.690693855 | 2.65487E−07 | 8.48934E−06 | 5.07112589 | LOC400043 | 1.078658104 | 0.001743225 | 0.023616459 | 1.62678521 |
| FRMD6 | 1.161721826 | 2.67041E−07 | 8.5279E−06 | 5.06915784 | DYNC1LI1 | 0.52790916 | 0.001751492 | 0.023715339 | 1.624970661 |
| CYBA | 0.445435137 | 2.76358E−07 | 8.81396E−06 | 5.054828684 | CD79B | 0.633413196 | 0.001759404 | 0.023796152 | 1.623493275 |
| CLIC4 | 0.648869514 | 2.77805E−07 | 8.84857E−06 | 5.05312706 | GULP1 | 0.438257515 | 0.001764009 | 0.023832124 | 1.622837254 |
| LINC01578 | 0.560521543 | 2.81061E−07 | 8.92906E−06 | 5.049194165 | CAPZA2 | 0.418174654 | 0.001763813 | 0.023832124 | 1.622837254 |
| VPS29 | 0.520443678 | 2.84255E−07 | 9.01884E−06 | 5.044849157 | SNX7 | 0.521920502 | 0.00176666 | 0.023841636 | 1.622663952 |
| APBA2 | 0.817653716 | 2.96304E−07 | 9.38898E−06 | 5.027381609 | NBAT1 | 0.443475693 | 0.00179327 | 0.024160817 | 1.616888388 |
| POLR2J | 0.567241371 | 3.04698E−07 | 9.64246E−06 | 5.015812002 | SLC16A4 | 0.837427855 | 0.001809975 | 0.024372479 | 1.613100299 |
| SEC62 | 0.468506604 | 3.4092E−07 | 1.07748E−05 | 4.967589925 | MIR21 | 1.491248727 | 0.001830474 | 0.024634964 | 1.60844807 |
| LAMC1 | 0.777239978 | 3.44292E−07 | 1.08674E−05 | 4.963874523 | RELB | 0.964757621 | 0.001831591 | 0.024636458 | 1.60842173 |
| DLC1 | 0.670153201 | 3.48509E−07 | 1.09722E−05 | 4.9597064 | BHLHE41 | 0.661944389 | 0.001833827 | 0.02465299 | 1.608130401 |
| PRR13 | 0.627445996 | 3.82628E−07 | 1.20309E−05 | 4.919700433 | TSC22D2 | 0.262163877 | 0.001839223 | 0.024711967 | 1.607092682 |
| C1orf122 | 0.569933176 | 3.83485E−07 | 1.20424E−05 | 4.919286266 | ACOT1 | 0.38824755 | 0.001850263 | 0.024833067 | 1.604969642 |
| NFIA | 0.643778682 | 3.894E−07 | 1.22125E−05 | 4.913194992 | AKIP1 | 0.631985545 | 0.001853546 | 0.024844558 | 1.604768718 |
| EMP2 | 1.414267302 | 3.93404E−07 | 1.23223E−05 | 4.909308445 | LRP10 | 0.543503106 | 0.001853444 | 0.024844558 | 1.604768718 |
| ARL6IP4 | 0.489957392 | 3.95811E−07 | 1.23819E−05 | 4.907213785 | SF3B1 | 0.336248755 | 0.001854162 | 0.024844558 | 1.604768718 |
| TAF7 | 0.520955682 | 4.04611E−07 | 1.2641E−05 | 4.898219051 | SNRPB | 0.231769323 | 0.001865905 | 0.024988234 | 1.60226443 |
| HEYL | 1.657332659 | 4.05654E−07 | 1.26574E−05 | 4.8976541 | JOSD2 | 0.372889787 | 0.001869317 | 0.025006583 | 1.601945652 |
| EMC3 | 0.653958678 | 4.20249E−07 | 1.30962E−05 | 4.882855865 | NEK9 | 0.450864375 | 0.001891164 | 0.025271236 | 1.597373518 |
| SNAPIN | 0.750937462 | 4.21838E−07 | 1.3129E−05 | 4.881769738 | PSMC3 | 0.335276484 | 0.001892838 | 0.025279815 | 1.597226104 |
| RPS3A | 0.157440186 | 4.23359E−07 | 1.31596E−05 | 4.880757582 | TAF9B | 0.972216249 | 0.001923631 | 0.025677067 | 1.590454587 |
| CBX5 | 0.671901405 | 4.35612E−07 | 1.35233E−05 | 4.868917942 | DDX1 | 0.441210032 | 0.0019248 | 0.025678676 | 1.590427366 |
| GNG10 | 0.571611047 | 4.43475E−07 | 1.375E−05 | 4.861697821 | ARNT | 0.521976769 | 0.001933001 | 0.025760023 | 1.58905376 |
| RPL37A | 0.191981241 | 4.69178E−07 | 1.45285E−05 | 4.837779023 | NMT1 | 0.323164016 | 0.001932147 | 0.025760023 | 1.58905376 |
| HOTAIRM1 | 3.226736069 | 4.70904E−07 | 1.45489E−05 | 4.837169602 | GAMT | 0.662066102 | 0.001948854 | 0.025943055 | 1.585978882 |
| PEG3 | 1.116071224 | 4.71025E−07 | 1.45489E−05 | 4.837169602 | GNB2 | 0.368202955 | 0.001950252 | 0.025947566 | 1.585903383 |
| TIMM8B | 0.534235716 | 4.75835E−07 | 1.4679E−05 | 4.83330435 | TTC27 | 1.066931367 | 0.001959633 | 0.026058219 | 1.584055275 |
| FAM177A1 | 0.596907079 | 4.84233E−07 | 1.49192E−05 | 4.826253225 | MARCKSL1 | 0.019618055 | 0.001988243 | 0.026409988 | 1.578231804 |
| B3GLCT | 0.920603871 | 5.06353E−07 | 1.55812E−05 | 4.807400028 | NT5C | 0.422322452 | 0.001997461 | 0.026518055 | 1.576458341 |
| TCERG1L | 1.376517296 | 5.11859E−07 | 1.57308E−05 | 4.803248226 | MX1 | 1.221611619 | 0.001998781 | 0.026521205 | 1.576406741 |
| SSR3 | 0.629820704 | 5.12731E−07 | 1.57379E−05 | 4.803053268 | MAP3K1 | 0.458415598 | 0.00201535 | 0.026726576 | 1.573056683 |
| SCP2 | 0.530862868 | 5.25453E−07 | 1.61082E−05 | 4.792952925 | METTL9 | 0.406113267 | 0.002024795 | 0.026837299 | 1.571261189 |
| MRPL34 | 0.610886157 | 5.31205E−07 | 1.62642E−05 | 4.788768354 | KIAA1549L | 0.683491588 | 0.002036294 | 0.026964985 | 1.569199812 |
| PKIG | 0.761800766 | 5.3374E−07 | 1.63214E−05 | 4.787243059 | PTP4A2 | 0.317894071 | 0.00203663 | 0.026964985 | 1.569199812 |
| CLIC1 | 0.510033071 | 5.70773E−07 | 1.74321E−05 | 4.758651346 | DLST | 0.497977555 | 0.002063435 | 0.027260943 | 1.564459121 |
| C7orf55 | 0.621402442 | 5.72858E−07 | 1.7474E−05 | 4.757608784 | MBNL1 | 0.389479488 | 0.002072291 | 0.027363172 | 1.562833554 |
| PMM1 | 0.786223948 | 5.78324E−07 | 1.76187E−05 | 4.754025173 | IFI27L1 | 0.521007836 | 0.002091252 | 0.02756896 | 1.55957961 |
| ANAPC13 | 0.664336264 | 5.85425E−07 | 1.77908E−05 | 4.749803803 | RPF1 | 0.473244876 | 0.00209619 | 0.027604339 | 1.559022651 |
| FSTL1 | 0.645989895 | 5.85128E−07 | 1.77908E−05 | 4.749803803 | LRRK2 | 2.980920315 | 0.002097915 | 0.027612209 | 1.55889885 |
| SVIP | 0.77262193 | 6.02171E−07 | 1.82319E−05 | 4.739169111 | ST3GAL5 | 0.392668456 | 0.002120341 | 0.027892396 | 1.554514175 |
| MYO5A | 0.359496981 | 6.02171E−07 | 1.82319E−05 | 4.739169111 | 43896 | 0.309723228 | 0.002146745 | 0.028224563 | 1.549372779 |
| VAT1 | 0.313799381 | 6.07135E−07 | 1.83595E−05 | 4.736140165 | ATP6V1A | 0.546720564 | 0.002159114 | 0.028371959 | 1.547110675 |
| USP13 | 0.887583435 | 6.17495E−07 | 1.86497E−05 | 4.729327904 | IFI35 | 1.492541194 | 0.002164435 | 0.028426631 | 1.546274615 |
| HSPB11 | 0.732304573 | 6.23739E−07 | 1.88151E−05 | 4.725493094 | TMEM30A | 0.652688384 | 0.002167278 | 0.028448713 | 1.545937381 |
| ERH | 0.47988385 | 6.26487E−07 | 1.88748E−05 | 4.724118536 | SPPL3 | 0.40842846 | 0.002173092 | 0.028479253 | 1.54547141 |
| CSRP1 | 0.51681143 | 6.27653E−07 | 1.88867E−05 | 4.723844801 | HNRNPA1P10 | 0.231057435 | 0.002172617 | 0.028479253 | 1.54547141 |
| RELL1 | 0.857942224 | 6.46215E−07 | 1.93975E−05 | 4.712253121 | FUCA2 | 0.416654021 | 0.00218047 | 0.028560662 | 1.544231735 |
| FAM53B | 0.770201743 | 6.45725E−07 | 1.93975E−05 | 4.712253121 | CHI3L2 | 2.776178837 | 0.002204547 | 0.028845179 | 1.539926768 |
| LAPTM4A | 0.276757836 | 6.61445E−07 | 1.98304E−05 | 4.702668694 | HSF2 | 0.835441947 | 0.002224858 | 0.029079862 | 1.53640766 |
| ERP29 | 0.579482317 | 6.66935E−07 | 1.99706E−05 | 4.69960997 | RPL11 | 0.069800943 | 0.002234955 | 0.029196262 | 1.534672754 |
| ENPP1 | 1.316067815 | 6.9593E−07 | 2.08133E−05 | 4.681658319 | MRPS14 | 0.503073454 | 0.002247789 | 0.029348268 | 1.532417526 |
| SUCLG1 | 0.619225621 | 7.10551E−07 | 2.1206E−05 | 4.673541998 | VPS13C | 0.394675493 | 0.00225503 | 0.029427126 | 1.531252149 |
| BUD31 | 0.586570978 | 7.1079E−07 | 2.1206E−05 | 4.673541998 | DHX30 | 0.465827793 | 0.002277006 | 0.029682275 | 1.527502814 |
| COL6A1 | 0.745697081 | 7.11688E−07 | 2.12069E−05 | 4.673522541 | CYC1 | 0.435717493 | 0.002282966 | 0.029744133 | 1.526598691 |
| CDC42SE2 | 0.73008126 | 7.31961E−07 | 2.17845E−05 | 4.661852356 | PICALM | 0.540448427 | 0.00230632 | 0.030019741 | 1.522593065 |
| COPZ1 | 0.570719957 | 7.53169E−07 | 2.23885E−05 | 4.649975261 | SUN1 | 0.538053334 | 0.002306571 | 0.030019741 | 1.522593065 |
| CSDE1 | 0.621261656 | 7.79596E−07 | 2.3146E−05 | 4.635524501 | SRD5A3 | 0.648735225 | 0.002309459 | 0.030025417 | 1.522510957 |
| LOC103091866 | 1.496359468 | 7.86819E−07 | 2.33321E−05 | 4.63204524 | CYB5B | 0.393265545 | 0.00232964 | 0.03027172 | 1.518962906 |
| FAM111A | 0.929202676 | 8.12748E−07 | 2.40719E−05 | 4.61848991 | ELMSAN1 | 0.386649638 | 0.00234794 | 0.030493344 | 1.515794949 |
| ATAD3C | 0.55780375 | 8.40412E−07 | 2.48612E−05 | 4.60447861 | AEBP2 | 0.70911932 | 0.00235343 | 0.03054845 | 1.515010827 |
| ITM2C | 0.583308399 | 9.07824E−07 | 2.6823E−05 | 4.57149298 | PPP6R3 | 0.323021084 | 0.002359445 | 0.030610297 | 1.514132456 |
| ACTB | 0.362627536 | 9.18942E−07 | 2.71187E−05 | 4.566730462 | HS6ST2 | 1.486431003 | 0.002364703 | 0.030646049 | 1.51362551 |
| C9orf16 | 0.583729208 | 9.26941E−07 | 2.73219E−05 | 4.563489426 | FBXO25 | 0.47182554 | 0.002392841 | 0.030994316 | 1.508717949 |
| MDK | 1.207130194 | 9.43769E−07 | 2.77845E−05 | 4.556198134 | FOXN3 | 0.45862031 | 0.002410158 | 0.031202123 | 1.505815858 |
| ZFAND5 | 0.432817787 | 9.6113E−07 | 2.82277E−05 | 4.549324084 | CCPG1 | 0.536913157 | 0.002429037 | 0.031429915 | 1.502656788 |
| TP53TG1 | 0.838454783 | 9.70289E−07 | 2.84626E−05 | 4.545725578 | C19orf24 | 0.395685494 | 0.002439716 | 0.031551421 | 1.500981073 |
| EIF4EBP2 | 0.611932755 | 1.00118E−06 | 2.93336E−05 | 4.532634384 | SURF4 | 0.412420511 | 0.002449111 | 0.031639519 | 1.499770133 |
| EIF2S3 | 0.398995519 | 1.00318E−06 | 2.9357E−05 | 4.532287622 | YIF1B | 0.387237102 | 0.002455083 | 0.031699951 | 1.498941406 |
| MZT2A | 0.600753725 | 1.01466E−06 | 2.96222E−05 | 4.528382271 | GNA12 | 0.665707529 | 0.002472545 | 0.031908591 | 1.496092376 |
| RWDD1 | 0.532901943 | 1.01432E−06 | 2.96222E−05 | 4.528382271 | PPM1A | 0.415445209 | 0.002493217 | 0.032158427 | 1.492705199 |
| SHISA4 | 0.904652774 | 1.06671E−06 | 3.11048E−05 | 4.50717325 | PRELID2 | 1.035774112 | 0.002539966 | 0.032744176 | 1.484865934 |
| HIATL1 | 0.678709984 | 1.06866E−06 | 3.11245E−05 | 4.506898062 | GPAT4 | 0.404110909 | 0.00254779 | 0.032827752 | 1.483758852 |
| HEBP2 | 0.666354418 | 1.07034E−06 | 3.11365E−05 | 4.506730289 | LOC101927204 | 2.0321908 | 0.002556324 | 0.0329204 | 1.48253489 |
| UQCRB | 0.47621882 | 1.07568E−06 | 3.12546E−05 | 4.505086453 | SMIM12 | 0.442536324 | 0.002562811 | 0.032986599 | 1.481662461 |
| AK1 | 0.703350902 | 1.09283E−06 | 3.17154E−05 | 4.498730181 | CD151 | 0.378201485 | 0.002570283 | 0.033065396 | 1.480626272 |
| TRIB1 | 1.153262973 | 1.12393E−06 | 3.25792E−05 | 4.487059136 | FAAP20 | 0.335085392 | 0.002582224 | 0.033201571 | 1.478841372 |
| THRAP3 | 0.582136989 | 1.1422E−06 | 3.30697E−05 | 4.48056938 | MRPS33 | 0.488050103 | 0.002595753 | 0.033358012 | 1.476799842 |
| PDCD5 | 0.563004315 | 1.15435E−06 | 3.3382E−05 | 4.476487635 | ZCCHC24 | 0.632993579 | 0.002630213 | 0.033765429 | 1.471527732 |
| CD164 | 0.74019891 | 1.16662E−06 | 3.36971E−05 | 4.472407122 | RPL4 | 0.207107097 | 0.002638006 | 0.033847734 | 1.470470396 |
| ALKBH5 | 0.802211165 | 1.1699E−06 | 3.37521E−05 | 4.471699265 | PPP3CA | 0.350260198 | 0.002650092 | 0.033967216 | 1.468940052 |
| DYNLRB1 | 0.445166588 | 1.20072E−06 | 3.46006E−05 | 4.460916548 | SORT1 | 0.370125771 | 0.002678872 | 0.034318147 | 1.46447617 |
| SLC25A1 | 0.608487904 | 1.21607E−06 | 3.50018E−05 | 4.455909744 | NAGLU | 0.398744792 | 0.002693297 | 0.034484902 | 1.462371005 |
| NDUFA8 | 0.580642819 | 1.22554E−06 | 3.52328E−05 | 4.453053298 | KAT2B | 0.782295108 | 0.002695901 | 0.034500209 | 1.462178278 |
| MAP1LC3B | 0.474357158 | 1.29699E−06 | 3.72432E−05 | 4.428953117 | RNH1 | 0.388496369 | 0.002732453 | 0.03494971 | 1.456556422 |
| EMX2OS | 2.43697381 | 1.29934E−06 | 3.7267E−05 | 4.428675897 | RAB3D | 0.59209168 | 0.002744639 | 0.035087255 | 1.454850611 |
| HMGB2 | 0.855986059 | 1.30416E−06 | 3.73614E−05 | 4.42757646 | NUBP1 | 0.622347474 | 0.002764121 | 0.035299471 | 1.452231808 |
| SMIM3 | 1.702076077 | 1.32371E−06 | 3.78774E−05 | 4.421619953 | DAZAP1 | 0.452167124 | 0.002763529 | 0.035299471 | 1.452231808 |
| LOC283788 | 0.921700776 | 1.34399E−06 | 3.83681E−05 | 4.416030259 | GALT | 0.586820722 | 0.002775544 | 0.035408428 | 1.450893359 |
| ENY2 | 0.574177384 | 1.34388E−06 | 3.83681E−05 | 4.416030259 | FTCDNL1 | 0.668071628 | 0.002779411 | 0.0354393 | 1.450514861 |
| SRRM1 | 0.470174789 | 1.35954E−06 | 3.87216E−05 | 4.412046943 | PABPC1 | 0.134751782 | 0.002789548 | 0.035550043 | 1.449159874 |
| MARVELD1 | 0.767415941 | 1.38762E−06 | 3.94753E−05 | 4.403674033 | HNRNPR | 0.291469574 | 0.002792087 | 0.035563901 | 1.448990602 |
| NACA | 0.305664033 | 1.41997E−06 | 4.03489E−05 | 4.394167985 | SH3D19 | 0.493059903 | 0.002799718 | 0.035642565 | 1.448031052 |
| HMG20B | 0.606040835 | 1.44451E−06 | 4.09985E−05 | 4.387231972 | CASP1 | 3.093244791 | 0.002833612 | 0.036055319 | 1.443030655 |
| ATP5G1 | 0.512618959 | 1.45555E−06 | 4.12642E−05 | 4.384426074 | COL1A2 | 0.497699916 | 0.002841346 | 0.036116211 | 1.44229782 |
| MMP14 | 0.612986326 | 1 4626E−06 | 4.14162E−05 | 4.382829782 | ZNF106 | 0.250707746 | 0.002842865 | 0.036116774 | 1.442291048 |
| REEP5 | 0.488979369 | 1.48699E−06 | 4.20582E−05 | 4.376149288 | ADGRA2 | 0.625886917 | 0.002871098 | 0.036456539 | 1.438224568 |
| MLLT3 | 1.012093067 | 1.5202E−06 | 4.2948E−05 | 4.367057558 | GADD45A | 0.526783049 | 0.002892015 | 0.036684111 | 1.435522005 |
| PSMA4 | 0.48188132 | 1.55083E−06 | 4.37123E−05 | 4.359396092 | LOC653513 | 0.883711517 | 0.002895412 | 0.03669507 | 1.435392278 |
| SPARC | 1.060105085 | 1.59475E−06 | 4.48988E−05 | 4.347765592 | EFEMP2 | 0.843316615 | 0.002908105 | 0.036794894 | 1.434212448 |
| PTP4A1 | 0.730869174 | 1.60795E−06 | 4.52184E−05 | 4.344684756 | PCDH7 | 0.373351157 | 0.002979303 | 0.037674261 | 1.423955257 |
| SPCS1 | 0.482046902 | 1.67486E−06 | 4.70459E−05 | 4.327478443 | SERINC5 | 0.482226193 | 0.002999024 | 0.037904069 | 1.421314169 |
| AKR1A1 | 0.656924665 | 1.68608E−06 | 4.72797E−05 | 4.325324901 | PLXND1 | 0.683725119 | 0.003010849 | 0.038014299 | 1.420053014 |
| PTPRM | 0.642086685 | 1.68705E−06 | 4.72797E−05 | 4.325324901 | PLXNB1 | 0.522469521 | 0.003015655 | 0.038055365 | 1.419584105 |
| GNAL | 1.137399793 | 1.69448E−06 | 4.73836E−05 | 4.324372322 | ELAVL1 | 0.432554901 | 0.003028345 | 0.038195825 | 1.417984109 |
| VPS35 | 0.613831282 | 1.69462E−06 | 4.73836E−05 | 4.324372322 | ZHX1 | 0.325101823 | 0.003034144 | 0.038249269 | 1.417376861 |
| SPG20 | 0.685614407 | 1.72173E−06 | 4.80868E−05 | 4.317973735 | MTCH2 | 0.358595192 | 0.00304173 | 0.038319718 | 1.416577692 |
| GREB1 | 1.142894387 | 1.72608E−06 | 4.81534E−05 | 4.317373068 | PRKAR1A | 0.291071832 | 0.003042862 | 0.038319718 | 1.416577692 |
| DGCR6 | 0.651177108 | 1.72861E−06 | 4.81691E−05 | 4.317231329 | CTHRC1 | 1.216300726 | 0.003061015 | 0.038528519 | 1.414217683 |
| RPL7A | 0.217803985 | 1.73185E−06 | 4.82045E−05 | 4.316912386 | FAM127C | 0.380423516 | 0.003067522 | 0.038590594 | 1.413518541 |
| HSD17B14 | 0.810159087 | 1.74259E−06 | 4.84484E−05 | 4.314720467 | CSRNP3 | 0.832820177 | 0.003076327 | 0.038681493 | 1.412496767 |
| TPCN2 | 0.623597383 | 1.77116E−06 | 4.9187E−05 | 4.308150038 | TNFAIP3 | 0.510048568 | 0.00307894 | 0.038694488 | 1.412350892 |
| ARF5 | 0.516981959 | 1.77953E−06 | 4.93634E−05 | 4.306594694 | GPI | 0.458107591 | 0.003083354 | 0.038710243 | 1.4121741 |
| IFI6 | 1.131512523 | 1.78359E−06 | 4.94199E−05 | 4.30609796 | LAMP1 | 0.444698751 | 0.003082373 | 0.038710243 | 1.4121741 |
| TMX1 | 0.780679524 | 1.7862E−06 | 4.94363E−05 | 4.30595368 | OSBPL8 | 0.279816508 | 0.003092037 | 0.038799357 | 1.411175471 |
| FCRLA | 0.936919749 | 1.7907E−06 | 4.95051E−05 | 4.305350112 | LSM5 | 0.43433401 | 0.00309499 | 0.038816535 | 1.410983232 |
| RHOB | 0.673812032 | 1.87719E−06 | 5.18375E−05 | 4.285355884 | MAP3K13 | 0.44890359 | 0.003104361 | 0.038894226 | 1.410114863 |
| CLTA | 0.527830482 | 1.90927E−06 | 5.2664E−05 | 4.278485831 | UBE2V2 | 0.36830461 | 0.003104032 | 0.038894226 | 1.410114863 |
| ATP5EP2 | 0.571646273 | 1.94377E−06 | 5.35552E−05 | 4.271198 | FEM1A | 0.665272295 | 0.003124835 | 0.039110734 | 1.40770403 |
| PDZD11 | 0.707202017 | 1.99817E−06 | 5.49923E−05 | 4.259697745 | FBXW11 | 0.471875548 | 0.003127651 | 0.039125993 | 1.407534623 |
| DDR2 | 1.021381855 | 2.00871E−06 | 5.52202E−05 | 4.257901722 | BIN3 | 0.656350017 | 0.003138442 | 0.039240937 | 1.406260634 |
| FTH1P3 | 0.32716307 | 2.02502E−06 | 5.56063E−05 | 4.25487638 | FAM134A | 0.299172193 | 0.003156446 | 0.039405697 | 1.404440981 |
| TM7SF3 | 0.505150199 | 2.05653E−06 | 5.64083E−05 | 4.248657236 | GFM1 | 0.626196504 | 0.003189528 | 0.039778161 | 1.400355297 |
| ARPC1B | 0.578877628 | 2.06285E−06 | 5.6501E−05 | 4.24794424 | CHRM1 | 0.497038126 | 0.00319206 | 0.039789478 | 1.400231755 |
| SMARCB1 | 0.519715369 | 2.06452E−06 | 5.6501E−05 | 4.24794424 | ADNP | 0.397857726 | 0.003194932 | 0.039805017 | 1.400062182 |
| COX16 | 0.552364051 | 2.07104E−06 | 5.6616E−05 | 4.24706069 | C9orf89 | 0.463349402 | 0.00321527 | 0.040019809 | 1.397724993 |
| ARHGAP1 | 0.520226181 | 2.08699E−06 | 5.69886E−05 | 4.24421225 | EIF3I | 0.316451013 | 0.00321544 | 0.040019809 | 1.397724993 |
| BROX | 0.756592274 | 2.13643E−06 | 5.82736E−05 | 4.234528358 | CCDC115 | 0.425923645 | 0.003252691 | 0.04044234 | 1.393163728 |
| FKBP3 | 0.55635047 | 2.14403E−06 | 5.84158E−05 | 4.233469837 | FAM161A | 0.584655821 | 0.003263874 | 0.040560792 | 1.391893576 |
| APOL2 | 1.100778341 | 2.15182E−06 | 5.85631E−05 | 4.23237596 | SAT2 | 0.360063672 | 0.003285478 | 0.040808563 | 1.389248698 |
| NDUFAF3 | 0.502129793 | 2.16715E−06 | 5.89148E−05 | 4.229775584 | RAB21 | 0.454829901 | 0.003293982 | 0.040893456 | 1.388346188 |
| HOXD-AS2 | 1.182919383 | 2.22755E−06 | 6.04895E−05 | 4.218320218 | RAP1GDS1 | 0.369626164 | 0.003295894 | 0.040896463 | 1.388314255 |
| PKNOX2 | 0.933417618 | 2.26335E−06 | 6.13937E−05 | 4.211876178 | DUSP22 | 0.450679511 | 0.003303203 | 0.040966404 | 1.387572161 |
| ERLEC1 | 0.663175762 | 2.26955E−06 | 6.14939E−05 | 4.211168115 | LACTB | 0.568296194 | 0.003324167 | 0.041205544 | 1.385044345 |
| COPZ2 | 1.170683861 | 2.30768E−06 | 6.24577E−05 | 4.204413706 | HIVEP3 | 2.307512522 | 0.003343139 | 0.041419752 | 1.382792509 |
| SELM | 1.07660079 | 2.31325E−06 | 6.25396E−05 | 4.203845236 | BOLA3 | 0.431530237 | 0.003347016 | 0.041446817 | 1.382508814 |
| RHOA | 0.333084881 | 2.38808E−06 | 6.43494E−05 | 4.191455591 | IDH1 | 0.592092335 | 0.003360439 | 0.041571012 | 1.3812094 |
| EIF6 | 0.546013534 | 2.40947E−06 | 6.48543E−05 | 4.188060968 | PPDPF | 0.382709444 | 0.003372669 | 0.041701242 | 1.379851015 |
| SLC44A1 | 0.604143739 | 2.44199E−06 | 6.55856E−05 | 4.183191371 | GAA | 0.667565286 | 0.0033796 | 0.041728138 | 1.379570994 |
| RPL29 | 0.267658293 | 2.48648E−06 | 6.67073E−05 | 4.175826828 | EAPP | 0.34284845 | 0.003379955 | 0.041728138 | 1.379570994 |
| EVA1B | 0.631518245 | 2.49407E−06 | 6.68377E−05 | 4.174978636 | CCL28 | 0.634943128 | 0.003426675 | 0.042262322 | 1.374046649 |
| ALDH6A1 | 0.663765013 | 2.49787E−06 | 6.68664E−05 | 4.174791789 | DOCK10 | 0.569732726 | 0.003429738 | 0.042278814 | 1.373877202 |
| ARPC4 | 0.499104619 | 2.84185E−06 | 7.5826E−05 | 4.120181991 | MRPL9 | 0.391307741 | 0.003454879 | 0.042567306 | 1.370923839 |
| CS | 0.542897105 | 2.89884E−06 | 7.71785E−05 | 4.112503466 | SEPT11 | 0.38769123 | 0.003463781 | 0.042655536 | 1.370024594 |
| CELF2 | 0.45703882 | 2.92088E−06 | 7.76807E−05 | 4.109686941 | LSM12 | 0.38203001 | 0.00348037 | 0.042836999 | 1.368180959 |
| CAMTA1 | 0.493004918 | 2.93113E−06 | 7.7869E−05 | 4.108635559 | PTPRN2 | 0.817936897 | 0.003521881 | 0.043283976 | 1.363672856 |
| MPV17 | 0.58389622 | 2.99556E−06 | 7.94944E−05 | 4.099663686 | SSBP3 | 0.477746844 | 0.003523729 | 0.043284959 | 1.363662993 |
| FARP2 | 0.742182076 | 3.02768E−06 | 8.02598E−05 | 4.095502152 | SPCS2 | 0.324290901 | 0.003609279 | 0.044296055 | 1.353634954 |
| CHMP1B | 0.566385269 | 3.08604E−06 | 8.17182E−05 | 4.087681163 | MRPS28 | 0.464301765 | 0.003666144 | 0.044876826 | 1.347977868 |
| PXN | 0.562960863 | 3.11363E−06 | 8.22712E−05 | 4.084752352 | ARPIN | 0.514118254 | 0.003671319 | 0.044917723 | 1.347582265 |
| CALU | 0.552352667 | 3.14022E−06 | 8.28214E−05 | 4.081857429 | KEAP1 | 0.628715277 | 0.003694784 | 0.045182252 | 1.34503213 |
| SEC22B | 0.525937319 | 3.14122E−06 | 8.28214E−05 | 4.081857429 | RQCD1 | 0.25835976 | 0.003708697 | 0.045329752 | 1.343616659 |
| RHEB | 0.515273213 | 3.17534E−06 | 8.3631E−05 | 4.077632823 | RPS5 | 0.001271536 | 0.003712182 | 0.045349725 | 1.343425346 |
| RPS21 | 0.296932012 | 3.1857E−06 | 8.38136E−05 | 4.076685509 | ZNF702P | 1.39259398 | 0.003739784 | 0.045641393 | 1.340641113 |
| TANGO2 | 0.790221989 | 3.27153E−06 | 8.59795E−05 | 4.065604992 | LPAR6 | 0.601944029 | 0.003754046 | 0.045792635 | 1.339204365 |
| APP | 0.640009165 | 3.28714E−06 | 8.62971E−05 | 4.064003974 | TMEM167A | 0.423893303 | 0.003764824 | 0.045901249 | 1.338175497 |
| NONO | 0.457201421 | 3.439E−06 | 9.00907E−05 | 4.045319902 | QRICH1 | 0.498756528 | 0.003773074 | 0.045978941 | 1.337441037 |
| MLIP | 3.438048601 | 3.64304E−06 | 9.51196E−05 | 4.02172994 | HINT2 | 0.467810363 | 0.003791596 | 0.04615873 | 1.335746151 |
| POLE4 | 0.825977206 | 3.6465E−06 | 9.51196E−05 | 4.02172994 | LGALS7B | 1.066919088 | 0.003814568 | 0.046393247 | 1.333545227 |
| MAGEH1 | 0.599603951 | 3.67192E−06 | 9.56809E−05 | 4.019174596 | THNSL2 | 0.668342888 | 0.003817548 | 0.046393247 | 1.333545227 |
| ARMC9 | 0.685960114 | 3.77504E−06 | 9.81589E−05 | 4.008070129 | STRA13 | 0.381086618 | 0.003820331 | 0.046393247 | 1.333545227 |
| CHADL | 1.334148765 | 3.81182E−06 | 9.90103E−05 | 4.004319407 | URM1 | 0.368006617 | 0.003817349 | 0.046393247 | 1.333545227 |
| WASH3P | 0.656000912 | 3.8532E−06 | 9.99791E−05 | 4.000090828 | MPC1 | 0.284850359 | 0.003818939 | 0.046393247 | 1.333545227 |
| HNRNPC | 0.401104569 | 4.04113E−06 | 0.000104634 | 3.980328688 | CWC15 | 0.323568761 | 0.003826898 | 0.046449967 | 1.333014587 |
| OSTC | 0.524619758 | 4.05896E−06 | 0.000104984 | 3.978875298 | ARL3 | 0.42163524 | 0.00384326 | 0.046602387 | 1.331591842 |
| PTPMT1 | 0.672026157 | 4.06631E−06 | 0.000105063 | 3.978548377 | PMP22 | 0.128996819 | 0.003849263 | 0.046629 | 1.331343895 |
| EXT2 | 0.892300487 | 4.12748E−06 | 0.000106532 | 3.972521423 | KIAA0513 | 1.398183346 | 0.003862096 | 0.046738228 | 1.330327753 |
| GNB2L1 | 0.16289784 | 4.15415E−06 | 0.000107107 | 3.970181346 | SEC23A | 0.548068702 | 0.003915851 | 0.047365362 | 1.324539138 |
| RPS27A | 0.108491771 | 4.2004E−06 | 0.000108186 | 3.965829731 | HM13 | 0.345783412 | 0.003941551 | 0.047652686 | 1.321912615 |
| ATP6V1D | 0.552772045 | 4.2615E−06 | 0.000109624 | 3.960095686 | ARHGEF6 | 1.491312265 | 0.003963289 | 0.047891865 | 1.31973825 |
| QDPR | 0.497434944 | 4.26518E−06 | 0.000109624 | 3.960095686 | FN1 | 0.467027009 | 0.004006263 | 0.048315811 | 1.315910728 |
| UNC93B1 | 0.694101214 | 4.2759E−06 | 0.000109784 | 3.95946087 | IL34 | 2.281309128 | 0.004018113 | 0.048434876 | 1.314841806 |
| DHRS7 | 0.43849656 | 4.29035E−06 | 0.00011004 | 3.958450969 | SSH1 | 0.506762445 | 0.004034799 | 0.048612081 | 1.313255787 |
| CASP4 | 0.779087543 | 4.32549E−06 | 0.000110744 | 3.95568117 | ABCA2 | 0.641017556 | 0.004094118 | 0.049278289 | 1.307344382 |
| UBE4A | 0.579939842 | 4.32684E−06 | 0.000110744 | 3.95568117 | WBP11 | 0.367152512 | 0.004098567 | 0.049307607 | 1.307086072 |
| STX7 | 0.476404876 | 4.37185E−06 | 0.000111779 | 3.951640731 | CDCA7L | 0.770933807 | 0.004132821 | 0.049670906 | 1.303897917 |
| BST1 | 0.960110962 | 4.57712E−06 | 0.000116905 | 3.932166652 | SMG1P2 | 0.602243245 | 0.004142227 | 0.049735151 | 1.303336559 |
| MGP | 1.894036293 | 4.58608E−06 | 0.000117012 | 3.931769996 | ZNF749 | 0.404445171 | 0.004141157 | 0.049735151 | 1.303336559 |
| YPEL5 | 0.65289259 | 4.62534E−06 | 0.000117891 | 3.928520096 | SUN2 | 0.56682092 | 0.004154227 | 0.049830379 | 1.302505808 |

TABLE 2
DevMel classifier performance and program genes from FIG. 4B-C.
prg[NEO]

| | | correlation | Average expression in each developmental group | | | |
|---|---|---|---|---|---|---|
| gene | correlation | coefficient | MSC | FET | NEO | ADT |
| SLCO4A1 | positive | 0.090387866 | 1.2641387 | 2.2223482 | 4.4089575 | 2.9625027 |
| MAD1L1 | positive | 0.088423595 | 1.574745 | 2.4751017 | 3.734714 | 2.4787169 |
| ZNF330 | positive | 0.082257025 | 1.4132726 | 2.2405398 | 2.908652 | 1.4611392 |
| ARSI | positive | 0.080429129 | 0.45168975 | 0.32408157 | 1.913959 | 0.8914525 |
| BECN1 | positive | 0.071492493 | 1.1748694 | 1.7884659 | 3.2874742 | 1.8919536 |
| HPSE2 | positive | 0.068503164 | 0.08345231 | 0.32956576 | 0.36913574 | 0.019688668 |
| SERPINF1 | positive | 0.065788649 | 4.8836894 | 6.648149 | 6.917258 | 0.51705354 |
| RAB38 | positive | 0.057122298 | 1.5772153 | 4.2450595 | 5.9825363 | 3.1496892 |
| YIPF1 | positive | 0.056810245 | 0.8221121 | 0.91739696 | 1.6859776 | 0.6793742 |
| MICAL2 | positive | 0.053889818 | 0.055973977 | 0.23363082 | 0.6947131 | 0.18538292 |
| CRYM | positive | 0.045158569 | 1.5019817 | 2.1356192 | 4.3455877 | 3.3008857 |
| SPG21 | positive | 0.039370753 | 2.4692748 | 2.7790964 | 4.120765 | 2.6216724 |
| AIG1 | positive | 0.03902632 | 0.8168259 | 1.0098033 | 2.6221852 | 2.12121 |
| TMEM204 | positive | 0.03674816 | 1.6701108 | 2.921409 | 3.8727286 | 1.953419 |
| OSGEP | positive | 0.033427302 | 0.9557276 | 1.2769252 | 1.6479856 | 0.65386707 |
| PKIG | positive | 0.032207351 | 1.548999 | 2.218216 | 2.5356324 | 1.4581879 |
| XPNPEP1 | positive | 0.032172151 | 1.3597461 | 1.963225 | 2.756986 | 0.8228575 |
| PNP | positive | 0.031229842 | 0.59185636 | 1.2257788 | 0.8961103 | 0.35904828 |
| MDH1 | positive | 0.029996442 | 3.651967 | 4.0780215 | 5.0006723 | 3.1776147 |
| LOC100506207 | positive | 0.029380322 | 0.05932809 | 0.07873423 | 0.14590637 | 0.08275345 |
| TMEM192 | positive | 0.028370077 | 0.9477396 | 1.5735964 | 2.2449844 | 1.407366 |
| EDNRB | positive | 0.026418285 | 2.7628288 | 5.784856 | 6.4040513 | 4.214343 |
| TBC1D22A | positive | 0.025291344 | 0.58803004 | 0.9796431 | 1.603178 | 0.80322593 |
| SNAP29 | positive | 0.024973786 | 1.549973 | 1.7450494 | 2.1002898 | 1.0529053 |
| ATPAF2 | positive | 0.023587655 | 0.7748545 | 1.0627764 | 1.2049105 | 0.5101456 |
| PIGF | positive | 0.021800861 | 1.2482812 | 1.3904904 | 2.333466 | 0.9272668 |
| SMIM10 | positive | 0.020101931 | 0.07572515 | 0.72577757 | 1.076914 | 1.0465267 |
| RASSF3 | positive | 0.020095265 | 1.4990807 | 2.636765 | 3.3724935 | 1.1099204 |
| SURF1 | positive | 0.019730411 | 0.9887694 | 2.2500045 | 3.0078278 | 1.9837278 |
| CA14 | positive | 0.019581549 | 1.5182956 | 4.2730937 | 5.2114754 | 2.786004 |
| PSMC2 | positive | 0.018524449 | 2.3387544 | 2.5367527 | 3.2294247 | 1.8619167 |
| OGFRL1 | positive | 0.015771769 | 0.29537252 | 0.24070978 | 0.23712452 | 0.20464727 |
| CHAF1A | positive | 0.014102879 | 0.5736362 | 0.5822738 | 0.6266711 | 0.3423366 |
| TMEM120A | positive | 0.013812479 | 1.1263111 | 2.714449 | 3.186496 | 2.2905407 |
| C3orf79 | positive | 0.013760562 | 1.4406384 | 4.0078783 | 4.3267064 | 0.6375698 |
| ALDH9A1 | positive | 0.013119848 | 1.695161 | 1.8958144 | 2.7588363 | 0.9460103 |
| TM2D3 | positive | 0.012048749 | 2.0711215 | 1.5586205 | 2.470953 | 1.3085501 |
| NIT2 | positive | 0.012037567 | 1.4372725 | 2.274058 | 2.948152 | 1.2818611 |
| MFF | positive | 0.011782891 | 2.2005944 | 2.5820446 | 3.6633673 | 1.8016573 |
| SEC11C | positive | 0.011070455 | 1.7599106 | 3.025719 | 4.482602 | 2.413646 |
| WDSUB1 | positive | 0.010225798 | 0.51219493 | 0.9197637 | 1.0488262 | 0.43562293 |
| SWI5 | positive | 0.008850762 | 0.8228434 | 1.1968639 | 1.7355535 | 0.78589004 |
| LOC100128239 | positive | 0.007376726 | 0.05231487 | 0.061804365 | 0.2155686 | 0.048798792 |
| CIDEA | positive | 0.007167832 | 0 | 0.002457799 | 0.15804134 | 0.03021288 |
| SRA1 | positive | 0.006507153 | 2.0325682 | 1.9060123 | 2.996753 | 1.5445827 |
| CDSN | positive | 0.005387611 | 0 | 0.002998606 | 0.017288228 | 0.035618667 |
| PTN | positive | 0.005049676 | 1.2579261 | 0.8591071 | 1.7545797 | 0.64777094 |
| POLR3C | positive | 0.003192311 | 1.1535691 | 1.0163639 | 1.4843215 | 0.6954018 |
| HSD3B7 | positive | 0.001688014 | 0.33113748 | 0.57711715 | 0.75472283 | 0.28170633 |
| PRMT2 | positive | 0.000903664 | 3.6686363 | 4.337189 | 5.0091147 | 3.184627 |
| RSU1 | positive | 0.000878775 | 2.8472228 | 3.101452 | 4.2101083 | 2.657136 |
| CXorf65 | positive | 0.000235604 | 0.066014014 | 0.19770019 | 0.2718642 | 0.06323015 |
| PIGH | positive | 0.00012663 | 1.2143983 | 1.1305461 | 2.4842274 | 1.4027914 |
| TMEM47 | negative | −0.000138397 | 0.69669145 | 0.067746066 | 0.01837892 | 1.4113984 |
| COL4A1 | negative | −0.000919739 | 3.5493493 | 0.9515583 | 0.08015933 | 0.5023679 |
| GADD45B | negative | −0.003297595 | 2.5731995 | 1.2874947 | 0.070805416 | 0.553457 |
| ZNF292 | negative | −0.003308671 | 2.7704186 | 1.5917178 | 1.297286 | 1.192457 |
| DUSP1 | negative | −0.004282618 | 3.3223352 | 2.9337955 | 0.6719816 | 0.38588747 |
| ZNF611 | negative | −0.005172588 | 0.9284777 | 0.435658 | 0.40290838 | 0.30054748 |
| LGALS7B | negative | −0.006083135 | 0.22598274 | 0.17045487 | 0.2373416 | 0.6022198 |
| CRABP1 | negative | −0.010406487 | 3.9758992 | 0.15768035 | 0.35247055 | 4.6406727 |
| GIGYF1 | negative | −0.011564057 | 2.5608938 | 2.1389678 | 1.2187794 | 1.5760902 |
| NOTCH2NL | negative | −0.01182266 | 1.9714447 | 2.5837905 | 1.1206172 | 1.4002622 |
| S100A6 | negative | −0.019262059 | 4.9298663 | 3.5497944 | 2.7664566 | 5.426575 |
| KIF1A | negative | −0.020587912 | 1.215462 | 0.9164256 | 0.24859013 | 1.746996 |
| CLU | negative | −0.024498215 | 0.092144996 | 0.047891695 | 0.025947277 | 4.33635 |
| COL1A2 | negative | −0.04516096 | 7.69115 | 4.45441 | 0.6376246 | 0.38812172 |
| FOS | negative | −0.050811801 | 5.7766986 | 6.3614197 | 2.3022602 | 1.8408839 |
| LYPD1 | negative | −0.08372236 | 1.1204109 | 3.3728514 | 0.007065248 | 0.004874161 |

TABLE 3
Melanoma dedifferentiation analysis gene set from FIG. 6C-G.
All 511 genes in dedifferentiation analysis.
source: gene was identified via melanoma group differential expression analysis
(diff_exp), DevMel logistical regression analysis (logit) or by both methods (both).
pattern: type of dedifferentiation pattern

| gene | source | pattern | gene | source | pattern |
|---|---|---|---|---|---|
| DUSP6 | diff_exp | direct | TMEM120A | logit | sequential |
| EGR1 | diff_exp | direct | TMEM192 | logit | sequential |
| HMGA2 | diff_exp | direct | TMEM204 | logit | sequential |
| KRT18 | diff_exp | direct | WNT5A | logit | sequential |
| NT5DC3 | diff_exp | direct | XPNPEP1 | logit | sequential |
| PCOLCE | diff_exp | direct | YIPF1 | logit | sequential |
| SERPING1 | diff_exp | direct | ZNF330 | logit | sequential |
| AXL | logit | direct | ZNF517 | logit | sequential |
| CYR61 | logit | direct | APOE | both | no_pattern |
| DHRS3 | logit | direct | FOS | both | no_pattern |
| DUSP1 | logit | direct | TIMP3 | both | no_pattern |
| IER2 | logit | direct | ADRBK2 | diff_exp | no_pattern |
| MGP | logit | direct | AEBP1 | diff_exp | no_pattern |
| MOXD1 | logit | direct | ANXA1 | diff_exp | no_pattern |
| PSAT1 | logit | direct | APOC2 | diff_exp | no_pattern |
| RAB3B | logit | direct | ASAP1 | diff_exp | no_pattern |
| CHADL | logit | down regulated | ATP1A1 | diff_exp | no_pattern |
| CRABP1 | logit | down regulated | ATP5B | diff_exp | no_pattern |
| EMX2 | logit | down regulated | ATP5G2 | diff_exp | no_pattern |
| KIF1A | logit | down regulated | BRIX1 | diff_exp | no_pattern |
| KRT1 | logit | down regulated | BZW2 | diff_exp | no_pattern |
| MAP6 | logit | down regulated | C1QBP | diff_exp | no_pattern |
| POU3F3 | logit | down regulated | CALU | diff_exp | no_pattern |
| SEPT4 | logit | down regulated | CCT2 | diff_exp | no_pattern |
| SFTPC | logit | down regulated | CD47 | diff_exp | no_pattern |
| TMEM47 | logit | down regulated | CD58 | diff_exp | no_pattern |
| TRIM29 | logit | down regulated | CD63 | diff_exp | no_pattern |
| C3orf79 | logit | not_readopted | CD68 | diff_exp | no_pattern |
| COL21A1 | logit | not_readopted | CDH19 | diff_exp | no_pattern |
| COL23A1 | logit | not_readopted | CHCHD2 | diff_exp | no_pattern |
| COL5A1 | logit | not_readopted | CNRIP1 | diff_exp | no_pattern |
| LIMS2 | logit | not_readopted | CS | diff_exp | no_pattern |
| PAMR1 | logit | not_readopted | CSNK2B | diff_exp | no_pattern |
| RADIL | logit | not_readopted | CTSB | diff_exp | no_pattern |
| APOD | both | sequential | DDX5 | diff_exp | no_pattern |
| ARHGDIB | both | sequential | EEA1 | diff_exp | no_pattern |
| CA14 | both | sequential | EEF1B2 | diff_exp | no_pattern |
| CAPG | both | sequential | EEF1D | diff_exp | no_pattern |
| COL1A1 | both | sequential | EGFL8 | diff_exp | no_pattern |
| COL1A2 | both | sequential | EIF2S3 | diff_exp | no_pattern |
| FXYD3 | both | sequential | EIF3E | diff_exp | no_pattern |
| HLA-B | both | sequential | EIF3K | diff_exp | no_pattern |
| ID3 | both | sequential | EIF3M | diff_exp | no_pattern |
| IFI27 | both | sequential | EMP1 | diff_exp | no_pattern |
| LGALS3 | both | sequential | ESRP1 | diff_exp | no_pattern |
| MAD1L1 | both | sequential | FAM178B | diff_exp | no_pattern |
| MT2A | both | sequential | FARSA | diff_exp | no_pattern |
| NGFRAP1 | both | sequential | FBXO32 | diff_exp | no_pattern |
| NREP | both | sequential | FLNA | diff_exp | no_pattern |
| RAB38 | both | sequential | FOXRED2 | diff_exp | no_pattern |
| RASSF3 | both | sequential | GAS5 | diff_exp | no_pattern |
| S100A6 | both | sequential | GNB2L1 | diff_exp | no_pattern |
| SERPINF1 | both | sequential | GPM6B | diff_exp | no_pattern |
| TCF4 | both | sequential | GPNMB | diff_exp | no_pattern |
| TSC22D3 | both | sequential | GRN | diff_exp | no_pattern |
| A2M | diff_exp | sequential | HLA-G | diff_exp | no_pattern |
| ABR | diff_exp | sequential | HMGB1 | diff_exp | no_pattern |
| ACP5 | diff_exp | sequential | HN1 | diff_exp | no_pattern |
| ADSL | diff_exp | sequential | IGFBP7 | diff_exp | no_pattern |
| AHCY | diff_exp | sequential | ILF2 | diff_exp | no_pattern |
| AHNAK | diff_exp | sequential | IMPDH2 | diff_exp | no_pattern |
| ATP1B1 | diff_exp | sequential | ITGB3 | diff_exp | no_pattern |
| B2M | diff_exp | sequential | ITGB8 | diff_exp | no_pattern |
| BCAN | diff_exp | sequential | JUN | diff_exp | no_pattern |
| BTF3 | diff_exp | sequential | KCNAB2 | diff_exp | no_pattern |
| C12orf57 | diff_exp | sequential | KIF17 | diff_exp | no_pattern |
| C19orf53 | diff_exp | sequential | KLHDC8B | diff_exp | no_pattern |
| CAV1 | diff_exp | sequential | LAMP2 | diff_exp | no_pattern |
| CCT3 | diff_exp | sequential | LDHB | diff_exp | no_pattern |
| CD151 | diff_exp | sequential | LGALS1 | diff_exp | no_pattern |
| CD59 | diff_exp | sequential | LGALS3BP | diff_exp | no_pattern |
| CHP1 | diff_exp | sequential | LRPAP1 | diff_exp | no_pattern |
| CSPG4 | diff_exp | sequential | MAGEA4 | diff_exp | no_pattern |
| CST3 | diff_exp | sequential | MARCKS | diff_exp | no_pattern |
| CTSD | diff_exp | sequential | MDH2 | diff_exp | no_pattern |
| CYC1 | diff_exp | sequential | MID1 | diff_exp | no_pattern |
| DAG1 | diff_exp | sequential | MTRNR2L1 | diff_exp | no_pattern |
| DCT | diff_exp | sequential | MTRNR2L3 | diff_exp | no_pattern |
| DUSP4 | diff_exp | sequential | MTRNR2L6 | diff_exp | no_pattern |
| EEF1A1 | diff_exp | sequential | MTRNR2L8 | diff_exp | no_pattern |
| EEF1G | diff_exp | sequential | MYL6 | diff_exp | no_pattern |
| EIF3G | diff_exp | sequential | NAP1L1 | diff_exp | no_pattern |
| EIF3H | diff_exp | sequential | NDUFS5 | diff_exp | no_pattern |
| EIF3L | diff_exp | sequential | NEAT1 | diff_exp | no_pattern |
| EIF4EBP2 | diff_exp | sequential | NF2 | diff_exp | no_pattern |
| ERRFI1 | diff_exp | sequential | NME1 | diff_exp | no_pattern |
| FAM174B | diff_exp | sequential | NPM1 | diff_exp | no_pattern |
| FAU | diff_exp | sequential | NR4A1 | diff_exp | no_pattern |
| FBL | diff_exp | sequential | NSG1 | diff_exp | no_pattern |
| GAA | diff_exp | sequential | NUDT1 | diff_exp | no_pattern |
| GALE | diff_exp | sequential | OXA1L | diff_exp | no_pattern |
| GLOD4 | diff_exp | sequential | PABPC1 | diff_exp | no_pattern |
| GPC3 | diff_exp | sequential | PERP | diff_exp | no_pattern |
| GPI | diff_exp | sequential | PFN1 | diff_exp | no_pattern |
| GPR155 | diff_exp | sequential | PGAM1 | diff_exp | no_pattern |
| GSN | diff_exp | sequential | PKM | diff_exp | no_pattern |
| GSTO1 | diff_exp | sequential | PKNOX2 | diff_exp | no_pattern |
| H3F3A | diff_exp | sequential | PLTP | diff_exp | no_pattern |
| HLA-A | diff_exp | sequential | POLR2I | diff_exp | no_pattern |
| HLA-C | diff_exp | sequential | PPA1 | diff_exp | no_pattern |
| HLA-E | diff_exp | sequential | PPIA | diff_exp | no_pattern |
| HLA-F | diff_exp | sequential | PRDX2 | diff_exp | no_pattern |
| HLA-H | diff_exp | sequential | PSMB6 | diff_exp | no_pattern |
| HNRNPA1 | diff_exp | sequential | PTP4A3 | diff_exp | no_pattern |
| HNRNPA1P10 | diff_exp | sequential | RPL10 | diff_exp | no_pattern |
| IDH2 | diff_exp | sequential | RPL10A | diff_exp | no_pattern |
| IFI6 | diff_exp | sequential | RPL11 | diff_exp | no_pattern |
| IGF2BP1 | diff_exp | sequential | RPL12 | diff_exp | no_pattern |
| ITGA3 | diff_exp | sequential | RPL13A | diff_exp | no_pattern |
| LHFPL3-AS1 | diff_exp | sequential | RPL13AP5 | diff_exp | no_pattern |
| LSM7 | diff_exp | sequential | RPL14 | diff_exp | no_pattern |
| MATN2 | diff_exp | sequential | RPL17 | diff_exp | no_pattern |
| MFGE8 | diff_exp | sequential | RPL18A | diff_exp | no_pattern |
| MIA | diff_exp | sequential | RPL19 | diff_exp | no_pattern |
| MOB3B | diff_exp | sequential | RPL22 | diff_exp | no_pattern |
| NACA | diff_exp | sequential | RPL26 | diff_exp | no_pattern |
| NENF | diff_exp | sequential | RPL27 | diff_exp | no_pattern |
| NOP56 | diff_exp | sequential | RPL28 | diff_exp | no_pattern |
| NPC1 | diff_exp | sequential | RPL29 | diff_exp | no_pattern |
| NPC2 | diff_exp | sequential | RPL3 | diff_exp | no_pattern |
| NPL | diff_exp | sequential | RPL39 | diff_exp | no_pattern |
| OLA1 | diff_exp | sequential | RPL6 | diff_exp | no_pattern |
| PDE4DIP | diff_exp | sequential | RPL7A | diff_exp | no_pattern |
| PFDN5 | diff_exp | sequential | RPL8 | diff_exp | no_pattern |
| PIH1D1 | diff_exp | sequential | RPL9 | diff_exp | no_pattern |
| PPP1CB | diff_exp | sequential | RPLP0 | diff_exp | no_pattern |
| PPP2R1A | diff_exp | sequential | RPLP1 | diff_exp | no_pattern |
| PRMT1 | diff_exp | sequential | RPLP2 | diff_exp | no_pattern |
| PROS1 | diff_exp | sequential | RPS10 | diff_exp | no_pattern |
| PSAP | diff_exp | sequential | RPS12 | diff_exp | no_pattern |
| PTMA | diff_exp | sequential | RPS16 | diff_exp | no_pattern |
| RPL13 | diff_exp | sequential | RPS17 | diff_exp | no_pattern |
| RPL15 | diff_exp | sequential | RPS19 | diff_exp | no_pattern |
| RPL18 | diff_exp | sequential | RPS21 | diff_exp | no_pattern |
| RPL21 | diff_exp | sequential | RPS25 | diff_exp | no_pattern |
| RPL21P28 | diff_exp | sequential | RPS28 | diff_exp | no_pattern |
| RPL24 | diff_exp | sequential | RPS3 | diff_exp | no_pattern |
| RPL27A | diff_exp | sequential | RPS3A | diff_exp | no_pattern |
| RPL30 | diff_exp | sequential | RPS4X | diff_exp | no_pattern |
| RPL31 | diff_exp | sequential | RPS6 | diff_exp | no_pattern |
| RPL32 | diff_exp | sequential | RPS7 | diff_exp | no_pattern |
| RPL35A | diff_exp | sequential | RPS9 | diff_exp | no_pattern |
| RPL36 | diff_exp | sequential | RPSA | diff_exp | no_pattern |
| RPL36A | diff_exp | sequential | S100B | diff_exp | no_pattern |
| RPL37 | diff_exp | sequential | SAE1 | diff_exp | no_pattern |
| RPL4 | diff_exp | sequential | SCARB2 | diff_exp | no_pattern |
| RPL41 | diff_exp | sequential | SCCPDH | diff_exp | no_pattern |
| RPL7 | diff_exp | sequential | SCD | diff_exp | no_pattern |
| RPS11 | diff_exp | sequential | SCIN | diff_exp | no_pattern |
| RPS13 | diff_exp | sequential | SDC3 | diff_exp | no_pattern |
| RPS14 | diff_exp | sequential | SERPINA3 | diff_exp | no_pattern |
| RPS15 | diff_exp | sequential | SET | diff_exp | no_pattern |
| RPS15A | diff_exp | sequential | SGK1 | diff_exp | no_pattern |
| RPS18 | diff_exp | sequential | SLC25A6 | diff_exp | no_pattern |
| RPS20 | diff_exp | sequential | SLC26A2 | diff_exp | no_pattern |
| RPS23 | diff_exp | sequential | SLC5A3 | diff_exp | no_pattern |
| RPS24 | diff_exp | sequential | SNAI2 | diff_exp | no_pattern |
| RPS27 | diff_exp | sequential | SNHG6 | diff_exp | no_pattern |
| RPS27A | diff_exp | sequential | SNRPD1 | diff_exp | no_pattern |
| RPS5 | diff_exp | sequential | SNRPD2 | diff_exp | no_pattern |
| RPS8 | diff_exp | sequential | SNRPF | diff_exp | no_pattern |
| S100A1 | diff_exp | sequential | SORD | diff_exp | no_pattern |
| S100A13 | diff_exp | sequential | SQSTM1 | diff_exp | no_pattern |
| SAMM50 | diff_exp | sequential | SS18L1 | diff_exp | no_pattern |
| SERPINE2 | diff_exp | sequential | STOML2 | diff_exp | no_pattern |
| SLC25A5 | diff_exp | sequential | TIMP1 | diff_exp | no_pattern |
| SMC4 | diff_exp | sequential | TNFRSF14 | diff_exp | no_pattern |
| SNRPE | diff_exp | sequential | TPI1 | diff_exp | no_pattern |
| SOAT1 | diff_exp | sequential | TRMT112 | diff_exp | no_pattern |
| SOX4 | diff_exp | sequential | TUBB | diff_exp | no_pattern |
| SSBP1 | diff_exp | sequential | TUBB4A | diff_exp | no_pattern |
| SSR2 | diff_exp | sequential | TXNDC17 | diff_exp | no_pattern |
| TAPBP | diff_exp | sequential | TYR | diff_exp | no_pattern |
| TIMM50 | diff_exp | sequential | UBA52 | diff_exp | no_pattern |
| TIMP2 | diff_exp | sequential | UBC | diff_exp | no_pattern |
| TM4SF1 | diff_exp | sequential | UBL5 | diff_exp | no_pattern |
| TMC6 | diff_exp | sequential | UQCRH | diff_exp | no_pattern |
| TP53 | diff_exp | sequential | ABCG1 | logit | no_pattern |
| TPM2 | diff_exp | sequential | ADAT2 | logit | no_pattern |
| TPM4 | diff_exp | sequential | AFF3 | logit | no_pattern |
| TPP1 | diff_exp | sequential | ANK2 | logit | no_pattern |
| TRPM1 | diff_exp | sequential | ARSI | logit | no_pattern |
| U2AF1 | diff_exp | sequential | ASXL1 | logit | no_pattern |
| UQCRFS1 | diff_exp | sequential | ATPAF2 | logit | no_pattern |
| ZNF749 | diff_exp | sequential | CCBE1 | logit | no_pattern |
| AIG1 | logit | sequential | CCND1 | logit | no_pattern |
| ALDH9A1 | logit | sequential | CCND2 | logit | no_pattern |
| AMFR | logit | sequential | CD36 | logit | no_pattern |
| AUTS2 | logit | sequential | CDSN | logit | no_pattern |
| BDH1 | logit | sequential | CEBPD | logit | no_pattern |
| BECN1 | logit | sequential | CELF6 | logit | no_pattern |
| BEX5 | logit | sequential | CES1 | logit | no_pattern |
| C14orf132 | logit | sequential | CHAF1A | logit | no_pattern |
| CCDC3 | logit | sequential | CHCHD3 | logit | no_pattern |
| CD248 | logit | sequential | CHD3 | logit | no_pattern |
| CIDEA | logit | sequential | COL12A1 | logit | no_pattern |
| CLDN11 | logit | sequential | CRMP1 | logit | no_pattern |
| CLU | logit | sequential | CRYM | logit | no_pattern |
| COL3A1 | logit | sequential | CXorf65 | logit | no_pattern |
| COL4A1 | logit | sequential | FAM126A | logit | no_pattern |
| CXCL14 | logit | sequential | FAM183A | logit | no_pattern |
| DAZAP2 | logit | sequential | FSTL4 | logit | no_pattern |
| DCN | logit | sequential | FUT11 | logit | no_pattern |
| DNAJA1 | logit | sequential | GADD45B | logit | no_pattern |
| EDNRB | logit | sequential | GBX2 | logit | no_pattern |
| FBN1 | logit | sequential | GIGYF1 | logit | no_pattern |
| GAPDHS | logit | sequential | GTPBP2 | logit | no_pattern |
| GFRA1 | logit | sequential | HPSE2 | logit | no_pattern |
| GPX1 | logit | sequential | HSPB3 | logit | no_pattern |
| HSD3B7 | logit | sequential | IQGAP2 | logit | no_pattern |
| IGFBP3 | logit | sequential | ITGB5 | logit | no_pattern |
| IGFBP5 | logit | sequential | KLHL24 | logit | no_pattern |
| ITPA | logit | sequential | LDHA | logit | no_pattern |
| KIAA1549L | logit | sequential | LGALS7B | logit | no_pattern |
| KRT13 | logit | sequential | LOC100506207 | logit | no_pattern |
| LAMTOR5 | logit | sequential | LUM | logit | no_pattern |
| LOC100128239 | logit | sequential | LYPD1 | logit | no_pattern |
| LOC400043 | logit | sequential | MEST | logit | no_pattern |
| LOXL2 | logit | sequential | MICAL3 | logit | no_pattern |
| LRRN4CL | logit | sequential | MLANA | logit | no_pattern |
| MARCH9 | logit | sequential | MSX1 | logit | no_pattern |
| MDH1 | logit | sequential | NCKAP5 | logit | no_pattern |
| MEG3 | logit | sequential | NDRG2 | logit | no_pattern |
| MFAP4 | logit | sequential | NID1 | logit | no_pattern |
| MFF | logit | sequential | NOTCH2NL | logit | no_pattern |
| MICAL2 | logit | sequential | PIGF | logit | no_pattern |
| MIR205HG | logit | sequential | PIGH | logit | no_pattern |
| MMP2 | logit | sequential | PKIG | logit | no_pattern |
| MT1M | logit | sequential | PLAT | logit | no_pattern |
| MT1X | logit | sequential | PNP | logit | no_pattern |
| NIT2 | logit | sequential | PRKCE | logit | no_pattern |
| NKAPP1 | logit | sequential | PTGDS | logit | no_pattern |
| OGFRL1 | logit | sequential | PTN | logit | no_pattern |
| OLFML3 | logit | sequential | RASGEF1C | logit | no_pattern |
| OSGEP | logit | sequential | RIC3 | logit | no_pattern |
| OSR2 | logit | sequential | SEC11C | logit | no_pattern |
| PHB | logit | sequential | SEMA4D | logit | no_pattern |
| PITHD1 | logit | sequential | SESN3 | logit | no_pattern |
| PLAU | logit | sequential | SH3PXD2A | logit | no_pattern |
| POLR3C | logit | sequential | SLCO4A1 | logit | no_pattern |
| PPAP2B | logit | sequential | SPTSSA | logit | no_pattern |
| PRMT2 | logit | sequential | SRA1 | logit | no_pattern |
| PSMC2 | logit | sequential | SURF1 | logit | no_pattern |
| RSU1 | logit | sequential | SWI5 | logit | no_pattern |
| SH3BGRL | logit | sequential | TBX15 | logit | no_pattern |
| SMIM10 | logit | sequential | TMSB4X | logit | no_pattern |
| SNAP29 | logit | sequential | TULP2 | logit | no_pattern |
| SOX2 | logit | sequential | TWIST2 | logit | no_pattern |
| SPECC1 | logit | sequential | TXNDC15 | logit | no_pattern |
| SPG21 | logit | sequential | WDSUB1 | logit | no_pattern |
| SUSD3 | logit | sequential | WIF1 | logit | no_pattern |
| SYK | logit | sequential | WSCD1 | logit | no_pattern |
| TBC1D22A | logit | sequential | ZNF292 | logit | no_pattern |
| TGFBI | logit | sequential | ZNF582 | logit | no_pattern |
| THY1 | logit | sequential | ZNF611 | logit | no_pattern |
| TM2D3 | logit | sequential | | | |

52 melanoma specific genes. Subsequent tabs/sheets have
more information on each dedifferentiation pattern

| gene | pattern |
|---|---|
| TMEM212 | melanoma specific |
| MTRNR2L10 | melanoma specific |
| MTRNR2L6 | melanoma specific |
| MIR4461 | melanoma specific |
| MTRNR2L4 | melanoma specific |
| EIF3C | melanoma specific |
| MTRNR2L1 | melanoma specific |
| MTRNR2L3 | melanoma specific |
| DDX39B | melanoma specific |
| MTRNR2L2 | melanoma specific |
| SHISA9 | melanoma specific |
| SPC25 | melanoma specific |
| MTRNR2L8 | melanoma specific |
| PRAME | melanoma specific |
| HNRNPA1P10 | melanoma specific |
| U2AF1 | melanoma specific |
| BST2 | melanoma specific |
| ASTN2 | melanoma specific |
| UGDH-AS1 | melanoma specific |
| RPL21P28 | melanoma specific |
| UBE2V1 | melanoma specific |
| ORC4 | melanoma specific |
| PET100 | melanoma specific |
| CCNG1 | melanoma specific |
| SNHG8 | melanoma specific |
| METTL21A | melanoma specific |
| CBX3 | melanoma specific |
| HIST1H2BK | melanoma specific |
| SRSF1 | melanoma specific |
| UTP11L | melanoma specific |
| H3F3AP4 | melanoma specific |
| DDT | melanoma specific |
| SHC1 | melanoma specific |
| MRPS6 | melanoma specific |
| RPL13AP5 | melanoma specific |
| MRPS24 | melanoma specific |
| SEC61G | melanoma specific |
| SLIRP | melanoma specific |
| IVNS1ABP | melanoma specific |
| RPS29 | melanoma specific |
| UBAP2L | melanoma specific |
| TSNAX | melanoma specific |
| SNHG6 | melanoma specific |
| BZW1 | melanoma specific |
| PFDN2 | melanoma specific |
| NDUFB3 | melanoma specific |
| PDCD5 | melanoma specific |
| FAM96B | melanoma specific |
| LSM3 | melanoma specific |
| ROMO1 | melanoma specific |
| TBCA | melanoma specific |
| UXT | melanoma specific |

NEO

| | | relative expression (z-score of rank mean) | | | |
|---|---|---|---|---|---|
| | | normal melanocytes | | | |
| type | gene | MSC | FET | NEO | ADT |
| full_seq | DAZAP2 | −1.185888712 | −0.468149963 | 0.884384853 | 0.769653822 |
| full_seq | GAPDHS | −0.962526958 | −0.740975338 | 1.043502768 | 0.659999528 |
| full_seq | LHFPL3-AS1 | −1.345466334 | −0.170739255 | 0.832697553 | 0.683508037 |
| full_seq | SPG21 | −0.784557454 | −0.5329083 | 1.443787123 | −0.126321369 |
| full_seq | TBC1D22A | −1.092253982 | −0.272970341 | 1.315284947 | 0.049939375 |
| full_seq | TMEM192 | −1.143461446 | −0.266539881 | 1.265090185 | 0.144911142 |
| full_seq | SH3BGRL | −0.004906542 | −1.394233178 | 0.896158 | 0.502981721 |
| full_seq | ABR | −1.248274643 | 0.155848716 | 1.186847177 | −0.09442125 |
| full_seq | BCAN | −1.327707642 | 0.315554455 | 1.065268316 | −0.053115129 |
| full_seq | CA14 | −1.108787186 | 0.197794115 | 1.26682701 | −0.355833939 |
| full_seq | FAM174B | −0.927590307 | 0.804701905 | 0.9229778 | −0.800089398 |
| full_seq | LRRN4CL | −0.858698523 | 0.458008291 | 1.193468704 | −0.792778472 |
| full_seq | NPL | −0.705239094 | −0.09802555 | 1.443364477 | −0.640099834 |
| full_seq | TRPM1 | −1.260212523 | 0.647644875 | 0.94063426 | −0.328066612 |
| full_seq | ZNF749 | −0.787229167 | −0.336089816 | 1.466201117 | −0.342882133 |
| full_seq | TM2D3 | 0.294365733 | −1.057501847 | 1.248704233 | −0.485568118 |
| full_seq | RASSF3 | −0.681117389 | 0.204124803 | 1.329516733 | −0.852524148 |
| full_seq | EIF3L | 0.484039818 | 0.361578133 | 0.644297357 | −1.489915308 |
| full_seq | NOP56 | 0.306980883 | 0.225858424 | 0.898224465 | −1.431063772 |
| full_seq | TIMM50 | 0.627915975 | 0.08762429 | 0.7243677 | −1.439907965 |
| max_seq | BDH1 | −1.051377355 | −0.327433226 | 1.336256831 | 0.042553749 |
| max_seq | BECN1 | −1.014768858 | −0.443869291 | 1.324890897 | 0.133747251 |
| max_seq | CIDEA | −0.765898216 | −0.661316657 | 1.405557238 | 0.021657635 |
| max_seq | DCT | −1.402100891 | −0.010389704 | 0.841227342 | 0.571263252 |
| max_seq | GLOD4 | −0.932423751 | −0.244487383 | 1.418591688 | −0.241680554 |
| max_seq | LOC100128239 | −0.781779442 | −0.359744808 | 1.46738517 | −0.32586092 |
| max_seq | MAD1L1 | −1.279576057 | −0.22115138 | 1.056701573 | 0.444025863 |
| max_seq | PITHD1 | −1.081523843 | −0.551722738 | 1.126591837 | 0.506654744 |
| max_seq | RSU1 | −0.653480409 | −0.509212485 | 1.486260426 | −0.323567532 |
| max_seq | TMEM120A | −1.46818972 | 0.246324753 | 0.747435348 | 0.47442962 |
| max_seq | YIPF1 | −0.580473973 | −0.463518824 | 1.497506467 | −0.45351367 |
| max_seq | ACP5 | −1.066033642 | 0.524690336 | 1.119714066 | −0.57837076 |
| max_seq | CYC1 | −1.202325152 | 0.394202507 | 1.136347983 | −0.328225338 |
| max_seq | EDNRB | −1.11650433 | 0.365562994 | 1.193341481 | −0.442400145 |
| max_seq | GALE | −1.171372214 | 0.759215416 | 0.901255525 | −0.489098728 |
| max_seq | GPI | −1.046649233 | 0.466988893 | 1.161163545 | −0.581503205 |
| max_seq | GSTO1 | −1.000288989 | 0.170113727 | 1.316721641 | −0.486546379 |
| max_seq | LAMTOR5 | −1.143262916 | 0.826016247 | 0.851560684 | −0.534314015 |
| max_seq | MFF | −0.612449935 | −0.29791555 | 1.485175156 | −0.574809671 |
| max_seq | MICAL2 | −0.902892535 | −0.214765797 | 1.428909809 | −0.311251476 |
| max_seq | MOB3B | −0.862439448 | 0.092870169 | 1.372573811 | −0.603004531 |
| max_seq | POLR3C | −0.665411437 | −0.369535717 | 1.488327111 | −0.453379957 |
| max_seq | RAB38 | −0.955914532 | −0.117810382 | 1.40170508 | −0.327980166 |
| max_seq | SLC25A5 | −0.957842536 | 0.51927834 | 1.145758866 | −0.70719467 |
| max_seq | SOAT1 | −0.891183942 | 0.115200179 | 1.360474452 | −0.584490689 |
| max_seq | TMEM204 | −0.99411207 | −0.039354619 | 1.37585083 | −0.342384141 |
| max_seq | UQCRFS1 | −0.883010168 | 0.475678479 | 1.181178881 | −0.773847192 |
| max_seq | ZNF330 | −0.918258934 | −0.027804435 | 1.397290711 | −0.451227342 |
| max_seq | NENF | 0.571634256 | −1.462759305 | 0.706826344 | 0.184298704 |
| max_seq | ADSL | −0.602877048 | 0.688394617 | 0.996230024 | −1.081747593 |
| max_seq | AHCY | 0.369198053 | 0.437618684 | 0.679805844 | −1.486622581 |
| max_seq | ALDH9A1 | −0.502801875 | 0.111978687 | 1.348294864 | −0.957471675 |
| max_seq | CCT3 | −0.388105377 | 0.360291908 | 1.179920706 | −1.152107237 |
| max_seq | CHP1 | −0.569918301 | 0.348774548 | 1.235093367 | −1.013949614 |
| max_seq | DNAJA1 | 0.326410082 | 0.521423597 | 0.639606296 | −1.487439974 |
| max_seq | EIF3H | 0.304926235 | 0.443108642 | 0.728456288 | −1.476491165 |
| max_seq | GPX1 | −0.858853055 | 0.859212482 | 0.872782278 | −0.873141705 |
| max_seq | HSD3B7 | −0.609453959 | 0.185347759 | 1.331043503 | −0.906937304 |
| max_seq | ITPA | −0.491806299 | 0.756547915 | 0.904624311 | −1.169365928 |
| max_seq | MDH1 | −0.521985049 | −0.392847509 | 1.495381389 | −0.580548831 |
| max_seq | NIT2 | −0.693853422 | 0.075392207 | 1.38664332 | −0.768182106 |
| max_seq | OSGEP | −0.676890598 | −0.058057026 | 1.432456088 | −0.697508463 |
| max_seq | PHB | −0.2875177 | 0.727108779 | 0.850724483 | −1.290315562 |
| max_seq | PPP2R1A | −0.756792312 | 0.729438119 | 0.987024748 | −0.959670555 |
| max_seq | PRMT2 | −0.71718783 | 0.206552988 | 1.330632144 | −0.819997303 |
| max_seq | PSMC2 | −0.533586863 | −0.323004886 | 1.487559343 | −0.630967594 |
| max_seq | SAMM50 | −0.523591428 | −0.422759322 | 1.49770861 | −0.551357861 |
| max_seq | SERPINF1 | −0.002272055 | 0.589767297 | 0.819714108 | −1.40720935 |
| max_seq | TMC6 | −0.799251165 | 0.482158291 | 1.177969561 | −0.860876687 |
| max_seq | XPNPEP1 | −0.74810328 | 0.344155438 | 1.260348049 | −0.856400206 |
| max_seq | EEF1A1 | 0.352214988 | −0.408484934 | 1.191642924 | −1.135372979 |
| max_seq | SNAP29 | 0.555445472 | 0.060032448 | 0.809952835 | −1.425430755 |

NEO

| | | relative expression (z-score of rank mean) | | | |
|---|---|---|---|---|---|
| | | melanoma | | | |
| type | gene | MAL_MSC | MAL_FET | MAL_NEO | MAL_ADT |
| full_seq | DAZAP2 | −1.13797136 | −0.517567516 | 1.010444786 | 0.64509409 |
| full_seq | GAPDHS | −1.191780115 | −0.412809502 | 1.049169832 | 0.555419784 |
| full_seq | LHFPL3-AS1 | −0.970175463 | −0.391612944 | 1.380238554 | −0.018450146 |
| full_seq | SPG21 | −0.562754887 | −0.522257705 | 1.496950549 | −0.411937957 |
| full_seq | TBC1D22A | −1.046436931 | −0.622761864 | 1.08362259 | 0.585576205 |
| full_seq | TMEM192 | −1.273821593 | −0.300643546 | 0.941378231 | 0.633086908 |
| full_seq | SH3BGRL | −0.655697952 | −1.048261505 | 0.950643538 | 0.75331592 |
| full_seq | ABR | −0.836467297 | −0.081938497 | 1.426641252 | −0.508235459 |
| full_seq | BCAN | −1.029568156 | 0.319404168 | 1.245532426 | −0.535368438 |
| full_seq | CA14 | −0.905087914 | −0.007456413 | 1.395524977 | −0.48298065 |
| full_seq | FAM174B | −1.059675872 | 0.225290927 | 1.27691101 | −0.442526064 |
| full_seq | LRRN4CL | −1.337285705 | 0.224996631 | 1.076937186 | 0.035351888 |
| full_seq | NPL | −0.607336324 | −0.286981197 | 1.483711332 | −0.589393812 |
| full_seq | TRPM1 | −1.118168938 | 0.089522787 | 1.292984569 | −0.264338419 |
| full_seq | ZNF749 | −0.782826612 | 0.19423041 | 1.33742664 | −0.748830438 |
| full_seq | TM2D3 | 0.479637707 | −1.451600649 | 0.794564721 | 0.177398221 |
| full_seq | RASSF3 | −0.736826984 | 0.157969146 | 1.353523263 | −0.774665425 |
| full_seq | EIF3L | 0.709513803 | −0.624579427 | 0.982931217 | −1.067865593 |
| full_seq | NOP56 | 0.102050941 | −0.118372361 | 1.227882071 | −1.21156065 |
| full_seq | TIMM50 | 0.092161075 | −0.005363326 | 1.178834868 | −1.265632618 |
| max_seq | BDH1 | 0.679653151 | −1.351181973 | 0.829172655 | −0.157643833 |
| max_seq | BECN1 | −0.739863565 | −0.980814975 | 0.931881471 | 0.788797069 |
| max_seq | CIDEA | −0.558708742 | 0.123873048 | 1.352750171 | −0.917914476 |
| max_seq | DCT | −1.161560263 | 0.308038469 | 1.198654779 | −0.345132985 |
| max_seq | GLOD4 | 0.505605767 | −0.520863873 | 1.119597404 | −1.104339299 |
| max_seq | LOC100128239 | −1.359122574 | 0.71742444 | 0.785714507 | −0.144016373 |
| max_seq | MAD1L1 | −0.181723113 | −0.996607034 | 1.38901511 | −0.210684963 |
| max_seq | PITHD1 | −0.599904382 | −0.731980084 | 1.446122268 | −0.114237802 |
| max_seq | RSU1 | 0.469630412 | −0.591084463 | 1.161338421 | −1.039884371 |
| max_seq | TMEM120A | −0.502630228 | −1.046794072 | 1.249700146 | 0.299724155 |
| max_seq | YIPF1 | −0.007901981 | −0.813498724 | 1.410902288 | −0.589501584 |
| max_seq | ACP5 | −1.135256907 | −0.159403708 | 1.298371458 | −0.003710843 |
| max_seq | CYC1 | 0.094615849 | 0.181009748 | 1.070553573 | −1.346179171 |
| max_seq | EDNRB | −1.291437365 | −0.012069393 | 1.14291472 | 0.160592038 |
| max_seq | GALE | −0.65562153 | −0.422357603 | 1.490502824 | −0.412523691 |
| max_seq | GPI | −0.004581086 | 0.180139954 | 1.127152283 | −1.302711151 |
| max_seq | GSTO1 | 0.504557122 | −1.004563459 | 1.147575226 | −0.647568889 |
| max_seq | LAMTOR5 | 0.44307961 | −1.498794405 | 0.531660493 | 0.524054301 |
| max_seq | MFF | 0.347253073 | −0.90582153 | 1.254708059 | −0.696139601 |
| max_seq | MICAL2 | 0.179831964 | −0.704598071 | 1.342547923 | −0.817781816 |
| max_seq | MOB3B | −0.375929249 | −0.083928213 | 1.401657968 | −0.941800507 |
| max_seq | POLR3C | 0.830525926 | −0.498753803 | 0.835721387 | −1.167493509 |
| max_seq | RAB38 | −1.12223951 | −0.331375367 | 1.262657578 | 0.190957299 |
| max_seq | SLC25A5 | 0.288405146 | −0.159811548 | 1.136331075 | −1.264924673 |
| max_seq | SOAT1 | −0.906863132 | −0.267965925 | 1.42874736 | −0.253918303 |
| max_seq | TMEM204 | −0.150609172 | −0.243377012 | 1.38872963 | −0.994743446 |
| max_seq | UQCRFS1 | −0.286998638 | 0.16408148 | 1.262115636 | −1.139198478 |
| max_seq | ZNF330 | −0.810344027 | −0.542690568 | 1.429338059 | −0.076303464 |
| max_seq | NENF | −0.949136737 | −0.264153126 | 1.410798659 | −0.197508795 |
| max_seq | ADSL | 0.073114259 | −0.044598927 | 1.208905994 | −1.237421326 |
| max_seq | AHCY | 0.490200814 | −0.154621883 | 0.98968378 | −1.32526271 |
| max_seq | ALDH9A1 | −0.758302506 | −0.01948676 | 1.41897218 | −0.641182914 |
| max_seq | CCT3 | 0.609940782 | −0.224169201 | 0.925888216 | −1.311659797 |
| max_seq | CHP1 | −0.828917775 | 0.260568894 | 1.304833284 | −0.736484403 |
| max_seq | DNAJA1 | 0.749868573 | −0.786887006 | 0.971877136 | −0.934858703 |
| max_seq | EIF3H | 0.465689488 | −0.05374123 | 0.954929505 | −1.366877763 |
| max_seq | GPX1 | 0.649054486 | −1.425443868 | 0.738514415 | 0.037874967 |
| max_seq | HSD3B7 | −0.82926129 | −0.799506211 | 1.230721201 | 0.3980463 |
| max_seq | ITPA | 0.178264196 | −0.153313046 | 1.200868092 | −1.225819241 |
| max_seq | MDH1 | −0.44652868 | −1.059385845 | 1.274696897 | 0.231217628 |
| max_seq | NIT2 | −0.266842495 | −0.865219779 | 1.443358704 | −0.31129643 |
| max_seq | OSGEP | −0.110675375 | −0.972542707 | 1.394612833 | −0.31139475 |
| max_seq | PHB | 0.063487828 | −0.937692817 | 1.368387707 | −0.494182718 |
| max_seq | PPP2R1A | 0.445467491 | −0.29915875 | 1.089033143 | −1.235341884 |
| max_seq | PRMT2 | −1.178729215 | −0.479820755 | 0.879152236 | 0.779397734 |
| max_seq | PSMC2 | 0.675412059 | −1.367809169 | 0.811606842 | −0.119209732 |
| max_seq | SAMM50 | −0.088948927 | −0.364247298 | 1.400633586 | −0.947437361 |
| max_seq | SERPINF1 | 0.248903954 | 0.234265861 | 0.934518785 | −1.417688601 |
| max_seq | TMC6 | −0.726717754 | −0.205730438 | 1.464929878 | −0.532481687 |
| max_seq | XPNPEP1 | −0.087699455 | −0.720486063 | 1.440101138 | −0.631915621 |
| max_seq | EEF1A1 | 0.726306555 | −1.30029736 | 0.842410941 | −0.268420136 |
| max_seq | SNAP29 | −1.195274337 | 0.019890445 | 1.251077197 | −0.075693305 |

TABLE 4
MALDevMel enriched genes and gene lists from FIG. 8C, G-H. Differentially expressed genes were determined
by Two-sided Wilcoxon Rank Sum Test. Adjusted p-value computed using Benjamini-Hochberg multiple testing
Genes upregulated in MAL_NEO compared to rest of MAL groups

| gene | logfoldchange | pvals_adj | gene | logfoldchange | pvals_adj | gene | logfoldchange | pvals_adj | gene | logfoldchange | pvals_adj |
|---|---|---|---|---|---|---|---|---|---|---|---|
| SERPINF1 | 4.993237 | 1.05004E−72 | EXOC6 | 1.0273753 | 4.74894E−07 | COTL1 | 0.7078207 | 0.000237797 | ZNF346 | 0.38671035 | 0.00560084 |
| PABPC1 | 1.4770935 | 4.07258E−50 | RAD50 | 0.60402215 | 4.77383E−07 | RARS | 0.66638744 | 0.000237857 | TMEM144 | 1.2119049 | 0.005610278 |
| DCT | 4.361558 | 1.90234E−47 | FRMD4B | 1.1913458 | 4.78955E−07 | PATZ1 | 0.7964412 | 0.000238394 | PMPCB | 0.47400528 | 0.005617811 |
| TIMM50 | 2.2546065 | 6.63636E−44 | C10orf76 | 0.6018952 | 4.87762E−07 | INTS4 | 0.64605004 | 0.000238983 | PRKCA | 0.11275025 | 0.005625216 |
| CA14 | 3.0168433 | 8.54592E−40 | PHC1 | 0.6281601 | 4.89231E−07 | CDAN1 | 0.23122744 | 0.000240339 | DPM2 | 0.48001775 | 0.00562604 |
| SCD | 2.423362 | 1.83385E−39 | ARPC1A | 1.0393581 | 4.91657E−07 | ATP6V0D1 | 0.7670744 | 0.000241497 | KIAA2026 | 0.14884436 | 0.005633593 |
| RPS19 | 1.8123778 | 4.99667E−38 | PDZRN3 | 1.2931554 | 4.94507E−07 | FBXO22 | 0.5861248 | 0.00024199 | NDUFA8 | 0.71607715 | 0.005644045 |
| RPL29 | 1.315249 | 4.43932E−37 | SMARCC1 | 0.5960095 | 4.96821E−07 | GTF2H1 | 0.52954686 | 0.000243134 | RPUSD4 | 0.40210626 | 0.00566017 |
| C17orf76-AS1 | 2.2633824 | 5.16619E−37 | CRELD2 | 1.1421188 | 4.9887E−07 | C9orf72 | 0.7952383 | 0.000243579 | CTR9 | 0.46758485 | 0.00566017 |
| BCAN | 3.055299 | 8.16218E−37 | CDS2 | 0.64481163 | 4.9954E−07 | MRPL28 | 0.7758654 | 0.000245303 | SSTR2 | 0.61892486 | 0.005664864 |
| TRPM1 | 2.6077154 | 1.20004E−35 | AK3 | 0.5488157 | 5.03968E−07 | CLP1 | 0.9187167 | 0.000248008 | CYB5A | 0.5893577 | 0.005672465 |
| KIF17 | 2.547519 | 5.10735E−35 | TMEM165 | 0.9960775 | 5.05485E−07 | UTP6 | 0.59848946 | 0.000250772 | TMEM25 | 1.4717149 | 0.005675232 |
| RPS24 | 1.3673493 | 4.45449E−34 | THAP9-AS1 | 0.7266423 | 5.14359E−07 | ZNF446 | 0.8128664 | 0.000251123 | TMTC3 | 0.06458122 | 0.005687694 |
| RPL28 | 1.3948458 | 1.1329E−33 | CCT6A | 0.9529907 | 5.1705E−07 | RAB11FIP2 | 0.6871822 | 0.000251261 | EDC3 | 0.26907957 | 0.005715755 |
| ADRBK2 | 1.4972956 | 1.2389E−33 | MRPL45 | 0.9010318 | 5.17889E−07 | CCDC58 | 0.35267478 | 0.000251346 | ST3GAL3 | 0.3570663 | 0.005748701 |
| KCNAB2 | 2.1000416 | 3.12254E−33 | NKIRAS2 | 0.75091773 | 5.19447E−07 | DHRS4 | 0.61167526 | 0.000253305 | LYSMD4 | 1.2798384 | 0.005804514 |
| PFN1 | 1.3591379 | 3.85214E−33 | PMS2P1 | 0.71415675 | 5.19857E−07 | RENBP | 1.1141849 | 0.000254469 | PIGW | 0.22865053 | 0.005817237 |
| TMC6 | 2.3626826 | 1.87919E−32 | EXOC1 | 0.8923078 | 5.22432E−07 | NEK5 | 0.31084585 | 0.000254933 | NFATC1 | 0.7409362 | 0.005824033 |
| NENF | 2.3568993 | 2.04185E−32 | DCUN1D5 | 0.30153832 | 5.26765E−07 | RLF | 0.3363122 | 0.000255019 | GAS8 | 0.28457308 | 0.005824884 |
| ACP5 | 2.7189853 | 6.95157E−32 | ZNF518A | 0.9937619 | 5.29812E−07 | PPID | 0.48550284 | 0.000256634 | BROX | 0.41888216 | 0.005829703 |
| RPLP0 | 1.3337592 | 8.75021E−32 | FSCN1 | 0.82145774 | 5.34944E−07 | AARSD1 | 0.63686574 | 0.000256634 | SMARCD1 | 0.42864498 | 0.00583552 |
| RPS6 | 1.3269582 | 2.20846E−31 | RAB5C | 0.73942333 | 5.37889E−07 | RHOG | 0.79227597 | 0.000256634 | GNG10 | 0.60945517 | 0.005850014 |
| GAS5 | 2.07283 | 3.87466E−31 | TRUB1 | 1.1435124 | 5.42025E−07 | SHPK | 0.5530312 | 0.000256667 | SNUPN | 0.48708588 | 0.005852861 |
| RPL13AP5 | 1.5734402 | 5.35045E−31 | NUCKS1 | 0.5653603 | 5.44988E−07 | TLCD1 | 1.4828973 | 0.000257244 | PRKACB | 0.32452998 | 0.005856705 |
| SNAI2 | 2.5798244 | 6.92092E−31 | PRKDC | 0.6402711 | 5.46324E−07 | PIN1 | 0.6605195 | 0.000260072 | C18orf56 | 1.3710518 | 0.005870251 |
| FAM174B | 2.2155108 | 7.46431E−31 | FARSA | 1.082976 | 5.52983E−07 | TBXA2R | 0.27765468 | 0.000261298 | UBL7 | 0.674087 | 0.005875105 |
| RASSF3 | 1.2727015 | 3.21356E−30 | FKRP | 0.841667 | 5.64375E−07 | ANKS6 | 0.5548058 | 0.000261441 | MLL4 | 0.66289145 | 0.005889973 |
| PTP4A3 | 2.497336 | 5.22417E−30 | BCL2L1 | 0.7872485 | 5.73458E−07 | H2AFY | 0.48085195 | 0.000261917 | UFL1 | 0.30795798 | 0.00589785 |
| RPS7 | 1.2569237 | 5.53995E−30 | PIH1D1 | 0.9258179 | 5.76927E−07 | RNF4 | 0.5811294 | 0.000262506 | TSPAN9 | 0.36039752 | 0.005901723 |
| GPM6B | 2.2160745 | 8.35276E−30 | ZHX1 | 0.7194651 | 5.77203E−07 | LOC595101 | 0.53110015 | 0.000264716 | BRD8 | 0.3499636 | 0.005931615 |
| FOXRED2 | 1.684949 | 4.9492E−29 | PPIL2 | 0.58239084 | 5.77203E−07 | VRK2 | 0.64070755 | 0.000264861 | FIGN | 0.39962006 | 0.005957738 |
| SOAT1 | 1.7153088 | 4.9492E−29 | ICMT | 0.55789536 | 5.80214E−07 | C11orf70 | 0.056712184 | 0.000267146 | COPS7A | 0.55989325 | 0.005964686 |
| RPS16 | 1.3649871 | 1.02208E−28 | PLIN2 | 0.9405383 | 5.88224E−07 | HES2 | 0.43223614 | 0.000267916 | PSMB6 | 0.90931696 | 0.005965557 |
| PKNOX2 | 1.7719697 | 5.19508E−28 | GNAL | 0.65186274 | 5.88224E−07 | B3GALNT1 | 0.8871954 | 0.00026846 | GGPS1 | 0.22624263 | 0.005970484 |
| RPL18 | 1.3628294 | 1.1265E−27 | FAM198B | 1.4925305 | 5.92106E−07 | SLC39A9 | 0.48280907 | 0.000269062 | TMEM64 | 0.31501994 | 0.005979476 |
| EIF4A1 | 1.5431958 | 1.53604E−27 | NOB1 | 0.9565738 | 5.98151E−07 | TMEM189 | 0.503764 | 0.000269324 | ST3GAL1 | 0.18143994 | 0.005982382 |
| RPS27 | 0.9855995 | 1.76975E−27 | BANP | 0.8548973 | 6.02076E−07 | SLC25A19 | 0.9981983 | 0.000270156 | BUD13 | 0.7071883 | 0.005983256 |
| NOP56 | 1.8435454 | 2.41099E−27 | EID2B | 0.48082367 | 6.03549E−07 | ZC3H7B | 0.42280445 | 0.000271106 | PHF8 | 0.3096597 | 0.005995319 |
| CHP1 | 1.233344 | 2.46476E−27 | LRAT | 1.0567288 | 6.09204E−07 | ANKRD32 | 0.6031002 | 0.000271656 | PTMA | 0.22625369 | 0.006024481 |
| TNFRSF14 | 2.0868564 | 2.46476E−27 | UQCRB | 0.46590903 | 6.09687E−07 | SSNA1 | 0.6572709 | 0.000274321 | GAPVD1 | 0.14893256 | 0.00602843 |
| UQCRFS1 | 2.1952944 | 3.12019E−27 | FAM222B | 0.7597491 | 6.10002E−07 | C1orf35 | 0.58566463 | 0.00027482 | TKTL1 | 1.4343877 | 0.006052889 |
| KLHDC8B | 2.256294 | 1.17305E−26 | C17orf103 | 0.329471 | 6.10486E−07 | SGSM3 | 0.5679212 | 0.000274831 | ZNF3 | 0.5179154 | 0.006068024 |
| RPS9 | 1.0238451 | 1.96977E−26 | GCN1L1 | 0.55205715 | 6.18412E−07 | FBXO17 | 0.5052481 | 0.000274831 | SNX32 | 1.7692441 | 0.00607303 |
| SCIN | 2.869981 | 2.68472E−26 | NUBP2 | 1.0314504 | 6.24718E−07 | CRYZ | 0.6424467 | 0.00027504 | PRKACA | 0.41916874 | 0.006099578 |
| RPL4 | 0.96124005 | 2.74016E−26 | IGHMBP2 | 0.9425247 | 6.24869E−07 | MIR181A2HG | 1.4825237 | 0.000275365 | SMARCAD1 | 0.4047767 | 0.006132177 |
| ASAP1 | 1.311485 | 3.10441E−26 | ZNF518B | 0.85954005 | 6.39054E−07 | GOLPH3L | 0.7023818 | 0.000277092 | CNPY3 | 0.53629756 | 0.006136192 |
| RAB38 | 2.283032 | 3.29041E−26 | PAG1 | 0.75727415 | 6.39054E−07 | DPMI | 0.71412313 | 0.000279005 | ACACB | 0.07840544 | 0.006152705 |
| AHCY | 2.0247157 | 3.80727E−26 | HNRPDL | 0.7029047 | 6.39054E−07 | KIAA1432 | 0.74495834 | 0.000279217 | FAM214B | 0.6283879 | 0.006156732 |
| LOC100133445 | 1.7879609 | 5.67093E−26 | WDR75 | 0.81076854 | 6.39385E−07 | AQP4-AS1 | 0.028895244 | 0.000281298 | TMEM214 | 0.31242055 | 0.006162849 |
| CCT3 | 1.5656347 | 6.87453E−26 | KIAA0196 | 0.62660104 | 6.48743E−07 | CUBN | 0.3840313 | 0.000281298 | RAB2A | 0.47975934 | 0.006165837 |
| RPS11 | 1.2937845 | 7.12199E−26 | ZBTB40 | 0.5881537 | 6.51044E−07 | EPN1 | 0.4671361 | 0.000283538 | PPP2R2D | 0.9334883 | 0.006176145 |
| GPI | 1.653474 | 8.53165E−26 | TRAF3IP2 | 0.47662205 | 6.59299E−07 | ISOC2 | 0.7244495 | 0.000283634 | ZSCAN22 | 0.19935052 | 0.006179139 |
| RPL8 | 1.1438296 | 9.01651E−26 | CBX5 | 0.607953 | 6.59459E−07 | GTPBP8 | 0.79689205 | 0.000283789 | TYW5 | 0.08985625 | 0.00619142 |
| C1QBP | 2.2327676 | 1.35287E−25 | ZNF790-AS1 | 1.071254 | 6.59781E−07 | UST | 0.15579657 | 0.000283884 | GAL3ST4 | 0.10300357 | 0.00619218 |
| TUBB4A | 2.640942 | 1.50694E−25 | MRPS11 | 0.94634324 | 6.59781E−07 | ZNF274 | 0.8765215 | 0.0002853 | LOC100505687 | 0.2646095 | 0.00619218 |
| PLTP | 2.1910825 | 2.22205E−25 | C19orf25 | 0.8039543 | 6.86139E−07 | TMTC4 | 0.8411696 | 0.000285493 | SPATS2 | 0.24725066 | 0.006209882 |
| SS18L1 | 1.6894603 | 2.49136E−25 | CCNF | 0.9525166 | 6.90458E−07 | POP4 | 0.5717206 | 0.000285493 | ASPM | 0.7061438 | 0.006232758 |
| SORD | 1.7535323 | 5.93004E−25 | ZBTB8OS | 0.8588657 | 6.94995E−07 | NFU1 | 0.6822976 | 0.000286071 | LOC150776 | 0.28924137 | 0.006245282 |
| RPS17L | 0.9169573 | 5.93004E−25 | NBPF1 | 0.3989643 | 7.05485E−07 | EPB41L4A | 0.87266177 | 0.000286409 | ECI2 | 0.5703439 | 0.006247251 |
| RPS17 | 0.9169573 | 5.93004E−25 | SURF2 | 1.0239604 | 7.05485E−07 | RNF115 | 0.609154 | 0.000287715 | MSANTD4 | 0.35822532 | 0.006256625 |
| MARCKS | 1.2647879 | 7.33919E−25 | LUST | 0.5768675 | 7.21681E−07 | SSSCA1 | 0.63679034 | 0.000290309 | COA5 | 0.345587 | 0.006266927 |
| RPS4X | 0.93998635 | 3.58791E−24 | GIPC1 | 0.8515646 | 7.39653E−07 | RMND1 | 0.54740006 | 0.000291632 | TSFM | 0.4282849 | 0.006266927 |
| ESRP1 | 2.0238092 | 4.76897E−24 | GALNT18 | 2.1497326 | 7.58449E−07 | NCOA2 | 0.53731465 | 0.000292653 | NPHP4 | 0.43618926 | 0.006292268 |
| NF2 | 1.187182 | 4.87099E−24 | CROCC | 0.588696 | 7.58449E−07 | HHLA3 | 0.25355375 | 0.000292752 | PRPSAP1 | 0.36341232 | 0.006306887 |
| CNRIP1 | 2.2585275 | 4.95775E−24 | HMBS | 1.0836414 | 7.78133E−07 | ZNRD1 | 0.7013682 | 0.000292789 | MERTK | 1.2972305 | 0.006306887 |
| OXA1L | 1.8294839 | 5.83134E−24 | PEX11A | 0.910748 | 7.81304E−07 | U2SURP | 0.37079874 | 0.000293072 | LOC283585 | 0.10487416 | 0.006315276 |
| NPL | 2.1170392 | 6.2817E−24 | AP3M1 | 0.56828576 | 7.88567E−07 | CUTA | 0.7253154 | 0.000293542 | TTI1 | 0.31357348 | 0.006348934 |
| LHFPL3-AS1 | 2.9002924 | 7.61502E−24 | ZFYVE16 | 0.54955834 | 7.90484E−07 | PSMC6 | 0.64582145 | 0.000294482 | SLC38A9 | 0.59266865 | 0.006353861 |
| CD68 | 1.8744749 | 8.58449E−24 | CWF19L1 | 0.53764254 | 7.90893E−07 | FAM102A | 0.54948354 | 0.000294519 | PRSS33 | 2.0581758 | 0.006353861 |
| SAE1 | 1.8725004 | 1.46562E−23 | DDT | 0.8878769 | 7.93899E−07 | TAF2 | 0.49629146 | 0.000295239 | L3MBTL1 | 0.63045496 | 0.006353861 |
| MID1 | 2.0847409 | 1.86169E−23 | PSMC1 | 0.9375807 | 8.03442E−07 | COX10-AS1 | 0.22641063 | 0.000295836 | BRPF3 | 0.37819242 | 0.006375215 |
| ZNF749 | 1.5363317 | 1.8732E−23 | PPFIBP2 | 1.0620967 | 8.03442E−07 | HNRNPA1L2 | 0.43500376 | 0.000296434 | TBC1D13 | 0.6306721 | 0.00639217 |
| PPP2R1A | 1.6789838 | 1.96163E−23 | CLN6 | 0.8052726 | 8.05175E−07 | WDR24 | 0.6776009 | 0.000300488 | ATXN7L2 | 0.8499752 | 0.00639217 |
| RPS8 | 0.9207014 | 1.97628E−23 | YARS | 0.7907411 | 8.09778E−07 | ZNF384 | 0.54447067 | 0.000303956 | FAM200A | 0.44742396 | 0.006394183 |
| GSTO1 | 1.8520924 | 2.10478E−23 | TSPAN14 | 0.7713678 | 8.10419E−07 | LY86-AS1 | 0.4456599 | 0.000303994 | GTSE1 | 0.53869134 | 0.006398356 |
| SAMM50 | 1.7988638 | 2.10478E−23 | TMEM168 | 0.8203361 | 8.21498E−07 | RIN3 | 0.7615683 | 0.000304864 | WDR45B | 0.3701504 | 0.006407938 |
| EIF3K | 2.0324342 | 2.10478E−23 | PLCB4 | 1.0783652 | 8.21498E−07 | HN1L | 0.44242114 | 0.000305069 | COPS3 | 0.5591724 | 0.006430544 |
| SNHG6 | 1.868701 | 2.10478E−23 | BNIP2 | 0.8134605 | 8.30028E−07 | LAPTM4B | 0.5272409 | 0.00030566 | ZNF678 | 0.5002233 | 0.006447781 |
| FAM178B | 1.77855 | 2.9775E−23 | SF3B2 | 0.7104331 | 8.31365E−07 | HSF1 | 0.5441 | 0.00030566 | CARS | 0.4297722 | 0.006467241 |
| RPL6 | 1.0374544 | 2.9939E−23 | OLA1 | 0.66110224 | 8.31797E−07 | CCT5 | 0.7005977 | 0.000306019 | STK3 | 0.39380017 | 0.006499894 |
| TP53 | 1.5220754 | 3.08603E−23 | CNTLN | 0.43618405 | 8.39972E−07 | MRPL42 | 0.5036687 | 0.000306315 | MDN1 | 0.11623077 | 0.006502886 |
| IDH2 | 2.066723 | 3.38605E−23 | MCM3AP | 0.55185056 | 8.41096E−07 | TSEN54 | 0.55359346 | 0.00030807 | ADAMTS7 | 1.2285048 | 0.006502886 |
| SLC25A5 | 1.5623211 | 3.38605E−23 | ERCC2 | 1.0980902 | 8.41533E−07 | NKIRAS1 | 0.5054932 | 0.000308835 | CWF19L2 | 0.035594143 | 0.00652235 |
| RPS5 | 1.5625103 | 5.43E−23 | MTSS1L | 0.7967436 | 8.43348E−07 | GSE1 | 0.335427 | 0.000308835 | LOC389634 | 1.0340954 | 0.006523301 |
| MOB3B | 1.9657133 | 6.00663E−23 | PRKAR1B | 0.89663255 | 8.56728E−07 | RBM27 | 0.30309445 | 0.000309003 | NUDT8 | 0.89303553 | 0.006533054 |
| MAD1L1 | 1.6609297 | 6.00663E−23 | TASP1 | 0.8102557 | 8.56728E−07 | ACP1 | 0.7435208 | 0.000309497 | MRPL55 | 0.7097252 | 0.00654157 |
| IMPDH2 | 1.7756917 | 6.00663E−23 | C10orf118 | 0.4981388 | 8.57874E−07 | RNF144B | 0.4219376 | 0.000309641 | FBXL4 | 0.1234223 | 0.006549141 |
| BZW2 | 1.8954881 | 8.08185E−23 | PRSS21 | 3.0848598 | 8.62066E−07 | CCDC144A | 0.66424084 | 0.000310135 | CT49 | 0.6674941 | 0.00655893 |
| GLOD4 | 1.7151943 | 1.0647E−22 | DPCD | 1.1187634 | 8.76946E−07 | BABAM1 | 0.7464264 | 0.000313839 | MAPK12 | 1.1235336 | 0.006559887 |
| ADSL | 1.9364839 | 1.1342E−22 | POLR3E | 0.82520044 | 8.79075E−07 | BTBD7 | 0.20621213 | 0.000314604 | UCA1 | 0.7996554 | 0.006581427 |
| GALE | 1.9373039 | 1.19122E−22 | LOC284581 | 0.4795695 | 8.86495E−07 | SIRT6 | 0.7227519 | 0.000314815 | TAOK2 | 0.23800391 | 0.006581427 |
| EIF2S3 | 1.3998122 | 1.36118E−22 | RAB32 | 1.1659971 | 8.8766E−07 | CCNB2 | 1.2583257 | 0.000314815 | FIS1 | 0.7415657 | 0.006584605 |
| ABR | 1.6884952 | 1.90909E−22 | SYNPO | 1.0893025 | 8.8766E−07 | DNAJC8 | 0.748202 | 0.000315358 | SPCS2 | 0.55371404 | 0.006588746 |
| PPA1 | 1.9970354 | 2.4764E−22 | NRBF2 | 0.8233838 | 8.95861E−07 | RECQL | 0.3492747 | 0.000315358 | VDAC1 | 0.66830367 | 0.006588746 |
| ILF2 | 1.8700619 | 2.52843E−22 | EXOC3 | 0.9443912 | 8.97303E−07 | HDAC4 | 0.494586 | 0.000315861 | GLYR1 | 0.1518465 | 0.006591927 |
| EIF3L | 1.4480551 | 2.70838E−22 | PTPN14 | 0.5030877 | 8.9997E−07 | ZNF440 | 0.2697694 | 0.000318293 | C11orf82 | 1.1595792 | 0.006592889 |
| EIF4EBP2 | 1.161519 | 3.18598E−22 | LOC100131096 | 0.45696026 | 9.03625E−07 | ATG16L2 | 1.1495674 | 0.000318641 | ZBTB10 | 0.47879988 | 0.006619431 |
| RPL13A | 1.5419121 | 6.45382E−22 | ABI1 | 0.70375985 | 9.12981E−07 | SEC11A | 0.686194 | 0.000319056 | USP2 | 1.1428809 | 0.006624707 |
| CS | 1.4410021 | 7.03529E−22 | URB2 | 1.019021 | 9.19429E−07 | MASTL | 0.4718275 | 0.000319056 | BOLA1 | 0.57576793 | 0.006624707 |
| EGFL8 | 2.5306191 | 8.36848E−22 | ZNF410 | 0.7228844 | 9.24165E−07 | LOC100130370 | 0.40898728 | 0.000319365 | EXOC6B | 0.599106 | 0.006626789 |
| PRDX3 | 1.7506256 | 9.49225E−22 | S100PBP | 0.48090595 | 9.27916E−07 | PIP4K2B | 0.56271553 | 0.000320142 | TRIM68 | 0.329492 | 0.006628872 |
| GSTP1 | 1.3304653 | 1.17442E−21 | PARP2 | 1.1490626 | 9.28401E−07 | GNAI3 | 0.56849974 | 0.000320358 | C4orf46 | 0.35195306 | 0.00662984 |
| CYP51A1 | 1.3197622 | 1.19571E−21 | TGDS | 0.90209705 | 9.33688E−07 | PNKP | 0.7842982 | 0.00032091 | TTC37 | 0.100421 | 0.006635273 |
| HPS4 | 1.4951177 | 1.21504E−21 | PARVA | 0.5962159 | 9.42815E−07 | ST20 | 0.67761177 | 0.00032091 | ZCCHC10 | 0.5053184 | 0.006686665 |
| PAFAH1B3 | 2.0179596 | 1.37514E−21 | NELFCD | 0.9584945 | 9.42815E−07 | RFWD2 | 0.60971 | 0.000321934 | ZNHIT2 | 0.6808121 | 0.006693117 |
| TULP4 | 1.3052988 | 1.51511E−21 | PLCB2 | 1.8283519 | 9.44827E−07 | NMB | 1.0246868 | 0.000321934 | SIPA1L2 | 0.2679117 | 0.006699575 |
| ATP5A1 | 1.490966 | 1.82293E−21 | SCAP | 0.72753 | 9.44827E−07 | SHISA2 | 0.37689418 | 0.000322447 | FLJ25363 | 0.008181858 | 0.006699575 |
| SNHG16 | 1.19418 | 1.9322E−21 | EIF2S2 | 0.7258009 | 9.57706E−07 | PCBP1 | 0.5671493 | 0.000324316 | MCM8 | 0.6277339 | 0.006721838 |
| REV3L | 1.1748437 | 2.06943E−21 | FARP1 | 0.7794841 | 9.63417E−07 | DPH2 | 0.6947629 | 0.000324629 | GJB2 | 2.1900208 | 0.006725079 |
| YWHAZ | 0.94035554 | 2.7602E−21 | HDGF | 0.7519366 | 9.6495E−07 | ABCC2 | 1.1079868 | 0.000325351 | RPL35A | 0.36513472 | 0.006737374 |
| GPR143 | 1.8431433 | 2.99695E−21 | MTERFD1 | 0.9738278 | 9.6495E−07 | MCM10 | 0.797315 | 0.000325953 | FAM173B | 0.044008438 | 0.006746287 |
| BANCR | 2.0898807 | 3.09021E−21 | EPS15 | 0.73303604 | 9.7859E−07 | UGDH | 0.5298411 | 0.000325953 | MTMR14 | 0.34556374 | 0.006790176 |
| RPS3 | 1.006379 | 3.22276E−21 | PWP2 | 0.8639256 | 9.7859E−07 | RPS20 | 0.3186876 | 0.000325953 | HELZ2 | 0.13843372 | 0.006819731 |
| USP22 | 1.2714279 | 3.27792E−21 | COX15 | 0.41630685 | 9.82025E−07 | KLF12 | 0.40907457 | 0.000326814 | EIF4G3 | 0.24295244 | 0.00684366 |
| PRAME | 1.5701514 | 3.33747E−21 | RPL22L1 | 0.7029695 | 9.86273E−07 | TIMM9 | 0.60905594 | 0.00032985 | C1QTNF3 | 0.34073266 | 0.006845805 |
| RPSA | 0.9831066 | 5.46113E−21 | TAF11 | 0.69379807 | 9.91076E−07 | FBXL15 | 0.546178 | 0.00032985 | CCDC77 | 0.62567097 | 0.006861603 |
| RPL18A | 1.402937 | 5.89604E−21 | SYAP1 | 0.6200662 | 9.93208E−07 | SMC5 | 0.26277882 | 0.000330375 | PLEKHG2 | 0.15983634 | 0.006861603 |
| TUFM | 1.6547658 | 6.56101E−21 | PPP2R5D | 0.80626047 | 1.00319E−06 | COMT | 0.7675084 | 0.000331359 | TM2D3 | 0.5204204 | 0.006868362 |
| TRA2A | 1.5646719 | 7.50644E−21 | PRPF40A | 0.6277499 | 1.00998E−06 | RPRD1B | 0.3703346 | 0.000331375 | LOC286467 | 0.16427222 | 0.00689129 |
| METTL9 | 1.612061 | 1.18109E−20 | VCL | 0.7883561 | 1.0182E−06 | CISD1 | 0.5234091 | 0.000331375 | EHMT2 | 0.3882202 | 0.006922255 |
| SMS | 1.7002423 | 1.28088E−20 | DDX10 | 0.8237063 | 1.02315E−06 | ZNF544 | 0.37140092 | 0.000331556 | OCEL1 | 0.59825665 | 0.006946511 |
| TRIB2 | 1.441539 | 1.54733E−20 | EXOG | 0.36013672 | 1.02424E−06 | IK | 0.66957474 | 0.000332154 | MPHOSPH8 | 0.21654767 | 0.006949852 |
| APP | 1.6011953 | 1.59018E−20 | TBP | 0.60051394 | 1.02782E−06 | EXOSC7 | 0.7098625 | 0.000332474 | NIPSNAP3A | 0.5097556 | 0.00697186 |
| RPL11 | 0.85676026 | 1.72597E−20 | DLD | 0.803792 | 1.03562E−06 | UQCRC1 | 0.9978369 | 0.00033356 | RGL2 | 0.21077977 | 0.006972875 |
| RPL21 | 3.168274 | 2.21981E−20 | LSMD1 | 0.72333837 | 1.04431E−06 | TOP3B | 0.53847355 | 0.000334231 | C1orf51 | 0.9621317 | 0.006983242 |
| SKAP2 | 1.53858 | 2.38463E−20 | WDR74 | 0.9827448 | 1.0585E−06 | FAM153C | 0.005328791 | 0.000335393 | DNAJC30 | 0.30078527 | 0.00698777 |
| C1orf198 | 1.3860836 | 2.40749E−20 | 44446 | 0.71693695 | 1.06421E−06 | BMPR1A | 0.8930318 | 0.000336207 | C12orf76 | 0.41577804 | 0.00700402 |
| WSB2 | 1.2752829 | 2.4589E−20 | MARCKSL1 | 0.9249625 | 1.06678E−06 | LINC00477 | 0.41017872 | 0.000336997 | ARG2 | 0.47678348 | 0.007017801 |
| RPL3 | 0.71686274 | 2.47671E−20 | DKFZp686O1327 | 0.89854246 | 1.07284E−06 | ADNP2 | 1.0093008 | 0.000337745 | STK16 | 0.4877117 | 0.007017801 |
| SF3B4 | 1.2582308 | 4.27359E−20 | ST6GALNAC3 | 1.3572377 | 1.07601E−06 | CNST | 0.41139588 | 0.000337929 | C1orf74 | 1.0556656 | 0.007018823 |
| KIAA0020 | 1.3316151 | 5.93052E−20 | SSBP3 | 0.81473386 | 1.07658E−06 | FDX1L | 0.8039774 | 0.000339245 | KIAA1731 | 0.004167877 | 0.00702102 |
| XPNPEP1 | 1.6361142 | 6.47155E−20 | ACOX1 | 0.49267697 | 1.09444E−06 | EHHADH | 0.10844173 | 0.000339288 | BTBD1 | 0.5383385 | 0.007024394 |
| MRPS16 | 1.2053376 | 6.71742E−20 | IWS1 | 0.82166773 | 1.09501E−06 | PAK1 | 0.7528548 | 0.000339616 | CCNI | 0.47235745 | 0.007039544 |
| TPCN2 | 1.7517785 | 7.11689E−20 | RRP12 | 0.91008395 | 1.09648E−06 | FBXO21 | 0.433468 | 0.000340697 | FAM207A | 0.6592344 | 0.007040569 |
| PI4KB | 1.4379435 | 7.81745E−20 | HSD3B7 | 0.5525132 | 1.10091E−06 | SUPT20H | 0.42497635 | 0.000340697 | LIMD1 | 0.22674748 | 0.007043952 |
| HSPA8 | 0.9328129 | 8.96801E−20 | RFK | 0.596929 | 1.10626E−06 | KDM3A | 0.42130306 | 0.000341025 | BRD1 | 0.35996515 | 0.007044978 |
| NDST1 | 1.4439476 | 9.22123E−20 | METTL5 | 0.94072163 | 1.11103E−06 | MTMR2 | 0.42761013 | 0.00034363 | WDR41 | 0.2861897 | 0.007050569 |
| TTYH3 | 1.5114995 | 1.0032E−19 | HKR1 | 0.7488349 | 1.12731E−06 | FIP1L1 | 0.5333452 | 0.00034363 | MED28 | 0.52836674 | 0.007069166 |
| NAPRT1 | 1.8237065 | 1.03303E−19 | APC | 0.507304 | 1.15123E−06 | RAB20 | 1.2821954 | 0.000343818 | MRPL18 | 0.6046123 | 0.007072562 |
| ZNF330 | 1.6783754 | 1.05325E−19 | PMS2P4 | 0.74826443 | 1.15463E−06 | MIA3 | 0.3240851 | 0.000344436 | PSMD14 | 0.66235507 | 0.007086468 |
| MAZ | 1.2879483 | 1.23706E−19 | IPO13 | 1.0531936 | 1.17216E−06 | DHX15 | 0.67005146 | 0.000344481 | FAM188A | 0.7025145 | 0.007088687 |
| UBL3 | 1.5054482 | 1.26975E−19 | NSUN5P1 | 0.5397838 | 1.18006E−06 | SLC37A4 | 0.9197862 | 0.000345145 | ABRACL | 0.41363445 | 0.007125225 |
| ITPKB | 1.4863359 | 1.28497E−19 | TCERG1 | 0.6402951 | 1.18737E−06 | C10orf88 | 0.94381136 | 0.000346487 | MRPL14 | 0.69251966 | 0.007144155 |
| OBSCN | 1.3561561 | 1.42201E−19 | CD2BP2 | 0.6441987 | 1.18864E−06 | SNX4 | 0.5860267 | 0.000346866 | ACP6 | 0.50688154 | 0.007157149 |
| EIF3D | 1.4930276 | 1.64457E−19 | LRRC28 | 0.9347186 | 1.19279E−06 | RCBTB2 | 0.7578273 | 0.000346866 | NISCH | 0.300869 | 0.007161781 |
| PPM1H | 1.5763603 | 1.89889E−19 | LINC00439 | 0.91660565 | 1.19825E−06 | LOC100287042 | 0.21516034 | 0.000346983 | CEP250 | 0.27431062 | 0.007167305 |
| GABARAP | 1.1681566 | 2.30935E−19 | SRD5A3 | 0.8073066 | 1.20859E−06 | BCAS4 | 1.1503575 | 0.000348623 | SMARCA1 | 0.23932846 | 0.007212796 |
| TSPAN10 | 2.0655854 | 2.34272E−19 | SRGAP1 | 0.52846813 | 1.21868E−06 | YEATS4 | 0.6542485 | 0.000351 | VTI1A | 0.20274065 | 0.007216256 |
| RSL1D1 | 1.056359 | 3.20589E−19 | METTL10 | 0.8185635 | 1.22854E−06 | LOC100506123 | 0.15890647 | 0.00035251 | ZSCAN2 | 0.23695923 | 0.007220923 |
| EEF2 | 1.3253703 | 3.24917E−19 | PLP1 | 0.899951 | 1.24615E−06 | FAR2 | 0.7556751 | 0.000352629 | PIBF1 | 0.3146033 | 0.007259295 |
| C1orf85 | 1.5456499 | 3.69244E−19 | EIF2B3 | 0.9340949 | 1.26365E−06 | DR1 | 0.50384605 | 0.000352868 | ZNF91 | 0.04671344 | 0.007283254 |
| HADHB | 1.6258421 | 3.88477E−19 | FAM212A | 1.7868636 | 1.26804E−06 | CDC5L | 0.40549812 | 0.000352868 | PPM1D | 0.103459746 | 0.007291611 |
| RPL10A | 0.8788381 | 4.29414E−19 | TSN | 0.80555516 | 1.27008E−06 | CCSAP | 0.16925389 | 0.000353575 | ZNF85 | 0.48609388 | 0.007319486 |
| QARS | 1.509548 | 4.59396E−19 | MRPS14 | 0.74247223 | 1.27725E−06 | PANK1 | 0.17686978 | 0.000354948 | CDK2AP2 | 0.5876367 | 0.007326658 |
| UBAP2L | 1.3846632 | 4.59396E−19 | FAM21B | 0.7066753 | 1.28067E−06 | MTPAP | 0.3796141 | 0.00035603 | KIF2C | 0.8788732 | 0.007330168 |
| CLCN5 | 1.1128309 | 4.59404E−19 | GYG2 | 0.9827586 | 1.28305E−06 | RNF25 | 0.81213284 | 0.000356373 | ZNF227 | 0.59474367 | 0.007344695 |
| FTL | 0.9564364 | 4.86043E−19 | HCFC1 | 0.578353 | 1.28305E−06 | CXXC1 | 0.5849063 | 0.00035907 | INTS8 | 0.28328133 | 0.007346988 |
| ZFP106 | 1.183511 | 4.94978E−19 | DCUN1D1 | 0.6728958 | 1.28305E−06 | BLOC1S6 | 0.4386639 | 0.000361411 | TRIM2 | 0.29690006 | 0.007357707 |
| RPL26 | 1.0417194 | 6.2418E−19 | SIN3B | 0.5872754 | 1.29619E−06 | GOLGA7 | 0.6692014 | 0.000362361 | WRAP53 | 0.80663574 | 0.007357707 |
| SMIM15 | 1.38306 | 8.27957E−19 | RANBP10 | 0.74442285 | 1.29653E−06 | RHOT2 | 0.6286453 | 0.000367183 | KIF13A | 0.3036649 | 0.007372127 |
| MSTO1 | 1.513 | 8.97468E−19 | SPDYE8P | 0.59990525 | 1.30384E−06 | DUSP22 | 0.68164444 | 0.000367383 | VPS54 | 0.54438794 | 0.00738042 |
| DLL3 | 2.0907133 | 1.2357E−18 | PUF60 | 1.0509573 | 1.31505E−06 | LSM10 | 0.7586721 | 0.000367507 | ZNF567 | 0.6196563 | 0.007382724 |
| LIMD2 | 0.97843176 | 1.54473E−18 | RANGRF | 1.0093777 | 1.3161E−06 | IRF2BP2 | 0.34615344 | 0.00036809 | ANKRD26 | 0.041834265 | 0.007391185 |
| PAICS | 1.567536 | 1.62064E−18 | TXNDC17 | 0.71406794 | 1.33098E−06 | SLC24A4 | 0.5008729 | 0.000368903 | HERC2P10 | 0.715052 | 0.007424353 |
| RAB6B | 1.8389091 | 1.82217E−18 | PAF1 | 0.7466867 | 1.33098E−06 | PKD1P1 | 0.27556732 | 0.000369104 | SLC36A1 | 0.5032635 | 0.007430224 |
| RPL41 | 0.57405716 | 1.9125E−18 | PTOV1-AS1 | 0.25718078 | 1.33098E−06 | MEF2D | 0.45614338 | 0.00037115 | MIIP | 0.42570496 | 0.007430224 |
| RPS12 | 0.70726454 | 1.93574E−18 | UBE2C | 1.1398761 | 1.37518E−06 | PYCR2 | 0.4514688 | 0.00037143 | PARP3 | 0.46010366 | 0.007444775 |
| CECR5 | 1.6564658 | 1.95692E−18 | BPNT1 | 0.5492736 | 1.38219E−06 | SRRD | 0.7993262 | 0.000371555 | IDI1 | 0.50106984 | 0.007445858 |
| RPS15 | 1.0349993 | 2.24153E−18 | RASA3 | 0.7025863 | 1.40079E−06 | STARD13 | 0.4903098 | 0.000371603 | FBXL12 | 0.3772849 | 0.007464322 |
| P2RX7 | 1.3709153 | 2.37422E−18 | E2F3 | 1.0764371 | 1.40566E−06 | ZNF559 | 0.77877593 | 0.000371883 | ATG16L1 | 0.4557678 | 0.007465251 |
| FXYD5 | 1.8134081 | 2.71378E−18 | ITGAE | 0.48180944 | 1.40603E−06 | CROCCP2 | 0.61557865 | 0.00037472 | WDPCP | 0.09144125 | 0.007465251 |
| DDX39B | 1.3009882 | 3.16581E−18 | SNRPA1 | 0.878689 | 1.41811E−06 | EPB41 | 0.40511537 | 0.000375236 | EID2 | 0.3996524 | 0.00746758 |
| RGS12 | 1.2998537 | 3.38502E−18 | VAPB | 0.46441582 | 1.43334E−06 | RCL1 | 0.7778932 | 0.000376503 | C9orf123 | 0.2738171 | 0.007488585 |
| RPS15A | 0.8634042 | 4.52317E−18 | RMND5A | 0.48463634 | 1.44256E−06 | NCKAP5L | 0.46784478 | 0.000377412 | IGFBP4 | 0.9236694 | 0.007489675 |
| FAM126A | 1.3625453 | 4.81557E−18 | GPN1 | 0.9880409 | 1.44449E−06 | NOL10 | 0.46862972 | 0.00037848 | PTCHD4 | 0.21340607 | 0.007491387 |
| WDR81 | 1.6037195 | 4.82935E−18 | SNAPC3 | 0.44266272 | 1.4468E−06 | CNOT2 | 0.47604057 | 0.000378922 | BTRC | 0.46077564 | 0.007491387 |
| RUVBL2 | 1.732086 | 6.71515E−18 | TMEM254 | 0.87878823 | 1.45144E−06 | DDA1 | 0.18657659 | 0.000380073 | COL11A2 | 0.6317049 | 0.007501211 |
| SRCAP | 1.054528 | 7.01473E−18 | C17orf58 | 0.97980607 | 1.46273E−06 | UBOX5 | 0.37601882 | 0.000381277 | RHOBTB1 | 0.89451325 | 0.007504801 |
| EEF1G | 0.98925585 | 7.01473E−18 | HOXA5 | 1.756779 | 1.47252E−06 | C12orf52 | 0.73452157 | 0.000381722 | C10orf35 | 1.4844519 | 0.007562131 |
| MAP1LC3A | 1.4533904 | 8.18112E−18 | HECTD3 | 0.89151484 | 1.49111E−06 | DNM1L | 0.34754226 | 0.000383277 | METTL12 | 0.2934937 | 0.007578337 |
| ALDH1B1 | 1.2310492 | 8.20542E−18 | GHITM | 0.9761845 | 1.49191E−06 | FADS1 | 0.41829127 | 0.000384839 | TMEM115 | 0.51395243 | 0.00762873 |
| PPP5C | 1.3325665 | 8.65E−18 | EXOSC3 | 0.8334782 | 1.4935E−06 | PSEN2 | 0.65163666 | 0.000385368 | HSPBAP1 | 0.4400104 | 0.007632374 |
| EDNRB | 1.6154726 | 8.65E−18 | PRPF4 | 0.68518066 | 1.50711E−06 | TRMT10B | 0.14929157 | 0.000388243 | KPNB1 | 0.31573495 | 0.007638242 |
| RPL17 | 0.7992215 | 8.93744E−18 | DHX33 | 0.32636 | 1.50711E−06 | CHFR | 0.67166907 | 0.000388776 | ROBO3 | 0.509544 | 0.007638242 |
| CDKN2A | 2.2412555 | 9.44422E−18 | SLC39A1 | 0.972211 | 1.50751E−06 | PI15 | 1.0874262 | 0.000389632 | GPX7 | 0.73985445 | 0.007639353 |
| NHEJ1 | 1.4838881 | 9.96609E−18 | STEAP1 | 1.3782257 | 1.518E−06 | HMGN4 | 0.67200905 | 0.000391483 | AP3S1 | 0.62785363 | 0.007651896 |
| SNHG15 | 1.7390565 | 1.05213E−17 | ZSWIM8 | 0.7744544 | 1.53346E−06 | GPKOW | 0.14085211 | 0.000391483 | C4orf27 | 0.4045331 | 0.007677038 |
| WDR12 | 1.563391 | 1.1705E−17 | GALNT1 | 0.37940863 | 1.54577E−06 | CRNDE | 0.6087535 | 0.000394542 | SPIN4 | 1.344299 | 0.007697302 |
| AEN | 1.5470423 | 1.26076E−17 | NDUFS1 | 0.7256497 | 1.54866E−06 | MED4 | 0.50579685 | 0.000396393 | RNF20 | 0.3524182 | 0.007704813 |
| SLC45A2 | 1.9899346 | 1.35426E−17 | SLC25A32 | 0.6226493 | 1.54866E−06 | SNORD76 | 1.7552718 | 0.000396855 | MORC2-AS1 | 0.40157902 | 0.007709772 |
| RPL35 | 0.700817 | 1.47426E−17 | CDCA7L | 1.3275464 | 1.5732E−06 | SLC50A1 | 0.46254453 | 0.000397071 | RIMKLA | 0.0614346 | 0.007726267 |
| C1orf43 | 1.3137906 | 1.65275E−17 | POPDC3 | 0.9345238 | 1.5732E−06 | TRMT1 | 0.613224 | 0.00040042 | NDUFS4 | 0.5983366 | 0.007738781 |
| NDUFS2 | 1.5983849 | 1.80773E−17 | CBLL1 | 0.7692527 | 1.58794E−06 | PHYH | 0.87392116 | 0.000400886 | UBAC2 | 0.43823394 | 0.007742316 |
| TYRP1 | 2.845604 | 1.87425E−17 | DSG2 | 0.60733837 | 1.61522E−06 | AP1G2 | 0.41902015 | 0.000401104 | LOC96610 | 0.17425191 | 0.007767889 |
| RPS18 | 0.8839341 | 2.17602E−17 | ABT1 | 0.84835225 | 1.62601E−06 | SPESP1 | 1.2338417 | 0.000401405 | ELAVL1 | 0.30673462 | 0.007770307 |
| RPL36 | 1.1909131 | 2.18501E−17 | DIP2A | 0.5288197 | 1.63034E−06 | CD320 | 0.8444137 | 0.000406044 | TEAD1 | 0.27122697 | 0.007776595 |
| SLC25A3 | 1.0551683 | 2.48672E−17 | DDX59 | 0.818538 | 1.64122E−06 | USP54 | 0.3003058 | 0.000409126 | GORASP1 | 0.45578244 | 0.007788052 |
| VANGL2 | 1.7326217 | 2.77738E−17 | TTC4 | 0.8862285 | 1.6456E−06 | CC2D1B | 0.32394102 | 0.000410364 | USP8 | 0.26951352 | 0.007805674 |
| PDCD11 | 1.169518 | 2.96188E−17 | PAM | 1.4138454 | 1.65042E−06 | RPS10-NUDT3 | 0.2554101 | 0.00041271 | POLR3GL | 0.535306 | 0.007805674 |
| DAP3 | 1.4289392 | 2.96188E−17 | MCM6 | 0.9472533 | 1.65042E−06 | RBM23 | 0.6380763 | 0.000413787 | LOC645513 | 0.31119037 | 0.007818466 |
| EEF1A1 | 0.6683643 | 3.24373E−17 | ULK3 | 0.9132138 | 1.65131E−06 | PRR14L | 0.2841871 | 0.000413926 | SPATC1L | 0.6414215 | 0.0078209 |
| UCK2 | 1.1164464 | 3.29652E−17 | GTPBP3 | 0.4552511 | 1.6641E−06 | HNRNPA3 | 0.4639915 | 0.000418098 | SCLT1 | 0.576642 | 0.007833715 |
| RPS10 | 0.89578134 | 3.57813E−17 | PPP1R12B | 0.48106357 | 1.6641E−06 | POLR2M | 0.42836976 | 0.000418584 | KLHL18 | 0.60137135 | 0.007853053 |
| SSR2 | 1.4435607 | 3.58063E−17 | EMC6 | 0.93105114 | 1.6672E−06 | ZNF431 | 0.04533551 | 0.000420888 | RBM19 | 0.25127822 | 0.00786331 |
| HNRNPC | 1.0212173 | 3.84705E−17 | ASB9 | 1.2500592 | 1.67565E−06 | TRAPPC6B | 0.44891477 | 0.000421116 | FAM105A | 0.11372442 | 0.007889086 |
| HMG20A | 1.2937242 | 4.07879E−17 | CCDC74A | 2.432246 | 1.70258E−06 | C14orf142 | 0.72633046 | 0.000422562 | NRSN2 | 0.36881897 | 0.007889086 |
| RPL12 | 0.76266694 | 4.2507E−17 | IL11RA | 1.0629244 | 1.72489E−06 | CTBS | 0.5393204 | 0.000422965 | BRE | 0.4716875 | 0.007894155 |
| NOP2 | 1.5333383 | 4.45328E−17 | SHQ1 | 0.7377462 | 1.72581E−06 | FAM168A | 0.3965966 | 0.000423282 | C16orf95 | 0.9504902 | 0.007907079 |
| RCHY1 | 1.1738905 | 4.50802E−17 | NOP10 | 1.0325469 | 1.72628E−06 | PARL | 0.7530777 | 0.000423948 | TBC1D9B | 0.3144213 | 0.007930521 |
| GALNT2 | 1.2891084 | 4.53545E−17 | LOC285033 | 0.5741945 | 1.72904E−06 | XXYLT1 | 0.7332362 | 0.000424091 | PHIP | 0.2817581 | 0.00794087 |
| MYO5A | 1.0525825 | 5.60283E−17 | MAU2 | 0.5817113 | 1.73549E−06 | NUP93 | 0.6154864 | 0.000424234 | TTC21A | 0.5966715 | 0.007947284 |
| RPL37A | 0.7095295 | 6.50544E−17 | FAM32A | 1.0489243 | 1.73549E−06 | TSSC4 | 0.67031246 | 0.000425865 | SLC2A10 | 0.2510277 | 0.007956337 |
| RQCD1 | 1.2714312 | 6.50544E−17 | MAML1 | 0.9283 | 1.7503E−06 | CLN3 | 0.622367 | 0.000426622 | TROAP | 0.9761756 | 0.007973312 |
| RPL31 | 0.7664481 | 8.16678E−17 | GTF2E1 | 0.41509926 | 1.76478E−06 | LOC152217 | 0.7327521 | 0.000426678 | TCF19 | 0.8907029 | 0.007984872 |
| TSTA3 | 1.5219445 | 8.20601E−17 | ARHGAP35 | 0.64612305 | 1.769E−06 | LGI4 | 0.93725 | 0.000430463 | ALG2 | 0.3973048 | 0.008009848 |
| GPATCH4 | 1.3619831 | 8.29612E−17 | RRAGC | 0.45230532 | 1.77984E−06 | MARS2 | 0.9951995 | 0.000432473 | GZMM | 1.6400149 | 0.008022945 |
| YY1AP1 | 1.3085543 | 8.38367E−17 | UBXN2A | 0.6756499 | 1.77986E−06 | FABP3 | 1.0275667 | 0.000435801 | TLE1 | 0.35464302 | 0.008026765 |
| SLC25A44 | 1.4407662 | 8.72368E−17 | UBE2D4 | 0.5211867 | 1.79218E−06 | PRKCSH | 0.46196604 | 0.00043912 | PROSER2-AS1 | 0.08800444 | 0.008030587 |
| TTC39A | 2.011597 | 9.45364E−17 | DZIP1 | 0.4777356 | 1.82624E−06 | TIMM21 | 0.7126104 | 0.00043912 | RWDD3 | 0.5364209 | 0.00803441 |
| THEM4 | 0.89594173 | 9.45364E−17 | ZNF581 | 1.1099159 | 1.83304E−06 | OXLD1 | 0.6714737 | 0.000439991 | CHMP1A | 0.412526 | 0.008052868 |
| KIRREL | 1.4129268 | 9.81966E−17 | KIF11 | 0.3545916 | 1.83403E−06 | ZNF611 | 0.43123353 | 0.000441407 | RPS14 | 0.24858405 | 0.008072375 |
| HAGHL | 1.8041626 | 1.13228E−16 | SUGT1 | 0.76870966 | 1.83403E−06 | MLH1 | 0.6581514 | 0.000441646 | SLC18A2 | 1.1701008 | 0.008131969 |
| CDC42EP1 | 1.7771724 | 1.36495E−16 | MRPS9 | 1.0260855 | 1.83648E−06 | TPST1 | 0.3866375 | 0.000442612 | ATP5S | 0.21592708 | 0.008131969 |
| SRP14 | 1.1384493 | 1.52071E−16 | ABHD14A | 0.9617355 | 1.83844E−06 | MGST2 | 0.8745988 | 0.000443307 | TUB | 0.7697337 | 0.00816782 |
| NOLC1 | 1.2316265 | 1.80756E−16 | SMAP1 | 0.557768 | 1.8404E−06 | GMNN | 0.81020176 | 0.000445922 | ZNF644 | 0.27926868 | 0.008216338 |
| TUBB | 1.290004 | 1.81637E−16 | MAPK1IP1L | 0.5404446 | 1.84334E−06 | NAPEPLD | 0.32466224 | 0.000446714 | TSR2 | 0.5163226 | 0.008220243 |
| DDX21 | 1.0263484 | 2.00897E−16 | COX20 | 0.6828383 | 1.84924E−06 | NFRKB | 0.37162492 | 0.000449717 | NCS1 | 0.6099383 | 0.008256778 |
| HNRNPUL1 | 0.8908143 | 2.18592E−16 | SAFB2 | 0.67178166 | 1.85661E−06 | RASGRP4 | 0.047128662 | 0.00044996 | DHRS7B | 0.526581 | 0.008271608 |
| FAM53B | 1.461902 | 2.21281E−16 | DROSHA | 0.4619051 | 1.86352E−06 | MRPL10 | 0.65700024 | 0.000450574 | KBTBD2 | 0.192836 | 0.008280997 |
| PYGL | 1.713895 | 2.23139E−16 | SGK3 | 0.6817369 | 1.86403E−06 | COMMD8 | 0.7526949 | 0.000451281 | CTNNBL1 | 0.4595672 | 0.008299804 |
| CCT4 | 1.5418782 | 2.34115E−16 | WDR82 | 0.44634813 | 1.8695E−06 | EEF2K | 0.353525 | 0.00045236 | CMTM6 | 0.35439393 | 0.008302376 |
| ENOSF1 | 0.8759226 | 2.49354E−16 | TRMT6 | 1.0004864 | 1.8695E−06 | SNORD80 | 1.5403174 | 0.000452605 | TDP1 | 0.4363875 | 0.008304949 |
| RPL37 | 0.77000725 | 2.62281E−16 | NMRAL1 | 0.82854164 | 1.88692E−06 | C19orf35 | 0.00668466 | 0.000452757 | SLC9A3R1 | 0.59453166 | 0.008308892 |
| COX6B1 | 1.3487247 | 2.67349E−16 | RPE | 0.7699075 | 1.88894E−06 | CASP6 | 0.3305506 | 0.000453374 | NUSAP1 | 0.8389899 | 0.008330669 |
| CCNB1IP1 | 1.6603248 | 2.82535E−16 | SERPINH1 | 0.91603196 | 1.89747E−06 | HOXC9 | 0.7701778 | 0.000454831 | RBM28 | 0.28029338 | 0.008335997 |
| FBL | 1.5909128 | 2.90479E−16 | MPRIP | 0.2329079 | 1.89799E−06 | ARFRP1 | 0.53229797 | 0.000455264 | SNX33 | 0.66708684 | 0.008363183 |
| GNB2L1 | 0.93074524 | 2.96221E−16 | RWDD1 | 0.83821756 | 1.89853E−06 | USPL1 | 0.6060736 | 0.000455697 | NCAPH2 | 0.40462884 | 0.008386472 |
| CPSF1 | 1.0793723 | 3.00655E−16 | MED19 | 0.762599 | 1.89853E−06 | PTRH1 | 0.77751553 | 0.000458569 | RNF219 | 0.36361837 | 0.008401513 |
| TSR1 | 1.1277523 | 3.10975E−16 | VPS28 | 1.0960836 | 1.90867E−06 | MED25 | 0.480834 | 0.000458596 | LOC285000 | 0.9960435 | 0.008423511 |
| SETDB1 | 1.2613009 | 3.21191E−16 | USP39 | 0.8833163 | 1.91122E−06 | PRKAR2A | 0.5950257 | 0.000459187 | TXLNG | 0.38846552 | 0.008424731 |
| PEPD | 1.5689926 | 3.35507E−16 | C2orf88 | 0.9629919 | 1.91478E−06 | FBXO9 | 0.11918597 | 0.000460189 | EXTL2 | 0.33647785 | 0.008430115 |
| ISYNA1 | 1.4431341 | 3.5575E−16 | LRRN4CL | 0.70641935 | 1.94387E−06 | ALK | 1.326808 | 0.000462235 | LINC00476 | 0.11019325 | 0.00843689 |
| COPA | 1.1672865 | 3.9639E−16 | PAR-SN | 0.6635598 | 1.96248E−06 | ITFG2 | 0.53526425 | 0.00046365 | HSD17B7P2 | 0.06218642 | 0.008452014 |
| PQBP1 | 1.518507 | 3.98019E−16 | RMDN3 | 0.7824585 | 1.96509E−06 | FAM63A | 0.55124414 | 0.00046365 | ATF7 | 0.32414228 | 0.00845463 |
| ATP5B | 1.2859535 | 4.58635E−16 | ANKFY1 | 0.6162422 | 1.96824E−06 | MAD2L1 | 0.91318685 | 0.00046365 | CSRP2 | 0.7767704 | 0.008506094 |
| ZBED3-AS1 | 2.9992902 | 4.60174E−16 | ZNF480 | 0.33336303 | 1.96826E−06 | LAMTOR3 | 0.39185825 | 0.000464092 | IFFO1 | 0.332573 | 0.008526939 |
| RPL30 | 0.731641 | 4.60543E−16 | AKR7A2 | 0.7608214 | 1.97819E−06 | BRMS1 | 0.64199775 | 0.000464819 | HERC1 | 0.13174498 | 0.008543445 |
| POLR1D | 1.6579156 | 4.69052E−16 | STK24 | 0.7062291 | 1.99658E−06 | LRRC47 | 0.3243687 | 0.000466658 | SLC26A6 | 0.48022354 | 0.008544682 |
| LYPLA1 | 1.3693916 | 5.1758E−16 | AP1S2 | 0.85103244 | 2.01249E−06 | RASSF1 | 0.5905814 | 0.000469403 | DYM | 0.32209972 | 0.008571247 |
| LZTS2 | 1.4199854 | 5.18451E−16 | ANGEL2 | 0.4447336 | 2.0205E−06 | PLEKHB2 | 0.52097917 | 0.000469753 | SCN8A | 1.167821 | 0.008578126 |
| PLEKHJ1 | 1.5947064 | 6.14649E−16 | TTC19 | 0.5343576 | 2.03551E−06 | LTB4R | 0.27623186 | 0.000470296 | ETS2 | 0.75950074 | 0.008580777 |
| LMCD1 | 1.814228 | 6.79743E−16 | PIK3R3 | 0.87167007 | 2.04361E−06 | RNF141 | 0.34969166 | 0.000471287 | MOSPD3 | 0.48793942 | 0.008590149 |
| ALDH18A1 | 1.4167343 | 7.16467E−16 | PDLIM7 | 0.7750068 | 2.06313E−06 | RRM2 | 0.64707214 | 0.000471446 | TRIM46 | 1.4834161 | 0.008602696 |
| WASH1 | 1.1225293 | 7.17082E−16 | ZNF880 | 0.847789 | 2.06588E−06 | CERKL | 1.4933789 | 0.000473732 | SRSF3 | 0.60792655 | 0.008613843 |
| RPL13 | 0.6524236 | 7.24126E−16 | IDE | 0.778427 | 2.06809E−06 | PRIM2 | 0.5888931 | 0.000474183 | SGK196 | 0.46824598 | 0.00861509 |
| MPZL1 | 1.2751448 | 7.30316E−16 | RNMTL1 | 1.0676414 | 2.07468E−06 | SIRT3 | 0.23297942 | 0.000474343 | PRKY | 0.26448646 | 0.008616337 |
| GATAD2A | 0.94666564 | 8.13677E−16 | LTBP4 | 0.79352623 | 2.08467E−06 | HSBP1L1 | 0.6849293 | 0.000474405 | EAF1 | 0.08670827 | 0.008651627 |
| WASH2P | 0.92259306 | 8.3082E−16 | UVRAG | 0.83091235 | 2.08467E−06 | TMEM141 | 0.55579215 | 0.000474856 | ZBTB17 | 0.46693176 | 0.008668355 |
| RNF2 | 1.1170307 | 8.3082E−16 | SNORA70 | 1.3354545 | 2.08467E−06 | SRPRB | 0.5838069 | 0.000476281 | MRPL17 | 0.39317513 | 0.008690818 |
| BCCIP | 1.4148864 | 8.76002E−16 | PRR14 | 0.9659545 | 2.1018E−06 | RBMX | 0.8348355 | 0.000477221 | ASH1L-AS1 | 0.3052053 | 0.008696358 |
| RPL36A | 0.77876544 | 9.02007E−16 | KIF3A | 0.43323952 | 2.10184E−06 | SIPA1L3 | 0.8099122 | 0.00047787 | FOXJ3 | 0.26001114 | 0.008746115 |
| CCT2 | 1.572516 | 1.01449E−15 | GTF2H2D | 0.78650266 | 2.10742E−06 | TUBA3FP | 0.20073445 | 0.000478912 | TCEB1 | 0.38437423 | 0.008751687 |
| CDH3 | 1.7702986 | 1.2097E−15 | EFCAB2 | 0.3970619 | 2.11134E−06 | PLXNA1 | 0.40087044 | 0.000479171 | PUS3 | 0.7260218 | 0.008757262 |
| UBA52 | 1.0153921 | 1.4588E−15 | PABPC4 | 0.6857123 | 2.11364E−06 | CHCHD4 | 0.81158864 | 0.000479528 | GMPPA | 0.40530446 | 0.00876284 |
| SUCLG2 | 1.377502 | 1.52536E−15 | KCNJ13 | 0.8905148 | 2.11479E−06 | SDHD | 0.73395133 | 0.00047995 | TNFAIP2 | 0.59878474 | 0.008772738 |
| GPR89A | 1.3854978 | 1.78154E−15 | PAK1IP1 | 0.86004627 | 2.13497E−06 | NAA16 | 0.34601605 | 0.000481059 | 44447 | 0.2688253 | 0.008784086 |
| KLHL9 | 1.3846745 | 1.81995E−15 | DYNC1I2 | 0.46050766 | 2.1412E−06 | PITPNB | 0.6168243 | 0.000481059 | VRK1 | 0.66155136 | 0.00878968 |
| VDAC2 | 1.5908885 | 1.81995E−15 | TP53I3 | 1.2180699 | 2.15254E−06 | NDUFAF7 | 0.29320282 | 0.000481418 | EGFL7 | 0.9325981 | 0.008800709 |
| HSPA2 | 1.9735177 | 1.93571E−15 | GTF3A | 1.1013631 | 2.15542E−06 | AQP6 | 0.07942233 | 0.00048194 | SLC2A1 | 0.6313298 | 0.008803426 |
| SLC19A1 | 1.319492 | 1.99103E−15 | RNF44 | 0.744441 | 2.17712E−06 | COX19 | 0.20646116 | 0.00048194 | APBB3 | 0.21942706 | 0.008809032 |
| AFG3L2 | 1.1712917 | 1.9997E−15 | TMEM183B | 0.8292074 | 2.18347E−06 | POLG2 | 0.56936723 | 0.000482201 | PPAN | 0.62314653 | 0.008820254 |
| LINC00662 | 0.87215143 | 2.09941E−15 | ZMAT3 | 0.38994518 | 2.18466E−06 | LYRM2 | 0.29450616 | 0.000485824 | ACADS | 0.49775895 | 0.008820254 |
| ILVBL | 1.537774 | 2.28936E−15 | NOC4L | 0.9553158 | 2.20027E−06 | C16orf80 | 0.7264173 | 0.000487479 | MAN2A1 | 0.6128026 | 0.008830042 |
| PRCC | 0.94656545 | 2.37343E−15 | AASDH | 0.91800606 | 2.22358E−06 | KIF20B | 0.58716375 | 0.000487643 | TUT1 | 0.44526803 | 0.008844358 |
| ARHGAP4 | 1.8118917 | 2.40479E−15 | ATIC | 1.0467994 | 2.23182E−06 | COQ3 | 1.1438069 | 0.000489404 | SS18L2 | 0.46694 | 0.008849819 |
| CINP | 1.5988017 | 2.47858E−15 | THUMPD3 | 0.53001887 | 2.23421E−06 | DGCR6L | 0.78556544 | 0.000489669 | ARMCX5-GPRASP2 | 0.79172957 | 0.008849819 |
| MICAL1 | 1.4025055 | 2.66881E−15 | WIPF3 | 0.38166207 | 2.26262E−06 | TIMMDC1 | 0.7180603 | 0.000490534 | RRM2B | 0.384404 | 0.008855453 |
| GALT | 1.4345373 | 2.68836E−15 | COPS8 | 0.888317 | 2.26564E−06 | MAST2 | 0.39818636 | 0.0004907 | DDX20 | 0.5185594 | 0.008875621 |
| SCAMP3 | 1.4112239 | 2.71937E−15 | MEF2BNB | 0.68957233 | 2.26866E−06 | TUBA1A | 0.8371746 | 0.000490765 | CFL1P1 | 0.10808803 | 0.008881883 |
| WDR6 | 1.1881061 | 2.76567E−15 | CSNK2A1 | 0.7253748 | 2.31324E−06 | KIAA0408 | 0.16613673 | 0.000494884 | NUP188 | 0.17207049 | 0.008881883 |
| SETDB2 | 1.0980233 | 2.98293E−15 | MTX2 | 0.9058461 | 2.31693E−06 | TMEM167A | 0.4106645 | 0.000494884 | PPA2 | 0.282831 | 0.008881883 |
| FES | 1.8897954 | 3.05019E−15 | BTF3L4 | 0.6320372 | 2.32917E−06 | NFATC3 | 0.49719593 | 0.00049596 | PIK3R2 | 0.1552584 | 0.008976405 |
| C22orf29 | 0.98278546 | 3.24673E−15 | URI1 | 0.68540955 | 2.37183E−06 | SLC7A6 | 0.18595928 | 0.000496228 | PUM2 | 0.17114612 | 0.008980643 |
| SOX4 | 1.1470406 | 3.24673E−15 | MYO19 | 0.6496899 | 2.37873E−06 | TECPR1 | 0.17508098 | 0.00049812 | C20orf194 | 0.24020064 | 0.008983413 |
| CTSK | 2.041855 | 3.31029E−15 | PPDPF | 0.50043344 | 2.39505E−06 | XPO4 | 0.3998855 | 0.000498389 | IPO7 | 0.3155493 | 0.009005319 |
| PHB2 | 1.4575746 | 3.32368E−15 | KLHDC3 | 0.9458614 | 2.3995E−06 | NAA20 | 0.8402332 | 0.000499066 | LIN28A | 0.15555757 | 0.009010874 |
| ZNF146 | 1.2274264 | 3.49149E−15 | NR2F2 | 0.74256516 | 2.40711E−06 | MX2 | 0.7664948 | 0.000500932 | WDYHV1 | 0.5497803 | 0.009041709 |
| CHCHD6 | 1.3479503 | 3.80384E−15 | C16orf89 | 1.3040226 | 2.41348E−06 | RBMS1 | 0.45799002 | 0.000500932 | TRMT11 | 0.3229072 | 0.009050244 |
| NDUFA3 | 1.4878757 | 3.87343E−15 | DNMT1 | 0.55641764 | 2.4205E−06 | PAIP1 | 0.52765954 | 0.000501305 | RNF169 | 0.18973596 | 0.009050244 |
| IDH3A | 1.3319203 | 4.00185E−15 | ZDHHC11 | 1.7516104 | 2.45634E−06 | CDK7 | 0.660013 | 0.000501372 | SECISBP2 | 0.20980114 | 0.009058958 |
| TRUB2 | 1.5774869 | 4.03814E−15 | MGC2752 | 0.3850359 | 2.48296E−06 | SPRYD4 | 0.3013643 | 0.000505575 | EDEM2 | 0.38585576 | 0.009064714 |
| POLR3C | 1.4168736 | 4.44806E−15 | MCRS1 | 0.78555596 | 2.51179E−06 | SWSAP1 | 0.24463995 | 0.000505607 | C14orf93 | 0.75608027 | 0.009066027 |
| C11orf24 | 1.4325162 | 4.5745E−15 | EBNA1BP2 | 0.9895262 | 2.51184E−06 | ACADVL | 0.53947157 | 0.000505607 | DCXR | 0.64766896 | 0.009073271 |
| IPO9 | 0.8573903 | 4.86244E−15 | LENG8 | 0.5154038 | 2.52707E−06 | PCBD1 | 0.63793683 | 0.000505777 | TTPAL | 0.08695073 | 0.009116039 |
| ZFAS1 | 1.350079 | 4.94923E−15 | COX7A2L | 0.9444463 | 2.54976E−06 | RFX7 | 0.34320402 | 0.000506361 | ZNF585A | 0.4270422 | 0.009132268 |
| ZCCHC9 | 1.20679 | 5.11083E−15 | ZNF273 | 0.19209838 | 2.54976E−06 | UBE2V2 | 0.6921199 | 0.000506428 | SDHB | 0.80446863 | 0.00915899 |
| TRIM28 | 1.1553061 | 5.76871E−15 | APOA1BP | 0.92311496 | 2.57125E−06 | DNM1P35 | 0.16119091 | 0.000507529 | THEM6 | 0.5909266 | 0.009160315 |
| TROVE2 | 1.3540554 | 5.8402E−15 | CIC | 0.3879853 | 2.57131E−06 | SF3A3 | 0.7073162 | 0.000508424 | NME7 | 0.40498093 | 0.009187112 |
| RPS28 | 0.91268826 | 5.9885E−15 | RANBP1 | 1.0042049 | 2.59371E−06 | ZNF276 | 0.0679182 | 0.00050856 | SLMAP | 0.28430438 | 0.009203456 |
| HNRNPF | 1.3926666 | 6.70499E−15 | LIPA | 0.92422086 | 2.60737E−06 | LOC202181 | 0.57787246 | 0.00050856 | LINC00641 | 0.054318633 | 0.009207793 |
| SLC5A4 | 2.7282388 | 6.82722E−15 | GINS1 | 0.7579302 | 2.61289E−06 | CDK5RAP1 | 0.6184623 | 0.000509526 | PTPN3 | 0.069576286 | 0.009211961 |
| PIP4K2A | 1.1515472 | 7.10455E−15 | ESRG | 0.5191969 | 2.615E−06 | IP6K1 | 0.5561456 | 0.000511602 | PCED1A | 0.36500117 | 0.009211961 |
| GNPDA1 | 1.4051727 | 8.96227E−15 | TYSND1 | 0.77825886 | 2.6219E−06 | ZNF79 | 1.2092481 | 0.000511602 | NDUFB7 | 0.77684796 | 0.009225162 |
| RPL14 | 0.7238382 | 9.11761E−15 | SETMAR | 0.42689416 | 2.67665E−06 | ANAPC1 | 0.26006824 | 0.000511602 | PKMYT1 | 0.9603803 | 0.009225162 |
| MTHFD1 | 1.341473 | 1.17479E−14 | FBXW4 | 1.1287831 | 2.68932E−06 | LINC00620 | 0.005335482 | 0.000512782 | LSM5 | 0.25992298 | 0.009226326 |
| LSM14A | 1.0176215 | 1.22315E−14 | FAM114A2 | 0.8622862 | 2.6943E−06 | XRRA1 | 0.54732627 | 0.000512782 | MAPK7 | 0.38358217 | 0.009226326 |
| LOC388796 | 1.3138323 | 1.23619E−14 | ATP5G3 | 0.6464931 | 2.69718E−06 | FXYD7 | 2.4876373 | 0.000513478 | BRF1 | 0.23016356 | 0.009250107 |
| SUPT5H | 1.1231506 | 1.37636E−14 | PIAS4 | 0.99675536 | 2.71987E−06 | PFDN4 | 0.45586744 | 0.000519431 | RNPEPL1 | 0.5156864 | 0.009266553 |
| LOC100132247 | 1.1697716 | 1.55359E−14 | LIMA1 | 0.8235228 | 2.72136E−06 | NSUN2 | 0.43812504 | 0.000521617 | HACL1 | 0.5260313 | 0.009269407 |
| HADHA | 1.2895776 | 1.63379E−14 | COG8 | 0.756809 | 2.72854E−06 | ZBTB1 | 0.65102774 | 0.000522643 | LOC283143 | 1.2639449 | 0.009294976 |
| SPG20 | 1.0663084 | 1.78981E−14 | RAB3GAP2 | 0.41722214 | 2.73788E−06 | CCDC114 | 0.04172406 | 0.000526836 | FAM134A | 0.13553762 | 0.009308459 |
| NIP7 | 1.0986999 | 1.80447E−14 | ATAD3A | 0.63658196 | 2.74009E−06 | PI4KAP1 | 0.3418755 | 0.000529697 | NDUFS3 | 0.68874794 | 0.009326176 |
| SNHG5 | 1.271492 | 1.87683E−14 | LOC100288778 | 0.46660173 | 2.74373E−06 | CCDC50 | 0.54000413 | 0.00053063 | POLD3 | 0.55660224 | 0.009350192 |
| KIAA1211 | 1.3563745 | 1.92696E−14 | PDHA1 | 0.7204795 | 2.74595E−06 | PHF14 | 0.25622153 | 0.000531672 | SULT1C4 | 0.91695166 | 0.009368327 |
| HIST3H2A | 1.4275537 | 2.03602E−14 | ZNF780A | 0.49148998 | 2.74745E−06 | OPRK1 | 0.36931866 | 0.000532679 | VASH1 | 0.28864396 | 0.009372737 |
| FAM45A | 1.4727566 | 2.19609E−14 | HHLA2 | 0.18906672 | 2.74896E−06 | MGA | 0.29673725 | 0.000535244 | CD302 | 0.4618089 | 0.009380034 |
| METTL13 | 1.2686888 | 2.19719E−14 | CERS6 | 1.014715 | 2.76485E−06 | GAB1 | 0.33932865 | 0.000538476 | MYO1E | 0.2267066 | 0.009381392 |
| VPS26A | 1.2133503 | 2.27775E−14 | TADA1 | 0.91588783 | 2.77359E−06 | CCT8 | 0.80513275 | 0.000544812 | MBNL2 | 0.23942158 | 0.009382749 |
| ISG20L2 | 1.1388953 | 2.42418E−14 | RABL2B | 0.6511992 | 2.82254E−06 | SLC25A24 | 0.4172801 | 0.0005454 | CXorf23 | 0.20542032 | 0.00939941 |
| HDGFRP3 | 1.4686797 | 2.55994E−14 | VWA9 | 0.78082997 | 2.86037E−06 | PLSCR3 | 0.7290042 | 0.000546543 | LOC100507377 | 1.5160795 | 0.009406728 |
| FGD1 | 1.3200495 | 2.60706E−14 | KPNA2 | 1.1166344 | 2.87389E−06 | HSDL1 | 0.44959813 | 0.000546543 | SLC25A30 | 0.4675783 | 0.009406728 |
| TIMM17B | 1.3104908 | 2.68729E−14 | TP53BP2 | 0.56201494 | 2.89349E−06 | MCU | 1.1395843 | 0.000547171 | LMAN2L | 0.586989 | 0.00941729 |
| SMNDC1 | 1.108973 | 3.10191E−14 | STRAP | 1.035244 | 2.90338E−06 | KIAA1324L | 0.43474507 | 0.000547688 | YTHDC2 | 0.24076223 | 0.009437079 |
| POLR2D | 1.2861328 | 3.28954E−14 | SELENBP1 | 1.1444612 | 2.91558E−06 | MRPL32 | 0.73799217 | 0.000548502 | ZDHHC8 | 0.6218027 | 0.009439983 |
| GBAS | 1.311973 | 3.39185E−14 | NFX1 | 0.59688926 | 2.94234E−06 | ZNF226 | 0.6016518 | 0.000550431 | RPAP3 | 0.3723926 | 0.009442887 |
| RPLP1 | 0.48468754 | 3.70703E−14 | SDAD1 | 0.6755492 | 3.00742E−06 | P4HA1 | 0.5941399 | 0.000552815 | ARL2BP | 0.46468002 | 0.009490152 |
| EEF1B2 | 1.0300751 | 4.13214E−14 | ZGPAT | 0.9005166 | 3.0075E−06 | FLJ30403 | 0.076414764 | 0.000556747 | POGLUT1 | 0.41617545 | 0.009499081 |
| ADCK3 | 1.4114106 | 4.78089E−14 | CES2 | 0.5297328 | 3.01934E−06 | CNOT10 | 0.42404786 | 0.000558817 | ZNF142 | 0.3135123 | 0.009537631 |
| ARHGEF1 | 1.052401 | 5.18266E−14 | PTBP2 | 0.34338874 | 3.02807E−06 | MTL5 | 0.2634997 | 0.000558817 | EFHD2 | 0.3824045 | 0.009548155 |
| CCNG1 | 1.4901537 | 5.18373E−14 | ERLIN1 | 0.91442287 | 3.03366E−06 | LOC644961 | 0.26939267 | 0.000560592 | PTOV1 | 0.21246007 | 0.009548155 |
| SMCR7L | 1.0728886 | 5.445E−14 | MICU1 | 0.77334833 | 3.04243E−06 | BAG5 | 0.21846032 | 0.000561576 | FOXK2 | 0.18248834 | 0.009568194 |
| FABP5 | 1.5463823 | 5.52036E−14 | USB1 | 0.8217596 | 3.05917E−06 | PCDHGA12 | 0.97478694 | 0.000561765 | HCN3 | 1.0515953 | 0.009597459 |
| PGAM4 | 1.7590581 | 5.58565E−14 | MRPL34 | 0.924084 | 3.1162E−06 | HAUS1 | 0.62605923 | 0.000561954 | ATAD2 | 0.86518645 | 0.009618983 |
| DDX50 | 1.1838949 | 6.17052E−14 | EPB41L4A-AS1 | 0.81253654 | 3.1171E−06 | UBAP1 | 0.3939296 | 0.000564423 | RAB14 | 0.18523893 | 0.009665478 |
| TTLL4 | 1.0199727 | 6.63689E−14 | DHX30 | 0.6741269 | 3.13098E−06 | MAGOH | 0.6055626 | 0.000567591 | ATM | 0.14183833 | 0.009665478 |
| SPG21 | 1.439938 | 6.86665E−14 | CYB5B | 0.5192946 | 3.14736E−06 | AMACR | 0.5854045 | 0.000572625 | CPNE7 | 1.3961015 | 0.009701141 |
| DCTN5 | 0.84385085 | 6.86665E−14 | CLIP3 | 1.1541024 | 3.16055E−06 | PUS7 | 0.40516877 | 0.000572934 | AGAP1 | 0.32639116 | 0.009701141 |
| CALM3 | 1.0031812 | 7.01142E−14 | LIN54 | 0.37557274 | 3.17379E−06 | DAZAP1 | 0.35965264 | 0.000574596 | TCFL5 | 1.3917886 | 0.009714985 |
| ACAT1 | 1.3174812 | 8.14079E−14 | HUS1 | 0.569781 | 3.19371E−06 | C5orf63 | 1.5347631 | 0.000574596 | PITPNM1 | 0.6922695 | 0.009714985 |
| TARS2 | 1.0372641 | 8.30877E−14 | GBAP1 | 0.7537686 | 3.22043E−06 | BRCA2 | 0.7687033 | 0.000574905 | MYH14 | 0.82599574 | 0.009724282 |
| PRMT7 | 1.3274492 | 8.55761E−14 | PABPC3 | 1.1982741 | 3.25157E−06 | CDH6 | 0.23210056 | 0.000575215 | ZFYVE9 | 0.81998885 | 0.00974624 |
| TIMM22 | 1.4730712 | 8.69628E−14 | CCS | 0.9085861 | 3.2975E−06 | BTG3 | 0.6235347 | 0.000577389 | TRIB3 | 0.68889713 | 0.009769654 |
| LDB3 | 1.338086 | 8.71591E−14 | DPF2 | 0.804873 | 3.30273E−06 | RAB3A | 1.0124253 | 0.0005777 | MED11 | 0.69932187 | 0.009809024 |
| MTAP | 0.9792609 | 8.96271E−14 | VLDLR | 1.0009634 | 3.30282E−06 | DZIP3 | 0.1495203 | 0.000580469 | STXBP3 | 0.3002447 | 0.009827791 |
| INPP4B | 1.4055736 | 9.55701E−14 | YWHAB | 0.6581668 | 3.3175E−06 | TEAD2 | 0.8525269 | 0.000582152 | C1orf159 | 0.291875 | 0.009827791 |
| BFAR | 1.0918003 | 9.81183E−14 | HEATR1 | 0.57390994 | 3.31759E−06 | ZNF576 | 0.86627924 | 0.000582152 | PTPDC1 | 0.8210554 | 0.009830805 |
| TBRG4 | 1.420308 | 1.05226E−13 | CCDC171 | 1.1928911 | 3.34271E−06 | APOO | 0.6712684 | 0.000583407 | ZNF497 | 0.17205568 | 0.009835417 |
| MIR4461 | 2.0905893 | 1.05226E−13 | PNRC2 | 0.86714995 | 3.34541E−06 | FKBP1A | 0.6113673 | 0.000587702 | ZNF605 | 0.30300575 | 0.009852632 |
| DNMT3A | 1.136066 | 1.09432E−13 | EMC2 | 0.88329214 | 3.36288E−06 | SNAPC2 | 0.66075486 | 0.000588494 | ARHGEF2 | 0.17520398 | 0.009852632 |
| SRM | 1.3238746 | 1.16759E−13 | C20orf112 | 0.6381485 | 3.39018E−06 | ADNP | 0.3851375 | 0.000593143 | THRAP3 | 0.41801405 | 0.009855654 |
| CCDC144C | 1.647167 | 1.20101E−13 | RAB3B | 0.28600165 | 3.39018E−06 | CSTF3 | 0.61518776 | 0.000596342 | LIG3 | 0.30979434 | 0.009871476 |
| TRIM63 | 1.435717 | 1.21516E−13 | B3GALTL | 0.82029176 | 3.39995E−06 | NSUN4 | 0.16369814 | 0.000597868 | RP9 | 0.2731365 | 0.00988091 |
| SIGMAR1 | 1.4369969 | 1.22901E−13 | MPP7 | 0.34981984 | 3.43988E−06 | CLNS1A | 0.7662812 | 0.000600122 | EXOSC10 | 0.4754374 | 0.009882339 |
| CRISPLD1 | 1.5016665 | 1.23668E−13 | MAGI2-AS3 | 0.54828846 | 3.44712E−06 | TRIO | 0.2531412 | 0.000601737 | CHSY1 | 0.08574306 | 0.009907657 |
| RPL7A | 0.7562907 | 1.32505E−13 | ARSB | 0.4409806 | 3.46152E−06 | MYL9 | 0.8114853 | 0.000601737 | STAU1 | 0.44215533 | 0.009931424 |
| MET | 1.1024193 | 1.5155E−13 | PHF20L1 | 0.6978129 | 3.46252E−06 | STK25 | 0.53407615 | 0.000601778 | TAF6 | 0.23702362 | 0.00993769 |
| AVL9 | 1.2158407 | 1.5155E−13 | SH3PXD2B | 0.7759415 | 3.46532E−06 | CD79B | 1.434826 | 0.000602345 | BAG3 | 0.3509046 | 0.00995847 |
| NARS2 | 1.3565582 | 1.61585E−13 | SUPV3L1 | 0.87632227 | 3.50689E−06 | PRPF8 | 0.45013514 | 0.000603276 | A1BG | 0.24439114 | 0.009995117 |
| SRSF7 | 1.1524453 | 1.67102E−13 | PXK | 0.9626854 | 3.56274E−06 | TOR2A | 0.9618009 | 0.000606731 | MLL | 0.1788541 | 0.009996562 |
| PFDN2 | 1.3878196 | 1.70194E−13 | FLYWCH1 | 0.6931465 | 3.57299E−06 | RUFY1 | 0.7164784 | 0.000607628 | TBL1X | 0.55809337 | 0.010056453 |
| GRWD1 | 1.1301931 | 1.83508E−13 | SNAP47 | 0.82358724 | 3.61394E−06 | SNRNP70 | 0.46110234 | 0.000607628 | WDR90 | 0.42143413 | 0.010057905 |
| TMEM204 | 2.5623136 | 1.90134E−13 | TCEAL8 | 0.9057985 | 3.64792E−06 | RPSAP9 | 0.16507256 | 0.00060865 | LOC148709 | 0.14686416 | 0.010067501 |
| MMS19 | 1.2712047 | 2.1487E−13 | SOGA1 | 0.6068656 | 3.65087E−06 | SAMD4B | 0.3510365 | 0.00060865 | RPAP2 | 0.4433215 | 0.01009506 |
| RPL10 | 0.6862957 | 2.20881E−13 | UTS2D | 0.28415313 | 3.67083E−06 | C15orf38 | 0.4406837 | 0.000609959 | LRP6 | 0.1949832 | 0.010111054 |
| MRPL37 | 1.4458843 | 2.20881E−13 | KCTD21 | 0.7880379 | 3.67758E−06 | ADRBK1 | 0.39976233 | 0.000611394 | AMOT | 0.7368322 | 0.010148564 |
| PYCARD | 1.9805158 | 2.44937E−13 | YWHAE | 0.6265214 | 3.68816E−06 | HARS | 0.66523874 | 0.00061382 | HSD17B11 | 0.527755 | 0.010263694 |
| DARS | 1.3457128 | 2.47613E−13 | ARHGAP39 | 0.81022894 | 3.69018E−06 | CCDC167 | 0.66566426 | 0.000614521 | FSTL1 | 0.5616551 | 0.01028177 |
| SHMT2 | 1.4637899 | 2.47999E−13 | SHOC2 | 0.71654576 | 3.69328E−06 | OSTF1 | 0.6023767 | 0.000615347 | NDOR1 | 0.19395809 | 0.010284912 |
| LAMTOR1 | 1.3180462 | 2.57415E−13 | NAT9 | 0.85643095 | 3.69328E−06 | QTRT1 | 0.6052252 | 0.000616422 | PPARD | 0.12421812 | 0.010286394 |
| R3HCC1L | 1.0281934 | 2.60836E−13 | C2orf47 | 0.90792704 | 3.6934E−06 | GUCD1 | 0.28899822 | 0.000618204 | LINC00518 | 0.41208917 | 0.010357888 |
| TRIM27 | 1.1938953 | 2.62057E−13 | GNPNAT1 | 0.48868588 | 3.70593E−06 | INO80E | 0.40437758 | 0.000618204 | GUSBP4 | 0.5926107 | 0.010367381 |
| MTG1 | 1.1716692 | 2.7658E−13 | SEC31A | 0.7278607 | 3.70988E−06 | NKAP | 0.55295974 | 0.000618536 | SLC43A1 | 0.64879245 | 0.010377064 |
| CTPS1 | 1.3868127 | 2.84853E−13 | AIFM2 | 0.32654983 | 3.73786E−06 | RIOK3 | 0.4197477 | 0.000623325 | RABL6 | 0.100814156 | 0.010383403 |
| TSNAX | 1.1143714 | 2.87411E−13 | CCDC86 | 0.8407825 | 3.75828E−06 | HTRA3 | 2.6834059 | 0.000624162 | FAM89A | 0.7984667 | 0.010394959 |
| RPS27A | 0.5578743 | 2.94748E−13 | NGRN | 0.96516013 | 3.7915E−06 | SNHG9 | 0.5847338 | 0.000624623 | MPDZ | 0.31236872 | 0.010398134 |
| UPF3A | 0.9421027 | 3.07354E−13 | FAM104B | 0.79664534 | 3.80632E−06 | TMEM39B | 0.6241325 | 0.000626134 | H2AFZ | 0.5239368 | 0.010408025 |
| RRS1 | 1.4065427 | 3.36875E−13 | PRR5 | 0.8559108 | 3.83315E−06 | VN1R2 | 0.20298067 | 0.000626134 | TRNT1 | 0.2815352 | 0.010411204 |
| BAX | 1.1980873 | 3.36875E−13 | ZNF512 | 0.81656414 | 3.83315E−06 | IFT52 | 0.72713643 | 0.000628363 | DEF6 | 1.2588936 | 0.010419426 |
| MYBBP1A | 1.1957812 | 3.37285E−13 | NACA | 0.44641134 | 3.83427E−06 | RPL19 | 0.25610694 | 0.000630979 | RBM12B-AS1 | 0.82533777 | 0.01043775 |
| ARPC5 | 1.1977361 | 3.4216E−13 | ZNF552 | 0.97463644 | 3.84727E−06 | C14orf178 | 0.17089638 | 0.000631064 | PPIL6 | 0.022994198 | 0.010460984 |
| SHKBP1 | 1.3790375 | 3.54715E−13 | XRCC1 | 0.8597 | 3.85931E−06 | HBS1L | 0.3698019 | 0.000632928 | NOSTRIN | 2.1322265 | 0.010502873 |
| TMEM55A | 1.2339615 | 3.54715E−13 | GYG1 | 0.8793216 | 3.87838E−06 | ZC3HAV1L | 0.21833487 | 0.00063365 | BCL2A1 | 0.7711934 | 0.010512674 |
| RPP25 | 1.5398225 | 4.01346E−13 | SLC41A1 | 0.7406373 | 3.90558E−06 | FAM76A | 0.121934935 | 0.00063501 | NDUFB9 | 0.74729836 | 0.010527754 |
| RPL39 | 0.81406575 | 4.23765E−13 | TOMM40 | 0.7382927 | 3.91578E−06 | ETV1 | 0.5274356 | 0.000638805 | PDCL | 0.19132674 | 0.010534362 |
| FASTKD2 | 0.86837184 | 4.63162E−13 | HELLS | 1.2317309 | 3.92717E−06 | TCN2 | 0.9275128 | 0.00064172 | GEMIN5 | 0.44903147 | 0.010569886 |
| RPA1 | 1.165819 | 4.76415E−13 | SURF6 | 0.81779456 | 3.9659E−06 | ZNF527 | 0.088197716 | 0.000641935 | TAF9 | 0.7369624 | 0.010590156 |
| SYNCRIP | 1.0627056 | 4.76415E−13 | TSC2 | 0.5554264 | 3.96706E−06 | FLYWCH2 | 0.56398803 | 0.000643312 | TMEM48 | 0.4617238 | 0.0105968 |
| IL6R | 0.99434173 | 4.91886E−13 | WDR26 | 0.45333967 | 3.9805E−06 | ADA | 1.2653879 | 0.000645815 | NAPG | 0.24456057 | 0.010601739 |
| TFIP11 | 1.2226466 | 4.94205E−13 | RBM5 | 0.67043555 | 4.02388E−06 | ANKRD30BL | 0.26359355 | 0.000645815 | RBM48 | 0.21722932 | 0.010623776 |
| WAPAL | 0.8466762 | 5.06931E−13 | RIOK1 | 0.79828805 | 4.04804E−06 | ABCD3 | 0.42086586 | 0.000646292 | SIMC1 | 0.61511445 | 0.010640713 |
| MRPL44 | 1.1467471 | 5.37819E−13 | PITRM1 | 0.6750491 | 4.04923E−06 | COQ5 | 0.71393687 | 0.000647028 | ZNF26 | 0.3030568 | 0.010652529 |
| GNL3L | 0.6860849 | 5.57448E−13 | NDUFA13 | 0.9662969 | 4.05355E−06 | RAI14 | 0.30835962 | 0.000647334 | NUF2 | 0.91362584 | 0.010678097 |
| TIMM17A | 1.3576809 | 5.57448E−13 | ZNF672 | 0.8856769 | 4.06936E−06 | STRN3 | 0.32660967 | 0.000647334 | OSBPL8 | 0.21831208 | 0.010681352 |
| NR2C1 | 0.9863706 | 5.91818E−13 | KDELR1 | 0.7897617 | 4.08523E−06 | EDEM3 | 0.2659896 | 0.000651459 | NOP14 | 0.45494312 | 0.010691306 |
| ATP5D | 1.3450178 | 6.03087E−13 | KPNA3 | 0.80895776 | 4.10432E−06 | KTI12 | 0.72216845 | 0.000652986 | CDCA8 | 0.86649096 | 0.01072195 |
| UROS | 1.354453 | 6.31718E−13 | STON2 | 0.17793255 | 4.18018E−06 | STRADA | 0.39403275 | 0.000653075 | ZDBF2 | 0.19544959 | 0.010754587 |
| RAC3 | 1.5460919 | 6.37862E−13 | KLHL6 | 0.12089547 | 4.24632E−06 | GEMIN8 | 0.41175082 | 0.000656445 | YJEFN3 | 0.86714524 | 0.010768249 |
| TOP1MT | 1.315769 | 6.54483E−13 | JRK | 0.42300054 | 4.30241E−06 | CAMSAP2 | 0.20650434 | 0.000656539 | MTO1 | 0.52575094 | 0.010823425 |
| SPDYE5 | 0.7952095 | 6.64392E−13 | ADK | 0.75806534 | 4.30478E−06 | PRRC1 | 0.34568545 | 0.000656539 | ARHGDIB | 0.8273995 | 0.010865067 |
| MORF4L1 | 1.0750368 | 6.76513E−13 | WDR35 | 0.7633365 | 4.31394E−06 | SEC24B-AS1 | 0.02064959 | 0.00065676 | MKS1 | 0.47735745 | 0.010881982 |
| TPP2 | 0.7528328 | 6.85969E−13 | RRP1 | 0.7742724 | 4.33407E−06 | HGS | 0.6357611 | 0.000657771 | UBE2Q1 | 0.3574173 | 0.010894041 |
| MAP3K13 | 0.9080094 | 7.26848E−13 | TMEM53 | 0.42591876 | 4.34425E−06 | TADA2B | 0.18485047 | 0.000658388 | CUL3 | 0.0415813 | 0.010928721 |
| MRPS21 | 1.0948908 | 7.34479E−13 | H1F0 | 0.77426183 | 4.34553E−06 | CORO2B | 0.9172796 | 0.000661217 | MBD1 | 0.31890878 | 0.010937314 |
| RINL | 0.64563555 | 7.35154E−13 | TMEM251 | 0.9609085 | 4.35462E−06 | TPMT | 0.34101796 | 0.000661217 | OTUD6B | 0.4112379 | 0.010968783 |
| RPL22 | 0.8590559 | 7.35553E−13 | PCCB | 0.91032946 | 4.41435E−06 | LLPH | 0.4149434 | 0.000661217 | STIP1 | 0.5885242 | 0.01097388 |
| EXOSC4 | 1.4915665 | 7.44416E−13 | FAM168B | 0.54376227 | 4.42925E−06 | SMA4 | 0.18563639 | 0.000661217 | EFCAB5 | 0.31563884 | 0.010980743 |
| TPM3 | 0.73510337 | 7.58991E−13 | AARS2 | 0.42979077 | 4.43965E−06 | TOMM40L | 0.7587198 | 0.0006625 | SULT1C2 | 0.3745289 | 0.011001726 |
| MCC | 0.8903962 | 7.93704E−13 | ZNF586 | 0.9771987 | 4.44324E−06 | ZNF543 | 0.39626107 | 0.00066352 | ZMPSTE24 | 0.35498518 | 0.011020605 |
| FUS | 0.84302646 | 8.67069E−13 | SSBP4 | 0.938901 | 4.50529E−06 | HIATL1 | 0.391139 | 0.000664675 | ESRRA | 0.3380711 | 0.011038115 |
| RBM12 | 0.78305966 | 8.79504E−13 | PART1 | 0.07003537 | 4.51951E−06 | NUDT16 | 0.5849266 | 0.000665698 | RACGAP1 | 0.6978217 | 0.011043243 |
| EIF3A | 0.98789513 | 9.53758E−13 | POLR1B | 0.6257989 | 4.52432E−06 | SRPK1 | 0.5151228 | 0.000667081 | RBM17 | 0.28436044 | 0.011158568 |
| MRPL12 | 1.3351558 | 9.66686E−13 | MOSPD1 | 0.8981193 | 4.5303E−06 | C17orf70 | 0.56450963 | 0.000669537 | PDCD6 | 0.65643734 | 0.011187027 |
| SDCBP | 0.9757397 | 9.67971E−13 | MSH6 | 0.61123204 | 4.59235E−06 | LNX1 | 1.7073323 | 0.000671508 | SPRYD3 | 0.4928015 | 0.011210163 |
| MTHFS | 1.0947375 | 9.80352E−13 | ARHGEF7 | 0.46170244 | 4.66237E−06 | C3orf38 | 0.56420004 | 0.000672137 | SRR | 0.6686637 | 0.011247745 |
| SEC24C | 1.0695899 | 1.01189E−12 | LOC100132832 | 0.47289723 | 4.70086E−06 | GBA2 | 0.5803765 | 0.000678037 | SERPING1 | 0.22835302 | 0.011272608 |
| MPDU1 | 1.2215967 | 1.02444E−12 | DECR2 | 0.9003342 | 4.75665E−06 | C11orf49 | 0.58263355 | 0.000678129 | ZNF280A | 1.4008563 | 0.01127964 |
| NOP16 | 1.4096624 | 1.08824E−12 | MGRN1 | 0.6157669 | 4.84886E−06 | MZT2A | 0.62755543 | 0.000679851 | GSS | 0.48102236 | 0.011284869 |
| GTF3C6 | 1.3458478 | 1.09923E−12 | POLR3B | 1.0089229 | 4.87387E−06 | 44444 | 0.921164 | 0.000680488 | RAB24 | 0.4283194 | 0.011380612 |
| GNL2 | 1.2359158 | 1.10782E−12 | ANKRD12 | 0.44122544 | 4.90778E−06 | NRIP1 | 0.42256594 | 0.000682625 | ZBTB24 | 0.31399122 | 0.011380612 |
| RPAIN | 1.0782744 | 1.14903E−12 | C10orf2 | 1.4540108 | 4.94483E−06 | FSTL5 | 1.1896893 | 0.000684495 | MCOLN2 | 0.102089055 | 0.011407181 |
| SMG5 | 0.9317683 | 1.19175E−12 | LOC440354 | 0.634729 | 4.94503E−06 | MRGPRX3 | 1.5194985 | 0.00069022 | LDHB | 0.4932459 | 0.011428901 |
| EML4 | 0.84526545 | 1.21017E−12 | ZNF692 | 0.7769663 | 4.9756E−06 | WDHD1 | 0.94696057 | 0.000691971 | AJUBA | 0.025009895 | 0.011472646 |
| IPO4 | 1.2227603 | 1.30745E−12 | TARBP1 | 0.80352825 | 4.97962E−06 | PPIAL4G | 0.37872314 | 0.000693588 | INTS4L2 | 0.53190315 | 0.011474094 |
| CD81 | 1.155306 | 1.31815E−12 | IREB2 | 0.3459148 | 4.99636E−06 | YAF2 | 0.1758422 | 0.000694792 | PASK | 0.824477 | 0.011474094 |
| BCL7A | 1.2746671 | 1.40025E−12 | PTBP1 | 0.7468022 | 5.00932E−06 | DGKE | 0.1313114 | 0.000695953 | ST13 | 0.43872714 | 0.011492255 |
| TBC1D16 | 0.905673 | 1.45162E−12 | TCF12 | 0.6921262 | 5.0738E−06 | TGIF2 | 0.46148172 | 0.000696048 | CC2D2A | 0.19549362 | 0.011506765 |
| MLLT1 | 0.99241436 | 1.50824E−12 | TSPAN4 | 0.8570219 | 5.09085E−06 | CISH | 1.5046462 | 0.000697535 | THAP3 | 0.45027292 | 0.01151945 |
| UFC1 | 1.2976983 | 1.52843E−12 | MTHFR | 0.3168047 | 5.09106E−06 | IAH1 | 0.70454603 | 0.000702432 | DFFB | 0.12331568 | 0.01153399 |
| C16orf88 | 1.0062616 | 1.52997E−12 | RNF123 | 0.51037264 | 5.09907E−06 | KIAA1467 | 0.2649164 | 0.000702528 | EPOR | 0.38011664 | 0.011537097 |
| ETS1 | 0.95009446 | 1.66606E−12 | FAM178A | 0.651569 | 5.09928E−06 | CIDEB | 1.2301711 | 0.000703186 | RAD54L2 | 0.28970832 | 0.011537097 |
| YTHDF3 | 0.9689488 | 1.69936E−12 | C15orf61 | 0.6848766 | 5.1583E−06 | RNF113A | 0.7131878 | 0.000703422 | MPI | 0.395439 | 0.011537097 |
| RPL9 | 0.56865984 | 1.73528E−12 | SFR1 | 0.57847446 | 5.16377E−06 | PHB | 0.7475877 | 0.000705486 | SMIM11 | 0.20618159 | 0.011557195 |
| RPS21 | 0.88488775 | 1.80008E−12 | SMIM7 | 0.7803652 | 5.16531E−06 | TMED5 | 0.32857022 | 0.000705583 | TUBA8 | 1.9519501 | 0.011558848 |
| BPHL | 1.1764526 | 1.94221E−12 | SC5DL | 0.4395683 | 5.19852E−06 | MRO | 0.031589583 | 0.000712325 | KIAA1524 | 0.6652751 | 0.011567506 |
| TUBGCP2 | 1.1246095 | 1.96028E−12 | LOC100128881 | 1.4427041 | 5.20537E−06 | DUSP11 | 0.5865925 | 0.000720041 | STK17A | 0.57508 | 0.011567506 |
| STT3A | 1.2056005 | 1.99022E−12 | KCNN2 | 0.6427513 | 5.20957E−06 | USP49 | 0.20788677 | 0.000721288 | BTN2A3P | 0.11215308 | 0.011567506 |
| NR2F6 | 1.5018033 | 2.01552E−12 | BIVM | 0.36638013 | 5.28056E−06 | LRSAM1 | 0.3577259 | 0.000724267 | SMTN | 0.3335768 | 0.011574707 |
| UQCRC2 | 1.3602647 | 2.35845E−12 | BTAF1 | 0.497391 | 5.28213E−06 | MRS2 | 0.42177907 | 0.000725665 | NASP | 0.44329658 | 0.011576364 |
| ITPA | 1.3047011 | 2.36266E−12 | ELMO2 | 0.6844158 | 5.33639E−06 | HIP1R | 0.66856927 | 0.000726777 | EML2 | 0.49151906 | 0.011598375 |
| NSL1 | 0.63896537 | 2.85387E−12 | STAG3L4 | 0.72554106 | 5.41317E−06 | ZNF250 | 0.2086239 | 0.00072818 | DERL1 | 0.4310649 | 0.011601888 |
| DEDD | 1.0523423 | 2.96399E−12 | RALB | 0.8393528 | 5.47871E−06 | TRIM37 | 0.43020576 | 0.00072857 | MLF1IP | 0.598261 | 0.011616332 |
| NCBP1 | 0.8706994 | 2.9881E−12 | MMS22L | 0.31119147 | 5.49568E−06 | MRPS17 | 0.6219527 | 0.000730558 | DHRSX | 0.38821077 | 0.01164027 |
| MAST3 | 0.7512601 | 3.0057E−12 | DHRS2 | 1.4311553 | 5.52953E−06 | PTPLAD1 | 0.6993687 | 0.000730657 | HINT2 | 0.46188897 | 0.011643794 |
| MMACHC | 0.7529287 | 3.05163E−12 | RPL23AP82 | 0.4728719 | 5.565E−06 | LZTR1 | 0.6791365 | 0.000731048 | MANBA | 0.40402636 | 0.011658478 |
| TRAPPC1 | 1.0085956 | 3.21073E−12 | ZNF140 | 0.9434969 | 5.57798E−06 | SLC39A11 | 0.50845176 | 0.000731877 | NRN1 | 0.48704594 | 0.01166573 |
| USE1 | 1.2823396 | 3.42721E−12 | RASIP1 | 1.0985986 | 5.63566E−06 | EZH2 | 0.602679 | 0.000733983 | ASB13 | 0.496502 | 0.011676712 |
| EIF1AX | 0.91859 | 3.45506E−12 | SUPT7L | 0.49065933 | 5.63566E−06 | ANP32A | 0.27371508 | 0.000733983 | TRAF3 | 1.0001625 | 0.011685648 |
| APH1A | 1.1147891 | 3.76057E−12 | LACTB2 | 0.83776546 | 5.63566E−06 | ITGB1BP1 | 0.6132862 | 0.000733983 | CEP170B | 0.57593536 | 0.011702055 |
| CRYL1 | 1.4882585 | 3.83202E−12 | YTHDC1 | 0.44138208 | 5.6445E−06 | PHKG1 | 0.27900985 | 0.0007354 | ZNFX1 | 0.020585971 | 0.011702055 |
| LOC728190 | 0.96179926 | 3.90193E−12 | RNASEH2B | 0.83624345 | 5.68094E−06 | PLAC4 | 0.48142943 | 0.00073902 | RHPN2 | 0.104518674 | 0.011705598 |
| PEX19 | 1.1270479 | 3.93652E−12 | FLJ36000 | 0.9964887 | 5.68119E−06 | SENP6 | 0.16715324 | 0.00074409 | DDB2 | 0.3831405 | 0.011720353 |
| MKI67IP | 1.2288995 | 3.95384E−12 | ARID3B | 1.1693662 | 5.70456E−06 | NARG2 | 0.094081536 | 0.000745331 | PLEKHG4 | 0.3959418 | 0.011729511 |
| C19orf48 | 1.4437742 | 4.04088E−12 | SACS | 0.6398291 | 5.72682E−06 | KRI1 | 0.2855907 | 0.000745331 | IGF2BP1 | 0.6421138 | 0.011742421 |
| FKBP4 | 1.101085 | 4.09484E−12 | NOL12 | 0.40987128 | 5.73872E−06 | SMG9 | 0.3250579 | 0.000746768 | AGO1 | 0.22594221 | 0.011760967 |
| WNK3 | 1.0595698 | 4.12049E−12 | MARS | 0.79754454 | 5.78574E−06 | GPCPD1 | 0.45422596 | 0.000747019 | RSBN1L-AS1 | 0.20666015 | 0.011783105 |
| ALDH3A2 | 0.8910249 | 4.19098E−12 | RABEPK | 0.980062 | 5.78601E−06 | HDAC7 | 0.5604612 | 0.000747418 | MLLT11 | 0.47413656 | 0.011786671 |
| C20orf27 | 1.0895972 | 4.67988E−12 | GAREML | 0.4043077 | 5.90032E−06 | LENG1 | 0.72050834 | 0.000749753 | CALM1 | 0.4702296 | 0.011801518 |
| VPS72 | 1.2275904 | 4.84637E−12 | ZFYVE19 | 0.89215285 | 5.90658E−06 | FAM120C | 0.4791311 | 0.000749856 | RPL34 | 0.31833744 | 0.011829379 |
| GYS1 | 1.0667409 | 4.8965E−12 | C1QTNF6 | 0.52781814 | 5.93234E−06 | KRR1 | 0.38478944 | 0.000750956 | SLC6A15 | 0.29844055 | 0.011829379 |
| MDH2 | 1.3007907 | 4.90902E−12 | DSCR3 | 0.62384194 | 5.97333E−06 | SLC5A10 | 1.125068 | 0.000750956 | RFX3 | 0.733297 | 0.011851825 |
| SNRNP40 | 1.3301884 | 5.00201E−12 | MAN2A2 | 0.5960157 | 6.039E−06 | PTPRG-AS1 | 0.15341914 | 0.000751657 | FAM120A | 0.24116568 | 0.011857299 |
| NUP62 | 1.0500511 | 5.43165E−12 | PTPRF | 0.9804748 | 6.04846E−06 | CNTROB | 0.5100922 | 0.000757456 | SLC46A1 | 0.53463495 | 0.011874118 |
| NCL | 0.77347755 | 5.43556E−12 | ADHFE1 | 1.0376794 | 6.05487E−06 | ZNF181 | 0.73468906 | 0.000758764 | ACAD9 | 0.33994755 | 0.011885279 |
| SH3PXD2A | 0.9654832 | 5.68481E−12 | CNOT8 | 0.73879224 | 6.05975E−06 | LOC100652772 | 0.15943061 | 0.000759019 | RAB12 | 0.3434541 | 0.011898344 |
| TMEM70 | 1.1710474 | 5.76044E−12 | PPARGC1B | 0.88485986 | 6.07539E−06 | YTHDF1 | 0.52545285 | 0.000759877 | HOGA1 | 0.013422023 | 0.011934201 |
| HMOX2 | 1.2673781 | 5.84566E−12 | HSPA12A | 0.45139694 | 6.09261E−06 | BMS1 | 0.22451584 | 0.00076119 | KIAA1161 | 0.16424115 | 0.011935717 |
| GGH | 1.4715025 | 5.89089E−12 | AP2S1 | 0.9314967 | 6.13311E−06 | CES1 | 2.7452967 | 0.000761597 | MORC3 | 0.17205653 | 0.011935717 |
| RNF167 | 1.0459962 | 6.00883E−12 | STXBP1 | 0.83604455 | 6.14427E−06 | ECHDC2 | 0.6835452 | 0.000767925 | TSHZ1 | 0.3769148 | 0.011944838 |
| RUSC1 | 1.2399246 | 6.18785E−12 | GOT1 | 0.9727548 | 6.16324E−06 | SYT14L | 2.0845568 | 0.000768183 | ANKRD30B | 1.2310939 | 0.011969387 |
| CHD1L | 1.1011697 | 6.47337E−12 | DDX54 | 0.73061967 | 6.17914E−06 | CHCHD10 | 0.97906536 | 0.000769967 | ZNF337 | 0.31447408 | 0.011982536 |
| TFCP2 | 1.3344347 | 6.49718E−12 | HNRNPM | 0.95668256 | 6.24857E−06 | TRAPPC12 | 0.57966155 | 0.000770073 | L3HYPDH | 0.39129147 | 0.012024359 |
| DDX39A | 1.4453456 | 6.54503E−12 | NUFIP1 | 0.87198424 | 6.29833E−06 | ZDHHC16 | 0.779902 | 0.000770331 | TICAM1 | 0.72666174 | 0.012035456 |
| FTSJ3 | 1.0946895 | 6.69549E−12 | MRPL38 | 0.7779241 | 6.30341E−06 | TSNARE1 | 0.80086994 | 0.000771355 | TMEM189-UBE2V1 | 0.62810767 | 0.012035456 |
| PGAM1 | 1.4706194 | 7.281E−12 | JMJD4 | 0.93852216 | 6.32445E−06 | MGEA5 | 0.2733803 | 0.000772533 | 44256 | 1.8517138 | 0.012054229 |
| MPHOSPH10 | 1.0711896 | 7.51933E−12 | UGP2 | 0.8753959 | 6.35037E−06 | NANOS1 | 0.22280657 | 0.000778174 | OGFOD2 | 0.34467828 | 0.012084359 |
| HRNR | 1.2240399 | 7.7256E−12 | FLJ31306 | 0.43575442 | 6.38477E−06 | GLMN | 0.6820842 | 0.000779825 | ADCK4 | 0.39127448 | 0.012091855 |
| ZNF362 | 0.9118718 | 7.74577E−12 | FAM127C | 0.7727817 | 6.39153E−06 | HMGXB4 | 0.46355015 | 0.000780147 | NEK2 | 0.15717211 | 0.012129972 |
| NNT | 0.9844377 | 7.74577E−12 | HCG18 | 0.24312074 | 6.39992E−06 | POLR2H | 0.69379455 | 0.000780147 | DCUN1D2 | 0.05320981 | 0.01220886 |
| PTRHD1 | 1.3759575 | 8.06372E−12 | PRRC2A | 0.42875174 | 6.45216E−06 | ZDHHC9 | 0.573 | 0.000780147 | GNB1L | 0.585828 | 0.012220306 |
| THOC5 | 1.1505822 | 8.19155E−12 | ANKLE1 | 0.44337326 | 6.47205E−06 | TRMT2A | 0.50724995 | 0.000783617 | REXO1 | 0.48233497 | 0.012227877 |
| APEX1 | 1.3865318 | 8.65283E−12 | GTPBP4 | 0.79592216 | 6.48709E−06 | ALG12 | 0.520933 | 0.000784346 | TXNDC12 | 0.44320285 | 0.012241284 |
| SYTL2 | 1.7043254 | 8.80912E−12 | DHX9 | 0.71879125 | 6.48905E−06 | KARS | 0.7346978 | 0.000786789 | N6AMT2 | 0.68643886 | 0.012252758 |
| WDR5 | 1.0968074 | 8.98782E−12 | PRPF39 | 0.5510901 | 6.57675E−06 | UFM1 | 0.48201048 | 0.000787833 | SDCCAG3 | 0.6220785 | 0.012254316 |
| DHRS4-AS1 | 0.8341031 | 9.7002E−12 | PRKXP1 | 0.44681463 | 6.70807E−06 | ZDHHC15 | 0.15194589 | 0.00079375 | C16orf91 | 0.4084259 | 0.012258015 |
| NOL6 | 1.0806606 | 9.97316E−12 | C12orf43 | 0.49813947 | 6.70839E−06 | SHE | 0.25460142 | 0.000794803 | ALDH3B2 | 0.70586485 | 0.012278861 |
| TDRD7 | 1.596483 | 9.98103E−12 | MAP4K4 | 0.35687676 | 6.72058E−06 | SUV420H1 | 0.5776608 | 0.000797386 | INVS | 0.31112015 | 0.012290367 |
| BCKDHA | 1.2885015 | 1.11144E−11 | ZC3H12B | 0.19700994 | 6.73108E−06 | C20orf96 | 0.4894149 | 0.000799344 | TBC1D14 | 0.23848608 | 0.012307742 |
| NAPA | 1.145038 | 1.12694E−11 | PTP4A1 | 0.7978493 | 6.79448E−06 | HSPH1 | 0.53682923 | 0.000799929 | ADAMTSL5 | 0.30797657 | 0.012315172 |
| PTPLA | 1.4072937 | 1.23908E−11 | SH3BP2 | 0.3343661 | 6.80681E−06 | ZNF529 | 0.24339926 | 0.00080004 | USP51 | 0.768985 | 0.012354119 |
| PNPLA3 | 1.1872301 | 1.23908E−11 | ACO2 | 0.9216018 | 6.86398E−06 | EIF3B | 0.36831236 | 0.000802798 | ANAPC10 | 0.35517243 | 0.012377077 |
| BNIP3 | 1.2962544 | 1.25044E−11 | CYBASC3 | 0.75841177 | 6.86778E−06 | SEPHS2 | 0.6612122 | 0.000803386 | PPP1R3C | 2.1264222 | 0.012396529 |
| IDH3B | 1.310444 | 1.28401E−11 | TUBG1 | 0.85025674 | 6.88718E−06 | SCO2 | 0.6651256 | 0.000806904 | ACOT8 | 0.41157162 | 0.012429795 |
| TUBA1B | 1.1022774 | 1.28784E−11 | PCTP | 0.9452628 | 6.9625E−06 | VPS33B | 0.5933563 | 0.000809909 | CDCA5 | 0.8120752 | 0.012431572 |
| VPS36 | 0.8938355 | 1.33489E−11 | PRKAG1 | 0.90355647 | 6.98919E−06 | TPM3P9 | 0.054140802 | 0.000809909 | EMD | 0.2300462 | 0.012510437 |
| RPL5 | 0.723892 | 1.36526E−11 | LOC92249 | 0.80797845 | 7.02304E−06 | IFIT1 | 0.5306698 | 0.000810342 | EGLN3 | 1.3053488 | 0.01252214 |
| RIT1 | 0.93771017 | 1.36983E−11 | CWC22 | 0.5799649 | 7.05955E−06 | MCMDC2 | 0.14541985 | 0.000811095 | MRPL43 | 0.5675921 | 0.012525913 |
| TOR3A | 1.3054363 | 1.4072E−11 | PFKFB2 | 0.9269702 | 7.05955E−06 | ZC3HC1 | 0.632002 | 0.000812812 | TEX101 | 0.00181203 | 0.012559298 |
| YDJC | 1.3577039 | 1.4617E−11 | GMPR | 0.7998096 | 7.05955E−06 | CEP68 | 0.22760741 | 0.000814533 | GRK5 | 1.0982953 | 0.012586978 |
| DCAF13 | 1.2548944 | 1.49668E−11 | LOC100271836 | 0.78454715 | 7.14746E−06 | LPPR2 | 0.50412315 | 0.000814807 | DHDDS | 0.37268272 | 0.012648704 |
| GART | 1.0700693 | 1.50224E−11 | ZNF507 | 0.4688481 | 7.15142E−06 | LRRC42 | 0.6455783 | 0.000815403 | SEPHS1 | 0.57720375 | 0.012650509 |
| ATP7A | 0.85736763 | 1.61039E−11 | PIK3CD | 0.52978224 | 7.21319E−06 | AAAS | 0.5692825 | 0.000821337 | PTPN1 | 0.2888832 | 0.012657735 |
| METTL16 | 0.80416137 | 1.61365E−11 | SLC6A17 | 1.318999 | 7.27363E−06 | ZNF271 | 0.19243194 | 0.000821613 | MUTYH | 0.43472698 | 0.012657735 |
| C3orf14 | 1.4985203 | 1.61365E−11 | THOC6 | 0.8541941 | 7.36041E−06 | USO1 | 0.5161925 | 0.000824002 | DPYSL4 | 1.6534219 | 0.012717761 |
| ATP11A | 1.2951267 | 1.62726E−11 | PLAA | 0.7901834 | 7.37004E−06 | CSNK1G1 | 0.091767825 | 0.000827377 | MPP6 | 1.3422763 | 0.012751608 |
| UBIAD1 | 0.8971509 | 1.65504E−11 | PSENEN | 0.91112787 | 7.37042E−06 | COPZ1 | 0.758224 | 0.000832078 | ZNF707 | 0.5991381 | 0.012815691 |
| HMGN1 | 1.1880448 | 1.65504E−11 | PAPSS1 | 0.8206486 | 7.4284E−06 | DPY19L2 | 1.4553931 | 0.000833508 | PIGA | 0.6636792 | 0.012821571 |
| CSK | 1.2255445 | 1.65504E−11 | TCEB3 | 0.7133218 | 7.46056E−06 | MRPL22 | 0.37585238 | 0.000836424 | ANKRD65 | 0.45590484 | 0.012833537 |
| HNRNPA1 | 1.0625536 | 1.67743E−11 | AZIN1 | 0.70613694 | 7.4797E−06 | LCMT2 | 0.9541667 | 0.000837366 | USP48 | 0.33389187 | 0.012851605 |
| SAFB | 1.0302978 | 1.67946E−11 | C9orf114 | 0.6601673 | 7.4876E−06 | BEND3P3 | 0.40938026 | 0.000840859 | DDI2 | 0.5835954 | 0.012922462 |
| RPS23 | 0.5265005 | 1.68029E−11 | UBAP2 | 0.59783596 | 7.52001E−06 | CLASP2 | 0.31701437 | 0.000841473 | DNAJC4 | 0.41007444 | 0.012928386 |
| ARMC1 | 1.0287769 | 1.68112E−11 | C1orf109 | 0.78302014 | 7.61534E−06 | MKL2 | 0.537845 | 0.000842039 | LOC729683 | 0.11071068 | 0.012954754 |
| SSU72 | 1.1965593 | 1.72732E−11 | LOC550112 | 0.28463978 | 7.63486E−06 | AKAP10 | 0.53499115 | 0.000846651 | ENOX2 | 0.031451713 | 0.012987119 |
| TNS1 | 1.4064006 | 1.73628E−11 | PNPLA4 | 0.4375085 | 7.70837E−06 | SHMT1 | 0.5814033 | 0.000854261 | ZNF30 | 1.4567513 | 0.013025924 |
| RPL32 | 0.5525642 | 1.78103E−11 | C2orf44 | 0.9307365 | 7.72619E−06 | THAP7 | 0.5540313 | 0.000856183 | NCCRP1 | 0.03226064 | 0.013085056 |
| MMP17 | 1.3393426 | 1.78103E−11 | UHMK1 | 0.5167661 | 7.74987E−06 | NSMAF | 0.28608137 | 0.000858109 | TMEM183A | 0.8451159 | 0.013086919 |
| FAM210A | 0.784821 | 1.83323E−11 | MRPL30 | 0.4547277 | 7.76E−06 | ARL16 | 0.49589434 | 0.000859481 | MYRF | 1.3581434 | 0.013163096 |
| EIF3E | 1.1531041 | 1.83405E−11 | SMC6 | 0.626714 | 7.82134E−06 | NHLRC3 | 0.4411889 | 0.000859481 | XRCC3 | 0.34297448 | 0.013214458 |
| PES1 | 0.88332427 | 1.83405E−11 | NAGK | 0.795892 | 7.84531E−06 | 44445 | 0.70052654 | 0.000859601 | DMAP1 | 0.45371467 | 0.013232806 |
| 44450 | 0.86928415 | 1.83405E−11 | PEAK1 | 0.29421502 | 7.84573E−06 | ISG20 | 0.935625 | 0.000863962 | ANXA11 | 0.43102333 | 0.01325138 |
| ARAP1 | 0.8280596 | 1.83962E−11 | DNAJC18 | 0.50295705 | 7.85008E−06 | LOC400657 | 0.07683575 | 0.000864763 | ZNF251 | 0.5959794 | 0.013273956 |
| PDCD5 | 1.4011129 | 1.84123E−11 | TPM4 | 0.82267576 | 7.97535E−06 | PSMF1 | 0.53397423 | 0.000868636 | TSACC | 1.0750872 | 0.013275845 |
| NDUFB8 | 1.4313183 | 1.951E−11 | ELOF1 | 0.8635611 | 8.01581E−06 | KANSL3 | 0.31595272 | 0.000869612 | AIF1L | 0.37766427 | 0.013286101 |
| SLC1A4 | 1.3311489 | 1.99234E−11 | ERF | 0.8609254 | 8.06252E−06 | PREB | 0.5821192 | 0.000870418 | IVD | 0.28055832 | 0.01328968 |
| FLAD1 | 1.1177883 | 2.00701E−11 | PPP3R1 | 0.8312628 | 8.08518E−06 | FAM50A | 0.503557 | 0.000871224 | BEX2 | 1.3406848 | 0.01328968 |
| GCNT2 | 0.9019198 | 2.01962E−11 | CCDC8 | 0.8407268 | 8.20174E−06 | FAM133B | 0.5960243 | 0.000874436 | IFIT5 | 0.47448453 | 0.013295759 |
| LDB1 | 1.2516624 | 2.04327E−11 | SNAPIN | 0.8226205 | 8.21244E−06 | IGBP1 | 0.6986793 | 0.000874579 | CCDC169 | 2.280992 | 0.013309619 |
| LOC100190986 | 1.0589269 | 2.04839E−11 | SQLE | 0.8309725 | 8.24168E−06 | FNIP2 | 0.68604964 | 0.000874579 | DOT1L | 0.34002036 | 0.013309619 |
| PCNA | 1.3981571 | 2.04839E−11 | PRADC1 | 1.0321765 | 8.24625E−06 | FBXW8 | 0.53921574 | 0.000874579 | WDR55 | 0.28994402 | 0.013353492 |
| RPS29 | 0.90277016 | 2.14487E−11 | TRIP6 | 0.5284952 | 8.28434E−06 | ESCO1 | 0.34064576 | 0.000874579 | TBCCD1 | 0.274724 | 0.013355393 |
| SLC5A6 | 1.2698373 | 2.14526E−11 | GRPEL1 | 0.75402695 | 8.30346E−06 | ENY2 | 0.46521726 | 0.000876423 | SAP130 | 0.3761075 | 0.013395186 |
| TIMM8B | 1.1578397 | 2.14719E−11 | COX11 | 0.7153407 | 8.44667E−06 | MEAF6 | 0.36889032 | 0.00087948 | SACM1L | 0.2736033 | 0.013397093 |
| DPP9 | 0.71879727 | 2.16611E−11 | TOM1 | 0.8301561 | 8.47038E−06 | FBXW5 | 0.6337002 | 0.000884457 | OLFM1 | 2.973835 | 0.013415876 |
| PRRC2C | 0.6922736 | 2.20842E−11 | PWP1 | 0.84465265 | 8.47719E−06 | HDAC8 | 0.5001552 | 0.000886742 | IQCH-AS1 | 0.098377086 | 0.013428142 |
| ESD | 1.446159 | 2.22789E−11 | MED9 | 0.91817653 | 8.51585E−06 | MRPL52 | 0.43796533 | 0.000887388 | DTX2P1-UPK3BP1- | 0.26070294 | 0.013430054 |
| | | | | | | | | | PMS2P11 | | |
| ECD | 1.2892475 | 2.23631E−11 | TMEM138 | 0.7445992 | 8.52483E−06 | RPS25 | 0.39847502 | 0.000888559 | CD3EAP | 0.12964477 | 0.013438105 |
| VPS53 | 0.7402004 | 2.31486E−11 | LOC100128682 | 0.93122417 | 8.59845E−06 | WASF1 | 0.76310235 | 0.000897101 | BLM | 0.73910165 | 0.013438105 |
| KBTBD6 | 0.76780444 | 2.34362E−11 | SNORD47 | 1.725617 | 8.62999E−06 | SCYL2 | 0.3588311 | 0.000899696 | BCL2L2-PABPN1 | 0.19429342 | 0.013490842 |
| AKT1S1 | 1.1582545 | 2.53294E−11 | CHMP3 | 0.642791 | 8.62999E−06 | SNX18 | 0.35433567 | 0.000902069 | TRIM65 | 0.13699424 | 0.013545894 |
| LAMTOR2 | 1.2315849 | 2.82039E−11 | CSPP1 | 0.49821553 | 8.62999E−06 | CUL5 | 0.34394747 | 0.000904144 | VCP | 0.550113 | 0.013577662 |
| PSMG1 | 1.3500952 | 2.82397E−11 | PEX16 | 0.7746085 | 8.64556E−06 | HSPD1 | 0.57904685 | 0.000909074 | RAP1B | 0.5452919 | 0.013630689 |
| RPL13AP3 | 0.704983 | 2.89169E−11 | COPS4 | 0.8704795 | 8.64869E−06 | ZNF526 | 0.21969004 | 0.000910272 | AURKAIP1 | 0.6211899 | 0.013632626 |
| SIRT2 | 1.1616269 | 2.9202E−11 | DCP1B | 0.9673266 | 8.64869E−06 | SUOX | 0.7405491 | 0.000912492 | LOC100652999 | 0.25427324 | 0.013647419 |
| NOA1 | 1.4169805 | 2.93643E−11 | MED16 | 0.7210082 | 8.66047E−06 | SNRPB2 | 0.5085664 | 0.000912492 | KIAA1191 | 0.3687595 | 0.013679197 |
| GTF2H2 | 0.8064633 | 2.93808E−11 | LNX2 | 0.40961447 | 8.71297E−06 | HUNK | 0.022240298 | 0.000912977 | KIAA1033 | 0.23462807 | 0.013709094 |
| ZFYVE21 | 1.4059027 | 2.98082E−11 | MCTP1 | 1.4324279 | 8.71347E−06 | TSC1 | 0.22880189 | 0.000913642 | TRAPPC2P1 | 0.5097513 | 0.013727862 |
| HSD17B14 | 1.761853 | 2.98782E−11 | C21orf33 | 0.77406174 | 8.79356E−06 | DYRK1A | 0.21344616 | 0.000913769 | LOC100130776 | 0.47266072 | 0.013744499 |
| ECH1 | 1.3921402 | 2.99656E−11 | RCC2 | 0.5252779 | 8.79356E−06 | SOSTDC1 | 1.6705114 | 0.000916177 | TRNAU1AP | 0.34889248 | 0.013744499 |
| C10orf90 | 1.4081469 | 2.99656E−11 | RPAP1 | 0.70616144 | 8.79356E−06 | HSD11B1L | 0.8744704 | 0.000923647 | ABCC10 | 0.289854 | 0.013765881 |
| EXTL1 | 1.2025124 | 3.06184E−11 | RUFY3 | 0.6795019 | 8.89993E−06 | DTX2 | 0.5833224 | 0.000923957 | CADM1 | 0.3882846 | 0.013802442 |
| CECR7 | 0.74290013 | 3.07779E−11 | CDYL | 0.44303718 | 8.91598E−06 | SUGP2 | 0.25278464 | 0.000928883 | RLIM | 0.1952238 | 0.013804404 |
| DPP7 | 1.1987042 | 3.15825E−11 | NFYA | 0.5278478 | 8.98116E−06 | MYO10 | 0.44835448 | 0.000928883 | USP1 | 0.25854728 | 0.013836512 |
| IFI16 | 0.96127963 | 3.16979E−11 | FKBP7 | 0.47010988 | 8.98839E−06 | LOC643837 | 0.5781234 | 0.000929559 | TMEM218 | 0.3964493 | 0.01386631 |
| BAHCC1 | 0.6854219 | 3.16979E−11 | DNPH1 | 1.0657072 | 8.98891E−06 | RRN3P3 | 0.3279582 | 0.000932242 | HRSP12 | 0.56479394 | 0.01386631 |
| PRMT5 | 1.220069 | 3.2434E−11 | MS4A7 | 0.41021648 | 9.11342E−06 | DHODH | 0.20075075 | 0.000937812 | KHDRBS1 | 0.28303394 | 0.01386631 |
| B4GALT3 | 1.0961558 | 3.26494E−11 | COL4A3BP | 0.58866143 | 9.1435E−06 | CASP7 | 0.7730126 | 0.000938127 | VILL | 0.5799413 | 0.013905086 |
| NME4 | 1.1432805 | 3.28081E−11 | FLII | 0.68165225 | 9.14403E−06 | PYCRL | 0.3902235 | 0.000942307 | SETD5-AS1 | 0.1251122 | 0.014004854 |
| TRAP1 | 1.297214 | 3.35576E−11 | CACYBP | 0.7627284 | 9.16736E−06 | FOXM1 | 0.7830581 | 0.000942992 | NUBPL | 0.10723846 | 0.014013426 |
| USP21 | 1.0816139 | 3.41056E−11 | OR7E156P | 0.4428831 | 9.17017E−06 | PYROXD2 | 0.80803186 | 0.000944232 | RPP40 | 0.6751542 | 0.01416278 |
| C1orf151-NBL1 | 1.1259803 | 3.42957E−11 | PIAS3 | 0.69926393 | 9.17985E−06 | ORC1 | 0.5666968 | 0.000945658 | PRPF19 | 0.28403822 | 0.014164788 |
| TMEM33 | 0.8252914 | 3.45971E−11 | SDHAF1 | 0.92751074 | 9.19868E−06 | C6orf1 | 0.6080208 | 0.000946531 | RAB4B | 0.9430461 | 0.014188978 |
| WDFY2 | 1.1094866 | 3.46423E−11 | RSL24D1 | 0.8245019 | 9.20151E−06 | SUMO1P3 | 0.37108395 | 0.000949074 | POLR2K | 0.57682675 | 0.014190781 |
| ACBD6 | 1.2015067 | 3.63538E−11 | JOSD2 | 0.7678425 | 9.21865E−06 | THAP1 | 0.8001892 | 0.000949392 | GPATCH3 | 0.5032012 | 0.014214804 |
| DMKN | 1.4211007 | 3.64273E−11 | BOP1 | 0.84486896 | 9.21865E−06 | HEMK1 | 0.32894045 | 0.000950454 | RPS6KA5 | 0.2549025 | 0.014241298 |
| LOC646762 | 0.82641673 | 3.64623E−11 | DET1 | 0.95629084 | 9.25192E−06 | C8orf76 | 0.6393077 | 0.000952395 | MIS18BP1 | 0.3375368 | 0.014285484 |
| MRPL9 | 1.2752459 | 3.65421E−11 | C2orf43 | 0.7942446 | 9.25192E−06 | LOC728554 | 0.51571107 | 0.000956207 | PTTG1 | 0.545308 | 0.014312097 |
| NRAS | 0.87359846 | 3.65421E−11 | TECPR2 | 0.52727324 | 9.31251E−06 | CERK | 0.22880591 | 0.000964683 | BCAS2 | 0.4591478 | 0.0143186 |
| TMEM147 | 1.37843 | 3.65421E−11 | CEP95 | 0.64809114 | 9.32234E−06 | HSPA9 | 0.63139623 | 0.000965761 | DBF4 | 0.2641376 | 0.014327346 |
| PLEKHH1 | 0.7374158 | 3.66131E−11 | APOLD1 | 1.1123242 | 9.36473E−06 | DPY19L3 | 0.39503703 | 0.000966031 | RPA2 | 0.56410164 | 0.014329168 |
| RPS3A | 0.5342802 | 3.66131E−11 | UBE2G2 | 0.6526059 | 9.40787E−06 | NUDT5 | 0.63579977 | 0.000966031 | PMS2P3 | 0.23159626 | 0.014329168 |
| HPS1 | 1.0817738 | 3.70269E−11 | DERL2 | 0.7889375 | 9.41312E−06 | IP6K2 | 0.6480671 | 0.000966166 | SULT1A1 | 0.7023448 | 0.014350746 |
| PRPF31 | 1.2080157 | 3.79523E−11 | SLC43A3 | 0.93219024 | 9.43947E−06 | OBSL1 | 0.24218658 | 0.000966301 | TIPIN | 0.7918485 | 0.014350746 |
| CDC16 | 1.2897289 | 3.84763E−11 | ALKBH4 | 0.38234785 | 9.51069E−06 | ISY1 | 0.27216372 | 0.000966761 | POLE | 0.42513257 | 0.014350746 |
| BLOC1S3 | 0.7136264 | 3.92009E−11 | B9D1 | 0.84619284 | 9.54442E−06 | LASP1 | 0.4176977 | 0.000966761 | DLEU2L | 0.6644989 | 0.014357267 |
| TXLNA | 1.004833 | 4.00663E−11 | ZNF419 | 0.7736609 | 9.56933E−06 | UQCC | 0.62138665 | 0.000969166 | PRKRIR | 0.59649235 | 0.014366035 |
| ZNF549 | 0.96089095 | 4.04329E−11 | PGS1 | 0.7991697 | 9.58181E−06 | RBM7 | 0.47186965 | 0.000972525 | DLGAP1 | 0.40716988 | 0.014374808 |
| RBFOX2 | 0.88143635 | 4.06733E−11 | BRCA1 | 0.39467463 | 9.58239E−06 | FAM63B | 0.32201457 | 0.000972798 | FAM45B | 1.6867464 | 0.014452973 |
| PLGRKT | 1.3275616 | 4.11325E−11 | SMYD5 | 0.772525 | 9.71961E−06 | PRMT3 | 0.41035703 | 0.000972798 | TNRC6A | 0.022247272 | 0.014536266 |
| YTHDF2 | 1.2476811 | 4.11701E−11 | KCTD12 | 0.88096154 | 9.84405E−06 | CARD14 | 0.64657426 | 0.000973124 | CXorf38 | 0.13230532 | 0.014542863 |
| TIMM44 | 0.9578252 | 4.11701E−11 | CDT1 | 0.13128814 | 9.85933E−06 | RGS10 | 0.85312474 | 0.000973451 | RNF207 | 0.112776935 | 0.014551733 |
| TRAF7 | 0.74088484 | 4.11963E−11 | SMPD2 | 1.280556 | 9.86483E−06 | BCL9 | 0.4147408 | 0.000974919 | ANKMY1 | 0.23437369 | 0.014569072 |
| EIF3I | 1.1794871 | 4.14123E−11 | OLFML2A | 0.032295298 | 1.0001E−05 | OSBPL10 | 0.903969 | 0.000978435 | CCNYL1 | 0.16019237 | 0.014600714 |
| POLR2E | 1.0130435 | 4.16416E−11 | TYRO3 | 0.8647714 | 1.00115E−05 | ISOC1 | 0.67494375 | 0.000978954 | PLXNB1 | 0.19357291 | 0.014602783 |
| PTRH2 | 1.2864828 | 4.16416E−11 | PTPN23 | 0.486169 | 1.00171E−05 | METTL21D | 1.0625533 | 0.000980622 | POTEE | 0.5502552 | 0.014611687 |
| MLANA | 1.265886 | 4.17126E−11 | C18orf21 | 0.86358154 | 1.00227E−05 | PRRT3-AS1 | 0.9523524 | 0.000986464 | NPR2 | 1.0830972 | 0.014618317 |
| G3BP2 | 1.1284549 | 4.33305E−11 | BLCAP | 0.7921103 | 1.00632E−05 | SMARCE1 | 0.5694307 | 0.000987265 | TMEM184B | 0.27571243 | 0.014654623 |
| EGLN2 | 1.1854205 | 4.36016E−11 | TGFBRAP1 | 1.2293278 | 1.00688E−05 | SETD6 | 0.6365125 | 0.000987265 | PHKA2 | 0.302207 | 0.014672494 |
| SFXN3 | 0.8669867 | 4.37996E−11 | KIF22 | 0.77576596 | 1.00869E−05 | KIAA1143 | 0.24977246 | 0.000999305 | UQCR11 | 0.47237155 | 0.014672494 |
| MCF2L | 1.3431724 | 4.44664E−11 | ZNF585B | 0.40170234 | 1.00875E−05 | COA1 | 0.4720062 | 0.001000561 | HIC2 | 0.5131053 | 0.014737706 |
| SUCLG1 | 1.2111906 | 4.48736E−11 | ZNF473 | 1.1318926 | 1.00982E−05 | CENPW | 0.9926064 | 0.001004359 | ANKRD13D | 0.4776832 | 0.014737706 |
| FBLN1 | 1.3306372 | 4.58304E−11 | SLC39A3 | 0.81636196 | 1.01389E−05 | ARCN1 | 0.42235512 | 0.00100935 | AIDA | 0.3239046 | 0.014737706 |
| ATXN10 | 1.1240182 | 4.71712E−11 | MTR | 0.34805262 | 1.01396E−05 | PSMD10 | 0.65511805 | 0.00100935 | ACVR1B | 0.79722357 | 0.014737706 |
| SNRNP27 | 1.0950309 | 4.72189E−11 | DNASE1 | 0.17066997 | 1.02673E−05 | SRGAP2C | 0.42035118 | 0.001010224 | CTIF | 0.28840348 | 0.01475567 |
| TMEM206 | 1.3489764 | 4.93928E−11 | ARHGEF39 | 0.30041167 | 1.03471E−05 | RNF6 | 0.5174942 | 0.001011154 | MPV17L2 | 0.43515882 | 0.014785165 |
| CDCA7 | 1.4835886 | 4.96345E−11 | MAGED4 | 1.2793841 | 1.04094E−05 | KIF4A | 1.1253408 | 0.001019546 | LAMA1 | 0.45067897 | 0.014810307 |
| GMPR2 | 1.2774285 | 4.965E−11 | TONSL | 0.46367562 | 1.04126E−05 | KIAA1609 | 0.2118025 | 0.001020483 | CCDC106 | 0.5057802 | 0.014826251 |
| GALC | 1.8729728 | 5.07068E−11 | SLC25A43 | 0.69665396 | 1.05169E−05 | TRIM16L | 0.1827855 | 0.001021223 | DEPDC1B | 0.91839105 | 0.014839901 |
| PITHD1 | 1.1369832 | 5.07228E−11 | LOC339975 | 0.20603897 | 1.06301E−05 | C15orf41 | 0.77502096 | 0.001021764 | MCOLN1 | 0.2707601 | 0.014848523 |
| NOC3L | 0.9563951 | 5.27371E−11 | C11orf92 | 0.055045072 | 1.06729E−05 | MIDN | 0.18791147 | 0.001021908 | RAB2B | 0.111481324 | 0.014873762 |
| NUDT14 | 1.3311676 | 5.44481E−11 | ZNF460 | 0.7513371 | 1.07159E−05 | CLN8 | 0.26728693 | 0.001023047 | SSRP1 | 0.3326567 | 0.014891878 |
| PAX3 | 1.2194217 | 5.54689E−11 | FAM189B | 0.94383854 | 1.07405E−05 | SREBF1 | 0.48399267 | 0.001029582 | MGAT2 | 0.6410283 | 0.014903263 |
| AP1M1 | 1.1772764 | 5.68067E−11 | SLC25A17 | 0.74522287 | 1.07651E−05 | PP7080 | 0.7462253 | 0.001031933 | GORASP2 | 0.44714785 | 0.014921413 |
| TMEM180 | 1.1347793 | 5.72853E−11 | MAGED4B | 1.3581516 | 1.08004E−05 | CBFA2T2 | 0.2096713 | 0.001032881 | TNIP3 | 0.017574487 | 0.014930496 |
| PRKD3 | 0.8571494 | 5.9432E−11 | IARS | 0.7076077 | 1.08312E−05 | COIL | 0.39656702 | 0.001038813 | UBA6 | 0.33729786 | 0.014953534 |
| DLAT | 1.105822 | 6.02272E−11 | ZNF470 | 0.26282895 | 1.08534E−05 | TRIM11 | 0.6222746 | 0.001038813 | HSF2 | 0.44635814 | 0.01496729 |
| SNRPA | 0.99224967 | 6.03948E−11 | SNX6 | 0.676562 | 1.09781E−05 | BRCC3 | 0.49284562 | 0.001040778 | COPS2 | 0.22166891 | 0.014974069 |
| TDG | 0.9153079 | 6.2612E−11 | NEK8 | 0.08609634 | 1.10006E−05 | DUT | 0.5673246 | 0.001046956 | N4BP2L2 | 0.38796887 | 0.014978315 |
| EEF1D | 0.9282289 | 6.44337E−11 | PEX2 | 0.54036677 | 1.10094E−05 | WDR18 | 0.6142696 | 0.001046956 | FAM167B | 0.43656123 | 0.014978315 |
| CYC1 | 1.5655866 | 6.47035E−11 | CLTA | 1.0111403 | 1.10156E−05 | TMEM223 | 0.6790766 | 0.001050047 | CAP1 | 0.47900718 | 0.015006086 |
| MED29 | 0.838347 | 6.4793E−11 | PGBD2 | 1.2626278 | 1.1019E−05 | SMARCA5 | 0.30140725 | 0.001051011 | ABCB7 | 0.46248865 | 0.015008214 |
| TATDN1 | 1.2636234 | 6.77818E−11 | UTP15 | 0.43016258 | 1.11182E−05 | EEF1E1 | 0.67764425 | 0.00105259 | MTA3 | 0.2651598 | 0.01501501 |
| NSUN5 | 1.021407 | 7.00676E−11 | IFT74 | 0.8382863 | 1.11629E−05 | LOC100128573 | 0.2981328 | 0.001053147 | MNS1 | 0.6533816 | 0.015033491 |
| XYLB | 1.1363814 | 7.01402E−11 | RAD23B | 0.41018304 | 1.11857E−05 | RAD17 | 0.27929887 | 0.001055548 | DHX29 | 0.1518282 | 0.015084368 |
| TRABD | 0.9559349 | 7.06305E−11 | ZNF511 | 0.9998989 | 1.1239E−05 | MFSD5 | 0.65206033 | 0.001059394 | CTSH | 0.61727977 | 0.015184851 |
| PDHB | 1.3618178 | 7.06305E−11 | BLVRB | 0.9910579 | 1.12731E−05 | WDR20 | 0.53981465 | 0.001062576 | MVB12B | 0.8355532 | 0.015236605 |
| ZCCHC17 | 1.1407851 | 7.31904E−11 | CENPH | 0.5771324 | 1.12905E−05 | DHRS4L2 | 0.36551335 | 0.001064171 | C19orf47 | 0.77848226 | 0.015274287 |
| NPM3 | 1.5319676 | 7.34203E−11 | CTNS | 0.55960935 | 1.13219E−05 | MRPS22 | 0.60585725 | 0.001065561 | MICALL1 | 0.5194825 | 0.015288309 |
| MYH10 | 0.99060786 | 7.53663E−11 | CHMP2B | 0.70675933 | 1.13451E−05 | PARP1 | 0.5590368 | 0.001065712 | CBX1 | 0.29553297 | 0.015361604 |
| DTNB | 1.1747807 | 7.62384E−11 | DARS2 | 0.7003797 | 1.13822E−05 | ZKSCAN7 | 0.02644825 | 0.001067725 | RNASEK | 0.60580885 | 0.015361604 |
| HIF1AN | 0.7875312 | 7.6265E−11 | AKIRIN1 | 0.6741425 | 1.13858E−05 | RRAGB | 0.698851 | 0.00107286 | REV1 | 0.4017451 | 0.015368544 |
| CCAR1 | 0.9766931 | 7.6879E−11 | MARVELD1 | 0.923008 | 1.14119E−05 | USP45 | 0.52307165 | 0.001075455 | NELFB | 0.40177846 | 0.015451765 |
| DNAJC11 | 1.0977669 | 7.88053E−11 | CNNM1 | 0.89912885 | 1.15087E−05 | SNHG4 | 0.22953553 | 0.001075815 | SH3GL1 | 0.28036076 | 0.01545853 |
| NINL | 1.0333258 | 8.19976E−11 | XPO1 | 0.45469174 | 1.15237E−05 | LOC283683 | 0.05372292 | 0.001078683 | GCSHP3 | 0.9253999 | 0.01545853 |
| ATP8B2 | 0.7312113 | 8.20837E−11 | FAIM | 0.36794093 | 1.15814E−05 | FBXO10 | 0.82495844 | 0.001083448 | CISD2 | 0.22065574 | 0.015467907 |
| PLD6 | 1.355266 | 8.32641E−11 | LDLRAD3 | 0.7670781 | 1.16652E−05 | GARNL3 | 0.8775038 | 0.001086756 | CCDC18 | 0.42730713 | 0.015486257 |
| TUBB6 | 1.2789156 | 8.60928E−11 | TDRD3 | 0.7580764 | 1.16948E−05 | INO80 | 0.7260536 | 0.001090919 | SLC2A11 | 0.12507111 | 0.015486257 |
| ACAA2 | 1.2926904 | 8.73906E−11 | TBRG1 | 0.3645268 | 1.17215E−05 | SNHG1 | 0.6635927 | 0.00109213 | TSPYL1 | 0.3145709 | 0.015486257 |
| XRCC5 | 1.0321577 | 8.88951E−11 | CHIC2 | 0.99329686 | 1.17425E−05 | BVES | 0.24708334 | 0.001092284 | DGCR2 | 0.37084737 | 0.015502857 |
| ALAS1 | 1.1499312 | 8.88951E−11 | C12orf5 | 0.44795746 | 1.17868E−05 | CEP85 | 0.52019876 | 0.001092438 | C7orf50 | 0.4799563 | 0.015517068 |
| EPS15L1 | 0.7571193 | 8.88951E−11 | XPR1 | 0.37182114 | 1.18079E−05 | ZNF587 | 0.26194528 | 0.001098323 | FAS | 0.73045236 | 0.015554952 |
| MRPS2 | 1.2497342 | 8.9237E−11 | MCM7 | 0.8350474 | 1.18175E−05 | ATG5 | 0.48041543 | 0.001099484 | GTF3C5 | 0.4302598 | 0.015556942 |
| HEATR2 | 1.0472653 | 9.15944E−11 | CDK10 | 0.7786464 | 1.18678E−05 | RNF38 | 0.41251513 | 0.001102624 | FBXO42 | 0.42755777 | 0.015571199 |
| ADAR | 0.87672436 | 9.75693E−11 | SNX27 | 0.53318006 | 1.18745E−05 | YIF1A | 0.77041405 | 0.00110278 | LOC100129250 | 0.5824319 | 0.015616876 |
| PVT1 | 0.8035155 | 9.81842E−11 | FANCF | 0.5911049 | 1.18878E−05 | ZNF205-AS1 | 0.011497824 | 0.001103577 | ENO3 | 1.0283778 | 0.015650344 |
| PGLS | 1.2942747 | 9.87327E−11 | QRICH1 | 0.6011862 | 1.19003E−05 | INTS5 | 0.8008701 | 0.001106028 | C14orf119 | 0.32044566 | 0.015705738 |
| THAP4 | 1.152656 | 9.89433E−11 | SUPT4H1 | 0.7696847 | 1.19363E−05 | PELO | 0.6631736 | 0.001106028 | ELP2 | 0.37994173 | 0.015787728 |
| KIAA1737 | 0.73543894 | 9.89433E−11 | STX12 | 0.624505 | 1.19371E−05 | RABGEF1 | 0.39763415 | 0.001106398 | MPLKIP | 0.5405898 | 0.015836423 |
| URM1 | 0.7157094 | 1.00571E−10 | ERCC1 | 0.70939326 | 1.20539E−05 | FLJ41941 | 0.03935884 | 0.001108582 | USMG5 | 0.59915864 | 0.015890166 |
| ETNK2 | 1.4152215 | 1.0376E−10 | FDXACB1 | 0.4262409 | 1.20726E−05 | TMEM192 | 0.3759776 | 0.001108582 | NIF3L1 | 0.5111986 | 0.015912073 |
| CABLES1 | 1.076517 | 1.06236E−10 | BAK1 | 0.72253555 | 1.21218E−05 | TSC22D2 | 0.31914666 | 0.001114274 | SLC41A3 | 0.4285665 | 0.015941395 |
| ANP32E | 1.0101403 | 1.08023E−10 | ABCC5 | 0.59925205 | 1.21414E−05 | TPR | 0.33353662 | 0.001116474 | FZR1 | 0.097648144 | 0.015953503 |
| STRADB | 1.2109668 | 1.08289E−10 | MTRF1L | 0.626055 | 1.21414E−05 | PNPT1 | 0.32954326 | 0.001116474 | MON1A | 0.51336086 | 0.016009646 |
| NDUFV1 | 1.2766967 | 1.08706E−10 | PCMTD2 | 0.5858375 | 1.21512E−05 | C5orf28 | 0.32284826 | 0.001116474 | RMND5B | 0.2886766 | 0.016009646 |
| RPS6KB2 | 0.99073225 | 1.09692E−10 | QTRTD1 | 0.37574735 | 1.2209E−05 | ITSN1 | 0.46161935 | 0.001117496 | ZNF329 | 0.039989986 | 0.016056477 |
| ELAC2 | 1.0743779 | 1.09697E−10 | SRRT | 0.58730173 | 1.22339E−05 | SPPL2B | 0.37711388 | 0.001118029 | C16orf55 | 0.02700477 | 0.016068447 |
| RPF2 | 1.2133638 | 1.10999E−10 | ASCC1 | 0.6121978 | 1.22377E−05 | DNAJC14 | 0.22951554 | 0.001122622 | SF3B14 | 0.64964724 | 0.0160705 |
| NHLRC2 | 0.78237796 | 1.12472E−10 | HIBADH | 0.8136035 | 1.22416E−05 | TEX11 | 0.34191063 | 0.001122622 | HOXA7 | 1.9554125 | 0.016119335 |
| RPL38 | 0.50178695 | 1.12516E−10 | FAM213A | 0.71697545 | 1.22455E−05 | RAB4A | 0.51483905 | 0.001122998 | NXN | 0.086983524 | 0.016119335 |
| CEBPZ | 0.87341964 | 1.14291E−10 | MYD88 | 0.79893893 | 1.22644E−05 | ULK2 | 0.2141287 | 0.001124244 | SNRNP48 | 0.36548448 | 0.016119335 |
| ABCE1 | 0.9247457 | 1.14291E−10 | TOR1AIP1 | 0.48259163 | 1.23015E−05 | PTPRJ | 0.2567872 | 0.001127392 | USP12 | 0.29790306 | 0.016196411 |
| UCHL5 | 1.0022389 | 1.16328E−10 | TOPORS | 0.7471863 | 1.23054E−05 | TAF1L | 0.07980975 | 0.00112809 | GMEB1 | 0.039668605 | 0.016236217 |
| KHSRP | 0.65590143 | 1.16576E−10 | UBE2J2 | 0.6843207 | 1.23821E−05 | DCAF8 | 0.45025566 | 0.00112809 | CPSF6 | 0.24102049 | 0.016251032 |
| DDX27 | 0.9881976 | 1.22025E−10 | EIF4E | 0.65958065 | 1.24134E−05 | SBDSP1 | 0.42083514 | 0.001133496 | GATC | 0.124441534 | 0.016260843 |
| LOC613037 | 0.8814614 | 1.22025E−10 | RDH13 | 1.1743621 | 1.24235E−05 | PALB2 | 0.58314615 | 0.001135411 | CYGB | 0.79176 | 0.016280698 |
| WDR3 | 0.8930213 | 1.25275E−10 | DCTN2 | 0.8908518 | 1.24366E−05 | ZMYM5 | 0.16853443 | 0.001135572 | FAM104A | 0.37610573 | 0.016282993 |
| WDR46 | 1.1076521 | 1.2892E−10 | ASB4 | 0.34008995 | 1.25141E−05 | PLRG1 | 0.5336792 | 0.001139029 | CEP89 | 0.41322502 | 0.016330324 |
| FEN1 | 1.3328639 | 1.32852E−10 | GPRC5B | 0.9492766 | 1.25211E−05 | CYB5R2 | 1.583716 | 0.001142276 | IGFBP5 | 0.65943044 | 0.016370425 |
| HNRNPL | 0.61418235 | 1.32859E−10 | ZNF689 | 1.0581632 | 1.25229E−05 | NUP160 | 0.48191285 | 0.001147524 | OBFC1 | 0.18220586 | 0.016410615 |
| ITM2C | 1.1617124 | 1.35036E−10 | MRPS31 | 0.79874635 | 1.25463E−05 | GAMT | 0.4340037 | 0.001149239 | RGS14 | 0.5727917 | 0.016450893 |
| RBM34 | 0.8414035 | 1.38291E−10 | GPR180 | 0.50885755 | 1.25463E−05 | SPIRE1 | 0.49889934 | 0.001152958 | SHB | 0.6209933 | 0.01646842 |
| GPR89B | 1.170247 | 1.40361E−10 | BOLA3-AS1 | 0.4259431 | 1.2612E−05 | KIAA1024 | 1.8540326 | 0.001153566 | VPS13C | 0.097912095 | 0.016574813 |
| EPS8 | 0.9625065 | 1.42561E−10 | WDR61 | 0.9889307 | 1.26284E−05 | ATAD3C | 0.005188969 | 0.001158863 | UFD1L | 0.50238156 | 0.01665106 |
| CAD | 0.8864779 | 1.44108E−10 | BCL2L12 | 0.67251736 | 1.26635E−05 | METTL1 | 0.692717 | 0.001159027 | CDC42EP3 | 0.40597865 | 0.01665106 |
| SEMA6A | 0.8651836 | 1.46516E−10 | PATL1 | 0.3427066 | 1.27027E−05 | PLK1S1 | 0.4998019 | 0.001173078 | VWA2 | 0.002525972 | 0.016663429 |
| ANKRD10 | 0.9747022 | 1.6048E−10 | TMEM69 | 0.71318674 | 1.27067E−05 | LINC00221 | 0.25769544 | 0.001173078 | FAM83D | 1.0940603 | 0.016670899 |
| AGPS | 0.8991442 | 1.6048E−10 | CDPF1 | 1.0417851 | 1.29882E−05 | KIF14 | 0.23691268 | 0.001174089 | RAB36 | 0.14806046 | 0.01668607 |
| SNHG8 | 1.2840865 | 1.6048E−10 | CSTF1 | 0.8601531 | 1.29955E−05 | LOC494141 | 0.12435391 | 0.001174089 | TOP1 | 0.17228168 | 0.016713438 |
| DAB2 | 1.0821974 | 1.66207E−10 | CCNC | 0.80320865 | 1.31029E−05 | C14orf169 | 0.62583613 | 0.001175328 | CPT1C | 0.64046043 | 0.016713438 |
| DDX6 | 0.8429761 | 1.70549E−10 | SRP9 | 0.8760853 | 1.31909E−05 | SV2A | 1.0177388 | 0.001177309 | CTNND1 | 0.34683028 | 0.016713438 |
| BOLA3 | 1.1982532 | 1.7339E−10 | MVB12A | 0.6322638 | 1.31983E−05 | SLC35C2 | 0.6142142 | 0.001177476 | ZSCAN9 | 0.63238925 | 0.016728644 |
| NOX4 | 0.9405605 | 1.86599E−10 | NEK4 | 0.5703395 | 1.32317E−05 | CUL7 | 0.40701792 | 0.001178779 | TSG101 | 0.6471497 | 0.016746437 |
| PPT2-EGFL8 | 1.0332422 | 1.90092E−10 | NUTF2 | 0.99480903 | 1.32716E−05 | TCAIM | 0.59541315 | 0.001189671 | CABIN1 | 0.19408245 | 0.016753942 |
| LY6K | 1.7264749 | 1.93467E−10 | SNRK | 0.70628995 | 1.34131E−05 | MTCH2 | 0.6334727 | 0.001194432 | SMAD9 | 0.23212688 | 0.016761232 |
| PMPCA | 1.2134651 | 1.93467E−10 | ANKZF1 | 0.7559004 | 1.34701E−05 | ZNF107 | 0.090047434 | 0.001197596 | APIP | 0.2891178 | 0.016791736 |
| MAPRE2 | 0.9209135 | 1.94012E−10 | MTA2 | 0.58388025 | 1.35571E−05 | PSMB4 | 0.50118 | 0.001199029 | RARG | 0.560015 | 0.01679926 |
| TIMM13 | 1.0708095 | 1.9456E−10 | OSGEP | 0.97870326 | 1.36113E−05 | CHD7 | 0.3852858 | 0.001199029 | TMEM67 | 0.04367755 | 0.016868654 |
| MEGF8 | 0.69022167 | 1.95309E−10 | TP53BP1 | 0.50534546 | 1.36389E−05 | NUP35 | 0.50721 | 0.001199029 | MAP7D2 | 1.296016 | 0.016873398 |
| MFF | 1.1062462 | 2.01631E−10 | ZNF234 | 0.3977598 | 1.37404E−05 | TBC1D20 | 0.23471524 | 0.001206315 | TSEN34 | 0.2801009 | 0.01689133 |
| DDX46 | 0.6674572 | 2.06544E−10 | UTP20 | 0.3884627 | 1.37549E−05 | CDK11A | 0.30853897 | 0.001207415 | BCAT1 | 0.73559535 | 0.01694805 |
| SLC18B1 | 1.4423625 | 2.07057E−10 | SLC35E2B | 0.5241927 | 1.3949E−05 | ACOT7 | 0.6993009 | 0.001209496 | ABCD4 | 0.22781764 | 0.017002551 |
| STK32A | 1.2380122 | 2.09064E−10 | CCDC51 | 0.93997395 | 1.40906E−05 | HDAC5 | 0.35073218 | 0.001209496 | TMEM60 | 0.44439757 | 0.017017994 |
| PET117 | 1.2151424 | 2.13937E−10 | NPIP | 0.5787673 | 1.41433E−05 | LOC100188947 | 0.18745588 | 0.001209496 | DHRS13 | 0.7420105 | 0.017090819 |
| GNL3 | 1.2353324 | 2.17859E−10 | PPM1F | 0.5166853 | 1.41651E−05 | CYB5RL | 0.73253125 | 0.001209496 | MGC16275 | 0.48114076 | 0.017122084 |
| RCC1 | 1.2458841 | 2.18549E−10 | YARS2 | 0.32284525 | 1.41984E−05 | E4F1 | 0.65023816 | 0.001210134 | ZRANB1 | 0.5055291 | 0.017129741 |
| ENOPH1 | 1.180776 | 2.26533E−10 | WDFY1 | 0.66730887 | 1.41984E−05 | NEDD4L | 0.13118885 | 0.001213509 | GMIP | 0.6946575 | 0.01714003 |
| PPAT | 1.1604028 | 2.27328E−10 | TMEM79 | 0.85847765 | 1.42492E−05 | KIAA0391 | 0.6149989 | 0.001215957 | CYB5R1 | 0.54659444 | 0.017192004 |
| ZNF74 | 0.77809775 | 2.29529E−10 | C5orf30 | 0.8862719 | 1.44158E−05 | EIF6 | 0.86455446 | 0.001215957 | PSMD6 | 0.61101985 | 0.017197054 |
| PHLDB1 | 1.1082191 | 2.29865E−10 | HSPA14 | 0.9115146 | 1.44158E−05 | C3orf17 | 0.44639468 | 0.001223401 | MAGEA2 | 0.41015136 | 0.017207158 |
| BNC2 | 0.8605634 | 2.3312E−10 | TAF1B | 0.6728832 | 1.44733E−05 | RPS13 | 0.29922652 | 0.001229232 | ZNF420 | 0.6878436 | 0.017262405 |
| PDE4D | 0.62050784 | 2.37792E−10 | CENPM | 1.3098348 | 1.45275E−05 | TMLHE | 0.38117227 | 0.001237465 | IL16 | 0.28055006 | 0.017275413 |
| MRPL4 | 1.0659838 | 2.42886E−10 | VPS 18 | 0.71243286 | 1.45581E−05 | MTERF | 0.8563492 | 0.001245032 | NDUFB1 | 0.5445524 | 0.017280486 |
| COMMD5 | 1.244057 | 2.4407E−10 | LTBR | 0.8242982 | 1.46125E−05 | ZNF639 | 0.15372798 | 0.001249937 | PHKG2 | 0.056005403 | 0.017295934 |
| ZFP36L1 | 0.96035236 | 2.50369E−10 | MLL2 | 0.29524204 | 1.46529E−05 | CLUH | 0.34834656 | 0.001249937 | DOLK | 0.4567094 | 0.017359187 |
| NT5DC2 | 1.2540836 | 2.50369E−10 | ZNF773 | 0.37461787 | 1.47797E−05 | SMAD5 | 0.26020622 | 0.001250594 | PCGF6 | 0.72840536 | 0.017401544 |
| RRP15 | 0.6461484 | 2.50369E−10 | EIF1AD | 0.454144 | 1.47916E−05 | WBP1L | 0.6168068 | 0.001250772 | ZNF397 | 0.45945555 | 0.017577734 |
| UBA2 | 0.74958897 | 2.63784E−10 | TAX1BP3 | 0.7691991 | 1.48614E−05 | KAT5 | 0.49879423 | 0.001254075 | ZNF770 | 0.25507024 | 0.017617877 |
| EPRS | 1.003976 | 2.63784E−10 | ZNF662 | 0.38840955 | 1.48843E−05 | GPSM1 | 0.79697555 | 0.001259013 | HERC4 | 0.2604484 | 0.017625733 |
| ALKBH7 | 1.0931436 | 2.66062E−10 | HSBP1 | 0.68197685 | 1.4929E−05 | LRRFIP1 | 0.35828346 | 0.001265246 | CNTRL | 0.4171437 | 0.017676779 |
| POLR3A | 0.8080483 | 2.70279E−10 | DOCK1 | 0.58130217 | 1.49411E−05 | CASP8AP2 | 0.3734797 | 0.001265426 | WDR67 | 0.7164936 | 0.017706068 |
| TRMT5 | 0.7155569 | 2.71689E−10 | STON1 | 0.38516498 | 1.50482E−05 | PI4KA | 0.2887917 | 0.001269252 | TFB1M | 0.34568062 | 0.01774105 |
| NTHL1 | 1.2905643 | 2.73046E−10 | PMS2P5 | 0.44167098 | 1.50603E−05 | ASB8 | 0.30876943 | 0.001271869 | HNRNPA1P10 | 0.64380527 | 0.017803041 |
| RPL7 | 0.6888881 | 2.73046E−10 | QRSL1 | 0.3566902 | 1.51056E−05 | HOXC10 | 0.45679644 | 0.001273758 | TRAPPC8 | 0.13114527 | 0.017835471 |
| CYP7B1 | 1.7348754 | 2.76058E−10 | RNF111 | 0.25429398 | 1.5199E−05 | ACSBG1 | 1.3852835 | 0.001274916 | ZNF350 | 0.8551578 | 0.017878409 |
| RAB7L1 | 0.79953676 | 2.79766E−10 | THUMPD2 | 0.6088777 | 1.5334E−05 | ZNF333 | 0.19033045 | 0.001276809 | PKD1L1 | 1.3435235 | 0.017910962 |
| NUDCD3 | 0.8280771 | 2.9333E−10 | PCNXL3 | 0.7944615 | 1.54589E−05 | DDX31 | 0.6448225 | 0.001280177 | STK32C | 1.364628 | 0.01792965 |
| ACTR1A | 1.0053842 | 2.99692E−10 | NUPL2 | 0.70407873 | 1.55848E−05 | NCALD | 0.6958746 | 0.001282323 | TCEAL1 | 0.49274758 | 0.017940369 |
| TTYH2 | 1.0290635 | 3.01153E−10 | HLX | 1.4980065 | 1.55935E−05 | UMPS | 0.28986546 | 0.001287923 | LOC284757 | 0.4786617 | 0.018019701 |
| FH | 1.203283 | 3.0447E−10 | APBB1 | 0.81140447 | 1.56175E−05 | NDUFAF1 | 0.32971653 | 0.001288783 | RNF19B | 0.4010029 | 0.018079111 |
| MECR | 0.9363095 | 3.06161E−10 | PDHX | 0.6793085 | 1.57331E−05 | SCRIB | 0.57979906 | 0.001288783 | ZNF236 | 0.27288938 | 0.018079111 |
| SLC25A6 | 0.6485073 | 3.08488E−10 | ACOT1 | 0.8216025 | 1.57803E−05 | CCT6P3 | 0.48994282 | 0.001290384 | ARID3A | 0.96380544 | 0.018095429 |
| PSMD4 | 1.2866951 | 3.09364E−10 | HDAC1 | 0.839765 | 1.58739E−05 | TWISTNB | 0.40791628 | 0.001290384 | ARMC8 | 0.21268846 | 0.018100717 |
| ADSS | 1.0606922 | 3.1182E−10 | UHRF1 | 1.0779032 | 1.5879E−05 | METTL20 | 0.073429205 | 0.001293538 | POLA2 | 0.6395455 | 0.018164063 |
| NLN | 0.7301245 | 3.29081E−10 | TET1 | 0.78388786 | 1.58918E−05 | PDE12 | 0.29307875 | 0.001294217 | NOP14-AS1 | 0.6329789 | 0.018172139 |
| CHTOP | 1.0167994 | 3.30281E−10 | ZFX | 0.3936024 | 1.60951E−05 | NSMF | 0.47400135 | 0.001298991 | CDADC1 | 0.46990258 | 0.018199622 |
| ZCCHC3 | 0.91814536 | 3.30281E−10 | OGG1 | 0.7595007 | 1.60964E−05 | PPIL1 | 0.659169 | 0.001298991 | C9orf69 | 0.5947682 | 0.018210487 |
| TRAK2 | 0.93183726 | 3.32116E−10 | GPSM3 | 0.55924875 | 1.61172E−05 | RPP38 | 0.47782606 | 0.001307159 | FOXRED1 | 0.28378415 | 0.018265595 |
| YIF1B | 1.0196247 | 3.34864E−10 | SMARCB1 | 0.67497844 | 1.61655E−05 | LINC00310 | 0.051882934 | 0.001308031 | ZNF143 | 0.4918647 | 0.01833155 |
| LOC375295 | 1.3998669 | 3.41993E−10 | PSTPIP2 | 0.30791205 | 1.62534E−05 | UBE2L3 | 0.5600572 | 0.001309405 | ZMYM3 | 0.02200383 | 0.018367637 |
| SERBP1 | 0.7961883 | 3.53181E−10 | DAPK1 | 0.5553489 | 1.62704E−05 | PRIM1 | 0.804624 | 0.001312286 | IGDCC4 | 1.0881517 | 0.018389786 |
| RIMKLB | 0.6925092 | 3.61667E−10 | CCDC28B | 0.90930223 | 1.62848E−05 | PIAS2 | 0.25014806 | 0.001313729 | DEK | 0.3586672 | 0.018411726 |
| TARS | 1.0879767 | 3.68858E−10 | MELK | 1.2197086 | 1.62848E−05 | CDC40 | 0.23482238 | 0.001314104 | RBM45 | 0.6029638 | 0.018453335 |
| PPP6R1 | 1.1894952 | 3.71404E−10 | ACBD5 | 0.34791854 | 1.70256E−05 | FGD6 | 0.5157292 | 0.001314291 | LOC100130357 | 0.36301294 | 0.018469956 |
| POLR1A | 0.8644284 | 3.85216E−10 | TAF1C | 0.6252744 | 1.70365E−05 | R3HDM4 | 0.67060655 | 0.001320398 | NDUFB10 | 0.7187017 | 0.018477923 |
| HAUS5 | 0.6014289 | 4.0946E−10 | RNF144A-AS1 | 0.3016856 | 1.70365E−05 | CERS6-AS1 | 1.5315001 | 0.001320398 | TMEM160 | 0.3405012 | 0.018477923 |
| LSM7 | 1.4484695 | 4.11174E−10 | TMEM173 | 0.77309674 | 1.70626E−05 | MESDC2 | 0.43301803 | 0.001323491 | STX8 | 0.44775498 | 0.018539393 |
| PCID2 | 1.1229569 | 4.1345E−10 | C11orf57 | 0.64347386 | 1.72607E−05 | ST6GALNAC4 | 0.8588095 | 0.001323933 | ADAM12 | 0.8961189 | 0.018547621 |
| GGA1 | 0.8995033 | 4.17981E−10 | SLC7A2 | 1.973502 | 1.72607E−05 | NCOR2 | 0.27442378 | 0.001324122 | IRF3 | 0.322506 | 0.018561495 |
| CCDC43 | 1.0908504 | 4.18463E−10 | MZT1 | 0.7170609 | 1.72648E−05 | ADARB1 | 0.55256027 | 0.001326338 | SLC3A2 | 0.65482706 | 0.018566909 |
| TRAK1 | 0.78489274 | 4.2762E−10 | KIF2A | 0.49841747 | 1.72648E−05 | AMMECR1L | 0.4861169 | 0.001327288 | GRINA | 0.23839359 | 0.018577972 |
| EIF2S1 | 0.88799727 | 4.34779E−10 | ADD3 | 0.7246797 | 1.72648E−05 | METRN | 0.6580716 | 0.001327986 | ANP32B | 0.4454498 | 0.018586215 |
| NPIPL3 | 0.9162915 | 4.40787E−10 | RNF138 | 0.7478923 | 1.728E−05 | FOXK1 | 0.24339518 | 0.001331988 | AP5B1 | 0.06856168 | 0.018644942 |
| SCO1 | 1.3142617 | 4.40787E−10 | OTUD7B | 0.75253147 | 1.728E−05 | RAE1 | 0.5808761 | 0.001332688 | PPM1A | 0.10838427 | 0.01871828 |
| GLUD1 | 0.9419546 | 4.43227E−10 | LHFPL5 | 0.17276011 | 1.73527E−05 | ASF1B | 1.1786702 | 0.001333133 | TRIOBP | 0.107994236 | 0.018780466 |
| PPIE | 0.95363224 | 4.48832E−10 | CENPC1 | 0.7190016 | 1.7413E−05 | KRBA1 | 0.5406008 | 0.001351205 | GINS2 | 0.61640793 | 0.01878879 |
| SLC25A13 | 0.8580746 | 4.51619E−10 | ZNF292 | 0.49046823 | 1.7482E−05 | E2F6 | 0.67041135 | 0.001353205 | TRMT1L | 0.27366567 | 0.018822355 |
| RRP9 | 1.182791 | 4.63265E−10 | REEP3 | 0.29498082 | 1.74847E−05 | HARS2 | 0.56284267 | 0.001360131 | IFT81 | 0.21216106 | 0.018822355 |
| NCOA4 | 1.0816562 | 4.63265E−10 | MRTO4 | 0.48727232 | 1.74847E−05 | ZBTB9 | 0.5884701 | 0.001360845 | CEP192 | 0.1810289 | 0.018850717 |
| AK1 | 0.66137743 | 4.63337E−10 | 44260 | 0.61724544 | 1.7554E−05 | ZSWIM1 | 0.33593178 | 0.001365461 | LOC441155 | 0.05381197 | 0.018864796 |
| TYMS | 1.3881396 | 4.86394E−10 | PAQR7 | 0.420739 | 1.77219E−05 | TRIT1 | 0.58651304 | 0.001366698 | ANKRD36B | 0.19351147 | 0.018878885 |
| PFAS | 0.6827101 | 4.99009E−10 | KIT | 2.5560722 | 1.77576E−05 | TRMT61B | 0.54393464 | 0.001368198 | ATP5F1 | 0.6103369 | 0.018913062 |
| MITF | 1.1496682 | 5.05306E−10 | FANCD2 | 0.13317607 | 1.77633E−05 | GID8 | 0.43026066 | 0.001368393 | C14orf166 | 0.57186186 | 0.018938673 |
| SET | 0.9126158 | 5.19442E−10 | GTF2H2B | 0.41768783 | 1.78855E−05 | TRMT61A | 0.64204067 | 0.001369829 | C16orf13 | 0.55146515 | 0.018998866 |
| CHD3 | 0.86242145 | 5.23011E−10 | TCF20 | 0.7661042 | 1.81089E−05 | INTS9 | 0.5221213 | 0.001369829 | USP31 | 0.22732937 | 0.019036125 |
| LYPLAL1 | 0.9178042 | 5.33159E−10 | SKA2 | 0.53425044 | 1.83084E−05 | RNF122 | 0.88798016 | 0.001377885 | GUSBP1 | 0.39174163 | 0.019124854 |
| SFXN1 | 0.76270497 | 5.50075E−10 | PTGFRN | 0.5767194 | 1.84297E−05 | GORAB | 0.65516764 | 0.001382489 | NIPAL2 | 0.7071034 | 0.019124854 |
| SCARB1 | 1.0318352 | 5.52743E−10 | NDUFS8 | 0.930502 | 1.85338E−05 | UBE2O | 0.29567894 | 0.00138295 | RNF214 | 0.1536015 | 0.019136215 |
| DUSP12 | 1.2124035 | 5.59145E−10 | DNAJA2 | 0.62490404 | 1.86026E−05 | CKS1B | 0.73275036 | 0.001388827 | HECTD2 | 0.7335258 | 0.019185102 |
| SNX5 | 0.9501143 | 5.6242E−10 | FHIT | 0.91428965 | 1.86085E−05 | LOC100506599 | 0.06580062 | 0.001390879 | TRAIP | 0.94251645 | 0.019199404 |
| IARS2 | 0.9922224 | 5.76578E−10 | UNC13B | 0.94154865 | 1.86911E−05 | DESI2 | 0.47740442 | 0.001398251 | TMEM234 | 0.50148726 | 0.019236778 |
| NFYC | 0.90940773 | 5.93257E−10 | FAM106A | 0.2260324 | 1.8839E−05 | PTBP3 | 0.32708552 | 0.001403387 | MEST | 0.88305336 | 0.019283211 |
| LSM4 | 1.1624392 | 5.98921E−10 | NPFFR2 | 0.28818002 | 1.89683E−05 | AQR | 0.46697974 | 0.001403387 | CDC45 | 1.1011461 | 0.019291736 |
| SAT2 | 1.0626986 | 6.11021E−10 | PGGT1B | 0.5213374 | 1.9034E−05 | GCDH | 0.6020515 | 0.001403387 | TRERF1 | 0.6639574 | 0.019300264 |
| MYC | 1.2660818 | 6.11021E−10 | BRD7 | 0.5287525 | 1.9106E−05 | SKIV2L | 0.49865952 | 0.001407334 | MAL | 1.0260917 | 0.019355622 |
| DPAGT1 | 1.207488 | 6.1943E−10 | INPP5K | 0.76373583 | 1.93907E−05 | PSMC5 | 0.8086382 | 0.001408875 | ZNF790 | 0.65466845 | 0.019425814 |
| LSS | 1.1153355 | 6.27636E−10 | LRRC14 | 0.7749379 | 1.94719E−05 | BMS1P4 | 0.16605842 | 0.00141203 | NDUFA9 | 0.5081276 | 0.019430978 |
| ECHS1 | 1.2076036 | 6.37756E−10 | PARN | 0.58958656 | 1.94828E−05 | PBDC1 | 0.5967603 | 0.001412165 | HPDL | 0.96726465 | 0.019430978 |
| IMPACT | 0.8158563 | 6.45128E−10 | MRPL2 | 0.7498275 | 1.95644E−05 | CDK1 | 1.0005844 | 0.001427224 | MPPE1 | 0.13121209 | 0.019430978 |
| EIF3J | 0.91797316 | 6.63306E−10 | HIGD1A | 0.66701967 | 1.98939E−05 | CSNK1E | 0.2949235 | 0.00142899 | LRRC27 | 0.10966662 | 0.019439088 |
| ARF6 | 0.8282203 | 6.86305E−10 | PHF20 | 0.371856 | 1.99243E−05 | CRIP1 | 1.6010255 | 0.00142899 | CSRP2BP | 0.3773558 | 0.019439088 |
| MSANTD3 | 1.059501 | 7.01179E−10 | VPS11 | 0.7212798 | 2.01575E−05 | LOC729609 | 0.076163076 | 0.001431643 | NCKAP5 | 0.8033742 | 0.019447676 |
| MLPH | 1.0995947 | 7.16606E−10 | APEX2 | 0.8169037 | 2.01931E−05 | UTP11L | 0.4526724 | 0.001432937 | RNF139 | 0.6698789 | 0.01947688 |
| NTPCR | 1.1151167 | 7.25833E−10 | ACACA | 0.41604757 | 2.0341E−05 | CYTL1 | 1.5416617 | 0.001436415 | RNF130 | 0.4322211 | 0.019517926 |
| GEMIN7 | 1.1870316 | 7.26819E−10 | LRPPRC | 0.5468132 | 2.03573E−05 | WASF2 | 0.41865292 | 0.00143908 | ZNF266 | 0.103775285 | 0.019532451 |
| GLO1 | 1.164711 | 7.31307E−10 | LOC284578 | 0.10218703 | 2.04408E−05 | MRPL47 | 0.6192411 | 0.001440313 | LRCH3 | 0.26960936 | 0.019552898 |
| GAB2 | 1.0561094 | 7.32548E−10 | MIOS | 1.0981138 | 2.04408E−05 | KIAA1614 | 0.17772894 | 0.001440313 | ASF1A | 0.49543414 | 0.019558575 |
| C8orf33 | 0.9523217 | 7.46918E−10 | RFX5 | 0.6855886 | 2.04408E−05 | KLHL7 | 0.59220004 | 0.001447379 | HDDC2 | 0.48861903 | 0.019608426 |
| FAM98A | 1.0343833 | 7.50285E−10 | ZNF696 | 0.1357458 | 2.05163E−05 | FADD | 0.38666403 | 0.001448411 | MOAP1 | 0.26360703 | 0.019611153 |
| POLR3G | 1.075817 | 7.81135E−10 | CTSL2 | 0.25145856 | 2.06218E−05 | SDF2L1 | 0.67279124 | 0.001460916 | CETN3 | 0.31040287 | 0.019625505 |
| TELO2 | 0.92257345 | 8.07324E−10 | OSBPL9 | 0.6615576 | 2.06831E−05 | NADK | 0.5612798 | 0.001461402 | TRAPPC13 | 0.1788626 | 0.019646039 |
| QPCT | 1.4589316 | 8.09886E−10 | TP73-AS1 | 0.1977541 | 2.08245E−05 | PRRC2B | 0.27945963 | 0.001466477 | HSD17B10 | 0.5875683 | 0.019698594 |
| PPP2R3C | 1.1592219 | 8.14611E−10 | MGME1 | 0.67332685 | 2.09567E−05 | UBA3 | 0.52911884 | 0.001468358 | ACVR2A | 0.3169976 | 0.01976321 |
| PHF23 | 1.279548 | 8.19521E−10 | ZFYVE26 | 0.45574123 | 2.12541E−05 | PCBP1-AS1 | 0.6692113 | 0.001471638 | HNRNPAB | 0.23620573 | 0.01976321 |
| NOSIP | 1.1992965 | 8.19521E−10 | OIP5-AS1 | 0.47885856 | 2.12985E−05 | SMEK1 | 0.48542967 | 0.001479127 | VTA1 | 0.38207218 | 0.019768944 |
| RMDN1 | 0.7780977 | 8.21308E−10 | CAP2 | 1.3305732 | 2.15005E−05 | WRAP73 | 0.50574744 | 0.001481586 | DHTKD1 | 0.048024923 | 0.019846454 |
| SMG7 | 0.7957817 | 8.24463E−10 | FAM69C | 2.3823128 | 2.15333E−05 | WDR91 | 0.30613106 | 0.001488841 | SCYL3 | 0.35983136 | 0.019864198 |
| PDXDC1 | 0.9618668 | 8.40599E−10 | TEX30 | 0.89133805 | 2.17236E−05 | FANCI | 0.79100233 | 0.00149188 | HK2 | 0.043796193 | 0.019893251 |
| MLXIP | 0.8354184 | 8.41877E−10 | C17orf80 | 0.66199493 | 2.18351E−05 | KIAA0754 | 0.032017138 | 0.001496556 | BACE1-AS | 0.07565246 | 0.019893251 |
| CUL4A | 0.8136336 | 8.62044E−10 | XRCC6 | 0.72739613 | 2.18683E−05 | SRGAP2 | 0.23156887 | 0.001499042 | MARK4 | 0.5550816 | 0.019893251 |
| CTSC | 0.8158276 | 8.7745E−10 | F2R | 0.63662493 | 2.19244E−05 | CBWD3 | 0.45636177 | 0.00150253 | ARFIP2 | 0.1753348 | 0.019937849 |
| COG2 | 1.0370327 | 8.87244E−10 | MLLT4-AS1 | 0.9109536 | 2.19244E−05 | TMCO6 | 0.8303996 | 0.001502745 | RIC8B | 0.27195457 | 0.020072894 |
| CCDC124 | 1.0360564 | 8.88008E−10 | RBM26 | 0.35163385 | 2.1942E−05 | COQ9 | 0.62919915 | 0.001503815 | NSA2 | 0.43331152 | 0.020199625 |
| TFAM | 0.5522611 | 9.00592E−10 | LINC00652 | 0.07877943 | 2.24062E−05 | PLCD1 | 0.9440365 | 0.001510598 | SLC30A9 | 0.28128284 | 0.020250455 |
| PTDSS1 | 1.1570103 | 9.0256E−10 | LOC100507117 | 0.2271449 | 2.25157E−05 | TPM2 | 0.7152307 | 0.001513036 | ACTL6A | 0.54734004 | 0.02026547 |
| G3BP1 | 1.0521603 | 9.10225E−10 | AGBL5 | 0.8027623 | 2.26365E−05 | MDGA2 | 1.3921695 | 0.001522956 | FANCE | 0.86704963 | 0.020292712 |
| POLR2I | 1.0870676 | 9.23728E−10 | PLEKHA1 | 0.265334 | 2.27144E−05 | BRAT1 | 0.41761768 | 0.001524328 | TOP2A | 0.7282649 | 0.020500468 |
| ZNF704 | 0.5961627 | 9.26665E−10 | ICAM4 | 0.45879567 | 2.27944E−05 | RAD21 | 0.32729855 | 0.001524476 | RECQL4 | 0.7179477 | 0.020540101 |
| BCOR | 1.1779792 | 9.31454E−10 | THUMPD1 | 0.5623245 | 2.28235E−05 | INCENP | 0.81959176 | 0.001524476 | KATNB1 | 0.42602882 | 0.02060791 |
| TMEM161A | 1.205652 | 9.53722E−10 | LOC100505876 | 0.38998604 | 2.28856E−05 | USP28 | 0.46511093 | 0.001528381 | ENTPD5 | 0.93348616 | 0.020610764 |
| CLCN7 | 0.9201709 | 9.55809E−10 | NAP1L4 | 0.7616099 | 2.29973E−05 | FAM20B | 0.113574594 | 0.001539339 | MRPL20 | 0.6283633 | 0.020631974 |
| ALKBH2 | 1.2959516 | 9.64556E−10 | ZNF561 | 0.39841765 | 2.30433E−05 | PHACTR1 | 0.4836398 | 0.001544155 | BSDC1 | 0.2942675 | 0.020631974 |
| HELZ | 0.66669375 | 9.6986E−10 | PMS2 | 0.57807595 | 2.31446E−05 | H3F3AP4 | 0.6702657 | 0.001544155 | WRNIP1 | 0.14382109 | 0.020678292 |
| DIEXF | 0.68646634 | 9.81642E−10 | PRR3 | 0.75877 | 2.32854E−05 | AKAP1 | 0.43815923 | 0.001545545 | PDCD7 | 0.11383819 | 0.020699809 |
| NARS | 1.1140763 | 9.96514E−10 | COMMD2 | 0.35857773 | 2.32985E−05 | CLN5 | 0.46945208 | 0.001547743 | STXBP5 | 0.19856821 | 0.020739787 |
| ACOT2 | 1.1271528 | 9.97054E−10 | CACUL1 | 0.4595869 | 2.33004E−05 | C9orf41 | 1.0781956 | 0.001547743 | SAYSD1 | 0.40997684 | 0.02076136 |
| LUZP1 | 0.7207525 | 1.01049E−09 | RPUSD1 | 0.88320065 | 2.33861E−05 | ZNHIT6 | 0.30698255 | 0.001548258 | NCAM2 | 1.5521085 | 0.020789198 |
| ABCF1 | 0.8732791 | 1.02928E−09 | CPNE1 | 0.85684454 | 2.35471E−05 | HNRNPA2B1 | 0.5228857 | 0.001550753 | PTCD2 | 0.5512807 | 0.02083583 |
| DUSP3 | 0.9538343 | 1.02928E−09 | ALS2CL | 0.8597314 | 2.37355E−05 | ANKRD49 | 0.6473663 | 0.001553839 | KPNA1 | 0.2329823 | 0.020860625 |
| TIAL1 | 0.62751675 | 1.03335E−09 | MDM4 | 0.4381157 | 2.37508E−05 | DCP1A | 0.30217236 | 0.001553839 | PHTF2 | 0.36736912 | 0.020891716 |
| PINX1 | 1.461218 | 1.03335E−09 | DNAJC9 | 0.9850901 | 2.37755E−05 | ZNF670-ZNF695 | 0.34169057 | 0.001561128 | PELP1 | 0.18582764 | 0.020935166 |
| VPS16 | 1.1856738 | 1.03335E−09 | GRAMD1A | 0.7955531 | 2.39963E−05 | ASB1 | 0.57055354 | 0.001561942 | SARNP | 0.51411897 | 0.020938062 |
| SPDYE3 | 0.7710866 | 1.05627E−09 | EMG1 | 0.8450252 | 2.41651E−05 | PLSCR1 | 0.63491714 | 0.001565712 | UBA1 | 0.55808866 | 0.020956675 |
| SNAP29 | 0.72720206 | 1.08063E−09 | CPM | 0.54802054 | 2.42018E−05 | ZNF84 | 0.16680863 | 0.001575729 | BRD9 | 0.008692611 | 0.020978451 |
| ZNF326 | 0.9385799 | 1.08943E−09 | FAM69B | 1.1083199 | 2.42038E−05 | HOMER3 | 0.3463207 | 0.001577742 | DPH5 | 0.1887897 | 0.020987404 |
| GTF2F1 | 0.9421066 | 1.10482E−09 | POR | 0.7891559 | 2.42637E−05 | TIMM10B | 0.44324178 | 0.00158933 | PAPD4 | 0.20231165 | 0.021006057 |
| MUM1 | 0.81056446 | 1.11528E−09 | HYLS1 | 0.42484823 | 2.43063E−05 | HEATR5A | 0.20922282 | 0.001592416 | SLTM | 0.26011643 | 0.021034187 |
| EI24 | 0.9634794 | 1.12794E−09 | HS1BP3 | 0.7945517 | 2.43084E−05 | PGAP2 | 0.54024667 | 0.001592944 | RNF121 | 0.319298 | 0.021046319 |
| RC3H1 | 0.6641144 | 1.12794E−09 | FLJ35282 | 0.06734989 | 2.4322E−05 | DAZAP2 | 0.6450992 | 0.001597688 | PHF3 | 0.26735196 | 0.021046319 |
| AKT2 | 0.5756431 | 1.2067E−09 | DDX41 | 0.85263205 | 2.45515E−05 | MAP7D1 | 0.5238836 | 0.001599426 | TTPA | 0.76920575 | 0.021058703 |
| PSMC4 | 1.2207077 | 1.24108E−09 | FAM86C1 | 0.9731633 | 2.4632E−05 | ZNF430 | 0.13570252 | 0.001604188 | DCAF17 | 0.17802452 | 0.021137536 |
| ACY1 | 1.2130564 | 1.24218E−09 | THOC1 | 0.66682273 | 2.4632E−05 | PRR13 | 0.7020174 | 0.001617176 | KIAA0226 | 0.015441174 | 0.021162651 |
| TARBP2 | 1.0602657 | 1.25638E−09 | TPX2 | 1.1733183 | 2.47224E−05 | LRRC39 | 1.7843604 | 0.001617639 | METAP1 | 0.22739604 | 0.021175095 |
| RAB8A | 1.0528785 | 1.26079E−09 | CTSF | 0.7619801 | 2.47481E−05 | KLRG1 | 0.76339084 | 0.001623296 | FAM81A | 2.2756941 | 0.021184371 |
| SDHC | 0.93147284 | 1.3185E−09 | GDAP1 | 1.1694305 | 2.4827E−05 | GPR125 | 0.5531762 | 0.001626284 | CYB561D2 | 0.5104807 | 0.021250148 |
| RAP2B | 0.7353592 | 1.3388E−09 | NBN | 0.5263065 | 2.48706E−05 | ASXL1 | 0.30249122 | 0.001626517 | PXN | 0.345365 | 0.021250148 |
| TRAF5 | 0.713487 | 1.34513E−09 | CSTF2 | 0.9005012 | 2.50035E−05 | SUFU | 0.82809883 | 0.00163644 | PRKCB | 1.5055223 | 0.021253087 |
| AGPAT6 | 0.83972377 | 1.34513E−09 | TRIM14 | 0.42390844 | 2.50675E−05 | RPL17-C18orf32 | 0.7316686 | 0.001649671 | ELOVL6 | 0.9092806 | 0.021281267 |
| SRP72 | 1.0687914 | 1.36762E−09 | NHLRC1 | 0.97784764 | 2.52977E−05 | OIP5 | 1.3206694 | 0.001651772 | PRAF2 | 0.405747 | 0.021287398 |
| MRPL24 | 1.1298013 | 1.38007E−09 | TMEM68 | 0.45855933 | 2.55117E−05 | SMARCD2 | 0.45446092 | 0.001654809 | PISD | 0.27386537 | 0.0213031 |
| GLT25D1 | 0.90719306 | 1.40773E−09 | KCNQ5 | 1.6983595 | 2.56296E−05 | RAP2C | 0.41237384 | 0.001656604 | MAP3K12 | 0.46074525 | 0.021376098 |
| ECHDC1 | 0.9646414 | 1.45277E−09 | LOC100506548 | 0.41216227 | 2.59595E−05 | SFXN2 | 0.41575438 | 0.001661838 | BCL11A | 0.7659258 | 0.021388656 |
| HMCN1 | 0.9815372 | 1.45692E−09 | CHKB | 0.91086316 | 2.63162E−05 | ODF2 | 0.42202434 | 0.001662389 | NPDC1 | 0.34289548 | 0.021391369 |
| CUEDC2 | 1.2434427 | 1.45869E−09 | AHRR | 0.7003103 | 2.63247E−05 | FBXO22-AS1 | 0.5011854 | 0.001669522 | AARS | 0.44697633 | 0.021435505 |
| GRHPR | 1.1589133 | 1.45904E−09 | ZWILCH | 1.1677334 | 2.64965E−05 | SNHG7 | 0.20611244 | 0.001670629 | RBM12B | 0.27583686 | 0.021451305 |
| CRK | 0.68596464 | 1.47856E−09 | ZNF791 | 0.59501654 | 2.65303E−05 | TTF1 | 0.30559757 | 0.001670629 | KAT6B | 0.116606474 | 0.021457482 |
| SEC23IP | 0.7036543 | 1.51062E−09 | ZC3H13 | 0.5536445 | 2.65642E−05 | ANKRD13C | 0.25968274 | 0.001672126 | RIN2 | 0.46580717 | 0.021476025 |
| OAT | 1.2403307 | 1.55483E−09 | LPGAT1 | 0.180112 | 2.65665E−05 | KANK1 | 0.3740414 | 0.001673309 | EEF1E1-MUTED | 0.5849432 | 0.021500775 |
| ATAD3B | 0.9180753 | 1.55483E−09 | ASNSD1 | 0.75056386 | 2.65877E−05 | MPHOSPH6 | 0.65582603 | 0.001678833 | YBEY | 0.3341646 | 0.021500775 |
| GALM | 0.8599518 | 1.56028E−09 | C10orf32 | 0.4327477 | 2.67612E−05 | PMM2 | 0.3894311 | 0.001678833 | TPI1 | 0.5597506 | 0.021500775 |
| DCLRE1C | 0.8496073 | 1.56576E−09 | PAPL | 0.06744969 | 2.67635E−05 | UBR1 | 0.32290843 | 0.001685565 | RAB8B | 0.2949279 | 0.02151662 |
| POLD2 | 1.2397075 | 1.57369E−09 | EBP | 0.81342506 | 2.68278E−05 | MYO5C | 0.28809333 | 0.001687709 | FLRT1 | 0.91921425 | 0.021560994 |
| TBCE | 1.0559337 | 1.57369E−09 | TRIM16 | 0.42513594 | 2.68278E−05 | F3 | 0.06554091 | 0.00169208 | SLC4A2 | 0.1674858 | 0.021560994 |
| FAM195B | 1.1066116 | 1.60723E−09 | NUMB | 0.5900842 | 2.68278E−05 | ALG1 | 0.3258001 | 0.001696142 | HOOK2 | 0.4644124 | 0.021570428 |
| HMGA1 | 0.8712865 | 1.6113E−09 | WDR77 | 0.7723406 | 2.69387E−05 | BTF3 | 0.40552422 | 0.001696704 | TMOD3 | 0.13326217 | 0.021592057 |
| MRPS26 | 1.1978308 | 1.69509E−09 | ATP5SL | 0.880465 | 2.71789E−05 | NFKBIB | 0.2152043 | 0.001700777 | MND1 | 0.73141223 | 0.021592057 |
| ABCB6 | 1.1429863 | 1.69993E−09 | RGS20 | 0.71578234 | 2.7207E−05 | NCAPD2 | 0.34264696 | 0.001707423 | PCSK7 | 0.3457626 | 0.021607963 |
| KCTD15 | 1.0645066 | 1.72037E−09 | CRTC1 | 0.23713925 | 2.75082E−05 | WDR48 | 0.4946127 | 0.001710556 | PPP1R16A | 0.33680698 | 0.021620648 |
| ATAD1 | 1.0412539 | 1.73259E−09 | NCOA5 | 0.664968 | 2.75106E−05 | COQ2 | 1.1710591 | 0.001713373 | PLA2G4C | 1.1642438 | 0.021665456 |
| MPV17 | 1.3298894 | 1.73866E−09 | ELK4 | 0.3601324 | 2.75326E−05 | LOC148413 | 0.6486475 | 0.001723943 | NDUFB11 | 0.5515812 | 0.021674933 |
| CEP170 | 0.81343085 | 1.80884E−09 | TANC2 | 0.498376 | 2.75529E−05 | NEK6 | 0.57699776 | 0.001724514 | LIPT1 | 0.34767473 | 0.021749062 |
| DUS3L | 1.1210762 | 1.83222E−09 | GLTSCR2 | 0.67366767 | 2.75529E−05 | DNAJC15 | 0.48778892 | 0.001728649 | RPL36AL | 0.46193898 | 0.021765072 |
| SCNM1 | 1.1035749 | 1.85362E−09 | CDC42 | 0.67559654 | 2.75529E−05 | BSCL2 | 0.6193177 | 0.00172987 | FMN1 | 0.21334398 | 0.021774588 |
| PPIA | 0.50762105 | 1.95033E−09 | AMOTL2 | 0.7291845 | 2.76059E−05 | KDM6A | 0.45842475 | 0.001734668 | TBC1D10B | 0.2998584 | 0.021777602 |
| PSME3 | 0.95613486 | 1.95399E−09 | GALK2 | 0.52621925 | 2.76477E−05 | SCFD1 | 0.53387356 | 0.00173484 | RFNG | 0.33683696 | 0.021790376 |
| RPS19BP1 | 1.1232412 | 1.96083E−09 | PGAP1 | 0.37759468 | 2.76895E−05 | SLC25A22 | 0.45251468 | 0.00173484 | MRPS30 | 0.4385884 | 0.021799903 |
| NIPA2 | 0.96021515 | 1.96515E−09 | FAM57A | 1.138478 | 2.7903E−05 | HHAT | 0.6049071 | 0.00173574 | LOC100127983 | 0.6309685 | 0.021802921 |
| GK | 1.3117907 | 1.99647E−09 | CIZ1 | 0.5594613 | 2.81114E−05 | NUP85 | 0.37307835 | 0.001740227 | PMS2L2 | 0.24668725 | 0.021812452 |
| PRPSAP2 | 1.1105281 | 2.04874E−09 | VPS37B | 0.3932156 | 2.81363E−05 | GPAA1 | 0.60101366 | 0.00174113 | ANKRA2 | 0.41028 | 0.021896327 |
| CAMTA2 | 0.7051094 | 2.13733E−09 | IGFBPL1 | 0.53601366 | 2.83262E−05 | MUL1 | 0.5657447 | 0.001743013 | FCGRT | 0.47310665 | 0.021974408 |
| RBFA | 0.71101874 | 2.14758E−09 | ARNT | 0.5445967 | 2.83825E−05 | USP7 | 0.18616028 | 0.001743263 | DNMBP | 0.82357794 | 0.021987287 |
| HNRNPD | 0.68911904 | 2.1544E−09 | MAPKBP1 | 0.38143155 | 2.83985E−05 | SLC25A12 | 0.44982013 | 0.001746785 | PPP4R1 | 0.36220694 | 0.02199361 |
| TOMM22 | 1.1456077 | 2.21428E−09 | FAM69A | 0.7425537 | 2.84212E−05 | KIAA1586 | 0.36908534 | 0.001752612 | ACER3 | 0.4150528 | 0.022002508 |
| NONO | 0.87733227 | 2.27947E−09 | MRPS18A | 0.7729409 | 2.85114E−05 | RP1-177G6.2 | 0.34894675 | 0.001758458 | ABCC1 | 0.50502986 | 0.022002508 |
| ZCCHC7 | 0.8888875 | 2.2836E−09 | PKD1 | 0.13026156 | 2.86698E−05 | PRKD2 | 1.0227649 | 0.001762669 | SIK2 | 0.2186494 | 0.022041214 |
| PKN1 | 0.7685632 | 2.2836E−09 | PLEKHO2 | 0.69824904 | 2.87812E−05 | UHRF1BP1 | 0.21182373 | 0.001762845 | SMYD3 | 0.29327586 | 0.022041214 |
| CLPP | 1.2639912 | 2.31838E−09 | GAPDHS | 0.98779666 | 2.88179E−05 | CDC37 | 0.81988686 | 0.001762845 | PAXBP1 | 0.2434541 | 0.022041214 |
| MRRF | 1.1022493 | 2.33854E−09 | CLDN1 | 2.6627462 | 2.90878E−05 | EIF3H | 0.574179 | 0.001763098 | CCDC125 | 0.15586516 | 0.022096918 |
| ZNF566 | 0.62402564 | 2.34671E−09 | ALDH16A1 | 0.68086624 | 2.91456E−05 | RANBP9 | 0.30805445 | 0.001768975 | M6PR | 0.51128376 | 0.022116456 |
| POLR1E | 1.2335416 | 2.3994E−09 | KDM4A | 0.6488048 | 2.93281E−05 | RAF1 | 0.512974 | 0.001771473 | NBPF11 | 0.12184675 | 0.022152276 |
| LEO1 | 1.1118977 | 2.40857E−09 | LRWD1 | 0.842531 | 2.93307E−05 | CDC42EP4 | 0.43359885 | 0.001771473 | FEM1A | 0.30226496 | 0.022287538 |
| EIF4B | 0.9214904 | 2.4092E−09 | PRPF38A | 0.62789094 | 2.95144E−05 | METTL22 | 0.6450908 | 0.001779042 | NBPF24 | 0.13910349 | 0.022287538 |
| ZNF687 | 1.0837733 | 2.4317E−09 | C2orf69 | 0.58820266 | 2.96991E−05 | SYT11 | 0.22552903 | 0.001787309 | MAP4K1 | 0.6410072 | 0.022306987 |
| MRPS18B | 1.1216007 | 2.43785E−09 | PRTG | 0.86871 | 2.97932E−05 | STX17 | 0.08917182 | 0.001802603 | LOC100499405 | 0.46064982 | 0.022369473 |
| MCCC2 | 0.7137333 | 2.44308E−09 | ACTR8 | 0.59632295 | 2.98735E−05 | MYL6B | 0.4972689 | 0.001805145 | LRRC57 | 0.19223703 | 0.022412329 |
| MRPS24 | 1.2550683 | 2.44308E−09 | RELA | 0.54238874 | 2.99752E−05 | TMEM164 | 0.57283497 | 0.001805404 | ZNF550 | 0.63726497 | 0.022432115 |
| C1orf131 | 0.93962395 | 2.44453E−09 | GOLGA3 | 0.34458438 | 3.00942E−05 | MTBP | 0.48014554 | 0.001808367 | CCDC101 | 0.6066569 | 0.022435217 |
| SMU1 | 0.6465484 | 2.45217E−09 | LRRC41 | 0.77596927 | 3.03828E−05 | EIF5A | 0.66916513 | 0.001809303 | FGL2 | 0.18328893 | 0.022458363 |
| RNF40 | 0.73990715 | 2.45217E−09 | TEX10 | 0.6215535 | 3.09022E−05 | MCAT | 0.76242274 | 0.001810239 | EPHA2 | 0.92867774 | 0.022481531 |
| C9orf142 | 1.0668036 | 2.45917E−09 | RPP30 | 0.421188 | 3.12673E−05 | TRAF2 | 0.51225287 | 0.001812532 | AATF | 0.4441461 | 0.022568177 |
| CNPPD1 | 1.0037509 | 2.50036E−09 | SLK | 0.46889582 | 3.12673E−05 | STAT6 | 0.45140737 | 0.001816524 | MAF1 | 0.4969399 | 0.022584727 |
| VEPH1 | 1.427075 | 2.50036E−09 | NXT1 | 0.7731401 | 3.1332E−05 | SLC7A6OS | 0.61493534 | 0.001819164 | GTPBP1 | 0.22702156 | 0.022591207 |
| ISG15 | 1.5329988 | 2.53757E−09 | UBL4A | 0.82704186 | 3.18566E−05 | MTSS1 | 0.29933092 | 0.001824875 | NUS1 | 0.33635753 | 0.022627951 |
| DCTPP1 | 1.1669793 | 2.54318E−09 | MDH1 | 0.91201484 | 3.1867E−05 | CHD8 | 0.29347226 | 0.001827184 | MEGF10 | 1.3026348 | 0.022654642 |
| PAN3 | 0.69862163 | 2.54552E−09 | ARID1B | 0.20764516 | 3.2046E−05 | RCBTB1 | 0.38748538 | 0.001831812 | FTO | 0.14998864 | 0.022674619 |
| AIFM1 | 0.7795367 | 2.55856E−09 | UAP1 | 0.67026347 | 3.2046E−05 | ZNF618 | 0.4357697 | 0.001836029 | IGF2R | 0.11512862 | 0.022677751 |
| DCAF7 | 0.8658248 | 2.60835E−09 | SMPD4 | 0.5258335 | 3.20971E−05 | DLST | 0.27563998 | 0.001836029 | SRD5A1 | 0.48746845 | 0.022778364 |
| ATP5G2 | 0.59591115 | 2.62244E−09 | PTTG1IP | 0.6836175 | 3.20971E−05 | ZBTB48 | 0.40324324 | 0.001843165 | DNPEP | 0.050249737 | 0.02278828 |
| BMP7 | 0.5064992 | 2.62244E−09 | NUP54 | 0.7393533 | 3.22365E−05 | AMDHD2 | 0.4366566 | 0.00184722 | EXTL3 | 0.3901737 | 0.022815139 |
| FHL2 | 1.2412854 | 2.6274E−09 | ELP5 | 0.56842107 | 3.24146E−05 | TUBGCP3 | 0.4519903 | 0.001848176 | CCDC23 | 0.3932086 | 0.022838635 |
| POLR3K | 1.2203659 | 2.75183E−09 | PTK7 | 0.678612 | 3.2494E−05 | DISC1 | 0.35224286 | 0.001850859 | MTHFD1L | 0.22761524 | 0.022913132 |
| NANP | 1.1277558 | 2.78106E−09 | ZMYM2 | 0.44902658 | 3.25276E−05 | SHROOM3 | 0.022605281 | 0.00185573 | AUTS2 | 0.60310256 | 0.022950101 |
| HN1 | 1.1016978 | 2.81241E−09 | ATE1 | 0.7202227 | 3.26072E−05 | SNORA67 | 1.3659245 | 0.00185573 | AKAP8 | 0.22249614 | 0.022950101 |
| MTIF3 | 1.0089964 | 2.81772E−09 | EHMT1 | 0.5899555 | 3.26256E−05 | DDX12P | 0.54001206 | 0.001859573 | MXRA7 | 0.20572609 | 0.02302834 |
| CDC6 | 0.9934474 | 2.86691E−09 | ANKS4B | 0.108653255 | 3.27749E−05 | TET2 | 0.071369596 | 0.001860883 | FNIP1 | 0.17606543 | 0.02304519 |
| ARMC6 | 1.1982886 | 2.95748E−09 | POLR2B | 0.6275543 | 3.29666E−05 | CASP10 | 0.046076372 | 0.001863583 | KIAA0753 | 0.55259407 | 0.023086015 |
| GARS | 1.1149759 | 2.96973E−09 | SLMO2 | 0.6310294 | 3.29666E−05 | LOC730183 | 0.93716717 | 0.001884044 | KANSL2 | 0.32281888 | 0.023099476 |
| HAS2 | 1.4910998 | 2.98576E−09 | LOC100288637 | 0.26522192 | 3.3024E−05 | GXYLT1 | 0.16037591 | 0.001889511 | MTMR6 | 0.37681684 | 0.02312642 |
| RBBP4 | 0.7558486 | 2.98576E−09 | TBL3 | 0.80959797 | 3.3027E−05 | CT64 | 0.013250005 | 0.001902859 | BLOC1S5 | 0.34808204 | 0.023221915 |
| C17orf85 | 0.58903605 | 3.0388E−09 | LOC400043 | 1.0962827 | 3.31078E−05 | CTPS2 | 0.5753968 | 0.001907747 | DCTN4 | 0.2933079 | 0.023238891 |
| SYMPK | 0.8398281 | 3.09176E−09 | CCDC148 | 0.17488447 | 3.31199E−05 | ANKRD40 | 0.34234953 | 0.001908377 | PPP1R37 | 0.7908697 | 0.023280021 |
| ASCC2 | 0.903743 | 3.09562E−09 | UBE2V1 | 0.67874986 | 3.31199E−05 | C16orf59 | 1.2658638 | 0.001912485 | NCAPG | 0.7564651 | 0.02331085 |
| FAM65A | 0.9113053 | 3.14442E−09 | ZNF761 | 0.28618488 | 3.31464E−05 | GYPC | 0.5432801 | 0.001912485 | NFATC4 | 0.59613395 | 0.023369399 |
| MKL1 | 0.87056994 | 3.19918E−09 | TUBA1C | 0.88146335 | 3.31806E−05 | KIF26B | 0.90442103 | 0.001914185 | CLK3 | 0.40985206 | 0.023420894 |
| SMC3 | 0.8100818 | 3.20627E−09 | FBXW2 | 0.2627596 | 3.32462E−05 | UBE2R2 | 0.46181017 | 0.001914817 | ADAL | 0.050539106 | 0.023455363 |
| PPP2R5C | 0.9074583 | 3.23816E−09 | TBC1D7 | 0.88257253 | 3.34061E−05 | RBM14 | 0.40629503 | 0.001919734 | SLC27A1 | 0.21474375 | 0.023548824 |
| C19orf60 | 1.1063645 | 3.33803E−09 | C19orf12 | 0.32850978 | 3.36093E−05 | SNHG11 | 0.6405886 | 0.00192494 | SLC38A6 | 0.21546659 | 0.023625379 |
| POLL | 1.1581198 | 3.35292E−09 | HAT1 | 0.8057415 | 3.36757E−05 | CDC23 | 0.5027677 | 0.001929163 | SLC39A4 | 0.27969584 | 0.023702155 |
| TRIAP1 | 1.2363561 | 3.35712E−09 | AMZ2P1 | 0.41748118 | 3.3801E−05 | 44441 | 0.61522985 | 0.001930159 | CTTNBP2NL | 0.3593819 | 0.023747276 |
| ALDH1L2 | 0.7443536 | 3.3721E−09 | GM2A | 0.6291309 | 3.38359E−05 | HCCS | 0.66605926 | 0.00195703 | CDC42SE1 | 0.3240036 | 0.023747276 |
| ANKRD36 | 0.7307839 | 3.45606E−09 | UCK1 | 0.8632418 | 3.38629E−05 | TBK1 | 0.35921034 | 0.00195703 | SPIN2B | 0.36072007 | 0.023761079 |
| RBM4 | 1.1338065 | 3.47139E−09 | EPB41L1 | 0.9979046 | 3.40368E−05 | DBR1 | 0.25588658 | 0.001958039 | CAPNS1 | 0.28491914 | 0.023816862 |
| TP53RK | 0.82531697 | 3.47139E−09 | CLOCK | 0.94809747 | 3.408E−05 | RPS6KA4 | 1.0525082 | 0.0019616 | METTL2A | 0.26775438 | 0.023989206 |
| RUVBL1 | 1.2236519 | 3.48243E−09 | MTIF2 | 0.4948588 | 3.45996E−05 | USP46 | 0.024259694 | 0.001961883 | CCDC112 | 0.3594167 | 0.024003134 |
| KRTAP19-1 | 4.0434117 | 3.52151E−09 | XG | 1.0527169 | 3.48966E−05 | FTSJ1 | 0.45344526 | 0.001967926 | E2F7 | 0.82047105 | 0.024041662 |
| SNX2 | 1.1175911 | 3.56444E−09 | VARS2 | 0.461002 | 3.49326E−05 | MED22 | 0.5960443 | 0.001983998 | LINC00189 | 0.11198179 | 0.024197588 |
| RPLP2 | 0.53616416 | 3.65061E−09 | TATDN3 | 0.5207519 | 3.52406E−05 | C11orf30 | 0.7036628 | 0.001988711 | USP32P1 | 0.093702845 | 0.024203972 |
| POM121C | 0.6093796 | 3.65631E−09 | MC1R | 0.8979935 | 3.54911E−05 | MINOS1 | 0.35766298 | 0.001989736 | SCML2 | 0.19118266 | 0.024228735 |
| NOS3 | 0.885115 | 3.65631E−09 | CYTH2 | 0.39895186 | 3.54911E−05 | SLC2A6 | 0.37139282 | 0.002005228 | RER1 | 0.45756528 | 0.024324677 |
| NDUFAF6 | 0.9497338 | 3.7198E−09 | HCG11 | 0.41993383 | 3.55028E−05 | SPRYD7 | 0.2017202 | 0.002008412 | HEATR6 | 0.13521814 | 0.024363917 |
| BCS1L | 1.0769149 | 3.90315E−09 | ATP5O | 0.9657239 | 3.55394E−05 | TVP23B | 0.55422544 | 0.002015048 | TACC3 | 0.88522935 | 0.02437085 |
| NDUFAB1 | 1.2695042 | 3.94183E−09 | FAM35A | 1.0962138 | 3.56513E−05 | 44448 | 0.29592395 | 0.002015711 | FAM210B | 0.2124643 | 0.024583456 |
| LAMC1 | 0.6992291 | 3.94806E−09 | MAP3K9 | 0.1275498 | 3.59097E−05 | NAA40 | 0.19020909 | 0.002026039 | KIAA0317 | 0.1684231 | 0.024586563 |
| PSPC1 | 0.97731584 | 4.11617E−09 | AAGAB | 0.6948285 | 3.59097E−05 | HSCB | 0.2901369 | 0.002031428 | PRKCI | 0.082605556 | 0.024586563 |
| NUDCD1 | 0.89452624 | 4.13712E−09 | RHBDF2 | 0.628054 | 3.60988E−05 | YWHAH | 0.5105907 | 0.002031428 | DHX57 | 0.41080713 | 0.024589672 |
| ACTG1 | 0.5794422 | 4.26535E−09 | DTNA | 1.2851684 | 3.63178E−05 | ZDHHC3 | 0.2354667 | 0.002038049 | PLIN3 | 0.24034815 | 0.024665593 |
| MRPL15 | 1.1059322 | 4.31577E−09 | AKAP11 | 0.56904685 | 3.63178E−05 | EIF4EBP3 | 0.6988304 | 0.002038049 | PKDCC | 1.010367 | 0.024741464 |
| CDK4 | 1.0533764 | 4.34186E−09 | PVR | 0.12737726 | 3.63723E−05 | TWF2 | 0.60768247 | 0.002039769 | GNG7 | 0.28975424 | 0.024781301 |
| MTX1 | 1.0034238 | 4.34319E−09 | LOC100506421 | 0.3372692 | 3.64525E−05 | GCSH | 0.57922894 | 0.002044984 | FUBP3 | 0.32401025 | 0.024907962 |
| FAM96A | 1.164707 | 4.34591E−09 | LINC00094 | 0.25209773 | 3.65843E−05 | LETMD1 | 0.5587911 | 0.002045657 | PRELID1 | 0.6468149 | 0.024962718 |
| KIAA0101 | 0.8135296 | 4.39029E−09 | SARS2 | 0.79435277 | 3.67509E−05 | APOOL | 0.29699853 | 0.002050421 | ARID4A | 0.1474856 | 0.025179641 |
| IFRD2 | 1.1184155 | 4.48326E−09 | DDX1 | 0.7633638 | 3.67802E−05 | ZNF788 | 0.041394304 | 0.002055196 | CSRNP3 | 0.18302388 | 0.025227515 |
| HMGB1 | 0.7913206 | 4.50606E−09 | AFMID | 0.46707028 | 3.68182E−05 | ZSCAN25 | 0.5112407 | 0.002059599 | TACC2 | 0.6884349 | 0.025290058 |
| PDS5B | 0.6387924 | 4.54482E−09 | UPK3B | 0.27052358 | 3.68389E−05 | BCYRN1 | 0.53643024 | 0.002059599 | NCOA6 | 0.06071361 | 0.025290058 |
| TAF1D | 0.86909884 | 4.62482E−09 | PPHLN1 | 0.6213144 | 3.7313E−05 | EPS8L2 | 0.65663564 | 0.002063715 | C16orf70 | 0.5327739 | 0.025389986 |
| TRMT10C | 1.0382756 | 4.62773E−09 | ANXA5 | 0.4857811 | 3.73952E−05 | CNOT11 | 0.60976785 | 0.002070051 | NSD1 | 0.12622611 | 0.025445416 |
| PSMA7 | 0.9323434 | 4.67938E−09 | ATP13A1 | 0.57681656 | 3.74724E−05 | RFC2 | 0.3547689 | 0.002070051 | PRC1 | 0.5729523 | 0.025486257 |
| RNPS1 | 1.0774953 | 4.70018E−09 | HOXA10 | 1.153871 | 3.74724E−05 | PPWD1 | 0.41097003 | 0.002071883 | HOXA4 | 2.307183 | 0.025489463 |
| FAM204A | 0.6696144 | 4.71959E−09 | SLC7A1 | 0.59167576 | 3.78991E−05 | RNF10 | 0.47277096 | 0.002076021 | CDC20 | 0.81096375 | 0.025489463 |
| TUBGCP5 | 0.86132634 | 4.73755E−09 | RAPGEF1 | 0.3678835 | 3.79027E−05 | NFATC2IP | 0.13400418 | 0.002085951 | ACTR5 | 0.32320434 | 0.025582317 |
| IFI30 | 1.4407456 | 4.73755E−09 | CYP27A1 | 0.82402426 | 3.79951E−05 | LOC100131067 | 0.100422196 | 0.002087796 | STRA13 | 0.6135618 | 0.025788352 |
| WASH7P | 0.69674915 | 4.73755E−09 | SPRTN | 0.76512593 | 3.81322E−05 | ACTB | 0.38963094 | 0.002092652 | METTL14 | 0.17019516 | 0.025795641 |
| KLHL12 | 0.92284775 | 4.87002E−09 | ZNF92 | 0.48589203 | 3.83415E−05 | PILRB | 0.36434546 | 0.002106071 | SNRPD3 | 0.4906791 | 0.025799152 |
| SBNO1 | 0.68973434 | 4.97607E−09 | ZC3H7A | 0.46074003 | 3.88049E−05 | NEK3 | 0.6232423 | 0.002106677 | TTC9C | 0.42296255 | 0.025851586 |
| INTS3 | 0.6830219 | 5.0204E−09 | RBBP9 | 0.31763658 | 3.88449E−05 | CPSF3L | 0.47159487 | 0.002118309 | LOC642846 | 0.14412606 | 0.025870259 |
| RABEP1 | 0.654077 | 5.0204E−09 | ORC3 | 0.5751648 | 3.88795E−05 | ZNF394 | 0.25081202 | 0.002119397 | LOC100505738 | 0.20656806 | 0.025885152 |
| HYOU1 | 0.618145 | 5.08118E−09 | SMC1A | 0.47174916 | 3.88795E−05 | ST7L | 0.39153028 | 0.0021314 | CXorf21 | 0.05358559 | 0.025892467 |
| NOP58 | 1.0164481 | 5.18024E−09 | FAM175B | 0.9577671 | 3.89196E−05 | ANKRD44 | 0.47070417 | 0.002137535 | CKMT2 | 1.5188853 | 0.02591822 |
| PPRC1 | 1.0942365 | 5.23131E−09 | ZBED4 | 0.8509595 | 3.9133E−05 | IRS1 | 0.70123714 | 0.002139027 | SRBD1 | 0.28283516 | 0.025966801 |
| MIPEP | 1.0411302 | 5.24625E−09 | ZNF551 | 0.39093107 | 3.93015E−05 | SMARCAL1 | 0.38318342 | 0.00214646 | EXOSC9 | 0.34848493 | 0.025989353 |
| MBD6 | 0.755002 | 5.28959E−09 | U2AF1L4 | 0.75607455 | 3.93788E−05 | MGAT5B | 1.2698712 | 0.002151443 | PET100 | 0.6223108 | 0.026014932 |
| MCM4 | 0.95780987 | 5.32658E−09 | SYF2 | 0.61243737 | 3.97056E−05 | SDHA | 0.54640883 | 0.002151443 | DDTL | 0.64987123 | 0.026014932 |
| ATXN2L | 0.6971447 | 5.32658E−09 | MRPS23 | 0.8040584 | 3.97465E−05 | NMD3 | 0.5347595 | 0.002168901 | TRIM6 | 0.15397976 | 0.026014932 |
| SGPL1 | 0.71664006 | 5.43203E−09 | GTF3C2 | 0.66140544 | 4.01043E−05 | PCYT2 | 0.6219226 | 0.002170013 | PARS2 | 0.81698537 | 0.026048957 |
| PLEKHF2 | 1.1509316 | 5.43551E−09 | SLC25A26 | 0.49146825 | 4.03804E−05 | BRD3 | 0.31272376 | 0.002179555 | NAAA | 0.109707355 | 0.026132412 |
| KIAA0907 | 0.6278924 | 5.45276E−09 | NAE1 | 0.76193315 | 4.12309E−05 | MTERFD3 | 0.7215718 | 0.00218027 | KRTCAP2 | 0.44248658 | 0.026158919 |
| AIMP2 | 1.1723899 | 5.51344E−09 | SEH1L | 0.6109329 | 4.14313E−05 | C9orf78 | 0.48717913 | 0.002186222 | EFHA1 | 0.3385057 | 0.02618162 |
| MGC57346 | 0.745805 | 5.53268E−09 | PNN | 0.55124617 | 4.15029E−05 | RAVER1 | 0.4447014 | 0.00219664 | PPIAP30 | 0.45139956 | 0.026272333 |
| MRPS31P5 | 0.80808926 | 5.56956E−09 | TXNL4B | 0.39691663 | 4.15553E−05 | CLPTM1 | 0.49548703 | 0.002197766 | RBM15B | 0.25978348 | 0.026272333 |
| CEP135 | 0.9746641 | 5.65467E−09 | RANBP17 | 0.81063396 | 4.16756E−05 | LOC100131089 | 0.068331055 | 0.002200109 | ELK1 | 0.14002326 | 0.026272333 |
| MYCBP2 | 0.61147887 | 5.77376E−09 | MRPS18C | 0.6664985 | 4.18254E−05 | C19orf55 | 0.69675213 | 0.002203266 | SLC35B2 | 0.5550586 | 0.026272333 |
| CHMP5 | 1.0080669 | 5.77747E−09 | ADPRHL2 | 0.8259636 | 4.18392E−05 | CARHSP1 | 0.085856326 | 0.002204713 | FSTL4 | 1.8773373 | 0.026325889 |
| FRA10AC1 | 0.39048156 | 5.98543E−09 | ATG9A | 0.7081823 | 4.19643E−05 | RAD51 | 0.9183085 | 0.002205843 | NABP2 | 0.25548252 | 0.026433292 |
| BCAS3 | 0.8808771 | 5.98543E−09 | SPAG7 | 0.8430864 | 4.19879E−05 | SOCS6 | 0.4695441 | 0.002211859 | FBXO11 | 0.043791126 | 0.026525584 |
| U2AF2 | 0.5084883 | 6.1306E−09 | COMTD1 | 1.4547616 | 4.20996E−05 | GCAT | 0.41866237 | 0.002212585 | GTF2H3 | 0.1656145 | 0.026529189 |
| FIBP | 1.1597058 | 6.15779E−09 | DDX19B | 0.74512005 | 4.21625E−05 | FAM86B1 | 0.8024499 | 0.002215759 | ATG4C | 0.4899874 | 0.026563809 |
| RRP1B | 0.7863595 | 6.15789E−09 | BAAT | 1.9965804 | 4.2196E−05 | DSCC1 | 0.9336647 | 0.002220574 | RHOBTB3 | 0.2055311 | 0.026575179 |
| DMXL1 | 0.70849305 | 6.2341E−09 | DPP3 | 0.8326864 | 4.23674E−05 | KIAA1429 | 0.4469822 | 0.002221302 | ALG13 | 0.09680033 | 0.026617353 |
| SLC24A5 | 1.2695807 | 6.23421E−09 | ACLY | 0.685795 | 4.24899E−05 | MYEF2 | 0.20137031 | 0.002225308 | ARL14EP | 0.24915615 | 0.026702419 |
| DCK | 1.2748634 | 6.32925E−09 | ARFGAP1 | 0.6032521 | 4.322E−05 | TUBGCP4 | 0.20657115 | 0.002235487 | RTF1 | 0.15039827 | 0.026702419 |
| OVCA2 | 1.0157379 | 6.45013E−09 | DNAJB12 | 0.5241787 | 4.32845E−05 | PGBD1 | 0.8172724 | 0.002252241 | ST6GAL1 | 0.27338716 | 0.026737239 |
| LYPLA2 | 1.1512693 | 6.55869E−09 | CCNJ | 0.91013414 | 4.36017E−05 | BIRC5 | 0.84242105 | 0.002255884 | ZNF710 | 0.61740273 | 0.026748675 |
| UTP3 | 1.1877844 | 6.57328E−09 | PPP1CC | 0.75731224 | 4.3854E−05 | LRRC3 | 0.29277858 | 0.002262027 | ZNF75A | 0.43472832 | 0.026752039 |
| BAG1 | 0.36675853 | 6.65034E−09 | LOC100130691 | 0.2454665 | 4.38787E−05 | DHFR | 0.5117427 | 0.002263601 | SCP2 | 0.51854706 | 0.026752039 |
| RAP1GAP | 1.5808489 | 7.00432E−09 | CRYM-AS1 | 0.031366285 | 4.50164E−05 | WIBG | 0.6314046 | 0.00226476 | CFH | 1.310398 | 0.026763482 |
| PMF1 | 1.0446268 | 7.0641E−09 | TMPO | 0.50710654 | 4.51865E−05 | SLC25A1 | 0.61100286 | 0.002272832 | UBP1 | 0.41529337 | 0.026848423 |
| SNRPF | 1.0120302 | 7.10428E−09 | SRSF1 | 0.5549012 | 4.51865E−05 | POSTN | 1.0269961 | 0.002281954 | PACS2 | 0.12949473 | 0.026879492 |
| TMOD2 | 0.8741692 | 7.1402E−09 | SERPINB9 | 0.7528661 | 4.51865E−05 | NSMCE1 | 0.65892416 | 0.002290926 | HAUS2 | 0.06821199 | 0.026922365 |
| ADAM15 | 1.0097857 | 7.15832E−09 | USP24 | 0.33735374 | 4.52539E−05 | SLC15A4 | 0.509841 | 0.002293783 | PACSIN3 | 0.64531285 | 0.026945386 |
| XRN2 | 0.9209107 | 7.22166E−09 | UBE2M | 0.8939565 | 4.54776E−05 | SPDYE2 | 0.20357253 | 0.002294352 | EIF4ENIF1 | 0.3348488 | 0.027086301 |
| TCEANC2 | 0.68046755 | 7.24908E−09 | DNTTIP2 | 0.65860593 | 4.54776E−05 | SPDYE2L | 0.20357253 | 0.002294352 | ABHD14A-ACY1 | 1.0796372 | 0.027093924 |
| NRP2 | 0.9399264 | 7.32468E−09 | PSAT1 | 1.0482687 | 4.54776E−05 | MED8 | 0.6044881 | 0.002294352 | CMTM7 | 0.44666946 | 0.027105497 |
| CCT7 | 1.1438491 | 7.33657E−09 | APOPT1 | 0.467755 | 4.56829E−05 | NAA15 | 0.36041978 | 0.002307789 | EMC9 | 0.4994998 | 0.027155791 |
| TCTN3 | 1.1352872 | 7.36695E−09 | PSIMCT-1 | 0.86472976 | 4.59362E−05 | POLR2C | 0.48570597 | 0.002311939 | ARHGAP10 | 0.6061795 | 0.027155791 |
| MINPP1 | 1.1189897 | 7.36695E−09 | THAP6 | 0.33088356 | 4.653E−05 | RDH11 | 0.5993936 | 0.002313546 | PPP1R21 | 0.1118592 | 0.027159477 |
| RAN | 0.765497 | 7.3741E−09 | KLHL20 | 0.6351902 | 4.68807E−05 | RNFT1 | 0.5756897 | 0.002326661 | CCDC107 | 0.39574444 | 0.027167121 |
| TMEM177 | 1.278135 | 7.39748E−09 | ATP5J2-PTCD1 | 0.76750815 | 4.73874E−05 | HNRNPKP3 | 0.358027 | 0.002335125 | GMPPB | 0.5470841 | 0.02720221 |
| WDR43 | 0.6740455 | 7.96048E−09 | CAMK2D | 0.46965155 | 4.80149E−05 | FAT3 | 0.03243977 | 0.002335889 | YY1 | 0.18165818 | 0.027205904 |
| SPDYE1 | 0.538673 | 8.07865E−09 | TCEA1 | 0.66937256 | 4.81642E−05 | DSN1 | 0.45498648 | 0.002361896 | ZCCHC8 | 0.046058036 | 0.027209599 |
| UBLCP1 | 0.9701845 | 8.09915E−09 | CENPL | 0.45048195 | 4.8314E−05 | RTN4IP1 | 0.13146245 | 0.00236995 | RALGAPB | 0.3067721 | 0.0272162 |
| OLIG1 | 2.5221765 | 8.09941E−09 | ZNF24 | 0.48070621 | 4.88241E−05 | ZWINT | 0.8044797 | 0.002377253 | WBP11 | 0.25535122 | 0.0272162 |
| PRDM2 | 0.5859392 | 8.16836E−09 | DEGS1 | 0.742461 | 4.8861E−05 | TMEM186 | 0.7389323 | 0.002380211 | HMOX1 | 0.78303087 | 0.0272162 |
| USF2 | 0.96395296 | 8.25077E−09 | NSUN5P2 | 0.5174887 | 4.8861E−05 | ZNF100 | 0.077834226 | 0.002387011 | IST1 | 0.38337475 | 0.0272162 |
| PYCR1 | 1.0924586 | 8.25105E−09 | COG1 | 0.72350127 | 4.8861E−05 | LOC100128252 | 1.0313114 | 0.002387011 | RAB40C | 0.84115213 | 0.027266956 |
| DIABLO | 1.0101653 | 8.2979E−09 | ACN9 | 0.5965136 | 4.90129E−05 | HMG20B | 0.61238956 | 0.002390856 | DCLK1 | 1.1968653 | 0.0272786 |
| POLDIP3 | 0.9469187 | 8.33198E−09 | CRKL | 0.50899065 | 4.92886E−05 | PCBP2 | 0.45564732 | 0.00239242 | USP5 | 0.19060755 | 0.027341392 |
| KIAA0930 | 0.5629102 | 8.48997E−09 | UBQLN4 | 0.4757819 | 4.99353E−05 | MLLT10 | 0.27536628 | 0.002396523 | HNRPLL | 0.15270561 | 0.027341392 |
| MRPS12 | 1.1958286 | 8.49293E−09 | ZNF280B | 0.50481826 | 5.00094E−05 | PARPBP | 1.0360447 | 0.002396523 | TMC8 | 1.138397 | 0.027361025 |
| WAC-AS1 | 0.5480447 | 8.94287E−09 | MRPL46 | 0.7972808 | 5.06768E−05 | MRPL1 | 0.4781603 | 0.002400727 | GABPA | 0.24842803 | 0.027460163 |
| SCML4 | 0.5059976 | 8.97395E−09 | CDC42BPA | 0.5216727 | 5.11163E−05 | MICAL3 | 0.19073007 | 0.002400727 | C21orf2 | 0.3198491 | 0.027611739 |
| MRPL19 | 0.59488076 | 9.02765E−09 | TUFT1 | 0.4220593 | 5.11213E−05 | SNRPB | 0.6847607 | 0.002401512 | KIFC3 | 0.29268202 | 0.02775949 |
| EIF3G | 1.4268057 | 9.07034E−09 | MRPL13 | 0.82784814 | 5.11499E−05 | KLF16 | 0.50402963 | 0.002404499 | TM9SF2 | 0.42890677 | 0.027779388 |
| MGST3 | 0.856394 | 9.19104E−09 | CCT6P1 | 0.58279693 | 5.14203E−05 | HIBCH | 0.43336922 | 0.002406166 | MST1R | 1.6581962 | 0.027790959 |
| SKP2 | 0.69016206 | 9.19143E−09 | CDK20 | 0.9555346 | 5.15203E−05 | ERAL1 | 0.668717 | 0.002417458 | TOR1AIP2 | 0.23746835 | 0.027814916 |
| ZFHX3 | 0.60920614 | 9.29555E−09 | TRAPPC2L | 0.9210343 | 5.1692E−05 | ZNF211 | 1.0274215 | 0.002418248 | PPIB | 0.5899834 | 0.027834583 |
| GLUL | 1.1313407 | 9.31916E−09 | NCSTN | 0.76003635 | 5.1738E−05 | SLC35B4 | 0.15756312 | 0.002419039 | CHST10 | 0.52428013 | 0.027866397 |
| MED17 | 0.7617569 | 9.34866E−09 | GPHN | 0.3637109 | 5.17669E−05 | FUZ | 0.42111033 | 0.002425944 | PFKM | 0.3724513 | 0.027882052 |
| CENPF | 0.9516153 | 9.40166E−09 | ASB11 | 0.19634372 | 5.18676E−05 | ASAH1 | 0.6159532 | 0.002425944 | GTF2A1 | 0.29115242 | 0.027905815 |
| CRTC3 | 0.8573955 | 9.56498E−09 | PRDM15 | 1.365051 | 5.19087E−05 | THOP1 | 0.377864 | 0.002430737 | CALML4 | 0.39924356 | 0.027905815 |
| ASUN | 1.0095742 | 9.56589E−09 | RNF8 | 0.32294932 | 5.19737E−05 | JAGN1 | 0.57610595 | 0.002434202 | C21orf58 | 0.042905178 | 0.027909602 |
| NBPF15 | 0.7745722 | 9.63511E−09 | ZNF772 | 0.20153451 | 5.22912E−05 | USP35 | 1.3653053 | 0.002443381 | PTPN4 | 0.09762446 | 0.027929597 |
| TRIM32 | 1.2833277 | 9.64759E−09 | C6orf203 | 0.76311076 | 5.23688E−05 | RB1CC1 | 0.26839063 | 0.002456726 | SOCS4 | 0.16342172 | 0.027937441 |
| EIF2D | 1.049364 | 9.67914E−09 | UNG | 0.7640693 | 5.39033E−05 | IQCC | 0.021852616 | 0.002470495 | PMS2CL | 0.5625079 | 0.027941233 |
| TSTD1 | 2.7002058 | 9.67914E−09 | COLEC11 | 1.8581232 | 5.39334E−05 | PLD2 | 0.7765228 | 0.002470495 | LOC100506474 | 0.5036911 | 0.027960987 |
| DGCR14 | 0.9465173 | 9.67914E−09 | FEM1B | 0.7032594 | 5.40381E−05 | ADI1 | 0.5678013 | 0.002471754 | CIRBP | 0.4774467 | 0.028061519 |
| RREB1 | 0.64439845 | 9.7068E−09 | MLF2 | 0.77126306 | 5.55026E−05 | ZFP90 | 0.07177055 | 0.002472561 | TTC28-AS1 | 0.10132231 | 0.028073468 |
| MRP63 | 0.6627519 | 9.76844E−09 | SHCBP1 | 0.57879734 | 5.56102E−05 | SMG8 | 0.4986606 | 0.002481874 | NDUFAF5 | 0.37975407 | 0.02812921 |
| ARRB1 | 0.7427293 | 9.76844E−09 | VPS45 | 0.5466878 | 5.57309E−05 | SLC7A5P1 | 0.9341646 | 0.002501345 | RABGGTB | 0.5017245 | 0.02812921 |
| IDH1 | 1.0647503 | 9.79332E−09 | SNRPD1 | 0.73791546 | 5.63932E−05 | SLC38A8 | 3.5178607 | 0.002518199 | TTLL1 | 0.38429657 | 0.028137108 |
| PSMD8 | 1.0868429 | 1.01221E−08 | SCFD2 | 0.8777719 | 5.73781E−05 | SAR1A | 0.4881748 | 0.002525003 | ADAMTS4 | 0.16147573 | 0.028267118 |
| GRSF1 | 0.88020843 | 1.01826E−08 | RBM3 | 0.48046985 | 5.74364E−05 | WTAP | 0.3819593 | 0.002527112 | DHPS | 0.49962047 | 0.028352979 |
| NID1 | 0.7133903 | 1.05248E−08 | NUMBL | 0.33632925 | 5.78768E−05 | ANKRD53 | 2.064636 | 0.002529782 | MAP3K3 | 0.13855684 | 0.028442926 |
| HSF4 | 0.935537 | 1.05843E−08 | UBE2N | 0.6950156 | 5.78768E−05 | HOXB13 | 1.1717062 | 0.00253328 | RAD51AP1 | 1.0102149 | 0.028616092 |
| LOC100294145 | 0.9489488 | 1.05948E−08 | NOL11 | 0.71117586 | 5.7949E−05 | MRPS7 | 0.6538348 | 0.002534569 | ADAM33 | 0.57101655 | 0.028631597 |
| RPRD2 | 0.63366836 | 1.06449E−08 | TMEM5 | 0.5798865 | 5.80802E−05 | ZNF764 | 0.18756779 | 0.002535396 | LTN1 | 0.20106658 | 0.028631597 |
| TCEA3 | 1.6455119 | 1.07651E−08 | ZNF22 | 0.7983721 | 5.80802E−05 | ZNF786 | 0.4627889 | 0.002543073 | C19orf43 | 0.21759698 | 0.028631597 |
| ANAPC5 | 1.1045703 | 1.08732E−08 | XPO6 | 0.48254368 | 5.8086E−05 | FYTTD1 | 0.46141592 | 0.002543073 | ZW10 | 0.49535584 | 0.028664488 |
| COA6 | 1.0643642 | 1.08941E−08 | DNAJC17 | 0.8483194 | 5.8533E−05 | CREB1 | 0.119200446 | 0.002546589 | LRR1 | 0.6944672 | 0.028714019 |
| USP3 | 0.5922604 | 1.09354E−08 | YLPM1 | 0.3470566 | 5.85388E−05 | CTNNB1 | 0.5149414 | 0.002548349 | RPL13AP6 | 0.18327105 | 0.028717907 |
| CA5BP1 | 1.0084946 | 1.10007E−08 | DIP2B | 0.4203109 | 5.85849E−05 | LPCAT3 | 0.30538625 | 0.00255011 | DNA2 | 0.9165843 | 0.028750621 |
| DDX55 | 0.7433108 | 1.11733E−08 | TRA2B | 0.57930523 | 5.85908E−05 | OAF | 0.79046017 | 0.002550477 | NAA50 | 0.20285632 | 0.028750621 |
| BOLA2 | 0.6310455 | 1.12512E−08 | ME2 | 0.45809487 | 5.89392E−05 | ATPAF2 | 0.54795605 | 0.002550845 | STK36 | 0.2525935 | 0.028804447 |
| BOLA2B | 0.6310455 | 1.12512E−08 | HAUS6 | 0.55322295 | 5.89392E−05 | PFN2 | 0.6260385 | 0.002551677 | ANGEL1 | 0.3102678 | 0.028841418 |
| MKKS | 0.7363322 | 1.13852E−08 | FAM21C | 0.43134448 | 5.90668E−05 | POLR2L | 0.56626284 | 0.002564906 | DGCR8 | 0.19711378 | 0.028857833 |
| METAP2 | 0.81569546 | 1.13973E−08 | GCFC2 | 0.5331879 | 5.92625E−05 | COMMD9 | 0.32783017 | 0.002564906 | ALKBH6 | 0.47094363 | 0.028886779 |
| TOMM20 | 0.86235327 | 1.13973E−08 | ZNF7 | 0.7895781 | 5.94724E−05 | ZNF280D | 0.45211217 | 0.002564906 | RANBP6 | 0.6112431 | 0.028894865 |
| ANKRD39 | 1.2267026 | 1.14689E−08 | ACSF3 | 0.825599 | 5.97788E−05 | ZNF280C | 0.16475585 | 0.00256892 | RAB28 | 0.21652564 | 0.028919664 |
| SCMH1 | 0.80171967 | 1.18928E−08 | TRIM26 | 0.7799224 | 5.98122E−05 | HMGB3 | 0.52773017 | 0.00256892 | MBOAT7 | 0.17592072 | 0.02893612 |
| UROD | 1.1471205 | 1.19268E−08 | MCM2 | 0.80636704 | 6.01202E−05 | LMNB1 | 0.67378604 | 0.002576222 | ORC2 | 0.25171754 | 0.02896932 |
| LOC100128593 | 0.3547972 | 1.19645E−08 | GALNT3 | 0.97040415 | 6.02977E−05 | ZNF93 | 0.91058135 | 0.002576594 | TCHP | 0.335872 | 0.028989988 |
| VPS4B | 0.6954554 | 1.20003E−08 | USP4 | 0.48830664 | 6.03866E−05 | ATP5EP2 | 0.2839438 | 0.002581193 | LTA4H | 0.4149766 | 0.029048403 |
| SNRNP25 | 1.1571875 | 1.20003E−08 | C12orf10 | 0.8309742 | 6.05034E−05 | SAP25 | 1.7652792 | 0.002583446 | VAMP3 | 0.4375495 | 0.029069121 |
| MRPS15 | 1.1570284 | 1.21845E−08 | WDR89 | 0.5230109 | 6.05095E−05 | LINC00668 | 1.0568006 | 0.002585231 | TRIP13 | 0.114134006 | 0.029202662 |
| POP1 | 1.2133256 | 1.24251E−08 | KIAA0195 | 0.45291644 | 6.08054E−05 | SPATA2 | 0.5054729 | 0.002594092 | ZNF8 | 0.9219088 | 0.029231649 |
| SFXN4 | 1.2237587 | 1.2449E−08 | UAP1L1 | 0.45236844 | 6.08054E−05 | GLE1 | 0.2762241 | 0.002597774 | SLC27A4 | 0.9503346 | 0.029231649 |
| APTX | 1.0782176 | 1.24816E−08 | PPP4C | 0.67356503 | 6.08673E−05 | C16orf87 | 0.59656876 | 0.002599568 | STK40 | 0.4771583 | 0.029273594 |
| MOCS3 | 0.6326547 | 1.25095E−08 | SRSF10 | 0.59559685 | 6.10547E−05 | NUDC | 0.63573104 | 0.002601265 | KANK3 | 0.47015607 | 0.029290229 |
| POLRMT | 0.9432498 | 1.26895E−08 | FBXO28 | 0.4384861 | 6.11028E−05 | HEATR3 | 0.2236297 | 0.002607803 | PTK6 | 0.057041906 | 0.029319558 |
| DDX56 | 1.0371356 | 1.28086E−08 | SIKE1 | 0.3123073 | 6.12489E−05 | MPP3 | 1.5601256 | 0.002611504 | FAHD2CP | 0.35335413 | 0.029374322 |
| TPM1 | 1.0620828 | 1.30774E−08 | AP1B1 | 0.5860794 | 6.12972E−05 | CHEK2 | 0.70711076 | 0.002620251 | PRMT10 | 0.45262167 | 0.029378293 |
| LCOR | 0.84188277 | 1.30783E−08 | GTF2F2 | 0.67899686 | 6.13595E−05 | SNRNP35 | 0.46726814 | 0.002620251 | PDLIM1 | 0.41777217 | 0.029394453 |
| BCKDK | 1.0558158 | 1.35602E−08 | TMEM9 | 0.8456769 | 6.20003E−05 | FBXO38 | 0.33670822 | 0.002622816 | MAPKAPK5-AS1 | 0.3019634 | 0.029436604 |
| ANXA6 | 0.9450211 | 1.35653E−08 | CENPT | 0.4339198 | 6.20633E−05 | ZNF765 | 0.001297718 | 0.002622816 | RPL23AP7 | 0.17067084 | 0.02955965 |
| YIPF1 | 1.098639 | 1.35999E−08 | DPP8 | 0.49237612 | 6.21548E−05 | YME1L1 | 0.3157179 | 0.002622816 | TMEM91 | 0.7757761 | 0.029571372 |
| GPR19 | 2.3714664 | 1.37189E−08 | CLK2 | 0.64901954 | 6.26364E−05 | ANKRD27 | 0.39273152 | 0.002630263 | HIRA | 0.58042747 | 0.029571372 |
| PPIF | 1.0303297 | 1.37601E−08 | UBQLN2 | 0.5639736 | 6.27287E−05 | MAP3K7 | 0.42829683 | 0.002634374 | PARK7 | 0.59078795 | 0.029571372 |
| ZSWIM7 | 0.69641083 | 1.37601E−08 | MYO1B | 0.57302856 | 6.30512E−05 | FAN1 | 0.37677956 | 0.002635135 | PUS1 | 0.7827818 | 0.029583894 |
| NUP88 | 1.1283058 | 1.37601E−08 | DNAH14 | 0.5325114 | 6.34975E−05 | BECN1 | 0.6740404 | 0.002635995 | ZNF530 | 0.07185369 | 0.029587894 |
| VPS33A | 0.64581805 | 1.37761E−08 | ICT1 | 0.8438725 | 6.34975E−05 | CENPB | 0.4191442 | 0.002640214 | POLE4 | 0.42376792 | 0.029634296 |
| C19orf70 | 0.97665215 | 1.37761E−08 | GLS | 0.37822077 | 6.3504E−05 | ATG12 | 0.33762932 | 0.002643477 | AFF1 | 0.13380866 | 0.029723803 |
| RHBDD3 | 1.0167578 | 1.37857E−08 | SOX10 | 0.582174 | 6.35105E−05 | PJA1 | 0.5460266 | 0.00264434 | ARHGAP5-AS1 | 0.21013908 | 0.029779232 |
| PDK3 | 0.68240225 | 1.38507E−08 | TMEM87A | 0.61364263 | 6.3517E−05 | LOC158435 | 0.12740661 | 0.002662752 | TEN1 | 0.37708429 | 0.029839046 |
| DCAF12 | 0.8643665 | 1.38518E−08 | CBY1 | 0.7221337 | 6.39162E−05 | ZBTB41 | 0.3939966 | 0.002664105 | LOC730102 | 0.34973514 | 0.02987316 |
| FASTKD1 | 0.896941 | 1.38743E−08 | MRPL3 | 0.86318433 | 6.39665E−05 | OTUD3 | 0.12322504 | 0.002664974 | CEBPG | 0.2962062 | 0.029877194 |
| SUPT16H | 0.7927022 | 1.38926E−08 | SAP18 | 0.63852227 | 6.43244E−05 | LSG1 | 0.34596863 | 0.002668751 | DEPDC5 | 0.15034316 | 0.029910815 |
| GOT2 | 1.0573976 | 1.39078E−08 | HYPK | 0.6574431 | 6.44338E−05 | RAI1 | 0.26284724 | 0.002681283 | AURKB | 0.84128404 | 0.029953091 |
| REXO2 | 1.0868942 | 1.39078E−08 | ZNF664 | 0.5796484 | 6.46465E−05 | RAMP2 | 0.9652951 | 0.002684982 | ACAD8 | 0.44076404 | 0.02998248 |
| GPX1 | 0.9062926 | 1.39402E−08 | PNMAL1 | 0.4185822 | 6.46827E−05 | SPIRE2 | 0.093716465 | 0.002688587 | ZNF684 | 0.8770203 | 0.02998248 |
| NTAN1 | 1.1631495 | 1.39402E−08 | EXOSC8 | 0.64515 | 6.52077E−05 | CEP164 | 0.22800612 | 0.00269093 | ARFGAP2 | 0.3054407 | 0.030102932 |
| IMMT | 0.9825181 | 1.41273E−08 | DHX34 | 0.5889711 | 6.64362E−05 | SLC39A10 | 0.3919194 | 0.002694741 | TESK2 | 0.7631188 | 0.030154647 |
| EPN2 | 0.66089326 | 1.43181E−08 | NEFH | 1.5509679 | 6.64362E−05 | CRCP | 0.22620778 | 0.002699047 | GYG2P1 | 0.041308086 | 0.030175799 |
| PLCG1 | 0.72264194 | 1.43181E−08 | CYB561D1 | 0.081107885 | 6.66097E−05 | TUBG2 | 0.29524085 | 0.002707187 | ANKDD1A | 0.04332396 | 0.03029667 |
| CBWD1 | 0.8258648 | 1.44878E−08 | ARIH2 | 0.54351294 | 6.67228E−05 | INTS2 | 0.08892256 | 0.002709937 | TMX3 | 0.2464152 | 0.030300759 |
| MAGED1 | 1.033442 | 1.49511E−08 | SRRM1 | 0.46452993 | 6.73555E−05 | ENTPD7 | 0.30523995 | 0.002711313 | USP42 | 0.33523756 | 0.030322003 |
| HSD17B7 | 0.8670827 | 1.50123E−08 | ZSCAN21 | 0.8629713 | 6.74461E−05 | RGS19 | 0.9005787 | 0.002714166 | RSBN1L | 0.031124532 | 0.030322003 |
| PA2G4 | 0.93623817 | 1.51016E−08 | RCN3 | 0.6753934 | 6.74461E−05 | C11orf83 | 0.25185987 | 0.002715545 | ZNF324 | 0.78854513 | 0.030360965 |
| ILF3 | 0.9207874 | 1.54077E−08 | VARS | 0.638936 | 6.78225E−05 | DTWD1 | 0.35951 | 0.002718895 | CTDSPL2 | 0.23925328 | 0.030417437 |
| WASH3P | 0.7095325 | 1.54091E−08 | PRPF4B | 0.30069146 | 6.86125E−05 | DDX23 | 0.4870121 | 0.00271929 | TK1 | 0.76509166 | 0.030539161 |
| NUP133 | 0.9348656 | 1.54295E−08 | COX10 | 0.8491338 | 6.94515E−05 | GCLC | 0.7909056 | 0.002723632 | SLC2A4RG | 0.6037844 | 0.03056082 |
| C9orf139 | 0.41655126 | 1.57083E−08 | CENPJ | 0.8493657 | 6.95218E−05 | ZNF584 | 0.9629064 | 0.002726004 | CTDNEP1 | 0.28702268 | 0.030578103 |
| ANKRD52 | 0.6835913 | 1.5729E−08 | C12orf23 | 0.54314333 | 6.95922E−05 | RFC4 | 0.81119627 | 0.002727388 | KIAA1279 | 0.3455399 | 0.030621482 |
| ZBED6 | 0.8840001 | 1.57305E−08 | MBD3 | 0.67966986 | 7.02349E−05 | LAMA5 | 0.4449862 | 0.002727784 | TTC28 | 0.04604904 | 0.030673981 |
| EIF2B4 | 1.0842135 | 1.579E−08 | C2orf68 | 0.42394304 | 7.08657E−05 | ZNF283 | 0.2367838 | 0.002731644 | ECSIT | 0.47673488 | 0.030691321 |
| PPP2R1B | 0.7414353 | 1.58595E−08 | KIAA1704 | 0.75739425 | 7.08891E−05 | MTF1 | 0.038832776 | 0.002736501 | ZNF555 | 0.026724573 | 0.030695459 |
| PPCS | 0.9906769 | 1.58658E−08 | ZNF317 | 0.5350937 | 7.13809E−05 | HDAC10 | 0.5340111 | 0.002746239 | PPIP5K2 | 0.09935111 | 0.030716951 |
| FARSB | 1.0303934 | 1.59455E−08 | NGLY1 | 0.6303595 | 7.14207E−05 | ZFP82 | 0.84525126 | 0.002746239 | IQGAP3 | 0.57638425 | 0.03074313 |
| SNHG3 | 0.9375541 | 1.61604E−08 | CBX8 | 0.8951299 | 7.14444E−05 | LRRFIP2 | 0.3371266 | 0.002757508 | FLJ44635 | 0.11107366 | 0.030791133 |
| MRPS5 | 0.9474922 | 1.63164E−08 | ADPRM | 1.0741948 | 7.17517E−05 | LIAS | 0.04803353 | 0.002757908 | SEPT7P2 | 0.03912904 | 0.030817368 |
| CTBP2 | 0.6728679 | 1.64789E−08 | ERI3 | 0.5675814 | 7.17517E−05 | POC1A | 1.0804402 | 0.002761808 | PPP6R3 | 0.29429996 | 0.030932165 |
| PIR | 1.210221 | 1.65109E−08 | CDC25B | 0.55286926 | 7.24955E−05 | LCMT1 | 0.5949378 | 0.002768219 | HAUS3 | 0.16689497 | 0.0309452 |
| TIA1 | 0.71235085 | 1.66821E−08 | CCSER2 | 0.38049096 | 7.25359E−05 | LOC644936 | 0.1410163 | 0.002772535 | LOC100240735 | 0.6259072 | 0.030984601 |
| TIPRL | 0.8361758 | 1.67452E−08 | CERS4 | 0.77323395 | 7.30056E−05 | CBWD2 | 0.4477752 | 0.002775449 | FBXO34 | 0.53840894 | 0.030993216 |
| UBE2G1 | 0.64281654 | 1.68395E−08 | ABHD11 | 0.3968184 | 7.3096E−05 | TFCP2L1 | 2.1265976 | 0.002777863 | RABAC1 | 0.6206067 | 0.031006274 |
| ELP6 | 1.0492748 | 1.69603E−08 | FKBP1AP1 | 0.55152196 | 7.34025E−05 | IFIT3 | 0.14610773 | 0.002789252 | SLC39A6 | 0.4374008 | 0.031014895 |
| INTS7 | 0.63811463 | 1.70141E−08 | DRG2 | 0.80817205 | 7.35267E−05 | CLPB | 0.6032212 | 0.002790667 | ARPC4 | 0.5375153 | 0.031072436 |
| C19orf54 | 0.577065 | 1.70419E−08 | TTL | 0.08985418 | 7.37757E−05 | CDCA2 | 0.91295624 | 0.002795622 | GPATCH2L | 0.34227607 | 0.031081073 |
| ZNF562 | 0.6077259 | 1.71116E−08 | IBTK | 0.4740237 | 7.42194E−05 | C12orf4 | 0.64199144 | 0.002800585 | UBN1 | 0.16502006 | 0.031165219 |
| ANAPC13 | 1.135116 | 1.75325E−08 | LARS | 0.50030166 | 7.43449E−05 | KBTBD7 | 0.9772398 | 0.002803526 | VKORC1 | 0.6249046 | 0.031178078 |
| MTFR1 | 0.7781613 | 1.75611E−08 | MYO9B | 0.39404595 | 7.44032E−05 | ECT2L | 0.004069156 | 0.00281919 | LOC100505817 | 0.5217544 | 0.031204605 |
| PPAP2C | 2.358574 | 1.75845E−08 | NAA60 | 0.5284752 | 7.46642E−05 | ZNF519 | 0.5232889 | 0.002824702 | LOC728323 | 0.28202644 | 0.03128931 |
| HDAC2 | 0.7547584 | 1.79342E−08 | LUC7L | 0.5652433 | 7.47905E−05 | CCP110 | 0.2693344 | 0.00283176 | UBASH3B | 0.63603264 | 0.031301951 |
| RHOJ | 0.7633507 | 1.80351E−08 | PRRG1 | 0.5786541 | 7.53423E−05 | LOC90784 | 0.2679578 | 0.002833707 | ABHD5 | 0.2994892 | 0.031301951 |
| BLOC1S2 | 0.6074125 | 1.80922E−08 | GPS1 | 0.88267964 | 7.56235E−05 | TMEM185B | 0.2928685 | 0.002836168 | ZNF621 | 0.04386697 | 0.031301951 |
| ZNF197 | 0.8517431 | 1.81495E−08 | TAF1A | 0.4401354 | 7.582E−05 | SPHK2 | 0.49619377 | 0.00284675 | DEPDC7 | 1.27875 | 0.031306166 |
| EIF4E2 | 1.0774661 | 1.81625E−08 | C17orf49 | 0.758139 | 7.59732E−05 | TAF1 | 0.24938884 | 0.00284675 | RPGRIP1L | 0.42359257 | 0.031341756 |
| MRPL50 | 1.1428171 | 1.83937E−08 | RNF34 | 0.677492 | 7.59732E−05 | G2E3 | 0.36152005 | 0.002851282 | CSNK2B | 0.6128286 | 0.031345977 |
| MYADM | 0.8926605 | 1.85311E−08 | ZNF557 | 0.44733888 | 7.61361E−05 | ATAT1 | 0.3177243 | 0.002856853 | LIX1L | 0.19478515 | 0.031521035 |
| CHAMP1 | 1.3282903 | 1.87955E−08 | UQCRH | 0.8713349 | 7.61773E−05 | PARP16 | 0.6706313 | 0.002871134 | ALDH4A1 | 0.511858 | 0.031611014 |
| DKC1 | 1.0396743 | 1.88205E−08 | TMEM38A | 0.14626005 | 7.61773E−05 | PRPF38B | 0.36880758 | 0.002872588 | NADSYN1 | 0.47436526 | 0.031714543 |
| NDUFS7 | 1.2999194 | 1.90307E−08 | AES | 0.5755402 | 7.61773E−05 | FUBP1 | 0.43151653 | 0.002878095 | PEX14 | 0.28352967 | 0.031872942 |
| HAX1 | 1.1625515 | 1.92629E−08 | ZFP64 | 0.004305294 | 7.61853E−05 | ZNF768 | 0.82143295 | 0.002882571 | PGPEP1 | 0.16705208 | 0.031935899 |
| FAM86A | 1.0526968 | 1.95494E−08 | ZMAT5 | 0.7063946 | 7.64177E−05 | LBH | 0.85413986 | 0.002887156 | GMPS | 0.37243494 | 0.031953868 |
| DNAJA3 | 0.9792723 | 1.95635E−08 | MBP | 0.85197866 | 7.65296E−05 | C15orf57 | 0.50611335 | 0.002887995 | CTNNA2 | 1.5791153 | 0.031967284 |
| POLR1C | 1.0900111 | 1.9812E−08 | ALS2CR11 | 1.3307809 | 7.68058E−05 | DNAJB7 | 0.20451313 | 0.002891964 | CDK19 | 0.15686421 | 0.032026378 |
| EIF5AL1 | 0.40235233 | 1.98323E−08 | ZNF593 | 0.84095615 | 7.71005E−05 | U2AF1 | 0.6496788 | 0.002896359 | LRCH1 | 0.16535984 | 0.032094726 |
| TRO | 1.3792286 | 2.0023E−08 | SLC26A4 | 0.42551318 | 7.77387E−05 | LARS2 | 0.6482984 | 0.002913552 | HERC2P7 | 0.11308116 | 0.032158342 |
| UTP14A | 0.9588864 | 2.00558E−08 | TWF1 | 0.65409285 | 7.90399E−05 | GATAD2B | 0.17646384 | 0.002913873 | TSC22D4 | 0.05669151 | 0.032190192 |
| CHERP | 0.6294927 | 2.01438E−08 | F5 | 0.3937655 | 7.97834E−05 | CHTF18 | 0.630599 | 0.002915348 | C8orf46 | 0.59499925 | 0.032437844 |
| ALDH9A1 | 1.0825001 | 2.02076E−08 | TOE1 | 0.82239646 | 8.01348E−05 | GLRX2 | 0.5081855 | 0.002924095 | MMP2 | 0.5916085 | 0.032460411 |
| NKD1 | 0.28667566 | 2.03273E−08 | ANKRD45 | 0.32673255 | 8.02422E−05 | INPP5A | 1.341489 | 0.002924095 | OTX1 | 1.0782022 | 0.032460411 |
| LOC652276 | 0.4484891 | 2.07199E−08 | PRDM7 | 0.8080009 | 8.02422E−05 | KCNIP2 | 1.48904 | 0.002926103 | SYS1 | 0.24506083 | 0.032464761 |
| RUSC2 | 0.9685965 | 2.08998E−08 | ATF5 | 0.27481627 | 8.02506E−05 | CAPZA1 | 0.62333536 | 0.002928112 | MAP1LC3C | 0.088393696 | 0.032588755 |
| TRMT12 | 1.3685583 | 2.0966E−08 | PMEL | 0.7056346 | 8.03497E−05 | METAP1D | 0.73387456 | 0.002936997 | C22orf39 | 0.12577617 | 0.032588755 |
| TRAF4 | 0.50542134 | 2.14007E−08 | KDM5B | 0.4402699 | 8.04489E−05 | TP53I11 | 1.0439974 | 0.002937953 | PSMA4 | 0.5546805 | 0.032662531 |
| BIRC7 | 1.2421241 | 2.15536E−08 | C17orf51 | 0.72123826 | 8.10038E−05 | LOC282997 | 0.022443417 | 0.002953142 | DNAJC13 | 0.30871075 | 0.032718098 |
| IRAK1 | 0.6598749 | 2.16349E−08 | PTPRG | 0.4142818 | 8.11404E−05 | LZIC | 0.50960106 | 0.002956766 | TCF25 | 0.36299086 | 0.03275016 |
| FAM92A1 | 1.0371891 | 2.2063E−08 | RFPL2 | 3.0998993 | 8.16262E−05 | CHM | 0.118998155 | 0.002957196 | MKI67 | 0.52648926 | 0.032758933 |
| FDPS | 1.1645252 | 2.21126E−08 | MRPL11 | 0.6237577 | 8.22074E−05 | KNSTRN | 0.6695619 | 0.002959758 | EEFSEC | 0.39220506 | 0.032758933 |
| STARD10 | 1.4121931 | 2.24064E−08 | RNMT | 0.4130375 | 8.2569E−05 | SSB | 0.63704455 | 0.002963923 | EP400NL | 0.6025713 | 0.032819312 |
| MMADHC | 1.2123713 | 2.26694E−08 | PI4KAP2 | 0.51620865 | 8.30904E−05 | GGA3 | 0.33906522 | 0.002967025 | NDUFAF4P1 | 0.5203937 | 0.032851738 |
| RPL27A | 0.45232636 | 2.27965E−08 | IQCK | 0.6326589 | 8.30904E−05 | C12orf77 | 0.09655158 | 0.002992467 | VAMP4 | 0.13506745 | 0.032892726 |
| IPO5 | 0.6763819 | 2.33745E−08 | SLC35A4 | 0.70488125 | 8.32668E−05 | LARP4B | 0.17111756 | 0.002994519 | DYRK1B | 0.3922201 | 0.032892726 |
| ZNF536 | 1.8657131 | 2.3771E−08 | GIT2 | 0.6457225 | 8.39625E−05 | SPIN3 | 0.73911005 | 0.003010312 | MBIP | 0.36338562 | 0.032892726 |
| MLST8 | 0.97865623 | 2.37882E−08 | ALDH3B1 | 0.555665 | 8.45214E−05 | CCDC138 | 0.9255503 | 0.003017798 | PDRG1 | 0.5100797 | 0.032943677 |
| KAZN | 0.8380509 | 2.39287E−08 | SLC25A46 | 0.63998336 | 8.47018E−05 | TMEM117 | 0.52551556 | 0.003024214 | MAPK8IP3 | 0.105813615 | 0.032957188 |
| PRKCD | 0.93785095 | 2.39825E−08 | CNIH | 0.793087 | 8.47681E−05 | GEMIN6 | 0.63725084 | 0.003025198 | RPL23 | 0.31634635 | 0.033111609 |
| ZNF568 | 0.8798202 | 2.43402E−08 | ZNF556 | 0.86904943 | 8.49007E−05 | HELQ | 0.5172656 | 0.003028256 | C10orf32-AS3MT | 0.3561524 | 0.033134871 |
| H3F3A | 1.7632413 | 2.49942E−08 | SPATS2L | 0.30486935 | 8.49007E−05 | PIGB | 0.16784528 | 0.003032952 | SWT1 | 0.24381807 | 0.033256943 |
| TTLL12 | 1.0815798 | 2.50883E−08 | FTSJ2 | 0.7616766 | 8.57058E−05 | RWDD4 | 0.47776502 | 0.003032952 | FAM206A | 0.3906571 | 0.033355722 |
| SUCLA2 | 1.0408003 | 2.51218E−08 | LBR | 0.8149956 | 8.57342E−05 | MED6 | 0.21371275 | 0.003043121 | SEC61A2 | 0.36004466 | 0.033359907 |
| ZNF587B | 0.9259404 | 2.62168E−08 | RMI1 | 1.0524999 | 8.5849E−05 | METTL21A | 0.2711541 | 0.003044111 | ALDH2 | 1.7498164 | 0.033406752 |
| C14orf2 | 0.8391187 | 2.62168E−08 | MED27 | 0.8377631 | 8.5849E−05 | RPS2 | 0.35256976 | 0.003052224 | RECQL5 | 0.20259018 | 0.033524945 |
| CRTC2 | 0.8019103 | 2.65315E−08 | EMILIN2 | 1.5124612 | 8.61483E−05 | MTERFD2 | 0.47813296 | 0.003055412 | VIPAS39 | 0.36501375 | 0.033557707 |
| CHCHD1 | 0.9256344 | 2.67768E−08 | NSF | 0.68826944 | 8.65653E−05 | ZAK | 0.05480705 | 0.003055857 | TYW1 | 0.28519762 | 0.033562193 |
| MRPL48 | 1.1384494 | 2.69704E−08 | ZNF668 | 1.1561068 | 8.65745E−05 | RIOK2 | 0.3478866 | 0.003058499 | COX4I1 | 0.39365783 | 0.033585459 |
| PNO1 | 0.7293539 | 2.72478E−08 | XPO5 | 0.3825013 | 8.66422E−05 | STXBP2 | 0.64082456 | 0.003058945 | FNBP1 | 0.10032091 | 0.033585459 |
| GMFB | 0.79250973 | 2.74082E−08 | AZI1 | 0.7761293 | 8.6788E−05 | PAFAH1B2 | 0.4922336 | 0.00306104 | TPRA1 | 0.3929476 | 0.033604247 |
| SEMA4C | 0.893476 | 2.76364E−08 | C15orf39 | 0.33147654 | 8.72164E−05 | REEP6 | 0.85291934 | 0.003062036 | CCDC59 | 0.25010565 | 0.033703381 |
| STAMBP | 0.69211555 | 2.80609E−08 | CIAPIN1 | 0.70461065 | 8.72164E−05 | PPM1K | 0.21814172 | 0.003069641 | RCE1 | 0.40949333 | 0.033802775 |
| ICK | 0.5284269 | 2.85815E−08 | AIMP1 | 0.5289568 | 8.74118E−05 | MAD2L1BP | 0.561707 | 0.003070536 | TLK2 | 0.10249241 | 0.033802775 |
| DECR1 | 1.0908494 | 2.85815E−08 | GEN1 | 0.40617394 | 8.77842E−05 | AZI2 | 0.47598058 | 0.003070536 | SIVA1 | 0.55410945 | 0.033941424 |
| EARS2 | 1.1043705 | 2.94386E−08 | TRAPPC5 | 0.8451926 | 8.77842E−05 | NSMCE4A | 0.7560246 | 0.0030857 | XAB2 | 0.12607047 | 0.03399408 |
| LTV1 | 1.1232758 | 2.98539E−08 | HADH | 0.6891247 | 8.77842E−05 | GNAO1 | 1.7247804 | 0.0030857 | OSGIN1 | 0.8322517 | 0.034012794 |
| SLC25A11 | 1.0614007 | 3.02112E−08 | HNRNPK | 0.5845972 | 8.77842E−05 | DUXA | 0.112764694 | 0.0030866 | POLB | 0.4522249 | 0.034022155 |
| MLX | 0.9385755 | 3.0241E−08 | HSPB6 | 1.9938338 | 8.78726E−05 | BUB1 | 0.91156644 | 0.0030866 | INCA1 | 0.10247566 | 0.034127758 |
| PDSS2 | 0.6700496 | 3.0241E−08 | ALS2 | 0.41129026 | 8.80797E−05 | SYK | 0.19802488 | 0.003089268 | PPP3CA | 0.09401768 | 0.034200025 |
| SMARCA4 | 0.6731913 | 3.02944E−08 | ZNF776 | 0.5230358 | 8.82279E−05 | RND2 | 0.31435847 | 0.003109745 | SRP19 | 0.33092272 | 0.034209431 |
| GGCT | 1.1187465 | 3.11506E−08 | MOB1B | 0.27574578 | 8.82771E−05 | RPS15AP10 | 0.085793614 | 0.003110756 | FAM149B1 | 0.27638575 | 0.034238216 |
| C9orf64 | 0.54331166 | 3.12204E−08 | CCDC22 | 0.8654194 | 8.84652E−05 | AFF3 | 0.1510335 | 0.003117356 | HES6 | 0.6527916 | 0.034422504 |
| THOC7 | 1.0101869 | 3.14891E−08 | BCAT2 | 0.83977574 | 8.85941E−05 | PITPNA-AS1 | 0.5902302 | 0.003139693 | PQLC1 | 0.35643893 | 0.034427093 |
| TNKS2 | 0.7679569 | 3.15597E−08 | MOB4 | 0.36781478 | 8.86037E−05 | NHSL1 | 0.3463552 | 0.003158568 | PCOLCE | 0.54358214 | 0.034431405 |
| PRPF6 | 0.8991227 | 3.18408E−08 | BTBD10 | 0.8035443 | 8.93725E−05 | PHLPP1 | 0.3106107 | 0.003164694 | TMEM51 | 0.43911338 | 0.034431405 |
| TFAP2A | 0.74072766 | 3.19988E−08 | TPT1 | 0.2995425 | 8.97641E−05 | LZTS1 | 0.48265144 | 0.003173104 | CYP4A11 | 0.02094242 | 0.03443572 |
| SMIM4 | 0.9312826 | 3.20212E−08 | HINFP | 0.90337694 | 9.00862E−05 | GIGYF2 | 0.35111427 | 0.003175841 | ZDHHC7 | 0.27692854 | 0.034684037 |
| SLX1A | 0.8094968 | 3.20212E−08 | NCAPH | 0.8578311 | 9.01059E−05 | ATOH8 | 1.2173036 | 0.003177441 | SLC1A5 | 0.3549295 | 0.034688658 |
| SLX1B | 0.8094968 | 3.20212E−08 | PEX11B | 0.73703545 | 9.01059E−05 | RUNX3 | 0.45376772 | 0.003179043 | RHOU | 0.87972444 | 0.034712893 |
| RBM10 | 0.80354214 | 3.22967E−08 | PRPF3 | 0.54648715 | 9.05615E−05 | TIMM8A | 0.78368783 | 0.003199389 | POU6F1 | 0.08873535 | 0.034722423 |
| RRN3P2 | 1.4031755 | 3.25156E−08 | SNAPC5 | 0.6578292 | 9.11517E−05 | TSSC1 | 0.38561705 | 0.003199389 | C2orf49 | 0.3749914 | 0.034815457 |
| PREX1 | 1.1515453 | 3.30031E−08 | FOPNL | 0.45293468 | 9.13458E−05 | TTC27 | 0.495787 | 0.003215933 | LRP5 | 0.4608696 | 0.034825012 |
| TYK2 | 0.7600036 | 3.33916E−08 | TSTD2 | 0.45939627 | 9.14172E−05 | RALBP1 | 0.2708143 | 0.003222738 | WDR45 | 0.30744573 | 0.034864097 |
| TP53INP1 | 0.8109947 | 3.35875E−08 | DIS3 | 0.25483164 | 9.14477E−05 | ITGB3BP | 0.4415931 | 0.003223207 | CASC5 | 0.89917463 | 0.034883234 |
| DLEU1 | 1.3382348 | 3.42036E−08 | ALKBH5 | 0.55003035 | 9.1848E−05 | PRDX6 | 0.55982965 | 0.003224253 | POFUT1 | 0.13826992 | 0.034883234 |
| WDR73 | 0.9640024 | 3.43422E−08 | SPNS1 | 0.6733366 | 9.21467E−05 | SCAND2P | 0.1158299 | 0.003225299 | ZNF503-AS2 | 1.6058168 | 0.034927306 |
| DAGLA | 2.123878 | 3.44607E−08 | LOC100129361 | 0.46755695 | 9.22807E−05 | TMF1 | 0.2690321 | 0.003228078 | RBM22 | 0.22421327 | 0.034936889 |
| CELSR3 | 1.5840485 | 3.47253E−08 | THNSL1 | 0.38461232 | 9.231ISE−05 | SBF1 | 0.06018192 | 0.003232015 | HNRNPUL2-BSCL2 | 0.56364155 | 0.034946475 |
| LMNB2 | 0.86891544 | 3.49499E−08 | SMN1 | 0.72800106 | 9.23837E−05 | XPC | 0.33461162 | 0.003232486 | CYCS | 0.35483867 | 0.035029866 |
| UBE3A | 0.56488705 | 3.55903E−08 | TMEM11 | 0.8200194 | 9.31002E−05 | PHF5A | 0.55136216 | 0.003237586 | PLXNC1 | 0.35335758 | 0.035069153 |
| KLHL8 | 0.5756354 | 3.57451E−08 | ERICH1 | 0.5702852 | 9.38429E−05 | BET1L | 0.42820275 | 0.003249552 | POLA1 | 0.26883933 | 0.035093621 |
| PPOX | 0.9606977 | 3.64544E−08 | ADRM1 | 0.832277 | 9.39373E−05 | SNX30 | 0.31182086 | 0.003249552 | SOX12 | 0.01177846 | 0.035212067 |
| C10orf137 | 1.2438582 | 3.65522E−08 | PAM16 | 0.6718564 | 9.40524E−05 | NPRL2 | 0.48487124 | 0.003262142 | AHSA1 | 0.5815822 | 0.035231374 |
| SNX8 | 0.77689373 | 3.66891E−08 | PIP4K2C | 0.59730923 | 9.40524E−05 | RGMB | 0.470126 | 0.00326495 | FAM127A | 0.48904958 | 0.035355209 |
| LSM2 | 1.0421698 | 3.71202E−08 | CASP2 | 0.46713722 | 9.42524E−05 | AMPD2 | 0.5047723 | 0.003266009 | C1orf50 | 0.3816901 | 0.035394816 |
| AUNIP | 0.7795115 | 3.71202E−08 | CLSPN | 0.4238776 | 9.43472E−05 | DNAJC3 | 0.32640633 | 0.003275145 | ANXA13 | 2.0574856 | 0.035429468 |
| TRIM13 | 0.54702324 | 3.76523E−08 | CHST9 | 1.3522053 | 9.44632E−05 | UEVLD | 0.4407886 | 0.003287239 | SKIV2L2 | 0.13112861 | 0.035439173 |
| GSPT1 | 0.5158508 | 3.81804E−08 | MALL | 0.27766782 | 9.47277E−05 | MORN4 | 0.11205677 | 0.00328948 | TBC1D4 | 0.23653473 | 0.035473583 |
| NME6 | 0.78670955 | 3.83463E−08 | TRMU | 0.5257304 | 9.5014E−05 | HMBOX1 | 0.58621 | 0.003294556 | FAM195A | 0.3552049 | 0.035513306 |
| HENMT1 | 1.7051415 | 3.84553E−08 | DPH3 | 0.40559635 | 9.50671E−05 | ATG7 | 0.25749245 | 0.003294556 | AKR7L | 0.3973884 | 0.035543054 |
| DERA | 1.0279431 | 3.87903E−08 | ATP8A2 | 1.7754077 | 9.52478E−05 | PKNOX1 | 0.14144771 | 0.003300926 | PEX26 | 0.108940765 | 0.035602054 |
| C5orf22 | 0.97358125 | 3.87903E−08 | FDX1 | 0.7121141 | 9.5771E−05 | APPL1 | 0.12630625 | 0.003308862 | PIGN | 0.27224016 | 0.035602054 |
| MRPL21 | 1.1556834 | 4.08821E−08 | ACADM | 0.543516 | 9.58779E−05 | TCF3 | 0.43000352 | 0.003310417 | TAS2R20 | 0.41537672 | 0.03560651 |
| PHLDB2 | 1.7352943 | 4.10718E−08 | TSPAN6 | 0.715801 | 9.58779E−05 | GNAI1 | 0.555955 | 0.003312674 | TRIM5 | 0.15086676 | 0.035681233 |
| ATG4B | 0.8266604 | 4.11269E−08 | ARHGAP17 | 0.5378044 | 9.59958E−05 | SP3 | 0.13792774 | 0.003313049 | DYDC2 | 0.07438534 | 0.035685978 |
| MTFP1 | 1.1875484 | 4.14167E−08 | ALG8 | 0.8063924 | 9.60494E−05 | LSM12 | 0.4686339 | 0.003317083 | ZKSCAN5 | 0.37684038 | 0.035851264 |
| CDIP1 | 0.66536725 | 4.1696E−08 | EIF3F | 1.0009818 | 9.62535E−05 | ANAPC4 | 0.37507182 | 0.003327648 | PSRC1 | 0.43107033 | 0.035851264 |
| ZNF496 | 0.8780673 | 4.1952E−08 | BNIPL | 0.023487652 | 9.70418E−05 | EFEMP2 | 0.6266546 | 0.003343987 | FAM64A | 0.8651506 | 0.035856029 |
| RHOA | 0.6960909 | 4.22349E−08 | D2HGDH | 0.56103456 | 9.7411E−05 | GABPB1-AS1 | 0.25082523 | 0.003343987 | PTS | 0.36994445 | 0.035900899 |
| RBM4B | 0.9337568 | 4.23421E−08 | LANCL2 | 0.7201729 | 9.81314E−05 | UBE4A | 0.29166028 | 0.003348056 | MVK | 0.78664476 | 0.035900899 |
| GPATCH2 | 0.86228895 | 4.34113E−08 | ZNF131 | 0.5770868 | 9.81424E−05 | UBE2D1 | 0.6450137 | 0.003357406 | KIN | 0.08090562 | 0.0359256 |
| SCAF4 | 0.8671821 | 4.35735E−08 | MIS18A | 0.82361937 | 9.83727E−05 | PSMC2 | 0.62843764 | 0.003378914 | TGFBR1 | 0.18735619 | 0.0359256 |
| HJURP | 0.60682094 | 4.4156E−08 | L3MBTL2 | 0.71423346 | 9.9488E−05 | CHCHD5 | 0.48983756 | 0.003380009 | TOM1L2 | 0.10702826 | 0.035940485 |
| JAKMIP2 | 0.41223833 | 4.52562E−08 | MNF1 | 0.55503815 | 9.96101E−05 | CHAF1A | 1.0252943 | 0.003382913 | DBF4B | 0.3334588 | 0.035950319 |
| TFPT | 0.78957635 | 4.5371E−08 | GTSF1 | 1.376204 | 0.000100907 | METTL18 | 0.80520296 | 0.003385819 | RBBP7 | 0.56171316 | 0.036030463 |
| MAPRE1 | 0.9938454 | 4.59289E−08 | FUOM | 0.8328496 | 0.000101234 | GBE1 | 0.46209383 | 0.003396586 | PIK3AP1 | 1.0075992 | 0.036030463 |
| PRELP | 0.5845573 | 4.59289E−08 | POP7 | 0.7812177 | 0.000101585 | ADD1 | 0.40493488 | 0.003408599 | ZNF37A | 0.014543218 | 0.036030463 |
| HNRNPH3 | 0.9206407 | 4.72547E−08 | YAE1D1 | 0.7820064 | 0.000101641 | NCK2 | 0.48422495 | 0.003415173 | AP5M1 | 0.27693072 | 0.036182444 |
| FKBP11 | 1.1910377 | 4.79207E−08 | NGDN | 0.8174501 | 0.000101789 | ANLN | 0.9090551 | 0.003427857 | AADAT | 0.84797806 | 0.036238135 |
| ILKAP | 0.93942255 | 4.83722E−08 | CORO1B | 0.6861561 | 0.000101868 | RCOR3 | 0.30303088 | 0.003428856 | MAGOHB | 0.18696097 | 0.036257947 |
| POGK | 0.799263 | 4.86538E−08 | SPECC1L | 0.3881135 | 0.000101891 | IFT172 | 0.63607246 | 0.003439137 | MED24 | 0.27330983 | 0.036329045 |
| QPRT | 0.9577839 | 4.88352E−08 | TXNDC9 | 0.7295786 | 0.000102152 | RBMX2 | 0.30459973 | 0.003451289 | C6orf47 | 0.34523416 | 0.036344076 |
| OAZ2 | 1.0710675 | 4.9523E−08 | RPP25L | 0.93590343 | 0.000102255 | USP32P2 | 0.5460556 | 0.003459786 | SPAG5 | 0.8293421 | 0.036348344 |
| IMP4 | 1.1574746 | 4.96189E−08 | C21 orf91 | 0.83736455 | 0.000102358 | C3orf62 | 0.031241395 | 0.003460905 | CNOT3 | 0.21718192 | 0.036348344 |
| ZFAT | 0.59818685 | 4.98335E−08 | CPPED1 | 0.32825896 | 0.00010377 | LOC642852 | 0.5400298 | 0.003476106 | CCRN4L | 0.12968028 | 0.036353172 |
| EXO5 | 0.82006073 | 5.09879E−08 | HMHA1 | 0.4043404 | 0.000104186 | ATP6V1H | 0.5744547 | 0.003477231 | CDKN2AIP | 0.40185684 | 0.036516311 |
| CSE1L | 0.8973757 | 5.12235E−08 | SREK1IP1 | 0.07279836 | 0.000104186 | C16orf58 | 0.4212722 | 0.003505447 | LOC729603 | 0.009405023 | 0.036531411 |
| LIG1 | 0.748551 | 5.15366E−08 | LARP1 | 0.4138752 | 0.000104826 | MAMSTR | 1.0794193 | 0.003513442 | NEIL1 | 1.2149234 | 0.036551647 |
| ANXA7 | 1.0131824 | 5.19288E−08 | SCG3 | 0.7268959 | 0.000104931 | EGLN1 | 0.25526032 | 0.00351384 | COPS7B | 0.3004458 | 0.036586459 |
| ZBED1 | 0.72773665 | 5.23083E−08 | SLC47A1 | 0.48983142 | 0.000105107 | ZNF343 | 0.55520314 | 0.00351384 | ACVR2B | 0.22754672 | 0.036586459 |
| RPP21 | 1.0838091 | 5.26905E−08 | UBXN2B | 0.31163338 | 0.000106011 | ZNF573 | 0.05322448 | 0.003518101 | ZBTB4 | 0.3087364 | 0.036586459 |
| PAK4 | 0.9567208 | 5.29653E−08 | MOSPD2 | 0.8122705 | 0.000106047 | KDM1A | 0.34112307 | 0.003521628 | SNX1 | 0.24838582 | 0.036642692 |
| ZNF592 | 0.807727 | 5.29735E−08 | UTP23 | 0.36830676 | 0.000106437 | MTMR3 | 0.33657 | 0.003521628 | RAB3GAP1 | 0.13769805 | 0.036668127 |
| GGA2 | 0.68079424 | 5.35986E−08 | ZNF503-AS1 | 1.6565312 | 0.000106662 | C15orf38-AP3S2 | 0.7052571 | 0.003530911 | WDR70 | 0.29944667 | 0.03668843 |
| RRN3 | 0.7227789 | 5.3942E−08 | ACADSB | 0.2971768 | 0.000106674 | OSTC | 0.74682677 | 0.003531425 | EDC4 | 0.2487348 | 0.036780889 |
| CPSF3 | 1.0547193 | 5.44166E−08 | IMPA1 | 0.5116643 | 0.000106852 | RPL23AP32 | 0.88159853 | 0.003547656 | RGR | 0.5066572 | 0.036878165 |
| GLRX3 | 1.0431585 | 5.45382E−08 | SOX6 | 0.50782305 | 0.000106959 | CRTAC1 | 1.4910908 | 0.003550063 | RSAD1 | 0.32832736 | 0.036893121 |
| PMVK | 0.92338824 | 5.56336E−08 | ZNF766 | 0.4916687 | 0.000107043 | FAHD2B | 0.4605836 | 0.003552471 | EED | 0.3967501 | 0.036893121 |
| FAM101B | 0.73025435 | 5.56336E−08 | PPP1CA | 0.7724812 | 0.000107126 | DUSP23 | 0.6297265 | 0.003554138 | MMAA | 0.5016258 | 0.036908084 |
| PDCD4 | 0.7976972 | 5.57006E−08 | RPL7L1 | 0.69977486 | 0.000107592 | NT5C3 | 0.42572176 | 0.003556549 | MAP4K3 | 0.20754497 | 0.03693368 |
| APEH | 0.95839506 | 5.57006E−08 | FOXO3B | 0.45840043 | 0.000108348 | FEZ1 | 0.16711767 | 0.003574675 | LOC100289561 | 0.4417428 | 0.036995287 |
| ZNF574 | 1.2560191 | 5.59908E−08 | NARF | 0.7862652 | 0.000108577 | TTC21B | 0.004102779 | 0.003577734 | ZRSR2 | 0.27389443 | 0.036995287 |
| TIMM23 | 0.9769205 | 5.6701E−08 | TSEN15 | 0.7135706 | 0.000108879 | PGF | 0.95725644 | 0.003585132 | SNX17 | 0.60055214 | 0.037046879 |
| FAM217B | 0.52106774 | 5.671E−08 | AKAP6 | 1.1321793 | 0.000109012 | CCDC97 | 0.48120353 | 0.003587563 | ZC3H18 | 0.23949198 | 0.037072285 |
| RPP14 | 0.71986187 | 5.71068E−08 | COMMD6 | 0.51890236 | 0.000109073 | MAGEA4 | 1.0768204 | 0.003592544 | MED7 | 0.38752654 | 0.037149975 |
| ZNF345 | 0.7081066 | 5.74553E−08 | VCPIP1 | 0.34376243 | 0.000109219 | ZNF322 | 0.2309489 | 0.003604556 | MAP2K5 | 0.4719485 | 0.037263478 |
| CIRH1A | 0.9288039 | 5.75496E−08 | LINC00665 | 0.34168813 | 0.000109219 | SSH2 | 0.24201694 | 0.003617887 | DBP | 0.68933547 | 0.037325844 |
| ZEB2 | 0.6756155 | 5.761E−08 | BAP1 | 0.5611922 | 0.00011001 | MSL3 | 0.59300995 | 0.003619697 | CD40 | 0.8480121 | 0.037361874 |
| HNRNPR | 0.9677098 | 5.76285E−08 | ZNF121 | 0.84611124 | 0.000110905 | METTL3 | 0.42787233 | 0.003621508 | FAM86EP | 0.19456083 | 0.037402893 |
| CCL28 | 0.82274383 | 5.76285E−08 | ALX1 | 0.8070514 | 0.000111201 | DTX3 | 0.62671435 | 0.00362332 | LOC100506207 | 0.1048563 | 0.037580642 |
| TRAPPC4 | 0.9726262 | 5.80067E−08 | NCBP2 | 0.76761204 | 0.000111201 | ASCC3 | 0.18931173 | 0.003634783 | USP16 | 0.25416756 | 0.037664529 |
| ALG5 | 1.0730752 | 5.82916E−08 | MCM3 | 0.7805655 | 0.000112255 | POU2F1 | 0.018523348 | 0.003639825 | HDLBP | 0.3883469 | 0.037679771 |
| DNAJA1 | 1.1377294 | 5.87689E−08 | EID1 | 0.8606277 | 0.000112268 | RPL39L | 0.6066688 | 0.003644227 | POLM | 0.21343029 | 0.037789967 |
| MAP1S | 1.0685806 | 5.91449E−08 | POLR3H | 0.5591864 | 0.000112455 | HTR3B | 0.06431211 | 0.003651221 | NT5C3B | 0.41231567 | 0.037799973 |
| CDKN2B-AS1 | 0.27910885 | 5.98764E−08 | ZC3H4 | 0.22221033 | 0.000112493 | MYLK | 0.6781811 | 0.003654988 | C12orf45 | 0.4324734 | 0.037799973 |
| SCRN1 | 1.4734824 | 5.99039E−08 | ALG11 | 0.71277374 | 0.000112768 | FAM 103A1 | 0.53292876 | 0.003655521 | PLEKHM2 | 0.31486788 | 0.037852545 |
| NBL1 | 1.5030098 | 6.0038E−08 | IKZF5 | 0.635815 | 0.000113773 | BCL9L | 0.6021241 | 0.003660474 | NDUFA7 | 0.5536978 | 0.037904903 |
| ZC2HC1A | 0.6453533 | 6.13863E−08 | RFPL1-AS1 | 0.17936906 | 0.000113773 | SDCCAG8 | 0.29256877 | 0.003661008 | LOC100505865 | 0.4865616 | 0.037951747 |
| RRAGD | 0.8500528 | 6.25502E−08 | TXN2 | 0.847227 | 0.000113912 | NUDT2 | 0.5779919 | 0.003666733 | HMGCR | 0.3313483 | 0.037951747 |
| GDPD5 | 1.317646 | 6.28755E−08 | ARHGAP42 | 0.45152 | 0.000114152 | PPIL4 | 0.3861519 | 0.003669216 | MUS81 | 0.34672388 | 0.037951747 |
| RBM18 | 0.8186921 | 6.36144E−08 | ENG | 0.7037992 | 0.000114457 | LAMP1 | 0.35057873 | 0.003671051 | CDH15 | 1.1687511 | 0.037956771 |
| SLC16A1 | 0.78066176 | 6.37377E−08 | AAR2 | 0.6637488 | 0.000114457 | HIGD2A | 0.8174304 | 0.003688524 | PKIB | 0.86820453 | 0.038040834 |
| HSDL2 | 0.87914747 | 6.4088E−08 | UCHL3 | 0.76851296 | 0.000114546 | TMCO1 | 0.48989293 | 0.003703992 | C17orf100 | 0.43361697 | 0.038040834 |
| NUPL1 | 0.54288524 | 6.57987E−08 | CKAP2 | 0.78381115 | 0.000115157 | RTEL1 | 0.30627316 | 0.003706498 | BTBD2 | 0.21011111 | 0.038051182 |
| CRTAP | 0.7418161 | 6.61991E−08 | SLC35A1 | 0.64753324 | 0.000115184 | LGR4 | 0.9521462 | 0.003715574 | PSME4 | 0.16771474 | 0.038066845 |
| XIST | 0.95544076 | 6.66216E−08 | ORC6 | 0.39563894 | 0.000115184 | ROGDI | 0.7530035 | 0.003719403 | PCGF1 | 0.29754028 | 0.038146076 |
| DRG1 | 1.1215507 | 6.67114E−08 | CLASRP | 0.5015323 | 0.000115299 | IER3IP1 | 0.5689528 | 0.003719946 | WBP1 | 0.31389824 | 0.038156449 |
| TOP3A | 0.5214661 | 6.70185E−08 | DUSP7 | 0.76183075 | 0.000115414 | FAM84B | 0.08384265 | 0.003723664 | PGM3 | 0.10854381 | 0.0382249 |
| DIS3L | 0.5849725 | 6.79649E−08 | RNF26 | 0.7990138 | 0.000115812 | NUBP1 | 0.5864149 | 0.003724207 | UBFD1 | 0.14657249 | 0.038309761 |
| RAB3D | 0.5448143 | 6.83496E−08 | SHARPIN | 0.604187 | 0.000116842 | CHMP6 | 0.608543 | 0.003728045 | IDH3G | 0.42789948 | 0.038346631 |
| DPH1 | 0.8691803 | 6.93144E−08 | POLDIP2 | 0.62578714 | 0.000117452 | BAZ1A | 0.34783003 | 0.003764233 | SCYL1 | 0.3401535 | 0.038373103 |
| RABIF | 0.68367624 | 6.93466E−08 | PRMT6 | 0.89356494 | 0.000117543 | RSC1A1 | 0.9226012 | 0.003773439 | DDX11 | 0.1686159 | 0.038437087 |
| DCTD | 0.94385797 | 6.94198E−08 | RARS2 | 0.67032117 | 0.000117544 | NPRL3 | 0.3935535 | 0.003778659 | NDUFAF2 | 0.2954261 | 0.038485067 |
| GPS2 | 0.95038044 | 6.98213E−08 | C1orf233 | 2.1648831 | 0.000117544 | AKR1A1 | 0.59738624 | 0.00378778 | ZC3H11A | 0.17889473 | 0.038532277 |
| FAM161A | 0.5464586 | 7.07646E−08 | SNRPC | 0.899472 | 0.000117636 | PRR19 | 1.1908203 | 0.003795028 | GTF2H4 | 0.6587898 | 0.038532277 |
| BLZF1 | 0.5341576 | 7.12995E−08 | ZNF33A | 0.21225105 | 0.000119287 | EOGT | 0.1747044 | 0.003797592 | PCMTD1 | 0.13539374 | 0.038532277 |
| GNPAT | 0.93148625 | 7.15219E−08 | JARID2 | 0.3408516 | 0.000120082 | POLG | 0.40478355 | 0.003804858 | MSH5 | 0.21482673 | 0.038569337 |
| ZC3H12C | 1.0806198 | 7.16818E−08 | PCNP | 0.6664572 | 0.000121149 | MKRN2 | 0.47259364 | 0.003830345 | EMCN | 0.44335777 | 0.038595945 |
| AP2A1 | 0.5202521 | 7.16856E−08 | LOC100144602 | 2.084347 | 0.000121418 | RTBDN | 0.009986922 | 0.003831579 | ECI1 | 0.57760227 | 0.038703084 |
| SLC25A15 | 0.5084784 | 7.16856E−08 | HK1 | 0.6690787 | 0.000121418 | RBM6 | 0.47396168 | 0.003839581 | SLC2A9 | 1.6026437 | 0.038708203 |
| ERCC3 | 0.71337116 | 7.19516E−08 | PPIH | 0.75829583 | 0.000121607 | TAB1 | 0.23391043 | 0.003844207 | GPBP1L1 | 0.07855936 | 0.038815341 |
| ANKRD54 | 0.87574285 | 7.31378E−08 | TTF2 | 0.23643115 | 0.000121607 | TMEM41A | 0.26693523 | 0.003852677 | COX7B | 0.6834054 | 0.038836418 |
| C6orf48 | 1.2143489 | 7.31936E−08 | EXOSC2 | 0.35825965 | 0.000121702 | THBS3 | 0.68500954 | 0.003852677 | MIPOL1 | 0.2865556 | 0.038922738 |
| AKT1 | 0.88383853 | 7.34868E−08 | SHANK2-AS3 | 0.19535725 | 0.000121716 | CBX2 | 0.62662274 | 0.003854598 | ATP2C1 | 0.18385608 | 0.03894956 |
| KIFAP3 | 0.811299 | 7.34997E−08 | MAP2K2 | 0.48579773 | 0.000121891 | LCLAT1 | 0.2625949 | 0.0038572 | POP5 | 0.39825302 | 0.038998098 |
| HMGXB3 | 0.7090634 | 7.36641E−08 | FOXN2 | 0.24255393 | 0.000123749 | UNC13D | 0.80539817 | 0.003858442 | TTK | 0.9549258 | 0.039003252 |
| ZNF252P | 0.3087733 | 7.42641E−08 | PTEN | 0.49711597 | 0.000124557 | DDX42 | 0.18371153 | 0.003865815 | DAK | 0.18157372 | 0.039013835 |
| DDX19A | 0.73883265 | 7.53766E−08 | NDEL1 | 0.71024054 | 0.000124737 | COQ4 | 0.33408257 | 0.00386626 | FAM138F | 0.20782629 | 0.039089349 |
| ATF1 | 0.8297252 | 7.62272E−08 | TMED3 | 0.8868478 | 0.000125164 | DDX58 | 0.011997225 | 0.003874895 | RYBP | 0.16099104 | 0.039127155 |
| NDUFV3 | 0.6801064 | 7.81582E−08 | ZNF428 | 0.43211237 | 0.000125871 | KIAA1430 | 0.27627808 | 0.003880929 | STK39 | 0.94964194 | 0.039197683 |
| C9orf91 | 0.46477154 | 7.83558E−08 | ACO1 | 0.6074104 | 0.000126959 | SFPQ | 0.3087568 | 0.003893023 | PACRGL | 0.47230586 | 0.039229852 |
| CHML | 0.51272255 | 7.87617E−08 | PLD3 | 0.6850352 | 0.00012731 | EIF2AK2 | 0.31872693 | 0.00389359 | TM9SF4 | 0.2575544 | 0.03924049 |
| GEMIN4 | 1.199747 | 8.033E−08 | WIZ | 0.841069 | 0.000128338 | ARIH1 | 0.2996539 | 0.003896904 | PPT2 | 0.4057441 | 0.039475151 |
| HSD17B1 | 0.6746345 | 8.033E−08 | ENO1 | 0.7495346 | 0.000128707 | EXOC2 | 0.3299887 | 0.003901476 | TMEM194B | 0.33615845 | 0.039568205 |
| DEF8 | 0.99853724 | 8.08644E−08 | LAS1L | 0.70498496 | 0.000128707 | GSTA4 | 0.52849776 | 0.003901476 | COQ7 | 0.1691994 | 0.039628144 |
| PTCD3 | 0.6303322 | 8.09026E−08 | ARMCX1 | 0.8324348 | 0.000129121 | ADCY9 | 0.7015347 | 0.003906172 | SMIM1 | 0.70766217 | 0.039644379 |
| RAB9A | 0.91278535 | 8.1393E−08 | PYGO2 | 0.47867164 | 0.000129121 | BRMS1L | 0.8639104 | 0.003914319 | GALK1 | 0.43039823 | 0.039649608 |
| COA4 | 1.0392623 | 8.16466E−08 | SF3A2 | 0.63063914 | 0.000129121 | BPGM | 0.6432752 | 0.003923745 | TNFAIP1 | 0.3475379 | 0.039654838 |
| RSU1 | 0.6220599 | 8.25032E−08 | TTC5 | 0.9324977 | 0.000129265 | NARFL | 0.3899275 | 0.003924317 | LINC00338 | 0.19364862 | 0.039660069 |
| NOC2L | 0.93535185 | 8.25907E−08 | ABHD10 | 0.6687266 | 0.000129393 | ARRB2 | 0.67881984 | 0.003936655 | CSDA | 0.081311844 | 0.03972592 |
| PROSER1 | 1.0127962 | 8.26449E−08 | C6orf147 | 0.27909246 | 0.000129694 | TTN-AS1 | 0.3193737 | 0.003938497 | TMA16 | 0.086376965 | 0.039758744 |
| SNRPE | 0.842846 | 8.26449E−08 | SMCR8 | 0.43073583 | 0.000129966 | LOC100130872 | 0.00789233 | 0.003938497 | LOC650226 | 0.848488 | 0.03991851 |
| PKLR | 2.66519 | 8.27327E−08 | USP19 | 0.5466464 | 0.000131434 | THAP8 | 0.87409896 | 0.003938954 | NEURL4 | 0.39686668 | 0.040056893 |
| RPL13AP20 | 0.6263942 | 8.34781E−08 | HSPBP1 | 0.64662755 | 0.000132174 | INF2 | 0.32238594 | 0.003961659 | DHCR7 | 0.84630746 | 0.040084396 |
| POM121 | 0.5706567 | 8.4033E−08 | EPT1 | 0.27002284 | 0.000132743 | IDI2-AS1 | 0.87939185 | 0.003969214 | NEK1 | 0.18733917 | 0.040106076 |
| ARNT2 | 0.9664745 | 8.5754E−08 | TMEM50B | 0.4933405 | 0.000132758 | RAD54L | 1.1091844 | 0.003969794 | FREM1 | 1.7297304 | 0.04011692 |
| HDHD1 | 1.2435932 | 8.67795E−08 | LINC00516 | 0.5082875 | 0.000132876 | SLC30A8 | 0.9087113 | 0.003972469 | ARGLU1 | 0.3634033 | 0.040183433 |
| NADKD1 | 0.5101187 | 8.8332E−08 | LOC101101776 | 0.5082875 | 0.000132876 | ZBTB44 | 0.07834857 | 0.003975727 | SLC35E1 | 0.057236 | 0.040255063 |
| PGM2 | 0.90950906 | 8.88404E−08 | ZC3H15 | 0.6384475 | 0.000133272 | LINC00847 | 0.5822464 | 0.003977007 | GGACT | 1.022895 | 0.040260364 |
| MXI1 | 0.90512294 | 8.94205E−08 | PCBP4 | 0.680839 | 0.000133832 | DCAF5 | 0.3949079 | 0.003980387 | FAM21A | 0.5106517 | 0.040265666 |
| NCLN | 0.7254825 | 8.94205E−08 | NVL | 0.53805697 | 0.000133935 | MED21 | 0.49782538 | 0.003992185 | EAPP | 0.3507983 | 0.040269869 |
| SUSD5 | 1.0130986 | 9.0251E−08 | MAN2B1 | 0.60557926 | 0.000133951 | PAGR1 | 0.27364638 | 0.003993471 | PM20D2 | 0.18263282 | 0.040269869 |
| PRUNE | 1.0588404 | 9.02677E−08 | DSTYK | 0.40251482 | 0.000134941 | IKBKE | 0.67266196 | 0.003997914 | IPP | 0.044301428 | 0.040269869 |
| KXD1 | 1.003145 | 9.02845E−08 | NLE1 | 0.84728914 | 0.000136417 | ZHX3 | 0.09606709 | 0.003998499 | TMEM222 | 0.35552925 | 0.04029192 |
| PEMT | 1.0793391 | 9.03277E−08 | RPS26 | 0.6364052 | 0.000136973 | COASY | 0.43985212 | 0.004009641 | GTF2B | 0.39100274 | 0.040353094 |
| HDHD2 | 0.8622039 | 9.154E−08 | SCN2B | 0.0113198 | 0.000137698 | HINT3 | 0.3389269 | 0.004010933 | TBC1D2B | 0.26634336 | 0.040391965 |
| DIMT1 | 0.91808563 | 9.31481E−08 | NQO1 | 0.576298 | 0.000139616 | LAP3 | 0.55139375 | 0.004015751 | PMS1 | 0.18131657 | 0.040458874 |
| RNPEP | 0.9648624 | 9.42223E−08 | PTGDS | 2.0293229 | 0.000139878 | SNX20 | 0.13836043 | 0.004024109 | HIAT1 | 0.31645977 | 0.04047541 |
| SWAP70 | 0.7274951 | 9.42223E−08 | MINA | 0.52917445 | 0.000140109 | PRORSD1P | 0.2699336 | 0.00402682 | AMER1 | 0.9895235 | 0.040575853 |
| GFM2 | 0.8763746 | 9.45153E−08 | DTD2 | 0.262775 | 0.000140494 | KIAA0146 | 0.32852527 | 0.004045844 | SF1 | 0.17199527 | 0.040586539 |
| ZNF286A | 0.6277283 | 9.45607E−08 | TBC1D22A | 0.5469223 | 0.000140572 | ARMCX5 | 0.5732864 | 0.004052005 | FAM138A | 0.19460325 | 0.040586539 |
| ZDHHC6 | 1.0972103 | 9.54836E−08 | RCCD1 | 0.9810865 | 0.000140866 | COQ6 | 0.52141684 | 0.004052005 | ZNF219 | 0.4459062 | 0.040614363 |
| RBM8A | 0.6078917 | 9.54836E−08 | UPF3B | 0.54792184 | 0.000140961 | JAK2 | 0.80538166 | 0.00407114 | H2AFV | 0.26235247 | 0.040664707 |
| ZNF426 | 0.7930374 | 9.8518E−08 | YBX1 | 0.63135374 | 0.000140961 | GADD45GIP1 | 0.5443592 | 0.004076025 | HDAC6 | 0.28962827 | 0.040782755 |
| USF1 | 1.1207918 | 9.95758E−08 | RIC8A | 0.6502073 | 0.000141039 | LOC100505761 | 0.41790915 | 0.004085096 | NBPF7 | 0.005563724 | 0.040816341 |
| FAM136A | 0.9719927 | 9.97108E−08 | GTF3C3 | 0.60574406 | 0.000141117 | DDX3X | 0.3623673 | 0.004085096 | MAP3K11 | 0.18176138 | 0.040849952 |
| VAMP2 | 0.63946193 | 1.00459E−07 | CHRNA7 | 0.81696165 | 0.000141289 | UCKL1 | 0.3340561 | 0.00409215 | RBM25 | 0.25500336 | 0.040911313 |
| NUDT19 | 0.67823935 | 1.00683E−07 | FXN | 0.3764564 | 0.000141739 | SENP1 | 0.42350516 | 0.00411218 | ATRN | 0.17373277 | 0.040922353 |
| COPS5 | 1.0478833 | 1.01172E−07 | AURKA | 0.8364589 | 0.000141756 | C7orf13 | 1.0109024 | 0.004117833 | PSKH1 | 0.5108885 | 0.040944443 |
| PPP1R7 | 0.9345152 | 1.04059E−07 | SOLH | 0.7099804 | 0.000143633 | C20orf111 | 0.48347804 | 0.004122048 | GFM1 | 0.2373756 | 0.040989478 |
| CBX6 | 1.0734998 | 1.04808E−07 | BAG6 | 0.34438938 | 0.00014373 | LOC100506710 | 0.25637227 | 0.004131334 | ACAP2 | 0.039912846 | 0.041102389 |
| INPPL1 | 0.6358144 | 1.05042E−07 | ACTR10 | 0.6040438 | 0.000144125 | NAP1L1 | 0.41145015 | 0.004135561 | OXSM | 0.58237535 | 0.041346985 |
| MRPS10 | 0.76656294 | 1.05399E−07 | GAPDH | 0.42433378 | 0.000144236 | RNF144A | 0.16440332 | 0.004139671 | NPLOC4 | 0.09302753 | 0.04136356 |
| STOML2 | 1.1243829 | 1.05665E−07 | HERPUD2 | 0.2686126 | 0.000144601 | TRIM25 | 0.37642044 | 0.004161981 | CCNB1 | 0.56147015 | 0.04136356 |
| LRRC58 | 0.555747 | 1.05931E−07 | RGL1 | 0.6621877 | 0.000144808 | LARP4 | 0.35040745 | 0.004188801 | GPX8 | 0.07533577 | 0.041390743 |
| ZBTB45 | 1.0130877 | 1.07973E−07 | FASTKD5 | 0.90893614 | 0.000145096 | LOC401321 | 0.068087794 | 0.00420988 | NOD1 | 0.32407787 | 0.041390743 |
| THOC3 | 0.9821422 | 1.08686E−07 | NABP1 | 0.39697373 | 0.000145289 | TXNRD1 | 0.43697193 | 0.004231674 | HMGCS1 | 0.011960143 | 0.041390743 |
| PARVB | 0.5594564 | 1.08707E−07 | ACAA1 | 0.70268846 | 0.000145847 | OSGEPL1 | 0.3440747 | 0.004235996 | LDOC1L | 0.24336277 | 0.041481985 |
| OFD1 | 0.67893636 | 1.09266E−07 | FKBP15 | 0.58018196 | 0.000146344 | COMMD7 | 0.5153445 | 0.004238097 | SYNJ2BP | 0.13641512 | 0.041504617 |
| NOL8 | 0.69477344 | 1.09542E−07 | SGSH | 0.5213189 | 0.000146746 | MVD | 0.5116684 | 0.00424391 | ZKSCAN8 | 0.1723924 | 0.04151007 |
| WDR76 | 0.46729428 | 1.12008E−07 | C8orf44 | 0.26594833 | 0.000147052 | CHAC1 | 1.3720258 | 0.004246014 | GPC2 | 0.7766767 | 0.041521253 |
| TMEM220 | 0.4615739 | 1.1203E−07 | C16orf74 | 1.523185 | 0.00014723 | MINK1 | 0.11246783 | 0.004247997 | ZNF444 | 0.45938078 | 0.041543904 |
| EIF2AK4 | 0.3857598 | 1.13294E−07 | LEPR | 0.060233407 | 0.000148022 | STX7 | 0.47060353 | 0.004252212 | RDX | 0.23317945 | 0.041578038 |
| SMYD2 | 1.0002657 | 1.13844E−07 | SFSWAP | 0.39248362 | 0.000148623 | GID4 | 1.1850121 | 0.004252212 | CNNM3 | 0.003742871 | 0.041623679 |
| EIF5B | 0.6147787 | 1.14296E−07 | ZZEF1 | 0.5796751 | 0.000148738 | ZNF674-AS1 | 0.19773844 | 0.0042609 | GTF2IRD1 | 0.27177018 | 0.041680861 |
| DDX52 | 0.7354102 | 1.18607E−07 | SLC2A8 | 1.0138911 | 0.000149081 | CDK16 | 0.3859268 | 0.0042609 | DOCK6 | 0.32542682 | 0.041887726 |
| TRAPPC3 | 1.0366687 | 1.22012E−07 | MZT2B | 0.53405887 | 0.000149229 | SGTA | 0.47765264 | 0.004262768 | C1orf54 | 0.7737538 | 0.041979923 |
| KIAA1549 | 0.20952208 | 1.22106E−07 | LRP4-AS1 | 0.41449818 | 0.000150195 | ARFGEF2 | 0.12109223 | 0.004262768 | TPRKB | 0.44709542 | 0.042066502 |
| PPM1G | 0.9955811 | 1.22733E−07 | ORAOV1 | 0.48176 | 0.000150476 | VAC14 | 0.55023086 | 0.004267866 | TXNDC5 | 0.39955917 | 0.042211335 |
| MKNK1 | 0.9567583 | 1.2573E−07 | POLR3F | 0.74124664 | 0.000150888 | COL25A1 | 1.8663768 | 0.004269237 | DNM2 | 0.20219922 | 0.042344945 |
| SUGP1 | 0.7591944 | 1.26582E−07 | TBC1D17 | 0.44830236 | 0.000151699 | ARID5B | 0.37918913 | 0.00427422 | ADC | 0.28593555 | 0.042408827 |
| SRSF12 | 0.36580712 | 1.26582E−07 | MGST1 | 1.5427438 | 0.00015278 | AGPAT5 | 0.28588027 | 0.004278583 | ASH1L | 0.15191875 | 0.042426056 |
| HEBP1 | 1.0663912 | 1.27747E−07 | ADIPOR1 | 0.7908385 | 0.000153701 | LOC441454 | 0.3124398 | 0.004280705 | EIF4G1 | 0.30741337 | 0.042489759 |
| GAS2L3 | 1.1137242 | 1.27846E−07 | TAOK3 | 0.49608824 | 0.000154679 | RIPK2 | 0.4371456 | 0.004288072 | CHRNE | 2.6829364 | 0.042495321 |
| TTC38 | 1.1147673 | 1.27871E−07 | ARV1 | 0.65604407 | 0.000154731 | SRSF4 | 0.50334024 | 0.004294699 | CPT1A | 0.17108378 | 0.042652601 |
| STARD3NL | 1.0076691 | 1.29871E−07 | NIPSNAP1 | 0.6807907 | 0.000155734 | CGNL1 | 0.037766475 | 0.004306477 | ARID1A | 0.054744825 | 0.042652601 |
| ANKRD17 | 0.5944824 | 1.30386E−07 | VPS35 | 0.6688518 | 0.00015599 | HAUS4 | 0.4803585 | 0.004306477 | RNGTT | 0.21323153 | 0.042687525 |
| VRK3 | 1.0961908 | 1.31585E−07 | CYFIP1 | 0.45042711 | 0.000158062 | TRPV2 | 0.6005374 | 0.00430786 | NUP37 | 0.525645 | 0.042693111 |
| PPFIA3 | 1.5043696 | 1.33565E−07 | SULT1A4 | 0.3373659 | 0.00015834 | ARHGDIA | 0.34095687 | 0.004312255 | PFDN6 | 0.35799366 | 0.042757449 |
| EIF2B1 | 1.0602427 | 1.35221E−07 | SULT1A3 | 0.3373659 | 0.00015834 | CDV3 | 0.2646198 | 0.004323956 | PNPLA6 | 0.1350999 | 0.042845424 |
| EIF4A3 | 1.0914164 | 1.36227E−07 | KLHL30 | 0.3947681 | 0.000158739 | SAAL1 | 0.5832568 | 0.004331391 | CKB | 0.68555856 | 0.042921759 |
| SPATA20 | 0.9319548 | 1.37043E−07 | RFWD3 | 0.41804895 | 0.000159555 | RAP1GDS1 | 0.21833631 | 0.004332025 | SESN3 | 0.18609834 | 0.042927083 |
| MRPL35 | 0.75489855 | 1.40233E−07 | PHYHD1 | 0.36306003 | 0.000159608 | CCDC90B | 0.49440965 | 0.004334172 | CYB5R4 | 0.46681672 | 0.042927083 |
| TMEM97 | 0.87353003 | 1.40233E−07 | CPNE8 | 0.5376285 | 0.000159697 | ABLIM1 | 0.7912279 | 0.004364295 | KIF15 | 0.78368396 | 0.043068578 |
| GREB1 | 0.50591075 | 1.40233E−07 | PIK3R4 | 0.520928 | 0.000159994 | TUSC3 | 0.58128303 | 0.00436722 | ASB7 | 0.48283246 | 0.043074206 |
| ZNF281 | 0.80781454 | 1.40735E−07 | PAAF1 | 0.6726354 | 0.000160013 | LOC440288 | 0.034064926 | 0.004387594 | UNC119B | 0.55301225 | 0.04310351 |
| GBF1 | 0.6562771 | 1.40735E−07 | SLC7A4 | 2.08114 | 0.000160556 | LOC100505912 | 1.543446 | 0.004387594 | ZBED5 | 0.15570359 | 0.043109143 |
| NFS1 | 0.81259096 | 1.41373E−07 | MAX | 0.50772697 | 0.000160645 | FAM161B | 0.028508622 | 0.004392064 | TBCC | 0.29938155 | 0.043114491 |
| DDX49 | 1.0533769 | 1.41524E−07 | ZNF253 | 0.46124652 | 0.000162456 | ACD | 0.42020023 | 0.004398716 | MAPKAP1 | 0.34344772 | 0.043114491 |
| ITGA4 | 1.8026558 | 1.44105E−07 | YEATS2 | 0.33567193 | 0.000163701 | METTL2B | 0.27425352 | 0.004398716 | UBALD1 | 0.543618 | 0.043149745 |
| KIAA0141 | 0.6740788 | 1.44674E−07 | FAM86C2P | 1.0496459 | 0.000163827 | TKT | 0.5559779 | 0.004400127 | NUDT3 | 0.3192016 | 0.043155385 |
| CAMKK2 | 0.67848176 | 1.44745E−07 | SMCR5 | 0.6254097 | 0.000165421 | WBP4 | 0.35343954 | 0.004402306 | ZCCHC11 | 0.013557062 | 0.043308843 |
| SELRC1 | 0.7356986 | 1.45318E−07 | ZNF259 | 0.68991774 | 0.000165657 | CAAP1 | 0.36512035 | 0.004405131 | CACNB3 | 0.61522067 | 0.043320447 |
| OSBPL1A | 0.6762409 | 1.45628E−07 | TMEM201 | 0.682217 | 0.000165732 | TPD52L2 | 0.59022427 | 0.004405131 | SOCS7 | 0.027891738 | 0.043355848 |
| BBS4 | 0.8814437 | 1.46246E−07 | COX16 | 0.48236296 | 0.000165732 | DALRD3 | 0.52567095 | 0.004406545 | CDK5R1 | 0.5673322 | 0.043360662 |
| SETD9 | 1.1667547 | 1.48941E−07 | ZNF134 | 0.88633615 | 0.000165824 | NR2C2AP | 0.45888036 | 0.004428508 | HDAC3 | 0.34474993 | 0.043360662 |
| RBBP5 | 0.55079114 | 1.49314E−07 | C6orf136 | 0.88590264 | 0.000165971 | STAT5A | 0.57111144 | 0.004441399 | NDUFB6 | 0.45520493 | 0.043485204 |
| RUFY2 | 0.447756 | 1.49905E−07 | MRPL39 | 0.6255064 | 0.000165971 | PDCL3 | 0.5370732 | 0.004441399 | PGAM2 | 1.0902607 | 0.043485204 |
| TFB2M | 0.9318892 | 1.49935E−07 | LRRC45 | 0.82184374 | 0.000166553 | CDKN3 | 1.0828594 | 0.004445023 | NACAP1 | 0.23988008 | 0.04355001 |
| DENND4B | 0.68615675 | 1.50268E−07 | NELFA | 0.5011676 | 0.000166681 | ZNF114 | 0.15753081 | 0.004445023 | APBA3 | 0.39650455 | 0.043591553 |
| PRMT1 | 0.9581623 | 1.51633E−07 | CHTF8 | 0.6150737 | 0.000166955 | WDR62 | 1.0556812 | 0.004445551 | FAM203A | 0.5538819 | 0.043614903 |
| TGS1 | 0.5756155 | 1.51633E−07 | PDPK1 | 0.4215629 | 0.000167285 | CDCA4 | 0.48205492 | 0.004456283 | EIF1B | 0.46733603 | 0.043638548 |
| FAHD1 | 0.8044431 | 1.5376E−07 | GTF2H2C | 0.38483602 | 0.000167285 | KEAP1 | 0.5243627 | 0.004457713 | ZNF563 | 0.027425673 | 0.043674176 |
| MESDC1 | 0.7578235 | 1.54946E−07 | ALDH7A1 | 0.39732596 | 0.000167887 | IQGAP2 | 1.24631 | 0.004467694 | GFER | 0.2193897 | 0.043787497 |
| FBLN2 | 2.235834 | 1.56095E−07 | MAPK1 | 0.4781951 | 0.000168236 | SUV39H2 | 0.14256512 | 0.004474578 | ANO10 | 0.2411228 | 0.043973582 |
| ZMIZ1 | 0.53446186 | 1.57693E−07 | DOCK7 | 0.36805516 | 0.000168439 | ASNA1 | 0.5231673 | 0.004477572 | ABHD14B | 0.41444206 | 0.044039308 |
| NIT1 | 0.6708806 | 1.62508E−07 | TAF12 | 0.7462031 | 0.000168642 | LINC00381 | 0.39988354 | 0.004478884 | SENP2 | 0.23464502 | 0.04407522 |
| EXOSC1 | 1.1109642 | 1.63149E−07 | MPC1 | 0.72332454 | 0.000169322 | PRH1-PRR4 | 0.29019567 | 0.004480321 | GLB1L | 0.12388354 | 0.044099081 |
| HSPA4 | 0.85383755 | 1.67308E−07 | URB1 | 0.2559934 | 0.000170432 | SLC30A5 | 0.17366228 | 0.004493353 | TIE1 | 1.4633955 | 0.044110873 |
| WRB | 0.9869677 | 1.70882E−07 | SMAD2 | 0.29520267 | 0.000171102 | CLCN6 | 0.17862716 | 0.004507204 | SDK2 | 0.6379617 | 0.044116344 |
| FDXR | 1.3671317 | 1.71064E−07 | KANK2 | 0.45208323 | 0.000171383 | UBR5 | 0.23410738 | 0.004507204 | PANK2 | 0.38171706 | 0.044116344 |
| CLPX | 0.80931 | 1.71788E−07 | TOMM5 | 0.82447124 | 0.000172672 | PDCD2L | 1.057529 | 0.004519399 | ZMYM1 | 0.25336137 | 0.044134183 |
| RPUSD3 | 1.0452073 | 1.74503E−07 | FAM213B | 0.73068154 | 0.000172917 | DNAJB13 | 0.23693138 | 0.004550035 | STAG3 | 0.66690487 | 0.044157791 |
| FBXO7 | 1.0776892 | 1.7519E−07 | E2F4 | 0.83812726 | 0.000173163 | ANKRD13B | 0.56013256 | 0.004550702 | FAM83H | 0.23240362 | 0.04418774 |
| SNW1 | 0.85265356 | 1.7689E−07 | EMC8 | 0.7951554 | 0.000173259 | RAB1A | 0.6076694 | 0.00455216 | SUDS3 | 0.23142548 | 0.044265861 |
| CHEK1 | 0.3440831 | 1.79567E−07 | SH3GLB1 | 0.35424855 | 0.000173618 | SNX12 | 0.33479452 | 0.004556787 | IBA57 | 0.14613044 | 0.044265861 |
| GDE1 | 0.86351955 | 1.79567E−07 | LOC285762 | 0.041600775 | 0.000174242 | RNASEH1 | 0.49589226 | 0.004578521 | DNMT3B | 0.7357532 | 0.044289814 |
| C6orf211 | 0.9632474 | 1.80273E−07 | MSTO2P | 0.6616081 | 0.000174338 | RAD54B | 0.22342749 | 0.004579192 | PEF1 | 0.45628655 | 0.044295593 |
| RGS3 | 1.1105803 | 1.81346E−07 | BANF1 | 0.6796482 | 0.000174435 | C19orf40 | 0.1231347 | 0.004587829 | SLC35B3 | 0.32211104 | 0.044446771 |
| CYB561 | 0.70503914 | 1.82843E−07 | EBAG9 | 0.67109483 | 0.000174758 | FAM50B | 1.2985616 | 0.004588377 | GMDS | 0.40975136 | 0.044476047 |
| PUSL1 | 1.5679483 | 1.83247E−07 | RANBP3 | 0.42344528 | 0.00017512 | NEK7 | 0.22867812 | 0.004588377 | ZNF589 | 0.5040068 | 0.044506186 |
| EYA3 | 0.6092289 | 1.84971E−07 | C14orf105 | 0.039464537 | 0.000175369 | EIF4G2 | 0.5392157 | 0.004600776 | TMEM256 | 0.38066596 | 0.044511992 |
| CDK2 | 0.9983403 | 1.85486E−07 | MRPS27 | 0.57577574 | 0.000175657 | LOC493754 | 0.042327538 | 0.004600776 | QSER1 | 0.07981891 | 0.044560144 |
| UNC45A | 0.87572235 | 1.85844E−07 | RPS6KC1 | 0.48522052 | 0.00017602 | EMILIN1 | 2.1205778 | 0.004600776 | CACFD1 | 0.47813085 | 0.044614719 |
| POLD1 | 1.116516 | 1.87431E−07 | TCOF1 | 0.5375431 | 0.000176156 | DAPK3 | 0.39846155 | 0.004606249 | POMZP3 | 0.19834448 | 0.044619984 |
| DVL2 | 0.73209244 | 1.89084E−07 | RFXANK | 0.55667585 | 0.000176292 | ZNF302 | 0.41312137 | 0.004611604 | PDPR | 0.09748437 | 0.044619984 |
| HDDC3 | 1.0980347 | 1.91407E−07 | RGS16 | 1.0844514 | 0.00017639 | C11orf48 | 0.40734288 | 0.004612281 | NAB2 | 0.35073456 | 0.044747935 |
| FECH | 0.5555896 | 1.91776E−07 | RANGAP1 | 0.5338885 | 0.000176908 | MTMR10 | 0.55705374 | 0.004612958 | SALL4 | 1.0322448 | 0.044906862 |
| TACC1 | 0.71029913 | 1.9286E−07 | ATAD2B | 0.52206755 | 0.000177181 | SEMA5A | 0.1957112 | 0.004633581 | GRPEL2 | 0.19482063 | 0.044967946 |
| STX6 | 0.97755766 | 1.93232E−07 | COL22A1 | 0.96757174 | 0.000177471 | SLBP | 0.56097996 | 0.004635623 | PTK2 | 0.20087871 | 0.044973805 |
| SLC25A16 | 1.4776918 | 1.933E−07 | KIAA1841 | 0.11789367 | 0.000178108 | ICAM3 | 0.6181613 | 0.004635623 | ZNF720 | 0.30097595 | 0.044979665 |
| UBE2T | 1.277165 | 1.933E−07 | MAPK6 | 0.6049561 | 0.00017846 | ABHD8 | 0.8787502 | 0.004635623 | TMEM216 | 0.51317215 | 0.044997532 |
| MFAP1 | 0.93944657 | 1.933E−07 | DCDC2 | 0.17213875 | 0.00017846 | NT5C2 | 0.19525942 | 0.004647583 | HERC2P9 | 0.16356844 | 0.045138182 |
| PSMG3 | 1.0200125 | 1.93451E−07 | GUF1 | 0.40558714 | 0.000179682 | PIGC | 0.51842165 | 0.004653916 | FAF2 | 0.27632 | 0.045186925 |
| HAUS7 | 1.1901174 | 1.95827E−07 | CCDC41 | 0.61127174 | 0.000180444 | ABL1 | 0.30516782 | 0.00466592 | LACE1 | 0.624982 | 0.045235715 |
| PLEKHG3 | 0.95908034 | 1.96148E−07 | C10orf11 | 0.93658537 | 0.000180739 | SRP54 | 0.46273205 | 0.00467053 | STRIP1 | 0.26042306 | 0.045309545 |
| ZNF195 | 0.46277538 | 1.96526E−07 | TERF1 | 0.34903967 | 0.000180761 | ARPP19 | 0.38758472 | 0.004674458 | CHRM1 | 2.5194576 | 0.045358729 |
| NSMCE2 | 0.9897137 | 1.96681E−07 | ATN1 | 0.2597305 | 0.00018094 | CTU2 | 0.5507304 | 0.004678265 | CAPN2 | 0.3308462 | 0.045401769 |
| PCIF1 | 0.93986005 | 1.97004E−07 | OLAH | 0.10140943 | 0.000183249 | PREP | 0.33761686 | 0.0046822 | LIN37 | 0.61833775 | 0.045432174 |
| STRN4 | 0.7873804 | 1.97271E−07 | DAXX | 0.64541745 | 0.000183668 | GPATCH1 | 0.7761744 | 0.004714662 | WLS | 0.2963163 | 0.045432174 |
| WDR11 | 0.77978534 | 2.02329E−07 | ETFA | 0.8263928 | 0.000184525 | HEY1 | 0.5441609 | 0.004733811 | CRLF3 | 0.04778453 | 0.045444285 |
| SLC37A1 | 1.7176845 | 2.0324E−07 | KLHL29 | 0.912196 | 0.000185186 | TPRN | 1.311135 | 0.004736147 | PDIA6 | 0.6160679 | 0.045474995 |
| FAM120B | 0.8765058 | 2.04155E−07 | TMEM120A | 0.69664335 | 0.000185512 | KREMEN1 | 0.27319634 | 0.004766349 | HIST1H2BK | 0.4379867 | 0.045517299 |
| FANCG | 1.2666574 | 2.07957E−07 | HNRNPH2 | 0.5544272 | 0.000185512 | MTHFD2 | 0.5118458 | 0.004767623 | ALKBH1 | 0.46018794 | 0.045641253 |
| BUB3 | 0.959009 | 2.14666E−07 | RAB7A | 0.57033795 | 0.000186118 | ATXN2 | 0.34263712 | 0.00477799 | NR1H2 | 0.32333064 | 0.045684248 |
| EIF3M | 1.0774609 | 2.16677E−07 | SASH1 | 0.31667924 | 0.000186866 | DNAJC16 | 0.14363043 | 0.00477799 | ACAT2 | 0.41062346 | 0.045889486 |
| FOCAD | 0.7436694 | 2.18457E−07 | TRIP4 | 0.7029791 | 0.000188265 | RPS6KB1 | 0.24552684 | 0.004780674 | JMJD7 | 0.25359035 | 0.045939216 |
| MAPKAPK3 | 1.0361823 | 2.20314E−07 | ABCF2 | 0.55974054 | 0.000188473 | FLCN | 0.2546102 | 0.004780674 | STAM | 0.2235488 | 0.045970217 |
| SPR | 1.0713985 | 2.22108E−07 | TFDP1 | 0.7640038 | 0.000188473 | KDM1B | 0.4077111 | 0.004780674 | ZSCAN29 | 0.068382904 | 0.046151447 |
| C16orf62 | 0.8093996 | 2.27782E−07 | RNF185 | 0.6254109 | 0.000189107 | HIF3A | 0.05729493 | 0.004783861 | LHPP | 0.65342057 | 0.046263749 |
| CARKD | 0.9435522 | 2.28155E−07 | LOC100505776 | 0.001039125 | 0.000189171 | CARS2 | 0.64206004 | 0.004784441 | BBS9 | 0.06874971 | 0.046263749 |
| SNRPD2 | 1.0655247 | 2.297E−07 | MED 12 | 0.3307838 | 0.00018948 | ETHE1 | 0.6121573 | 0.004794139 | LPAR2 | 1.0634844 | 0.046383163 |
| STARD7 | 0.81637555 | 2.33565E−07 | MAEA | 0.69814605 | 0.00019065 | EMR2 | 0.15277433 | 0.004808971 | HIST2H2BC | 0.60618323 | 0.046420733 |
| WDR36 | 0.5331012 | 2.49047E−07 | STIL | 0.38028768 | 0.000191003 | KPTN | 0.86503375 | 0.004827894 | LPHN1 | 0.17987318 | 0.046470395 |
| PIPSL | 0.5767743 | 2.49047E−07 | SETD8 | 0.35566422 | 0.000191686 | CSRNP2 | 0.63010705 | 0.004827894 | SYT14 | 0.6663368 | 0.046697725 |
| TRPM6 | 0.44096452 | 2.50303E−07 | POLE3 | 0.6603965 | 0.00019233 | MSRB1 | 0.7559847 | 0.004853469 | RAD9A | 0.30883858 | 0.046805108 |
| PRKCE | 0.72317797 | 2.52924E−07 | C15orf52 | 0.21079955 | 0.000194736 | NDUFA11 | 0.6315875 | 0.00485645 | RNASEL | 0.32194975 | 0.046868139 |
| C7orf41 | 1.1234889 | 2.54848E−07 | RFC3 | 0.7384615 | 0.000195263 | UQCR10 | 0.52041084 | 0.00485645 | TMEM63A | 0.19509861 | 0.046868139 |
| PIK3C2B | 0.30955353 | 2.55626E−07 | PARP10 | 0.65882 | 0.000196003 | PSMD7 | 0.6081638 | 0.004868934 | C20orf26 | 2.1979556 | 0.046873646 |
| NECAP2 | 0.83178204 | 2.57133E−07 | ZNF461 | 0.4663547 | 0.000197594 | RPRD1A | 0.29039314 | 0.004884822 | PIK3CB | 0.05397103 | 0.046873646 |
| EXOSC5 | 1.098969 | 2.57773E−07 | AP3D1 | 0.31962568 | 0.000198257 | GBA | 0.54831195 | 0.004886385 | NHP2 | 0.63846403 | 0.046898542 |
| SIGIRR | 0.8182718 | 2.58121E−07 | SMIM12 | 0.3659968 | 0.000198367 | CYB5D1 | 0.5019758 | 0.004902073 | RHPN1-AS1 | 0.22615881 | 0.046930104 |
| ADAMTS2 | 2.2034132 | 2.58616E−07 | NME2 | 0.53127694 | 0.000199014 | LOC642361 | 0.32667434 | 0.004902073 | DHRS12 | 0.59122366 | 0.046942565 |
| ANKRD46 | 1.3122363 | 2.62284E−07 | ADAT2 | 0.46868154 | 0.000199252 | PLXNA2 | 0.8154689 | 0.004919634 | ZNF28 | 0.23028684 | 0.047011296 |
| ARHGAP11A | 0.5971787 | 2.62638E−07 | DCAF4 | 0.9875767 | 0.000199749 | CMSS1 | 0.45898828 | 0.004919634 | NUDCD2 | 0.38876823 | 0.047030159 |
| NDUFA10 | 0.67216396 | 2.64335E−07 | SEC22C | 0.5520702 | 0.00019984 | SPDYE7P | 0.32231227 | 0.00493397 | LOC100132356 | 0.25636488 | 0.047106519 |
| MEIS1 | 0.8036643 | 2.6725E−07 | CDC73 | 0.50867164 | 0.00019984 | ARAF | 0.41750124 | 0.004956037 | MSRB2 | 0.3156727 | 0.047150715 |
| HEATR5B | 0.56106246 | 2.67989E−07 | CEP57 | 0.52129006 | 0.000200018 | ASPSCR1 | 0.46844643 | 0.004961774 | MYH13 | 0.67259824 | 0.047150715 |
| IMP3 | 1.0378861 | 2.75886E−07 | EPHA5 | 2.12003 | 0.000200018 | LDHAL6A | 0.33639362 | 0.004961774 | NUP98 | 0.1486624 | 0.047175193 |
| MGAT1 | 0.93400955 | 2.85821E−07 | LOC389906 | 1.3842962 | 0.000201059 | WWP1 | 0.1600134 | 0.004997602 | KLHL35 | 1.5804954 | 0.047175193 |
| MRPS35 | 0.95884794 | 2.85821E−07 | FLG2 | 0.0176926 | 0.000201343 | FAM216A | 0.5618618 | 0.004999198 | SLC40A1 | 1.3728577 | 0.047175193 |
| RBM42 | 0.7942882 | 2.85964E−07 | FABP7 | 1.476073 | 0.000201585 | CCDC94 | 0.62921405 | 0.00500252 | LOXL1-AS1 | 0.3590313 | 0.047175193 |
| FAM175A | 1.0746298 | 2.87808E−07 | VPS4A | 0.63203055 | 0.000202391 | COMMD1 | 0.5186112 | 0.005011026 | SPATA2L | 0.76822996 | 0.047225853 |
| NIT2 | 0.97045183 | 2.90481E−07 | NREP | 1.028908 | 0.000203244 | PPP6R2 | 0.21504517 | 0.005017815 | SLC25A20 | 0.51826656 | 0.047289661 |
| GON4L | 0.5510276 | 2.93178E−07 | COMMD4 | 0.8139307 | 0.000203443 | DCP2 | 0.090362094 | 0.005018552 | SHANK3 | 0.9045629 | 0.047379227 |
| ESF1 | 0.5030365 | 2.95567E−07 | KATNBL1 | 0.5037821 | 0.000203556 | PDCD2 | 0.52602524 | 0.005020155 | CYP2U1 | 0.6442188 | 0.047391797 |
| BYSL | 1.0048703 | 3.02967E−07 | SORBS1 | 0.4091862 | 0.0002038 | YKT6 | 0.30674595 | 0.005034376 | FAM193A | 0.09396501 | 0.047430078 |
| SEC23B | 0.8634742 | 3.07503E−07 | LETM1 | 0.4013392 | 0.000203868 | ZNF445 | 0.17362761 | 0.005034376 | ERCC6L2 | 0.22975835 | 0.04772576 |
| C22orf28 | 1.0761061 | 3.0922E−07 | IGSF11 | 0.501302 | 0.000204464 | ABL2 | 0.293569 | 0.005035984 | URGCP | 0.24910337 | 0.047783682 |
| AASDHPPT | 0.77787936 | 3.09986E−07 | RNF216P1 | 0.5987507 | 0.000204489 | TAF7 | 0.53163797 | 0.005049761 | PSIP1 | 0.040134102 | 0.047822237 |
| MFN2 | 0.62292784 | 3.11347E−07 | TOX4 | 0.40233713 | 0.000205199 | FAH | 0.3752858 | 0.005085296 | ZRANB3 | 0.5270805 | 0.04788646 |
| AK2 | 0.85981655 | 3.11347E−07 | FADS2 | 0.81353366 | 0.000205602 | PTAR1 | 0.14190015 | 0.005101702 | ATXN7L3B | 0.36671305 | 0.047964022 |
| OCA2 | 1.712914 | 3.11592E−07 | GPAM | 0.8274373 | 0.000206272 | CCDC34 | 0.5239037 | 0.005101702 | ZFP1 | 0.6573801 | 0.047983218 |
| AKAP2 | 0.6919973 | 3.14125E−07 | LGALSL | 1.1748765 | 0.000206386 | RASA4 | 0.05447199 | 0.005110363 | RBBP8 | 0.16784632 | 0.048008918 |
| POLR2A | 0.6715966 | 3.1508E−07 | BTBD3 | 0.49213493 | 0.000206792 | EXOSC6 | 0.16394824 | 0.00513227 | UBXN1 | 0.5902076 | 0.048015134 |
| NCOR1 | 0.5823783 | 3.17285E−07 | ELL | 0.7824993 | 0.000206792 | CENPO | 0.81073225 | 0.005146289 | HYAL1 | 1.5036244 | 0.048118907 |
| GAR1 | 0.822547 | 3.18518E−07 | TMEM19 | 0.25291106 | 0.000208383 | USP10 | 0.4220182 | 0.005150587 | SH3YL1 | 0.33488348 | 0.048138158 |
| STK38L | 1.2351058 | 3.20565E−07 | YOD1 | 0.8393199 | 0.000208383 | PICALM | 0.2796078 | 0.005155776 | ANKHD1-EIF4EBP3 | 0.25262868 | 0.04815062 |
| PIP5K1A | 0.7497818 | 3.22589E−07 | TNFRSF19 | 0.5121931 | 0.000208383 | C1orf112 | 0.7340562 | 0.00515729 | HECTD1 | 0.10044944 | 0.048228271 |
| MEMO1 | 1.0022343 | 3.26583E−07 | DYX1C1 | 0.108679764 | 0.000208991 | NUDT16L1 | 0.5629213 | 0.00515729 | GAS2 | 1.8661059 | 0.048266857 |
| MRPS28 | 0.94790375 | 3.32301E−07 | METTL17 | 0.68307924 | 0.000209017 | TM9SF3 | 0.001897405 | 0.005161597 | RAB1B | 0.36382943 | 0.048305193 |
| MED20 | 0.9564214 | 3.34604E−07 | IP6K3 | 2.0534387 | 0.000209312 | INE1 | 0.16513184 | 0.00517302 | EXO1 | 0.6485916 | 0.048382507 |
| LSAMP | 0.68771535 | 3.46294E−07 | CCDC163P | 0.16237497 | 0.000211368 | EHD4 | 0.4122147 | 0.005205006 | FAM133DP | 0.1669097 | 0.048414663 |
| ENC1 | 1.2715873 | 3.52544E−07 | HNRNPH1 | 0.52502203 | 0.000211575 | BRAP | 0.5092528 | 0.005205638 | TACO1 | 0.46076265 | 0.048420932 |
| RTTN | 0.9311456 | 3.53712E−07 | RNASEH2A | 0.8783855 | 0.000211647 | CHUK | 1.2165911 | 0.005216254 | EXOC5 | 0.2278968 | 0.048466483 |
| IFT88 | 0.80303365 | 3.55381E−07 | AP5S1 | 0.43289477 | 0.0002119 | ONECUT1 | 1.7981732 | 0.005227788 | BCL7C | 0.7675306 | 0.048518625 |
| NDUFAF4 | 0.7149457 | 3.5586E−07 | TMX1 | 0.48164004 | 0.000212817 | ISCA1 | 0.40699977 | 0.005235746 | RAP2A | 0.17779824 | 0.048524906 |
| DCTN1 | 0.8005065 | 3.62378E−07 | HMGN3 | 0.80821013 | 0.000212817 | SRP68 | 0.49176645 | 0.005258014 | TPGS2 | 0.3404038 | 0.048649332 |
| MRPS34 | 1.1055993 | 3.63273E−07 | TMEM246 | 1.6069937 | 0.000213117 | AHCTF1 | 0.23048635 | 0.005259425 | DGKZ | 0.048750903 | 0.048681918 |
| MCMBP | 0.6159064 | 3.73458E−07 | ANAPC15 | 0.7780796 | 0.00021383 | BACE1 | 0.41959965 | 0.005259425 | DESI1 | 0.16563545 | 0.048727681 |
| TIMELESS | 0.90771025 | 3.76796E−07 | ZNF16 | 1.1834406 | 0.000214453 | SLC35G1 | 1.1112564 | 0.005259425 | NET1 | 0.36321193 | 0.048780067 |
| DLG5 | 0.9717419 | 3.77514E−07 | ALG9 | 0.59747624 | 0.00021637 | LYAR | 0.3473639 | 0.005265619 | ZNF814 | 0.079131715 | 0.048819319 |
| CCDC137 | 0.8231539 | 3.80354E−07 | RECK | 0.7096567 | 0.000217213 | EXOC8 | 0.40681228 | 0.005288136 | DFFA | 0.23352411 | 0.048838818 |
| SYNGR1 | 1.0124968 | 3.83926E−07 | ZNF124 | 0.4206097 | 0.000217213 | LAGE3 | 0.27215835 | 0.005297087 | TRAM1L1 | 1.9536455 | 0.048857775 |
| RPL36A-HNRNPH2 | 0.9358851 | 3.83926E−07 | RPUSD2 | 0.91040635 | 0.000218219 | GTPBP10 | 0.03898525 | 0.005300593 | LPCAT4 | 0.30592433 | 0.048857775 |
| SLC4A1AP | 0.75370055 | 3.94631E−07 | AAMP | 0.86492 | 0.000218526 | PTCSC3 | 0.101967596 | 0.00530228 | POMC | 0.56313604 | 0.048910281 |
| MCM5 | 1.1096343 | 3.95935E−07 | NAA25 | 0.56683916 | 0.000219236 | SAP30BP | 0.4392833 | 0.005303058 | ZNF675 | 0.26987472 | 0.049028943 |
| NUDT15 | 0.5538017 | 3.97576E−07 | DGUOK | 0.76014096 | 0.000219236 | SLC25A23 | 0.66190034 | 0.005307478 | COMMD3-BMI1 | 0.20863147 | 0.049035283 |
| MRPL16 | 0.93797904 | 3.99316E−07 | OR11A1 | 0.18701327 | 0.00022025 | PUM1 | 0.29872945 | 0.00531646 | SPDL1 | 0.50065106 | 0.049048241 |
| DDX28 | 1.3531691 | 3.99316E−07 | DENR | 0.4784627 | 0.000220797 | CASP3 | 0.37415412 | 0.005359223 | HUWE1 | 0.21957485 | 0.049246264 |
| B3GALNT2 | 0.4445938 | 4.00077E−07 | MSH2 | 0.5691742 | 0.000220824 | BRIX1 | 0.59459627 | 0.005379205 | NECAP1 | 0.30342862 | 0.049285841 |
| RTFDC1 | 1.0634643 | 4.01399E−07 | FAIM3 | 1.0251266 | 0.00022213 | UPRT | 0.89967823 | 0.005381838 | SH3KBP1 | 0.2767119 | 0.049371732 |
| NPAT | 0.72800404 | 4.05884E−07 | PRKRA | 0.6472222 | 0.000222205 | RPL27 | 0.31991538 | 0.005390803 | ENPP2 | 0.41863713 | 0.049384767 |
| TBCB | 0.9802087 | 4.07679E−07 | CCNK | 0.49259418 | 0.000225341 | A1BG-AS1 | 0.783914 | 0.005414469 | SLC25A39 | 0.5347427 | 0.049397805 |
| FGF13 | 1.7801492 | 4.11658E−07 | DYNC1LI1 | 0.5371398 | 0.000227351 | FAM49B | 0.42857572 | 0.005425475 | EIF4H | 0.66651946 | 0.049583965 |
| LONP1 | 0.67814773 | 4.11867E−07 | CEMP1 | 0.6179257 | 0.000227602 | TRIM59 | 0.19911133 | 0.005457818 | PCK2 | 0.42594895 | 0.049657206 |
| RNLS | 2.767921 | 4.12652E−07 | SLC25A51 | 0.40332952 | 0.00022763 | PSMD1 | 0.53284127 | 0.005465163 | POMP | 0.5144432 | 0.049703484 |
| CDC123 | 1.0330906 | 4.27974E−07 | SGOL1 | 0.29933882 | 0.000228437 | DCUN1D4 | 0.279564 | 0.005465963 | DTNBP1 | 0.43639317 | 0.049783579 |
| ZC3H3 | 0.81602126 | 4.32867E−07 | TCIRG1 | 0.6187273 | 0.000228514 | DNAJC2 | 0.38405234 | 0.005482558 | MED14 | 0.15237562 | 0.049809564 |
| ZFAND1 | 0.8346597 | 4.3369E−07 | PNMA1 | 0.73767203 | 0.000229863 | MSI1 | 0.6722083 | 0.005484299 | SLC52A2 | 0.3807664 | 0.049809564 |
| ANAPC7 | 0.8394057 | 4.34757E−07 | DTL | 1.2278926 | 0.000230215 | TSEN2 | 0.21156617 | 0.005492616 | UBE2F-SCLY | 1.2507169 | 0.049815994 |
| NBPF3 | 0.38099548 | 4.37629E−07 | SCLY | 0.7982044 | 0.000230215 | BRIP1 | 0.037665073 | 0.005507536 | RPTOR | 0.25186542 | 0.049949715 |
| CHKA | 0.8413042 | 4.37974E−07 | ZNF548 | 0.458791 | 0.000230588 | LYSMD2 | 1.2409658 | 0.005513055 | UPF1 | 0.15631709 | 0.049969331 |
| LINS | 0.872308 | 4.39663E−07 | RBP1 | 0.6401607 | 0.000230666 | NUDT22 | 0.5284902 | 0.005514806 | PRKX | 0.14496046 | 0.049969331 |
| RPL15 | 0.34823257 | 4.43554E−07 | WDR83 | 0.91254395 | 0.000230892 | SNN | 0.75694 | 0.005517501 | MYL6 | 0.05566979 | 0.045684248 |
| WDR4 | 0.8532374 | 4.48246E−07 | NRG3 | 1.3236904 | 0.000231858 | ATG10 | 0.13479058 | 0.005520198 | TMEM59 | 0.018837657 | 0.005836373 |
| UGCG | 0.925756 | 4.5085E−07 | SLC31A1 | 0.46536243 | 0.000233674 | NAV1 | 0.16272695 | 0.005533294 | MTRNR2L2 | 0.2547141 | 0.005426271 |
| PBRM1 | 0.5124916 | 4.51332E−07 | C19orf24 | 0.7427914 | 0.000234401 | RFPL3S | 2.8062913 | 0.005539784 | MTRNR2L8 | 0.13951954 | 0.000200301 |
| PIGM | 0.7690971 | 4.5219E−07 | PAPOLG | 0.22623034 | 0.000234481 | NUP214 | 0.25156242 | 0.005554821 | MTRNR2L10 | 0.014822896 | 0.000183187 |
| NPM1 | 0.48306182 | 4.55197E−07 | TPT1-AS1 | 0.29995823 | 0.00023486 | WBSCR22 | 0.67457914 | 0.005557535 | MTRNR2L1 | 0.00826583 | 3.12849E−05 |
| SOCS2 | 1.350618 | 4.55683E−07 | ZNF839 | 0.008801705 | 0.000237182 | SREBF2 | 0.20495689 | 0.005572616 | | | |
| ATP5L | 0.7998482 | 4.60371E−07 | PAFAH1B1 | 0.44363567 | 0.000237565 | DIDO1 | 0.32730576 | 0.005589507 | | | |

### Example 2

Acral melanoma (AM) is a deadly adult skin cancer that is the predominant cutaneous melanoma (CM) subtype in populations of African descent with the lowest disease specific survival rates in minority ethnic and racial populations (Hispanic, Asian, and African descent). The disease specific death rate from AM is more than twice as high than that of CM in general. When adjusted for stage, AM still has worse outcomes when compared to other forms of CM that predominately affect the white population. This has been postulated to be due to sociodemographic factors or delays in time to definitive treatment following initial diagnosis, though poorer response to adjuvant therapy would also suggest a biologic etiology for this discrepancy.

Recent research has discovered that AMs arise from a cell of origin distinct from other CMs. This observation suggests that the historic practice of either omitting AM or grouping AM with other CMs for the purpose of biomarker and therapeutic development, as opposed to investigating AM as a distinct and unrelated cancer, is one cause of the response and survival disparities experienced by AM patients.

Immune checkpoint inhibitor (ICI) therapies have garnered unprecedented response rates in patients with advanced stage CM. However, while efficacious in most subtypes of melanoma, single agent las demonstrate lower objective response rates (16-40%) and are less likely to achieve complete responses in AM patients. There are ongoing efforts to improve the therapeutic landscape for patients with advanced AM, with several eminent therapies on the horizon. However, the undeniable effectiveness of las at the population level has made ICI therapy the standard of care for all CM, even when less efficacious for AM, specifically. Additionally, 40% of non-responding patients experience treatment-related adverse side effects despite experiencing no benefit from the treatment. Identifying AM patients who are more likely to respond to ICI would provide critical information for clinicians and permit precision implementation of the next generation front-line therapies awaiting FDA approval.

It is hypothesized that transcriptomic deconvolution will permit a priori stratification of AM by likelihood of ICI response. The feasibility studies support this hypothesis. The objectives are (i) to validate a CLIA compatible biomarker for predicting ICI response; and (iii) to test the biomarker on an independent validation set.

An additional objective is to stratify acral melanoma (AM) patients by likelihood of response to immune checkpoint inhibitors (ICI). First, a biomarker assay is developed and analytically validated using NanoString—a cost-effective approach that is amenable to clinical specimens, simple interpretation, and CLIA certification. Second, the biomarker assay will undergo additional clinical validation using an independent dataset.

Deconvolution-Based Classifier that Stratifies AM by ICI Response

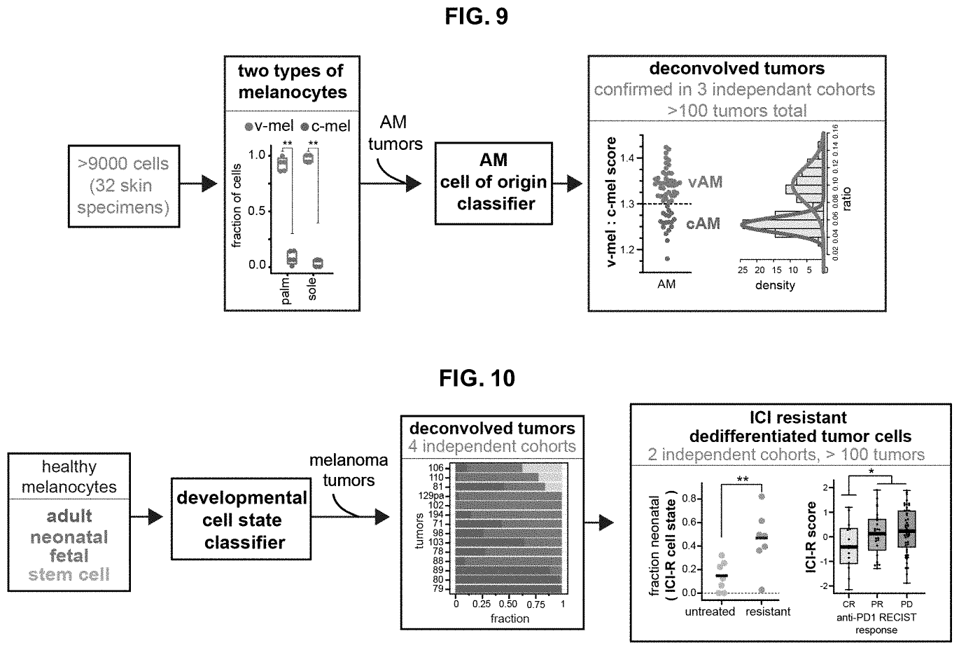

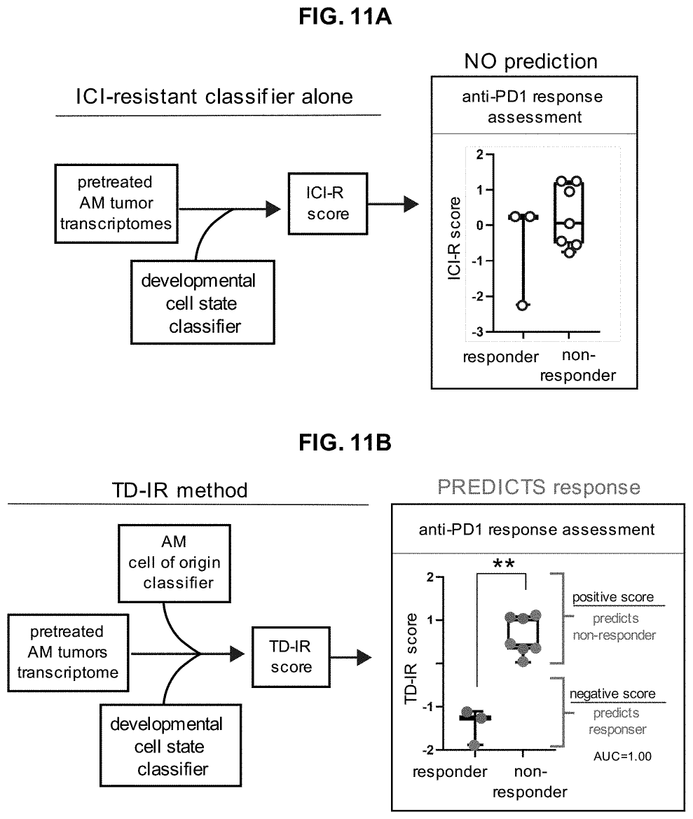

Using single cell RNA (scRNA) sequencing, over 9,000 cells were analyzed from 32 healthy skin specimens representing a diverse ethnic and racial cohort and discovered two distinct types of melanocytes (v-mels and c-mels), either of which can give rise to AM. A classifier was trained to deconvolute bulk transcriptomic data which successfully stratified AMs as v-mel derived (vAM) or c-mel derived (cAM) in three independent cohorts (FIG. 9). Utilizing scRNA-seq data of developing melanocytes and metastasized melanomas a classifier was similarly trained to deconvolute cutaneous melanoma (CM) bulk transcriptomic data and identify those tumors predominantly composed of dedifferentiated cells resistant to ICI (ICI-R). For non-AM CM, this classifier stratified tumors based upon ICI response (FIG. 10). Importantly, for AM, a two-step deconvolution was required—first stratifying by cell of origin and then identifying predominant dedifferentiation state (FIG. 11). This approach resulted in perfect classification of the largest and only publicly available cohort of matched AM transcriptomes with ICI (anti-PD1) response. That this cohort comprises only ten patients is indicative of the degree to which the AM patient population is under-represented and under-studied. Seeking to adapt the classifier to NanoString technology, the classifier was refined to 200 highly expressed genes that comprise minimal signatures for identifying the cAM, vAM, and ICI-R states (Table 5). These signatures provide sufficient information for deconvolution and high accuracy classification. Conceptionally, the cAM and vAM signatures permit identification of cell of origin and the ICI-R signature then allows estimation of the percentage of cells that are resistant to ICI. This established analytic pipeline that converts the expression of 200 genes to a single value as the transcriptomic deconvolution-based predictor of ICI-R (TD-IR). This estimate, converted to a single positive or negative “TD-IR score” (FIG. 11), is the ultimate and only readout of the approach.

TABLE 5
TD-IR NanoString Custom Panel

| | | | | | | | |
|---|---|---|---|---|---|---|---|
| SERPINF1 | GPM6B | RPS17L | GAS5 | CREBBP | MACF1 | ZNF263 | PEX10 |
| PABPC1 | FOXRED2 | RPS17 | RPL13AP5 | MYCBP2 | VPS13C | GGCT | NR2F6 |
| DCT | SOAT1 | MARCKS | SNAI2 | HIVEP2 | SYNE1 | ZFAT | EXTL2 |
| TIMM50 | RPS16 | RPS4X | FAM174B | NTRK2 | NOTCH2 | ARMC1 | ZMYND19 |
| CA14 | PKNOX2 | ESRP1 | RASSF3 | SNX29 | DYSF | DUS4L | CDK12 |
| SCD | RPL18 | NF2 | PTP4A3 | VPS13D | NBEAL1 | ZNHIT1 | ZNF146 |
| RPS19 | EIF4A1 | CNRIP1 | RPS7 | KMT2C | POLR3A | MRPL32 | SRR |
| RPL29 | RPS27 | OXA1L | EIF2S3 | WNK1 | NBAS | SLC25A36 | FNTA |
| BCAN | NOP56 | NPL | ABR | KDM5A | RANBP2 | SRM | COMMD5 |
| TRPM1 | CHP1 | CD68 | PPA1 | HUWE1 | KAT6B | TSNAX | ZNF24 |
| KIF17 | TNFRSF14 | SAE1 | CS | MYH9 | TRAPPC10 | CBX3 | MOB1B |
| RPS24 | UQCRFS1 | MID1 | EGFL8 | EP300 | REL | PLA2G12A | TOMM20 |
| RPL28 | KLHDC8B | ZNF749 | GPI | CEP128 | ATXN7 | SRSF6 | ARL10 |
| ADRBK2 | RPS9 | PPP2R1A | RPL8 | TRIP11 | ANKRD11 | MAD2L1BP | SHARPIN |
| KCNAB2 | SCIN | RPS8 | C1QBP | CHD8 | STAT3 | MED1 | TIGD5 |
| PFN1 | RPL4 | GSTO1 | TUBB4A | HERC1 | JMJD1C | NRSN2 | C8orf33 |
| TMC6 | ASAP1 | SAMM50 | PLTP | SETX | FLNA | LSM7 | COA5 |
| NENF | RAB38 | EIF3K | SS18L1 | EGFR | DYNC1H1 | TMEM128 | TSPYL4 |
| ACP5 | AHCY | SNHG6 | SORD | ASCC3 | SPTAN1 | TBRG4 | ZNF517 |
| RPLP0 | CCT3 | FAM178B | ILF2 | BIRC6 | ITSN2 | TSTD2 | ZNF121 |
| RPS6 | TP53 | RPL6 | EIF3L | ALMS1 | ZNF407 | MRPL15 | SPIN3 |
| EIF4EBP2 | IDH2 | MAD1L1 | MLANA | ASH1L | PLEKHM1 | SAT2 | TRIM27 |
| RPL13A | SLC25A5 | IMPDH2 | RPS11 | BAZ2B | ERCC6 | BOD1 | TRIM13 |
| PRAME | RPS5 | BZW2 | ADSL | NCOA3 | PARG | PURB | TMEM231 |
| MOB3B | GLOD4 | GALE | PRDX3 | MRPS21 | NDUFA7 | NDUFA3 | CYC1 |

### NanoString Platform

Assays performed on formalin fixed paraffin embedded (FFPE) tissue and that utilize a small number of sections are the most easily adopted clinical tests with regards to tissue requirements. NanoString provides a hybridization-based technology that permits targeted transcript counting, without amplification, on the poor-quality RNA extracted from FFPE blocks. A custom NanoString nCounter RNA expression panel of 200 highly-expressed transcripts was designed. The expression values inform the TD-IR classifier, resulting in a single score representing the presence of ICI-R resistant cells (henceforth referred to as the biomarker). Samples consist of 3-5 macrodissected 4 μm formalin-fixed paraffin-embedded (FFPE) sections per specimen. The NanoString pipeline, inclusive of sectioning, macrodissection, RNA extraction, and transcript counting, has produced highly reproducible gene expression patterns. See e.g., Leal et al., Neuropathology 38(5):475-483 (2018); Veldman-Jones et al., Cancer Res. 75(13): 2587-2593 (2015), each of which are incorporated by reference for the teachings thereof.

### RNAScope Platform

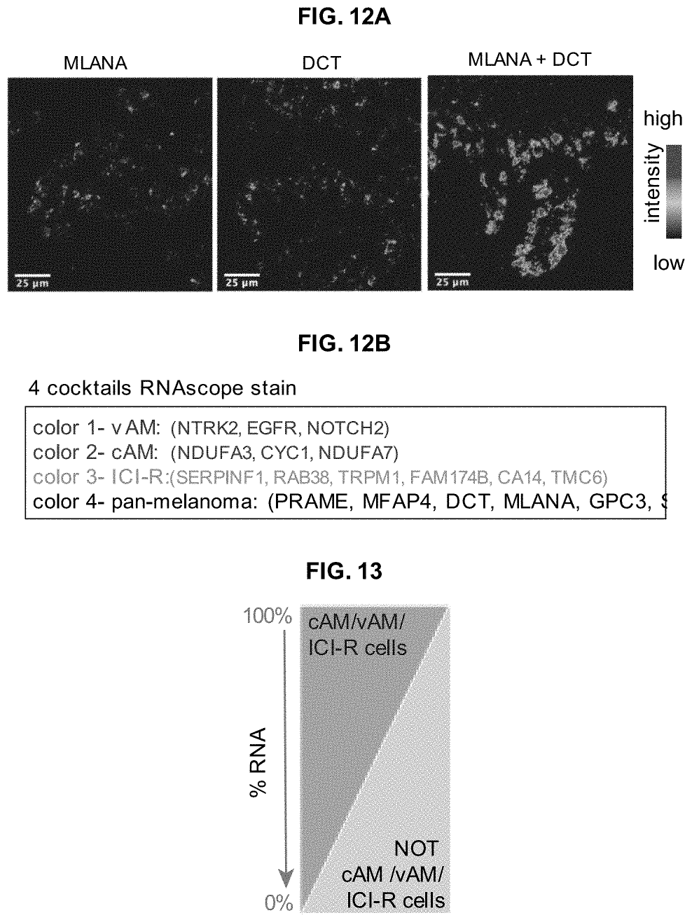

The TD-IR classifier is a bioinformatic method intended to deconvolute the noise introduced into bulk transcriptomic data by diverse cells of origin and tumor heterogeneity—ultimately successful, because it accurately infers the percentage of ICI-R cells. As both a complementary method of validation for the approach and to directly confirm this mechanism, a single molecule RNA in-situ hybridization assay called RNAScope is used. Since RNAScope retains spatial and single cell resolution, thereby substantially reducing non-specific transcriptomic noise from other cell-types/states within the tumor, only 18 gene probes are expected to be needed to determine the fraction of ICI-R cells. Previous work shows the combination of probes for a molecular signature into a single uni-colored “cocktail” permits a simplified and robust method for cell-type/state detection using total fluorescence intensity (FIG. 12A). Probes are pooled to generate a four-cocktail stain (FIG. 12B) for a 4 μm FFPE section compatible with the Leica BOND Fully Automated ISH Staining System. The percent of ICI-R cells are quantified relative to the total number of melanoma cells.

### Statistical Methods

There are separate biomarker development and validation data sets. Once established, the biomarker is analyzed using the validation data set to avoid overfitting and bias that can result from using the same data to both develop and validate the assay.

Receiver operator characteristic (ROC) curves and bootstrap confidence intervals are calculated on the development and validation sets using the R package pROC. An optimal binary classifier (Yes/No response to ICI) is constructed using the cases in the development set and evaluated on the validation set. For this application of TD-IR, the goal is to confidently identify those AM patients who will not benefit from ICI, but it is also critical not to erroneously identify ICI responsive patients as ICI-R. Thus, the threshold is tuned to minimize the false negative rate and tolerate a modest number of false positive estimates by the model. For this reason, false negatives are weighted twice as heavily as false positive in construction of the optimal binary classifier.

### Sample Size Justification

A variety of ICI-R predictive biomarkers based upon molecular profiling are currently in development, including measuring PD-L1 expression, mismatch repair deficiency, tumor mutation burden, tumor infiltrating lymphocyte load, and gene expression signatures. The best reported test characteristics achieve an area under the ROC curve (AUC) of 0.68-0.78 but are discouraged for use in clinical decision making by 2021 NCCN guidelines. Based upon these observations and modeling of clinical utility of melanoma biomarkers, the AUC must be at least 0.85 for the biomarker to be clinically useful. The achievable sample size for assay development is N1=50 and the sample size for assay validation is N2=200. The biomarker are adequately validated if the estimated AUC is 0.85 or higher, and the lower bound of a 95% one-sided confidence interval for the AUC is at least 0.80. The feasibility data (FIG. 11) completely separates cases from controls (AUC=100%) so the true AUC of the biomarker is expected to be 90% or larger. Approximately 30% of AM patients respond to ICI (based upon reported 16-40% and observed rate in feasibility cohort) to estimate proportion in the sample size justification. To evaluate the performance with N2=200 samples in the validation set is simulated normally distributed data with various values of AUC. Table 6 presents the probability of adequately validating the biomarker for various values of the AUC. There is greater than 80% probability of validation (power) if the true AUC=0.88. There is 5.0% probability of validation (Type I error) if the true AUC=0.80.

TABLE 6
Validation Probability for Various AUC Values

| True AUC | Probability the Biomarker is Validated* |
|---|---|
| 75% | 0.1% |
| 80% | 5.5% |
| 85% | 47.1% |
| 86% | 66.7% |
| 87% | 76.0% |
| 88% | 87.9% |
| 89% | 94.1% |
| 90% | 96.3% |
| 95% | >99% |

*The biomarker is considered adequately validated if the estimated AUC is at least 85% and the lower bound of a one-sided 95% confidence interval is at least 80%.

### Conversion and Analytical Validation of NanoString Assay

An assay development cohort (N1) for 50 AM specimens, inclusive of ˜30% ICI responsive patients, with sufficient archived material to obtain 3-5 4 μm FFPE sections is assembled. RNA is extracted from macrodissected sections and subjected to NanoString assessment of the 200 gene panel to inform the TD-IR classifier. The ultimate read-out of the classifier (the biomarker) is a single score that infers percent of ICI-R tumor cells. AUC is calculated and a threshold value for the TD-IR score is determined.

The assay characteristics of the NanoString panel are determined using engineered cell lines with known gene expression levels. To determine the specificity of each probe, lines that are uniformly either cAM or vAM and either ICI-R or not are used. The top 15 genes shared in these signatures (cAM, vAM, ICI-R) are overexpressed in a non-expressing line (lentivirus) or knocked out in an expressing line (CRISPR) using established methods. Each pair of lines are fixed in formalin, embedded in paraffin (FFPE) and assessed via NanoString and RNAScope. If individual discrepancies with probe specificity are observed, new probes can be designed.

To determine the sensitivity of NanoString probes, limited dilution series of uniform cAM, vAM or ICI-R cells are mixed with non-cAM/vAM/ICI-R melanocytes and FFPE processed (FIG. 13). TD-IR is assessed in quadruplicate on different days, providing the full range of relative expression of each signature.

To assess precision (reproducibility), technical variability is assessed. The same sample is run 3 times in the same assay (intra-assay variability). In addition, the sample is analyzed on three different days with three different operators (inter-assay variability). Precision is considered sufficient if the coefficient of variance is <11% for all sources of technical variability.

To assess limit of detection, FFPE-derived RNA from benign tissue types where melanoma frequently spreads (lymph nodes, liver, brain, lung, bone, intestine, kidney, adrenal gland, healthy skin, muscle, and fat) are generated. TD-IR is measured for each pure sample and an equal-molar mix of each is used to create a limited dilution series of pure ICI-R cell RNA. The lowest dilution that provides TD-IR signal greater than 2 standard deviations from the full negative cohort is considered the LOD, will define a positive classification and will inform the minimal amount (percent) of tumor cells to detect true signal over the background noise from non-tumor tissue. Total RNA from the lowest detectable dilution will then undergo a second limiting dilution series in water to determine the minimal amount of total RNA required for the assay.

As a complementary validation, 20 specimens of N1 spanning both cAM and vAM and representing the full working range of TD-IR (as assessed above) will undergo RNAScope analysis to directly assess the concordance between bioinformatically inferred ICI-R content (TD-IR) and actual ICI-R content.

### Clinical Validation of TD-IR Assay

In parallel, the larger N2 (200 specimens) cohort are assembled. The cohort is used for a retrospective study to determine if TD-IR reliably stratifies AM tumors into distinct responder vs non-responder groups in an independent cohort. The performance characteristics of the assay will define the potential utility for prospective clinical trials. Even if TD-IR does not perform sufficiently for trial incorporation, the assembled cohorts containing outcome, transcript data, and banked RNA are essential tools for investigating other candidate biomarkers aimed at addressing the disparities associated with the underrepresented and understudied AM population.

What is claimed:

1. A method of stratifying and evaluating melanoma treatment response in a subject using single cell RNA sequencing (scRNA-seq) and a two-step deconvolution analysis, the method comprising:
(a) obtaining a melanoma tumor sample from a subject;
(b) performing scRNA-seq of the melanoma tumor sample and obtaining scRNA-seq sequence data;
(c) on a processor, deconvoluting the scRNA-seq sequence data using a first gene signature to stratify the melanoma tumor sample into a specific melanoma cell subtype; and
(d) deconvoluting the scRNA-seq sequence data using a second gene signature to calculate an estimate of the total number of cells in the melanoma tumor sample that express the second gene signature;
wherein when the calculated estimate of total melanoma tumor expression of the second gene signature reaches a critical threshold value, the melanoma tumor will not respond to immune checkpoint inhibition (ICI) treatment.

2. The method of claim 1, wherein the melanoma is acral melanoma (AM).

3. The method of claim 1, further comprising:
when the calculated total melanoma tumor expression of the second gene signature is below the critical threshold value, an effective amount of an ICI treatment is administered to the subject; or
when the calculated total melanoma tumor expression of the second gene signature is above the critical threshold value, an effective amount of an alternative non-ICI therapy is administered to the subject.

4. The method of claim 1, further comprising:
calculating a transcriptomic deconvolution-based predictor of ICI resistance (TD-IR) score value;
wherein when the calculated TD-IR score value is positive, the melanoma tumor will not respond to ICI treatment; or
wherein when the calculated TD-IR score value is negative, the melanoma tumor will respond to ICI treatment.

5. The method of claim 3, wherein the ICI treatment comprises:
a PD-1 inhibitor selected from pembrolizumab, nivolumab, cemiplimab, or combinations thereof;
a PD-L1 inhibitor selected from atezolizumab, avelumab, durvalumab, or combinations thereof;
a LAG-3 inhibitor selected from relatlimab, relatlimab-RMBW, or combinations thereof; or
combinations thereof.

6. The method of claim 3, wherein the alternative non-ICI therapy comprises:
a PARP inhibitor selected from olaparib, niraparib, rucaparib, talazoparib, or combinations thereof;
a BRAF inhibitor selected from dabrafenib, encorafenib, vemurafenib, or combinations thereof;
a MEK inhibitor selected from trametinib, cobimetinib, binimetinib, or combinations thereof;
a KIT inhibitor selected from dasatinib, imatinib, nilotinib, or combinations thereof;
a tumor-agnostic therapy selected from larotrectinib, entrectinib, or combinations thereof;
a CTLA-4 inhibitor selected from ipilimumab;
aldesleukin (Interleukin-2; IL-2), Interferon alfa-2b, pegylated Interferon alfa-2b, or combinations thereof;
a chemotherapeutic agent selected from dacarbazine, temozolomide, cisplatin, carboplatin, fotemustine, lomustine, docetaxel, paclitaxel, vinblastine, or combinations thereof;
surgical excision; or
combinations thereof.

7. The method of claim 1, wherein the specific melanoma cell subtype comprises volar-like (v-mel) or non-volar cutaneous-like (c-mel) melanocyte-derived melanoma.

8. The method of claim 1, wherein the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18.

9. The method of claim 8, wherein when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma.

10. The method of claim 8, wherein when the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma.

11. The method of claim 1, wherein the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.

12. A method of stratifying and evaluating melanoma treatment response in a subject using single cell RNA sequencing (scRNA-seq) and a two-step deconvolution analysis, the method comprising:
(a) obtaining a melanoma tumor sample from a subject;
(b) performing scRNA-seq of the melanoma tumor sample and obtaining scRNA-seq sequence data;
(c) on a processor, deconvoluting the scRNA-seq sequence data using a first gene signature to stratify the melanoma tumor into a specific melanoma cell subtype;
(d) deconvoluting the scRNA-seq sequence data using a second gene signature to calculate an estimate of the total number of cells in the melanoma tumor sample that express the second gene signature; and
(e) calculating a transcriptomic deconvolution-based predictor of ICI resistance (TD-IR) score value;
wherein when the calculated estimate of total melanoma tumor expression of the second gene signature reaches a critical threshold value, the melanoma tumor will not respond to immune checkpoint inhibition (ICI) treatment;
when the calculated TD-IR score value is positive, the melanoma tumor will not respond to ICI treatment; or
when the calculated TD-IR score value is negative, the melanoma tumor will respond to ICI treatment.

13. The method of claim 12, wherein the melanoma is acral melanoma (AM).

14. The method of claim 12, further comprising:
when it is determined that the melanoma tumor will respond to ICI treatment, an effective amount of an ICI treatment is administered to the subject; or
when it is determined that the melanoma tumor will not respond to ICI treatment, an effective amount of an alternative non-ICI therapy is administered to the subject.

15. The method of claim 14, wherein the ICI treatment comprises:
a PD-1 inhibitor selected from pembrolizumab, nivolumab, cemiplimab, or combinations thereof;
a PD-L1 inhibitor selected from atezolizumab, avelumab, durvalumab, or combinations thereof;
a LAG-3 inhibitor selected from relatlimab, relatlimab-RMBW, or combinations thereof; or
combinations thereof.

16. The method of claim 14, wherein the alternative non-ICI therapy comprises:
a PARP inhibitor selected from olaparib, niraparib, rucaparib, talazoparib, or combinations thereof;
a BRAF inhibitor selected from dabrafenib, encorafenib, vemurafenib, or combinations thereof;
a MEK inhibitor selected from trametinib, cobimetinib, binimetinib, or combinations thereof;
a KIT inhibitor selected from dasatinib, imatinib, nilotinib, or combinations thereof;
a tumor-agnostic therapy selected from larotrectinib, entrectinib, or combinations thereof;
a CTLA-4 inhibitor selected from ipilimumab;
aldesleukin (Interleukin-2; IL-2), Interferon alfa-2b, pegylated Interferon alfa-2b, or combinations thereof;
a chemotherapeutic agent selected from dacarbazine, temozolomide, cisplatin, carboplatin, fotemustine, lomustine, docetaxel, paclitaxel, vinblastine, or combinations thereof;
surgical excision; or
combinations thereof.

17. The method of claim 12, wherein the specific melanoma cell subtype comprises volar-like (v-mel) or non-volar cutaneous-like (c-mel) melanocyte-derived melanoma.

18. A method of stratifying and evaluating melanoma treatment response in a subject using RNA hybridization, and a two-step deconvolution analysis, the method comprising:
(a) obtaining a melanoma tumor sample from the subject;
(b) performing RNA hybridization of the melanoma tumor sample using a targeted RNA probe panel to obtain targeted transcript expression data;
(c) on a processor, deconvoluting the targeted transcript expression data using a first gene signature from the targeted RNA probe panel to stratify the melanoma into a specific melanoma cell subtype; and
(d) deconvoluting the targeted transcript expression data using a second gene signature from the targeted RNA probe panel to calculate an estimate of the total number of cells in the tumor sample that express the second gene signature;
wherein when the calculated estimate of total tumor expression of the second gene signature reaches a critical threshold value, the tumor will not respond to immune checkpoint inhibition (ICI) treatment.

19. The method of claim 18, wherein the melanoma is acral melanoma (AM).

20. The method of claim 18, wherein the melanoma tumor sample comprises one or more biopsy samples or one or more formalin fixed paraffin embedded (FFPE) tumor tissue samples from the subject.

21. The method of claim 18, wherein the targeted RNA probe panel comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, and CYC1.

22. The method of claim 18, further comprising:
calculating a transcriptomic deconvolution-based predictor of ICI resistance (TD-IR) score value;
wherein when the calculated TD-IR score value is positive, the melanoma tumor will not respond to ICI treatment; or
wherein when the calculated TD-IR score value is negative, the melanoma tumor will respond to ICI treatment.

23. The method of claim 18, further comprising:
when it is determined that the melanoma tumor will respond to ICI treatment, an effective amount of an ICI treatment is administered to the subject; or
when it is determined that the melanoma tumor will not respond to ICI treatment, an effective amount of an alternative non-ICI therapy is administered to the subject.

24. The method of claim 23, wherein the ICI treatment comprises:
a PD-1 inhibitor selected from pembrolizumab, nivolumab, cemiplimab, or combinations thereof;
a PD-L1 inhibitor selected from atezolizumab, avelumab, durvalumab, or combinations thereof;
a LAG-3 inhibitor selected from relatlimab, relatlimab-RMBW, or combinations thereof; or
combinations thereof.

25. The method of claim 23, wherein the alternative non-ICI therapy comprises:
a PARP inhibitor selected from olaparib, niraparib, rucaparib, talazoparib, or combinations thereof;
a BRAF inhibitor selected from dabrafenib, encorafenib, vemurafenib, or combinations thereof;
a MEK inhibitor selected from trametinib, cobimetinib, binimetinib, or combinations thereof;
a KIT inhibitor selected from dasatinib, imatinib, nilotinib, or combinations thereof;
a tumor-agnostic therapy selected from larotrectinib, entrectinib, or combinations thereof;
a CTLA-4 inhibitor selected from ipilimumab;
aldesleukin (Interleukin-2; IL-2), Interferon alfa-2b, pegylated Interferon alfa-2b, or combinations thereof;
a chemotherapeutic agent selected from dacarbazine, temozolomide, cisplatin, carboplatin, fotemustine, lomustine, docetaxel, paclitaxel, vinblastine, or combinations thereof;
surgical excision; or
combinations thereof.

26. A method of stratifying and evaluating melanoma treatment response in a subject using bulk transcriptomic data and a two-step deconvolution analysis, the method comprising:
(a) obtaining one or more melanoma tumor samples from a subject;
(b) performing RNA sequencing of the one or more melanoma tumor samples and obtaining bulk transcriptomic data;
(b) performing transcript counting on the bulk transcriptomic data to obtain transcript expression data;
(c) on a processor, deconvoluting the transcript expression data using a first gene signature to stratify the melanoma into a specific melanoma cell subtype or origin; and
(d) deconvoluting the transcript expression data using a second gene signature to calculate an estimate of the total number of cells in the melanoma tumor sample that express the second gene signature or determine the cell differentiation state;
wherein when the calculated estimate of total expression of the second gene signature reaches a critical threshold value, the melanoma will not respond to immune checkpoint inhibition (ICI) treatment.

27. The method of claim 26, wherein the melanoma is acral melanoma (AM).

28. The method of claim 26, further comprising:
when the calculated total tumor expression of the second gene signature is below the critical threshold value, an effective amount of an ICI treatment is administered to the subject; or
when the calculated total tumor expression of the second gene signature is above the critical threshold value, an effective amount of an alternative non-ICI therapy is administered to the subject.

29. The method of claim 26, further comprising:
calculating a transcriptomic deconvolution-based predictor of ICI resistance (TD-IR) score value;
wherein when the calculated TD-IR score value is positive, the melanoma tumor will not respond to ICI treatment; or
wherein when the calculated TD-IR score value is negative, the melanoma tumor will respond to ICI treatment.

30. The method of claim 28, wherein the ICI treatment comprises:
a PD-1 inhibitor selected from pembrolizumab, nivolumab, cemiplimab, or combinations thereof;
a PD-L1 inhibitor selected from atezolizumab, avelumab, durvalumab, or combinations thereof;
a LAG-3 inhibitor selected from relatlimab, relatlimab-RMBW, or combinations thereof; or
combinations thereof.

31. The method of claim 28, wherein the alternative non-ICI therapy comprises:
a PARP inhibitor selected from olaparib, niraparib, rucaparib, talazoparib, or combinations thereof;
a BRAF inhibitor selected from dabrafenib, encorafenib, vemurafenib, or combinations thereof;
a MEK inhibitor selected from trametinib, cobimetinib, binimetinib, or combinations thereof;
a KIT inhibitor selected from dasatinib, imatinib, nilotinib, or combinations thereof;
a tumor-agnostic therapy selected from larotrectinib, entrectinib, or combinations thereof;
a CTLA-4 inhibitor selected from ipilimumab;
aldesleukin (Interleukin-2; IL-2), Interferon alfa-2b, pegylated Interferon alfa-2b, or combinations thereof;
a chemotherapeutic agent selected from dacarbazine, temozolomide, cisplatin, carboplatin, fotemustine, lomustine, docetaxel, paclitaxel, vinblastine, or combinations thereof;
surgical excision; or
combinations thereof.

32. The method of claim 26, wherein the specific melanoma cell subtype comprises volar-like (v-mel) or non-volar cutaneous-like (c-mel) melanocyte-derived melanoma.

33. The method of claim 26, wherein the first gene signature comprises one or more genes selected from ID3, NTRK2, ID2, LOC101930452, MEG3, LINC00473, RAB3B, IGDCC4, MIA, PDLIM4, AKAP12, SLC45A2, HPGD, MCOLN3, RGL1, SEMA5A, ACP5, APCDD1, LINC00462, or GALNT18.

34. The method of claim 33, wherein when the expression of one or more of ID3, NTRK2, ID2, LOC101930452, MEG3, LINC00473, RAB3B, IGDCC4, MIA, or PDLIM4 is upregulated, the melanoma is stratified as a volar-like (v-mel) melanocyte-derived melanoma.

35. The method of claim 33, wherein when the expression of one or more of AKAP12, SLC45A2, HPGD, MCOLN3, RGL1, SEMA5A, ACP5, APCDD1, LINC00462, or GALNT18 is upregulated, the melanoma is stratified as a non-volar cutaneous-like (c-mel) melanocyte-derived melanoma.

36. The method of claim 26, wherein the second gene signature comprises one or more genes selected from SERPINF1, GPM6B, RPS17L, GAS5, CREBBP, MACF1, ZNF263, PEX10, PABPC1, FOXRED2, RPS17, RPL13AP5, MYCBP2, VPS13C, GGCT, NR2F6, DCT, SOAT1, MARCKS, SNAI2, HIVEP2, SYNE1, ZFAT, EXTL2, TIMM50, RPS16, RPS4X, FAM174B, NTRK2, NOTCH2, ARMC1, ZMYND19, CA14, PKNOX2, ESRP1, RASSF3, SNX29, DYSF, DUS4L, CDK12, SCD, RPL18, NF2, PTP4A3, VPS13D, NBEAL1, ZNHIT1, ZNF146, RPS19, EIF4A1, CNRIP1, RPS7, KMT2C, POLR3A, MRPL32, SRR, RPL29, RPS27, OXA1L, EIF2S3, WNK1, NBAS, SLC25A36, FNTA, BCAN, NOP56, NPL, ABR, KDM5A, RANBP2, SRM, COMMD5, TRPM1, CHP1, CD68, PPA1, HUWE1, KAT6B, TSNAX, ZNF24, KIF17, TNFRSF14, SAE1, CS, MYH9, TRAPPC10, CBX3, MOB1B, RPS24, UQCRFS1, MID1, EGFL8, EP300, REL, PLA2G12A, TOMM20, RPL28, KLHDC8B, ZNF749, GPI, CEP128, ATXN7, SRSF6, ARL10, ADRBK2, RPS9, PPP2R1A, RPL8, TRIP11, ANKRD11, MAD2L1BP, SHARPIN, KCNAB2, SCIN, RPS8, C1QBP, CHD8, STAT3, MED1, TIGD5, PFN1, RPL4, GSTO1, TUBB4A, HERC1, JMJD1C, NRSN2, C8orf33, TMC6, ASAP1, SAMM50, PLTP, SETX, FLNA, LSM7, COA5, NENF, RAB38, EIF3K, SS18L1, EGFR, DYNC1H1, TMEM128, TSPYL4, ACP5, AHOY, SNHG6, SORD, ASCC3, SPTAN1, TBRG4, ZNF517, RPLP0, CCT3, FAM178B, ILF2, BIRC6, ITSN2, TSTD2, ZNF121, RPS6, TP53, RPL6, EIF3L, ALMS1, ZNF407, MRPL15, SPIN3, EIF4EBP2, IDH2, MAD1L1, MLANA, ASH1L, PLEKHM1, SAT2, TRIM27, RPL13A, SLC25A5, IMPDH2, RPS11, BAZ2B, ERCC6, BOD1, TRIM13, PRAME, RPS5, BZW2, ADSL, NCOA3, PARG, PURB, TMEM231, MOB3B, GLOD4, GALE, PRDX3, MRPS21, NDUFA7, NDUFA3, or CYC1.
